(19) United States
(12) Patent Application Publication
(10) Pub. No.: US 2025/0304918 A1
(43) Pub. Date: Oct. 2, 2025

(54) VIRAL PARTICLES RETARGETED TO SKELETAL MUSCLE

(71) Applicant: Regeneron Pharmaceuticals, Inc., Tarrytown, NY (US)

(72) Inventors: Leah Sabin, Goldens Bridge, NY (US); Michael Stec, Tarrytown, NY (US); Andrew Murphy, Croton-on-Hudson, NY (US); Christos Kyratsous, Irvington, NY (US); Sven Moller-Tank, White Plains, NY (US); Poulami Samai, New York, NY (US)

(21) Appl. No.: 18/707,217

(22) PCT Filed: Nov. 4, 2022
(86) PCT No.: PCT/US2022/079339

§ 371 Date: May 3, 2024

Related U.S. Application Data
(60) Provisional application No. 63/275,731, filed on Nov. 4, 2021.
(60) Provisional application No. 63/369,761, filed on Jul. 28, 2022.

(51) Int. Cl. C12N 7/00; A61K 35/76; A61K 47/64; A61P 21/00; C12N 5/077; C12N 5/10

(52) U.S. Cl. CPC C12N 7/00; A61K 35/76; A61K 47/64; A61P 21/00; C12N 5/0658; C12N 5/10; C12N 2510/00; C12N 2750/14123; C12N 2750/14133; C12N 2750/14145

(57) ABSTRACT

Provided herein are compositions and methods for retargeting viral particles. e.g. adeno-associated virus (AAV) particles, to muscle cells using muscle-specific surface proteins. AAV adapted accordingly may be a viable gene therapy platform for the treatment of a skeletal muscle related disorder (e.g., X-linked myotubular myopathy (XLMTM). Duchenne muscular dystrophy (DMD), myotonic dystrophy (DM1), Facioscapulohumeral muscular dystrophy Type 1 (FSHD), congenital muscular dystrophy type 1A (MDC1A), Limb girdle muscular dystrophy, dystroglycanopathy, etc.) in a patient in need thereof.

### TECHNICAL FIELDS

The disclosure herein relates to methods of making and using recombinant viral particles, e.g., recombinant AAV particles, comprising capsid proteins retargeted to a muscle-specific surface protein, e.g., Calcium Voltage-Gaged Auxiliary Subunit Gamma 1 (CACNG1) or Cadherin 15 (CAD15), useful for modification of muscle cells, such as skeletal muscle cells, in vitro or in vivo.

### SEQUENCE LISTING

A Sequence Listing in xml format entitled “11074WO01_xml,” which was created Nov. 4, 2022, and is 252 Kb, is incorporated herein by reference in its entirety.

### BACKGROUND OF THE INVENTION

The delivery of genes into particular target cells has become one of the most important technologies in modern medicine for the potential treatment of a variety of chronic and genetic diseases. Ideally, a gene delivery vehicle is able to stably introduce genetic material into desired cells and avoid introducing genetic material into non-target cells.

Viral particles, particularly those based on adeno-associated virus (AAV), as a gene delivery vehicles have been the focus of much research since AAVs are capable of transducing a wide range of primate species and tissues in vivo with no evidence of toxicity or pathogenicity. (Muzyczka, et al. (1992) Current Topics in Microbiology and Immunology, 158:97-129). Moreover, AAV safely transduces postmitotic tissues. Although the virus can occasionally integrate into host chromosomes, it does so very infrequently into a safe-harbor locus in human chromosome 19, and only when the replication (Rep) proteins are supplied in trans. AAV genomes rapidly circularize and concatemerize in infected cells, and exist in a stable, episomal state in infected cells to provide long-term stable expression of their payloads.

Additionally, manipulating and redirecting AAV infection to specific cells has been achieved in recent years. Many of the advances in targeted gene therapy using viral particles may be summarized as non-recombinatorial (non-genetic) or recombinatorial (genetic) modification of the viral particle, which result in the pseudotyping, expanding, and/or retargeting of the natural tropism of the viral particle. (Reviewed in Nicklin and Baker (2002) Curr. Gene Ther. 2:273-93; Verheiji and Rottier (2012) Advances Virol 2012:1-15).

In a direct recombinatorial targeting approach, a targeting ligand is directly inserted into, or coupled to, a viral capsid, i.e., protein viral capsid genes are modified to express capsid proteins comprising a heterologous targeting ligand. The targeting ligand than redirects, e.g., binds, a receptor or marker preferentially or exclusively expressed on a target cell. (Stachler et al. (2006) Gene Ther. 13:926-931; White et al. (2004) Circulation 109:513-519; see also Park et al., (2007) Frontiers in Bioscience 13:2653-59; Girod et al. (1999) Nature Medicine 5:1052-56; Grifman et al. (2001) Molecular Therapy 3:964-75; Shi et al. (2001) Human Gene Therapy 12:1697-1711; Shi and Bartlett (2003) Molecular Therapy 7:515-525).

In indirect recombinatorial approaches, a viral capsid is modified with a heterologous “scaffold”, which then links to an adaptor that includes a targeting ligand. The adaptor binds to the scaffold and the target cell. (Arnold et al. (2006) Mol. Ther. 5:125-132; Ponnazhagen et al. (2002) J. Virol. 76:12900-907; see also WO 97/05266) Scaffolds such as (1) Fc binding molecules (e.g., Fc receptors, Protein A, etc.), which bind to the Fc of antibody adaptors, (2) (strept) avidin, which binds to biotinylated adaptors, (3) biotin, which binds to adaptors fused with (strept) avidin, (4) a detectable label, which is useful for detection and/or isolation of viral particles, bound by a bispecific adaptor able to non-covalently bind the detectable label and target molecule, and recently (5) protein:protein binding pairs that form isopeptide bonds have been described for a variety of viral particles. (See, e.g., Gigout et al. (2005) Molecular Therapy 11:856-865; Stachler et al. (2008) Molecular Therapy 16:1467-1473; Quetglas et al. (2010) Virus Research 153:179-196; Ohno et al. (1997) Nature Biotechnology 15:763-767; Klimstra et al. (2005) Virology 338:9-21).

With the advances providing the ability to direct AAV infection, there remains a need to discover targets for the specific transfer of nucleic acids of interest to a cell of interest, e.g., a mammalian muscle cell.

### SUMMARY OF THE INVENTION

It is shown herein that an AAV capsid protein may be modified to allow for the targeted introduction of a nucleotide of interest into mammalian skeletal muscle cells.

Viral particles as described herein are particularly suited for the targeted introduction of a nucleotide of interest specifically to a muscle cell since the viral capsid or viral capsid protein(s) described herein comprise a targeting ligand that binds a muscle-cell specific surface protein. In some embodiments, a viral capsid or viral capsid protein comprises a first member of a binding pair, associated with its cognate second member of the binding pair, wherein the second member is linked (e.g., fused to) a targeting ligand that binds a muscle-cell specific surface protein. In some embodiments, the targeting ligand is operably linked to the second member, e.g., fused to the second member, optionally via a linker. In some embodiments, a targeting ligand may be a binding moiety, e.g., a natural ligand, antibody, a multispecific binding molecule, etc. In some embodiments, the targeting ligand is an antibody or portion thereof. In some embodiments, the targeting ligand is an antibody comprising a variable domain that binds a muscle-specific surface protein on a muscle cell and a heavy chain constant domain. In some embodiments, the targeting ligand is an antibody comprising a variable domain that binds a muscle-specific surface protein on a target cell and an IgG heavy chain constant domain. In some embodiments, the targeting ligand is an antibody comprising a variable domain that binds a muscle-specific surface protein on a target cell and an IgG heavy chain constant domain, wherein the IgG heavy chain constant domain is operably linked, e.g., via a linker, to a protein (e.g., second member of a protein:protein binding pair) that forms an isopeptide covalent bond with the first member. In some embodiments, a capsid protein described herein comprises a first member comprising SpyTag operably linked to the viral capsid protein, and covalently linked to the SpyTag, an second member comprising SpyCatcher linked to a targeting ligand comprising an antibody variable domain and an IgG heavy chain domain, wherein SpyCatcher and the IgG heavy chain domain are linked via an amino acid linker, e.g., GSGESG (SEQ ID NO: 253). In some embodiments, the muscle-specific surfrase protein comprises CACNG1. In some embodiments, the targeting ligand binds CACNG1, e.g., human CACNG1. In some embodiments, the targeting ligand comprises a heavy chain variable domain, light chain variable domain, heavy chain variable domain/light chain variable domain pair, HCDR1, HCDR2, HCDR3, LCDR1, LCDR2, LCDR3, and/or set of HCDR1-HCDR2-HCDR3-LCDR1-LCDR2-LCDR3 comprising an amino acid sequence of a heavy chain variable domain, light chain variable domain, heavy chain variable domain/light chain variable domain pair, HCDR1, HCDR2, HCDR3, LCDR1, LCDR2, LCDR3, and/or set of HCDR1-HCDR2-HCDR3-LCDR1-LCDR2-LCDR3 as set forth in any one of SEQ ID NOs: 1-240.

### BRIEF DESCRIPTION OF THE DRAWINGS

FIG. 1 shows AAV2-based retargeted virus infections delivering GFP to 293 cell lines genetically modified to express ASGR1 or CACNG1. (A) The scatter plots are obtained from flow cytometry evaluating green fluorescent protein (GFP) expression by cells positive (+) for hASGR1 expression after infection with AAV2 WT particles, AAV2 HBM de-targeted mutant particles, AAV2 SpyTag anti-ASGR1 particles, or AAV2 SpyTag anti-CACNG1 particles. Also shown are scatter plots obtained from flow cytometry evaluating green fluorescent protein (GFP) expression by cells positive (+) for hCACNG1 after infection with AAV2 WT particles, AAV2 HBM de-targeted mutant particles, AAV2 SpyTag anti-ASGR 1 particles, or AAV2 SpyTag anti-CACNG1 particles. Viruses express GFP as a marker of transduction. (B) The graph quantifies the percentage of GFP+ cells from the flow cytometry plots in (A).

FIG. 2 shows AAV9-based retargeted virus infections delivering GFP to 293 cell lines genetically modified to express ASGR1 or CACNG1. (A) The scatter plots are obtained from flow cytometry evaluating green fluorescent protein (GFP) expression by cells positive (+) for hASGR1 expression after infection with “AAV9 wt” particles, “AAV9 de-targeted mutant” particles, “AAV9 SpyTag anti-ASGR1” particles, or “AAV9 SpyTag anti-CACNG1” particles. Also shown are scatter plots obtained from flow cytometry evaluating green fluorescent protein (GFP) expression by cells positive (+) for hCACNG1 after infection with “AAV9 wt” particles, “AAV9 de-targeted mutant” particles, “AAV9 SpyTag anti-ASGR1” particles, or “AAV9 SpyTag anti-CACNG1” particles. Viruses express GFP as a marker of transduction. (B) The graph quantifies the percentage of GFP+ cells from the flow cytometry plots in 2A.

FIG. 3 shows AAV2- and AAV9-based retargeted virus infections (MOI 1×10⁶) delivering Luciferase to 293 cell lines genetically modified to express ASGR1 or CACNG1. (A) A luciferase assay was performed to evaluate Firefly luciferase expression by cells positive (+) for hCACNG1 after infection with AAV2 WT, AAV2 HBM+ anti-hASGR1, and AAV2 HBM+ anti-hCACNG1 mAb #1 particles. Also shown are results of a luciferase assay evaluating Firefly luciferase expression by cells positive (+) for hASGR1 after infection with AAV2 WT, AAV2 HBM+ anti-hASGR1, and AAV2 HBM+ anti-hCACNG1 mAb #1 particles. (B) A luciferase assay was performed to evaluate Firefly luciferase expression by cells positive (+) for hCACNG1 after infection with AAV9 WT, AAV9 N272A, AAV9 N272A+ anti-hASGR1 full antibody, AAV9 N272A+ anti-hASGR1 Fab, AAV9 N272A+ anti-CACNG1 mAb #1 full antibody, and AAV9 N272A+ anti-hCACNG1 mAb #1 Fab. Also shown are the results of a luciferase assay evaluating Firefly luciferase expression by cells positive (+) for hCACNG1 after infection with AAV9 WT, AAV9 N272A, AAV9 N272A+ anti-hASGR1 full antibody, AAV9 N272A+ anti-hASGR1 Fab, AAV9 N272A+ anti-CACNG1 mAb #1 full antibody, and AAV9 N272A+ anti-hCACNG1 mAb #1 Fab.

FIG. 4 shows AAV2-based retargeted virus transduction of human skeletal myotubes. Provided are (A) Representative immunofluorescence images and (B) transduction efficiency assessed by quantifying average GFP expression in myosin heavy chain (MyHC) positive areas of human skeletal myotubes after transduction for 3 days with 2E+5 vg/cell of the indicated AAV expressing eGFP under the control of the CAG promoter. Transduction efficiency was assessed in by quantifying the average GFP fluorescence intensity within the myosin heavy chain (MyHC) positive myotube areas.

FIG. 5 shows AAV9-based retargeted virus transduction of human skeletal myotubes. Provided are (A) representative immunofluorescence images and (B) transduction efficiency assessed by quantifying average GFP expression in myosin heavy chain (MyHC) positive areas of human skeletal myotubes after transduction for 3 days with 2E+5 vg/cell of the indicated AAV expressing eGFP under the control of the CAG promoter.

FIG. 6 shows AAV9-based retargeted virus transduction of differentiated mouse C2C12 myotubes. (A) representative immunofluorescence images and (B) transduction efficiency assessed by quantifying average GFP expression in myosin heavy chain (MyHC) positive areas of differentiated mouse C2C12 myotubes transduced for 3 days with 2E+5 vg/cell of the indicated AAV expressing eGFP under the control of the CAG promoter.

FIG. 7 shows systemically delivered AAV2 retargeted to CACNG1 demonstrates antibody-dependent transduction of skeletal muscles in vivo. The graphs provide average radiance values (photons/sec/cm2/sr) from luminescence images of (A) liver, (B) tongue, (C) diaphragm, or (D) quadriceps (quad) tissue imaged ex vivo and isolated from mice genetically modified to express human CACNG1 on skeletal muscle cells (CACNG1 Humanized mice) and wildtype 50500 mice that were injected intravenously with phosphate buffered saline (PBS) or with 5e11 viral genomes (vg)/animal of wildtype (wt) AAV2 particles, AAV2 detargeted particles or SpyTagged AAV2 particles carrying firefly luciferase nucleotides of interest and modified by (1) SpyCatcher-anti-human ASGR1 antibody or (2) SpyCatcher-anti-human CACNG1 antibody. These AAV2 viral particles are mosaic viral particles comprised of a 1:7 ratio between (a) “SpyTag” capsids proteins wherein the Spy Tag is inserted directly following residue G453 flanked on either side by a 10 amino acid linker and (b) capsids without Spy Tag but containing R585A and R588A mutation, which reduces natural receptor binding. Viruses express Firefly luciferase as a marker of transduction. Five weeks post IV injection, mice were anesthetized using isoflurane, injected with a Luciferin substrate and euthanized 7-10 minutes later. Organs were harvested and imaged using IVIS Spectrum in vivo Imaging System (PerkinElmer). The raw data was analyzed using living image software to determine average radiance (photons/sec/cm2/sr).

FIG. 8 shows systematically delivered AAV9 retargeted to CACNG1 demonstrates antibody-dependent transduction of skeletal muscles in vivo. The graphs provide average radiance values (photons/sec/cm2/sr) from luminescence images of (A) liver, (B) hindlimb, (C) quadriceps (quad), or (D) tongue tissue imaged ex vivo and isolated from mice genetically modified to express human CACNG1 (CACNG1 humanized mice) injected intravenously with phosphate buffered saline (PBS) or with 5e10 viral genomes (vg)/animal of wildtype (wt) AAV9 particles, AAV9 detargeted particles or SpyTagged AAV9 particles carrying firefly luciferase nucleotide of interest and modified by (1) SpyCatcher-anti-human ASGR1 full antibody, (2) SpyCatcher-anti-human ASGR1 Fab, (3) SpyCatcher-anti-human CACNG1 mAb #1 full antibody, or (4) SpyCatcher-anti-human CACNG1 mAb #1 Fab. These AAV9 viral particles are mosaic viral particles comprised of a 1:7 ratio between (a) “Spy Tag” capsids proteins wherein the SpyTag is inserted directly following residue G453 flanked on both sides by a 10 amino acid linker and (b) capsids without SpyTag but containing an N272A mutation which reduces natural receptor binding. Viruses express Firefly luciferase as a marker of transduction. Three weeks post IV injection, mice were anesthetized using isoflurane, injected with a Luciferin substrate and euthanized 7-10 minutes later. The following organs were harvested for ex vivo imaging: liver, hindlimb, quad, and tongue. The organs were imaged using IVIS Spectrum in vivo Imaging System (PerkinElmer). The raw data was analyzed using living image software to determine average radiance (photons/sec/cm2/sr).

FIG. 9 displays GFP gene expression analysis in the liver and quadriceps muscle of (A) CACNG1^{hu/hu}, (B) WT C57BL/6, and (C) D2-mdx mice 3 weeks after tail vein injection of 1E+11 vg/mouse of wildtype AAV9, de-targeted AAV9 N272A, and AAV9 conjugated to antibodies targeting CACNG1 (mAb #1 and mAb #2) or hASGR1 as a non-targeting control. GFP expression was quantified via a Taqman-based qPCR assay and normalized to Rplp0 as an endogenous control. GFP mRNA expression is displayed relative to WT AAV9 for each tissue/mouse strain.

FIG. 10 displays representative immunofluorescence images of the tibialis anterior and gastrocnemius/plantaris/soleus muscles of D2-mdx mice 3 weeks after tail vein injection of 1E+11 vg/mouse of wildtype AAV9, de-targeted AAV9 N272A, and AAV9 N272A conjugated to antibodies targeting CACNG1 (mAb #1, mAb #2, and mAb #3) or hASGR1 as a non-targeting control. D2-mdx mice injected with WT AAV9, de-targeted AAV9 N272A, AAV9 conjugated to an irrelevant antibody (hASGR1), and AAV9 conjugated to an antibody that binds to human and monkey CACNG1 (mAb #1), but that does not bind mouse CACNG1, show limited GFP fluorescence in these muscles, whereas mice injected with AAV9 particles conjugated to antibodies that bind to both human and mouse CACNG1 (mAb #2 and mAb #3) display robust GFP fluorescence in patches of myofibers throughout the muscle.

FIG. 11 shows immunohistochemistry staining for eGFP expression in the liver and quadriceps of D2-mdx mice following injection of wildtype AAV9, de-targeted AAV9 N272A, and AAV9 N272A conjugated to antibodies targeting CACNG1 or hASGR1 as a non-targeting control. AAV9 wildtype particles can transduce the liver of D2-mdx mice, while AAV9 N272A particles are detargeted from the liver and do not express GFP in the liver. D2-mdx mice injected with AAV9 N272A conjugated to an irrelevant antibody (hASGR1) or to a CACNG1-targeting antibody that binds human and monkey CACNG1, but that does not bind mouse CACNG1 (mAb #1), show very little staining in the liver or quad. AAV9 N272A particles conjugated to antibodies that bind to both human and mouse CACNG1 (mAb #2 and mAb #3) show very little GFP staining in the liver and strong GFP staining in the quadriceps.

FIG. 12 shows immunohistochemistry staining for eGFP expression in the gastrocnemius/plantaris/soleus of D2-mdx mice following injection of wildtype AAV9, de-targeted AAV9 N272A, and AAV9 N272A conjugated to antibodies targeting CACNG1 or hASGR1 as a non-targeting control. AAV9 wildtype particles can transduce the gastrocnemius/plantaris/soleus of D2-mdx mice at a low level, while AAV9 N272A particles transduce the gastrocnemius/plantaris/soleus with limited efficiency. D2-mdx mice injected with AAV9 N272A conjugated to an irrelevant antibody (hASGR1) or to a CACNG1-targeting antibody specific for human CACNG1 that does not bind mouse CACNG1 (mAb #1) show very little staining in the gastrocnemius/plantaris/soleus. AAV9 N272A particles conjugated to antibodies that bind to both human and mouse CACNG1 (mAb #2 and mAb #3) show very strong GFP staining in the gastrocnemius/plantaris/soleus.

FIG. 13 shows immunohistochemistry staining for eGFP expression in the tibialis anterior of D2-mdx mice following injection of wildtype AAV9, de-targeted AAV9 N272A, and AAV9 N272A conjugated to antibodies targeting CACNG1 or hASGR1 as a non-targeting control. AAV9 wildtype particles can transduce the tibialis anterior of D2-mdx mice at a low level. D2-mdx mice injected with AAV9 N272A particles alone, or AAV9 N272A conjugated to an irrelevant antibody (hASGR1) or to a CACNG1-targeting antibody that binds human and monkey CACNG1, but that does not bind mouse CACNG1, (mAb #1) show very little staining in the tibialis anterior. AAV9 N272A particles conjugated to antibodies that bind to both human and mouse CACNG1 (mAb #2 and mAb #3) show very strong GFP staining around the periphery of the tibialis anterior.

FIG. 14 shows immunohistochemistry staining for eGFP expression in the heart and tongue of D2-mdx mice following injection of wildtype AAV9, de-targeted AAV9 N272A, and AAV9 N272A conjugated to antibodies targeting CACNG1 or hASGR1 as a non-targeting control. AAV9 wildtype particles can transduce the tongue of D2-mdx mice at a low level, but can transduce the heart efficiently. D2-mdx mice injected with AAV9 N272A particles alone, or AAV9 N272A conjugated to an irrelevant antibody (hASGR1) or to a CACNG1-targeting antibody that binds human and monkey CACNG1, but that does not bind mouse CACNG1, (mAb #1) show very little staining in the tongue, and low levels of staining in the heart. AAV9 N272A particles conjugated to antibodies that bind to both human and mouse CACNG1 (mAb #2 and mAb #3) show very strong GFP staining in the tongue, with low but detectable levels of staining in the heart.

FIG. 15 shows immunohistochemistry staining for eGFP expression in the spleen and diaphragm of D2-mdx mice following injection of wildtype AAV9, de-targeted AAV9 N272A, and AAV9 N272A conjugated to antibodies targeting CACNG1 or hASGR1 as a non-targeting control. AAV9 wildtype particles can transduce the diaphragm of D2-mdx mice at a low level. D2-mdx mice injected with AAV9 N272A particles alone, or AAV9 N272A conjugated to an irrelevant antibody (hASGR1, or to a CACNG1-targeting antibody that binds human and monkey CACNG1 but not mouse CACNG1 (mAb #1) show very little staining in the diaphragm. AAV9 N272A particles conjugated to antibodies that bind to both human and mouse CACNG1 (mAb #2 and mAb #3) show very strong GFP staining in the diaphragm. Very little transduction of spleen is observed with any of the tested AAVs, as expected.

FIG. 16 provides an illustrative schematic (not to scale) of the single stranded (ss) viral genome comprising from 5′ to 3′: a 141 base pair inverted terminal repeat (ITR), a CAGG promoter, a sequence encoding enhanced green fluorescent protein (GFP), Woodchuck Hepatitis Virus Posttranscriptional Regulatory Element (WPRE), a 32 base pair barcode, human (h) grown hormone (GH) poly A tail, and the 141 base pair ITR.

FIG. 17 provides bar graphs that demonstrate enhanced transduction to various muscles in vivo in non-human primates (cynomolgus monkey) after administration of AAV9 viral particles comprising the viral genome depicted in FIG. 16, each with a unique barcode, and retargeted with anti-CACNG1 antibodies, compared to wildtype AAV9 viral particles (AAV) comprising the viral genome depicted in FIG. 16. Each candidate AAV was packaged with a unique barcoded genome as described in FIG. 16. Following IV dosing of the 12 candidate barcoded pool, the indicated tissues were collected and relative abundance of each barcode in the total RNA purified from each tissue was assessed using next generation sequencing (NGS). Shown are the percentage of NGS reads (Y-axis) mapped to each barcode and associated capsid in the several tissues (x-axis): liver (left lateral lobe) and a subset of skeletal muscles (diaphragm, biceps brachii, bicep femoris, extensor digitorum longus (EDL), gastrocnemius, intercostals, soleus, tibialis anterior, transverse abdominus, triceps, vastus lateralis, psoas, tongue); normalized to the injected virus pool. The data represented here is the mean of two animals in the study. In the liver, AAV9 alone and AAV9 W503A or N272A conjugated to an ASGR1 mAb represent the majority of all barcodes present in the tissue, as expected. In skeletal muscle tissues, detargeted AAV9 (N272A or W503A) capsids conjugated to CACNG1 targeting antibodies represent the majority of all barcodes present in the tissue, outperforming AAV9 alone, which accounted for a small percentage of total barcodes.

FIG. 18 shows AAV9-based retargeted virus transduction of human skeletal myotubes and C2C12 mouse myotubes using a vector genome construct expressing uDys5. The graphs provide transduction efficiency assessed by quantifying relative uDys5 mRNA expression in (A) human myotubes and (B) C2C12 mouse myotubes after transduction for 3 days with 2E+5 vg/cell of either AAV9 WT or a de-targeted AAV9 (N272A) conjugated to an antibody targeting CACNG1 (mAb #3) that express uDys5 under the control of the CK8 promoter. uDys5 expression was quantified via a Taqman-based qPCR assay and normalized to Hprt as an endogenous control. uDys5 mRNA expression is displayed relative to WT AAV9 for each cell type. AAV9 N272A particles conjugated to an antibody that binds to both human and mouse CACNG1 (mAb #3) produce higher levels of uDys5 mRNA relative to AAV9 WT in both human and mouse myotubes.

FIG. 19 displays uDys5 gene expression in multiple tissues from D2-mdx mice 5 weeks after tail vein injection of 1E+12 vg/mouse of either AAV9 WT or a de-targeted AAV9 (N272A) conjugated to an antibody targeting CACNG1 (mAb #3) that express uDys5 under the control of the CK8 promoter. uDys5 mRNA expression was quantified via a Taqman-based qPCR assay and normalized to Rplp0 as an endogenous control. uDys5 mRNA expression is displayed relative to WT AAV9 for each tissue. AAV9 N272A particles conjugated to an antibody that binds to both human and mouse CACNG1 (mAb #3) show reduced transduction of the liver relative to AAV9 WT as expected, and produce extremely low levels of uDys5 mRNA relative to AAV9 WT in the liver. AAV9 N272A particles conjugated to an antibody that binds to both human and mouse CACNG1 (mAb #3) produce lower levels of uDys5 mRNA relative to AAV9 WT in the heart, but the levels are detectable. AAV9 N272A particles conjugated to an antibody that binds to both human and mouse CACNG1 (mAb #3) produce higher levels of uDys5 mRNA relative to AAV9 WT in all skeletal muscles examined.

FIG. 20 displays representative immunofluorescence images of the gastrocnemius muscle and heart of wildtype DBA2/J and D2-mdx mice 5 weeks after tail vein injection of 1E+12 vg/mouse of wildtype AAV9 or de-targeted AAV9 N272A particles conjugated to antibodies targeting CACNG1 (mAb #3) expressing uDys5 under the control of the CK8 promoter. D2-mdx mice injected with WT AAV9 show a low level of expression of dystrophin at the myofiber membrane in the gastrocnemius, and robust expression in the heart; whereas mice injected with de-targeted AAV9 N272A particles conjugated to an antibody that binds to both human and mouse CACNG1 (mAb #3) display robust dystrophin expression at the myofiber membrane in the gastrocnemius, and mild expression in the heart.

FIG. 21 displays protein abundance of uDys5 in the quadriceps muscle of D2-mdx mice 5 weeks after tail vein injection of 1E+12 vg/mouse of wildtype AAV9 or de-targeted AAV9 N272A particles conjugated to antibodies targeting CACNG1 (mAb #3) expressing uDys5 under the control of the CK8 promoter. β-actin was used as a protein loading control, and protein abundance was quantified and plotted as arbitrary densitometry units (A.U.). Mice injected with de-targeted AAV9 N272A particles conjugated to an antibody that binds to both human and mouse CACNG1 (mAb #3) display substantially more uDys5 protein compared to mice injected with wildtype AAV9.

FIG. 22 displays serum creatine kinase (CK) levels in D2-mdx mice 4 weeks after tail vein injection of 1E+12 vg/mouse of wildtype AAV9 or de-targeted AAV9 N272A particles conjugated to antibodies targeting CACNG1 (mAb #3) expressing uDys5 under the control of the CK8 promoter. PBS treated and wildtype AAV9 treated mice did not display a decrease in serum CK following treatment, while all mice that were treated with de-targeted AAV9 N272A particles conjugated to an antibody that binds to both human and mouse CACNG1 (mAb #3) displayed a reduction of serum CK, indicating reduced muscle damage.

FIG. 23 displays maximal forelimb grip strength measurements in D2-mdx mice 12 weeks after tail vein injection of 1E+12 vg/mouse of wildtype AAV9 or de-targeted AAV9 N272A particles conjugated to antibodies targeting CACNG1 (mAb #3) expressing uDys5 under the control of the CK8 promoter. Dotted line represents grip strength of age-matched control DBA/2J mice. Mice injected with AAV9 N272A particles conjugated to an antibody that binds to both human and mouse CACNG1 (mAb #3) display enhanced maximum grip strength relative to the mice injected with WT AAV9 particles.

FIG. 24 shows serum levels of liver enzymes and complement pathway biomarkers in non-human primates (cynomolgous monkey) at the indicated timepoints following injection of wildtype AAV9 or AAV9 N272A conjugated to antibodies targeting CACNG1 (mAb #3) expressing eGFP under the control of a CAG promoter. Administration of AAV9 wildtype particles resulted in an elevation of (A) ALT, (B) AST, (C) Bb and (D) C3a 48 hours post-dosing, as expected, while administration of AAV9 N272A conjugated to antibodies targeting CACNG1 (mAb #3) did not result in an elevation of these markers, suggesting that liver-detargeted AAV9 N272A particles conjugated to antibodies targeting CACNG1 provide a safety advantage over liver-tropic wildtype AAV serotypes.

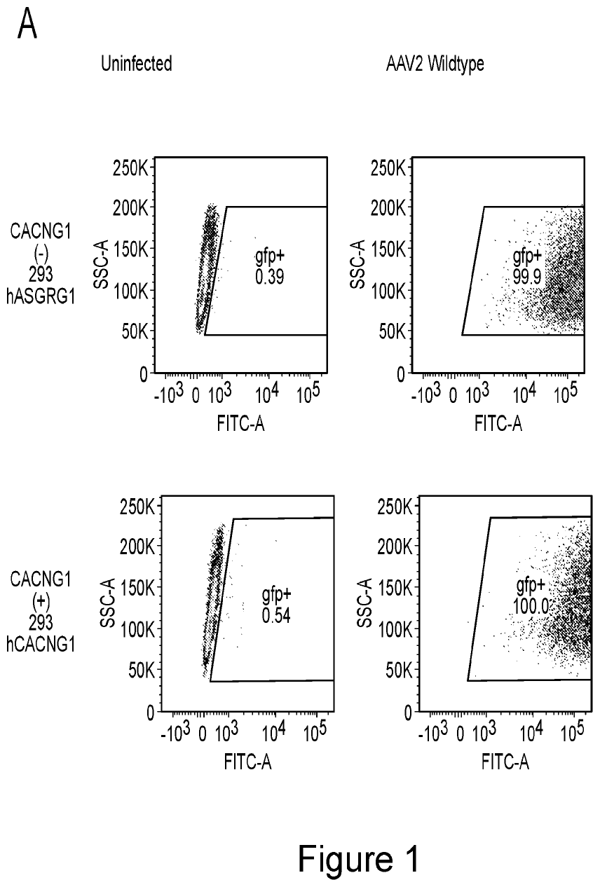

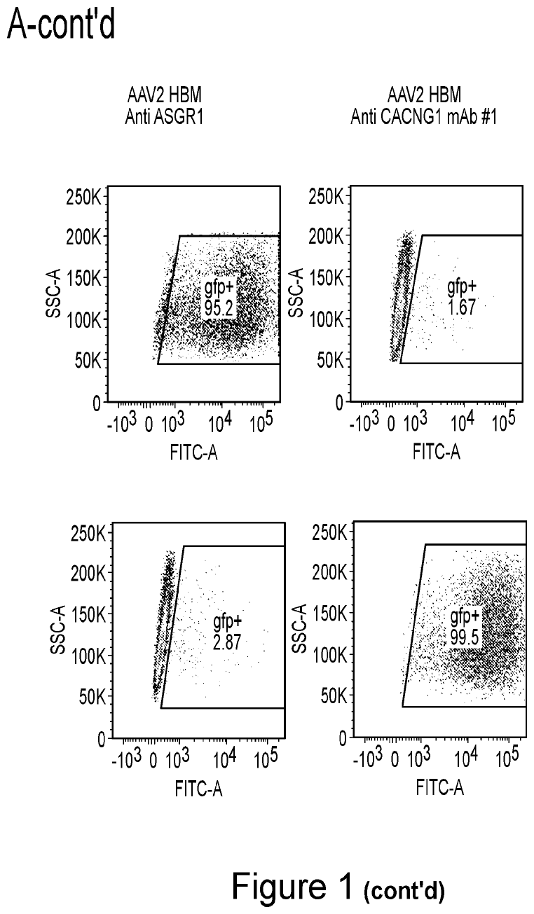

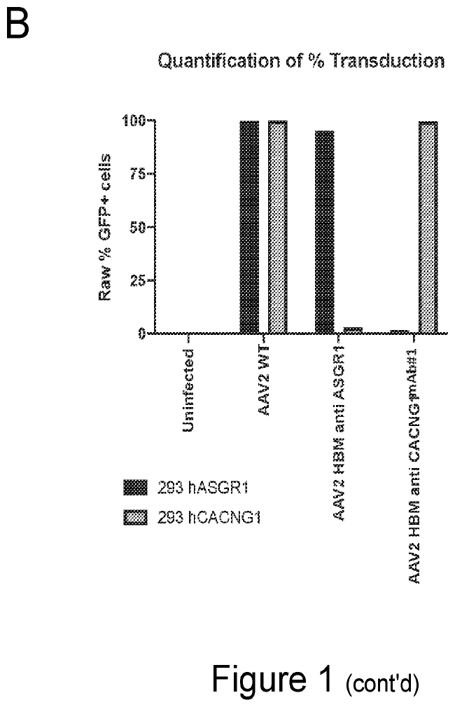

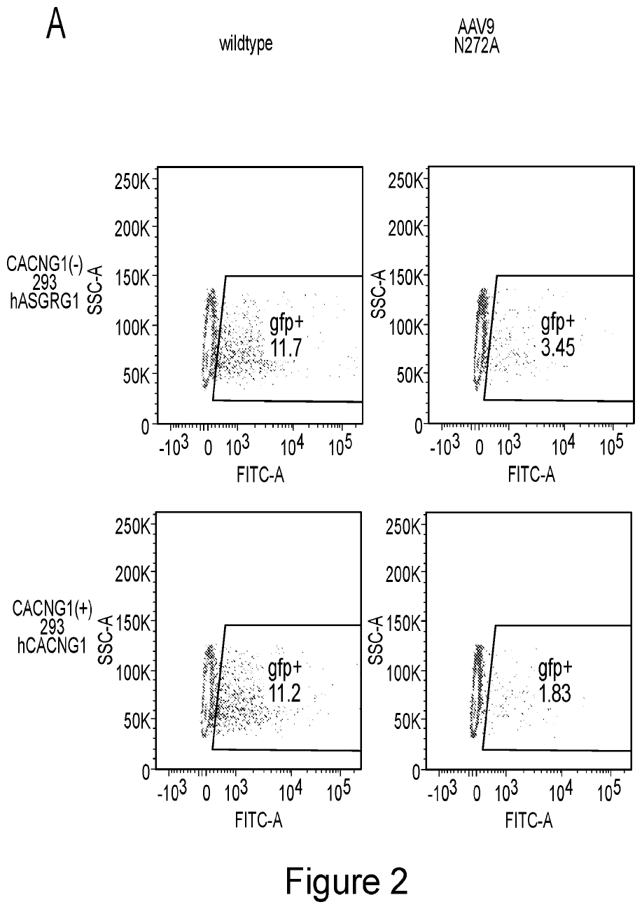

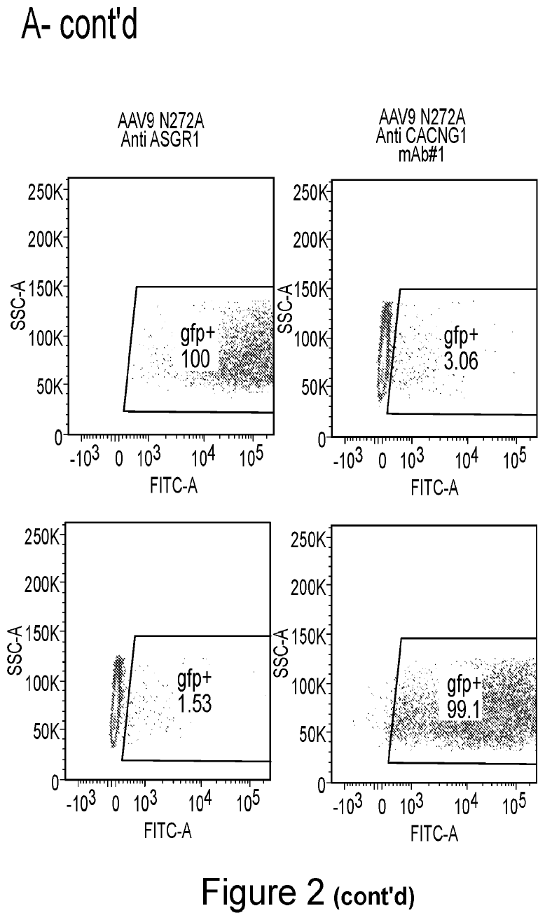

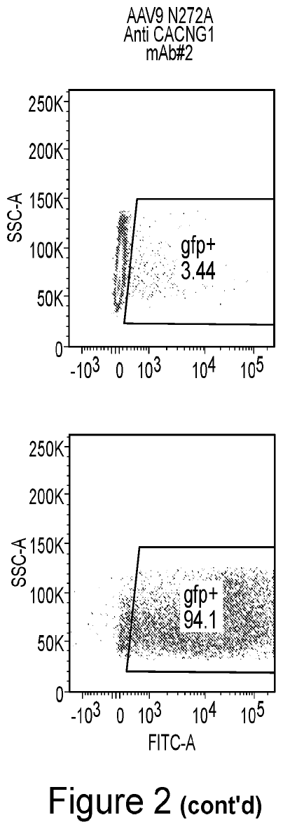

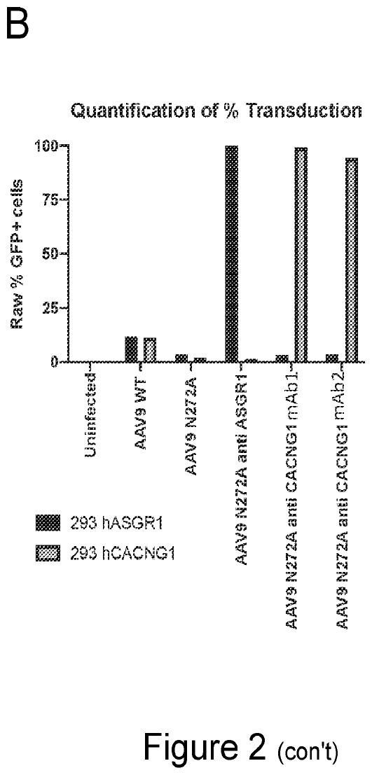

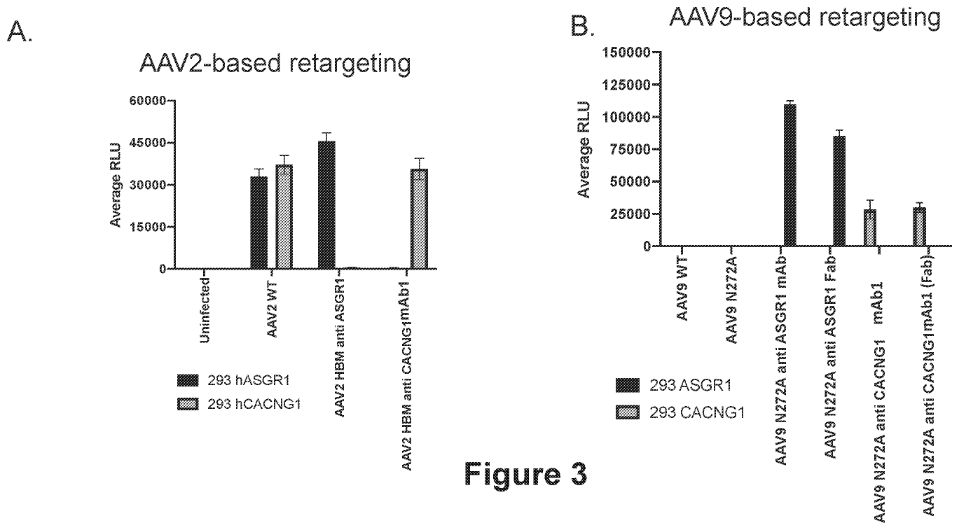

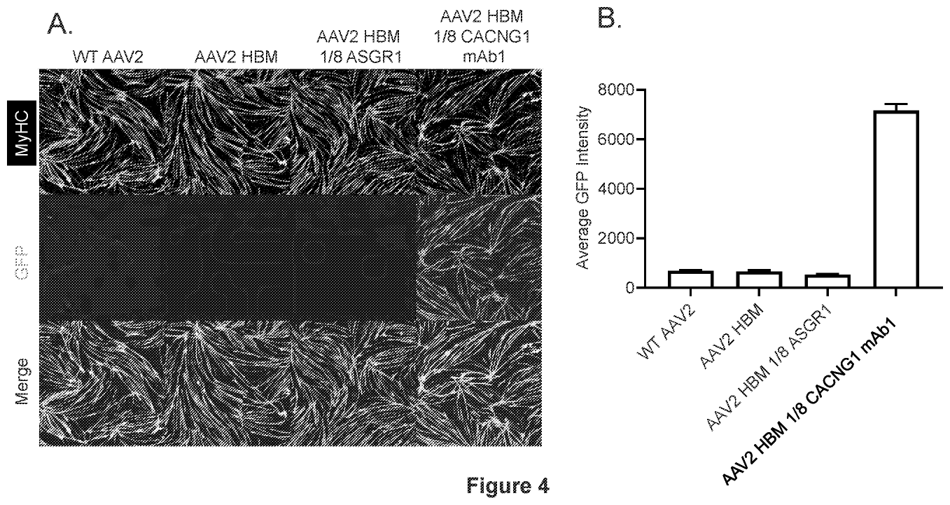

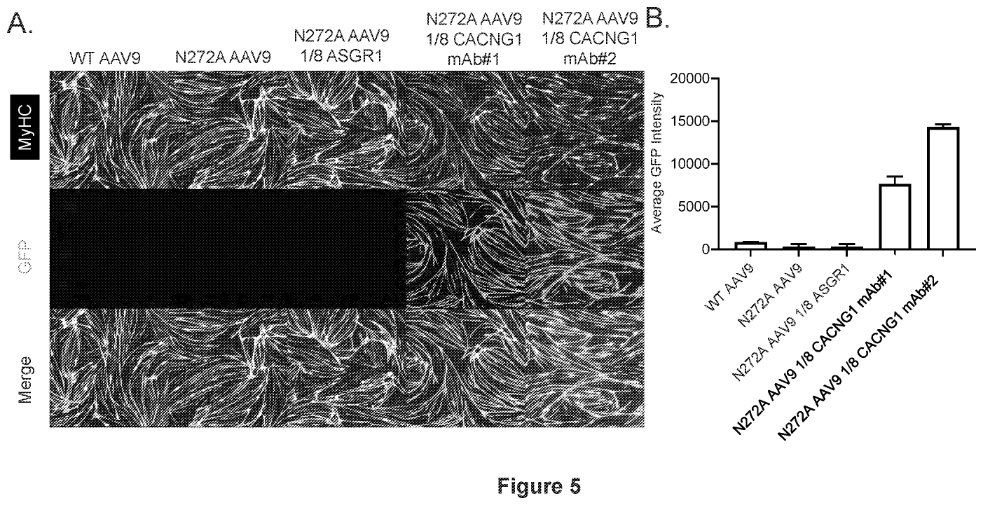

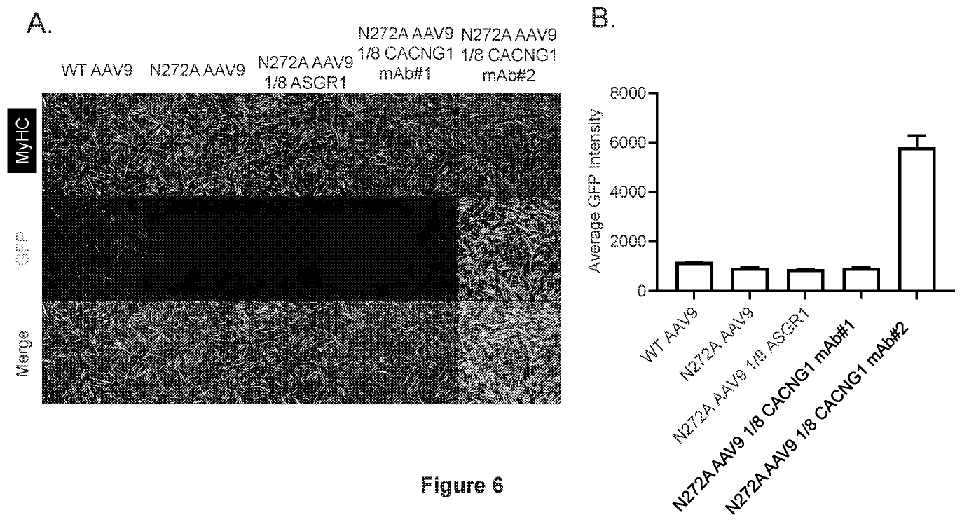

In each of the figures described above:
“WT” refers to wildtype, e.g., AAV capsid proteins with no mutations or modifications.
“HBM” refers capsid proteins comprising R585A and R588A. All “reference”/“detargeted” AAV2 capsid proteins, e.g., AAV capsid proteins without a Spy Tag modification comprise only R585A and R588A. All AAV2 capsid proteins in the figures described above that display Spy Tag bound to a SpyCatcher fused antibody further comprise R484A, R487A, R585A, R588A, and K532A mutations.
“N272A” refers capsids comprising an N272A mutations. In the figures described above, all “reference”/“detargeted” AAV9 capsid proteins, e.g., AAV9 capsid proteins without a SpyTag modification, comprise an N272A mutation. In the figures described above, all AAV9 capsid proteins with a SpyTag modification further comprise a W503A mutation but not the N272A mutation.
“anti CACNG1,” “CACNG1” or “CACNG1 mAb” in association with an AAV refers to AAV viral capsids comprising an insertion of the SpyTag peptide directly following residue G453 flanked on both sides by a 10 amino acid linker, where the SpyTag peptide is bound by an isopeptide bond to a SpyCatcher fused to an anti-CACNG1 antibody (or Fab fragment thereof) that specifically binds CACNG1.
   All AAV particles displaying the SpyTag peptide bound by an isopeptide bond to a SpyCatcher fused to an anti-CACNG1 antibody (or Fab fragment thereof) that specifically binds CACNG1 in in the figures described above comprise a mosaic viral capsid comprising a 1:7 ratio of SpyTag modified viral capsid proteins to “detargeted” viral proteins (denoted “1/8”).
“CACNG1 mAb #1” refers to an anti-CACNG1 antibody that binds human and monkey CACNG1, but does not bind mouse CACNG1.
“CACNG1 mAb #2” refers to an anti-CACNG1 antibody that binds human, monkey, and mouse CACNG1.
“CACNG1 mAb #3” refers to an anti-CACNG1 antibody that binds human, monkey, and mouse CACNG1.
“CACNG1 mAb #4” refers to an anti-CACNG1 antibody that binds human, monkey, and mouse CACNG1.
“CACNG1 mAb #5” refers to an anti-CACNG1 antibody that binds human and monkey, but not mouse CACNG1.
“CACNG1” in association with 293 cells or mice respectively refer to 293 cells or mice genetically modified to express human CACNG1.
Mice comprising a homozygous replacement of endogenous Cacng1 with human Cacng1 sequences are referred to as “CACNG1^{hu/hu}”.
“50500” in association with mice refers to a strain-matched control for CACNG1 mice.
“anti ASGR1,” “ASGR1” or “ASGR1 mAb” in association with an AAV refers to AAV viral capsids comprising an insertion of the SpyTag peptide directly following residue G453 flanked on both sides by a 10 amino acid linker, where the SpyTag peptide is bound by an isopeptide bond to a SpyCatcher fused to an anti-ASGR1 antibody or Fab fragment thereof that specifically binds ASGR1.
   All AAV particles displaying the SpyTag peptide bound by an isopeptide bond to a SpyCatcher fused to an anti-ASGR1 antibody (or Fab fragment thereof) that specifically binds ASGR1 in the figures described above comprise a mosaic viral capsid comprising a 1:7 ratio of SpyTag modified viral capsid proteins to “detargeted” viral capsid proteins. (denoted “1/8”).
“ASGR1” in association with 293 cells or mice respectively refer to 293 cells or mice genetically modified to express human ASGR1.
“h” refers to “human”
“Vh” refers to an antibody heavy chain
“Vk” refers to an antibody light chain
“D2-mdx” refers to a mouse model for Duchenne muscular dystrophy

### DETAILED DESCRIPTION

Skeletal muscle is the largest organ in the body, comprising ˜40% of total body mass. Skeletal muscle is one of the three significant muscle tissues in the human body. Each skeletal muscle consists of thousands of muscle fibers wrapped together by connective tissue sheaths.

The primary functions of the skeletal muscle take place via its intrinsic excitation-contraction coupling process. As the muscle is attached to the bone tendons, the contraction of the muscle leads to movement of that bone that allows for the performance of specific movements. The skeletal muscle also provides structural support and helps in maintaining the posture of the body. The skeletal muscle also acts as a storage source for amino acids that can be used by different organs of the body for synthesizing organ-specific proteins. The skeletal muscle also acts as a storage source of glucose in the form of glycogen. The skeletal muscle also plays a central role in maintaining thermostasis and acts as an energy source during starvation. Thus, skeletal muscle plays key roles in locomotion, thermoregulation, and in controlling whole body metabolism.

In many muscle diseases as well as during normal aging, the size and function of skeletal muscle tissue is reduced, resulting in impaired functional mobility; and in the case of severe muscle diseases, long-term disability and early mortality.

Treatments for muscle wasting and genetic muscle diseases typically consist of broad-acting therapies, such as testosterone therapy for muscle wasting, glucocorticoids for muscular dystrophies, and systemic AAV delivery for treatment of muscle diseases (e.g., X-linked myotubular myopathy (XLMTM), Duchenne muscular dystrophy (DMD), myotonic dystrophy (DM1), Facioscapulohumeral muscular dystrophy Type 1 (FSHD), congenital muscular dystrophy type 1A (MDC1A), Limb girdle muscular dystrophy, and dystroglycanopathy, etc.). Untargeted delivery of these therapies reduces efficiency of specific muscle uptake, while also causing significant detrimental off-target effects on other organs.

To enhance muscle delivery of therapeutic payloads and mitigate off-target effects, described herein are viral particles, e.g., AAV viral particles, that target muscle-specific surface proteins, such as Calcium Voltage-Gaged Auxiliary Subunit Gamma 1 (CACNG1) or mammalian Cadherin 15 (CAD15).

Voltage-dependent calcium channels are generally composed of five subunits. The protein encoded by the CACNG1 gene represents one of these subunits. Further, the protein encoded by the CACNG1 gene, gamma, is one of two known gamma subunit proteins. This particular gamma subunit is part of skeletal muscle 1,4-dihydropyridine-sensitive calcium channels and is an integral membrane protein that plays a role in excitation-contraction coupling. This gene is part of a functionally diverse eight-member protein subfamily of the PMP-22/EMP/MP20 family and is located in a cluster with two family members that function as transmembrane AMPA receptor regulatory proteins (TARPs). CACNG1 is highly and specifically expressed in skeletal muscle. The gene encoding human CACNG1 (CACNG1) is located on the long arm of chromosome 17. CACNG1 comprises 4 exons and is approximately 12,244 bases long. An exemplary sequence for human CACNG1 gene is assigned NCBI Accession Number NM_0007582.2 (SEQ ID NO:241). An exemplary human CACNG1 protein is assigned UniProt Accession No. 070578 (SEQ ID NO:242).

Unless defined otherwise, all technical and scientific terms used herein have the same meaning as commonly understood by one of ordinary skill in the art to which this invention belongs.

Singular forms “a”, “an”, and “the” include plural references unless the context clearly dictates otherwise. Thus, for example, a reference to “a method” includes one or more methods, and/or steps of the type described herein and/or which will become apparent to those persons skilled in the art upon reading this disclosure.

The “percent (%) identity” or the like may be readily determined for amino acid or nucleotide sequences, over the full-length of a protein, or a portion thereof. A portion may be at least about 5 amino acids or 24 nucleotides, respectively, in length, and may be up to about 700 amino acids or 2100 nucleotides, respectively. Generally, when referring to “identity”, “homology”, or “similarity” between two different adeno-associated viruses, “identity”, “homology” or “similarity” is determined in reference to “aligned” sequences. “Aligned” sequences or “alignments” refer to multiple nucleic acid sequences or protein (amino acids) sequences, often containing corrections for missing or additional bases or amino acids as compared to a reference sequence.

Alignments may be performed using any of a variety of publicly or commercially available Multiple Sequence Alignment Programs. Sequence alignment programs are available for amino acid sequences, e.g., the “Clustal X”, “MAP”, “PIMA”, “MSA”, “BLOCKMAKER”, “MEME”, and “Match-Box” programs. Generally, any of these programs are used at default settings, although one of skill in the art can alter these settings as needed. Alternatively, one of skill in the art can utilize another algorithm or computer program which provides at least the level of identity or alignment as that provided by the referenced algorithms and programs. See, e.g., J. D. Thomson et al, Nucl. Acids. Res., “A comprehensive comparison of multiple sequence alignments”, 27 (13): 2682-2690 (1999).

Multiple sequence alignment programs are also available for nucleic acid sequences. Examples of such programs include, “Clustal W”, “CAP Sequence Assembly”, “MAP”, and “MEME”, which are accessible through Web Servers on the internet. Other sources for such programs are known to those of skill in the art. Alternatively, Vector NTI utilities are also used. There are also a number of algorithms known in the art that can be used to measure nucleotide sequence identity, including those contained in the programs described above. As another example, polynucleotide sequences can be compared using FASTA™, a program in GCG Version 6.1. Fasta™ provides alignments and percent sequence identity of the regions of the best overlap between the query and search sequences. For instance, percent sequence identity between nucleic acid sequences can be determined using FASTA™ with its default parameters (a word size of 6 and the NOPAM factor for the scoring matrix) as provided in GCG Version 6.1, herein incorporated by reference.

“Significant identity” encompasses amino acid or nucleic acid sequences alignments that are at least 90%, e.g., at least 93%, e.g., at least 95%, e.g., at least 96%, e.g., at least 97%, e.g., at least 98%, e.g., at least 99%, or e.g., at least 100% identical.

The term “chimeric” encompasses a functional gene or polypeptide comprising nucleic acid sequences or amino acid sequences, respectively, from at least two different AAV serotype, e.g., portions of a gene or polypeptide of at least a first and second AAV, wherein the at least first and second portions are operably linked to form a functional chimeric AAV nucleic acid that encodes a functional amino acid. Unless specified as chimeric, nucleotide sequences, genes, polypeptides, and amino acids are considered non-chimeric in that the nucleotide sequences, genes, polypeptides, and amino acids comprise a nucleic acid sequence or amino acid sequence having significant identity to a nucleic acid sequence or amino acid sequence, respectively, of a single AAV serotype.

The term “antibody” includes immunoglobulin molecules comprising four polypeptide chains, two heavy (H) chains and two light (L) chains inter-connected by disulfide bonds. Each heavy chain comprises a heavy chain variable domain (V_{H}) and a heavy chain constant region (C_{H}). The heavy chain constant region comprises at least three domains, C_{H}1, C_{H}2, C_{H}3 and optionally C_{H}4. Each light chain comprises a light chain variable domain (C_{H}) and a light chain constant region (C_{L}). The heavy chain and light chain variable domains can be further subdivided into regions of hypervariability, termed complementarity determining regions (CDR), interspersed with regions that are more conserved, termed framework regions (FR). Each heavy and light chain variable domain comprises three CDRs and four FRs, arranged from amino-terminus to carboxy-terminus in the following order: FR1, CDR1, FR2, CDR2, FR3, CDR3, FR4 (heavy chain CDRs may be abbreviated as HCDR1, HCDR2 and HCDR3; light chain CDRs may be abbreviated as LCDR1, LCDR2 and LCDR3. Typical tetrameric antibody structures comprise two identical antigen-binding domains, each of which formed by association of the V_{H} and V_{L} domains, and each of which together with respective C_{H} and C_{L} domains form the antibody Fv region. Single domain antibodies comprise a single antigen-binding domain, e.g., a V_{H} or a V_{L}. The antigen-binding domain of an antibody, e.g., the part of an antibody that recognizes and binds to the first member of a specific binding pair of an antigen, is also referred to as a “paratope.” It is a small region (of 5 to 10 amino acids) of an antibody's Fv region, part of the fragment antigen-binding (Fab region), and may contain parts of the antibody's heavy and/or light chains. A paratope specifically binds a first member of a specific binding pair when the paratope binds the first member of a specific binding pair with a high affinity. The term “high affinity” antibody refers to an antibody that has a K_{D} with respect to its target first member of a specific binding pair about of 10⁻⁹ M or lower (e.g., about 1×10⁻⁹ M, 1×10⁻¹⁰ M, 1×10⁻¹¹ M, or about 1×10⁻¹² M). In one embodiment, K_{D} is measured by surface plasmon resonance, e.g., BIACORE™; in another embodiment, K_{D} is measured by ELISA.

The phrase “complementarity determining region,” or the term “CDR,” includes an amino acid sequence encoded by a nucleic acid sequence of an organism's immunoglobulin genes that normally (i.e., in a wild-type animal) appears between two framework regions in a variable region of a light or a heavy chain of an immunoglobulin molecule (e.g., an antibody or a T cell receptor). A CDR can be encoded by, for example, a germ line sequence or a rearranged or unrearranged sequence, and, for example, by a naive or a mature B cell or a T cell. A CDR can be somatically mutated (e.g., vary from a sequence encoded in an animal's germ line), humanized, and/or modified with amino acid substitutions, additions, or deletions. In some circumstances (e.g., for a CDR3), CDRs can be encoded by two or more sequences (e.g., germ line sequences) that are not contiguous (e.g., in an unrearranged nucleic acid sequence) but are contiguous in a B cell nucleic acid sequence, e.g., as the result of splicing or connecting the sequences (e.g., V-D-J recombination to form a heavy chain CDR3).

The phrase “light chain” includes an immunoglobulin light chain sequence from any organism, and unless otherwise specified includes human κ and λ light chains and a VpreB, as well as surrogate light chains. Light chain variable domains typically include three light chain CDRs and four framework (FR) regions, unless otherwise specified. Generally, a full-length light chain includes, from amino terminus to carboxyl terminus, a variable domain that includes FR1-CDR1-FR2-CDR2-FR3-CDR3-FR4, and a light chain constant region. A light chain variable domain is encoded by a light chain variable region gene sequence, which generally comprises V_{L} and J_{L} segments, derived from a repertoire of V and J segments present in the germ line. Sequences, locations and nomenclature for V and J light chain segments for various organisms can be found in IMGT database, www.imgt.org. Light chains include those, e.g., that do not selectively bind either a first or a second first member of a specific binding pair selectively bound by the first member of a specific binding pair-binding protein in which they appear. Light chains also include those that bind and recognize, or assist the heavy chain or another light chain with binding and recognizing, one or more first member of a specific binding pairs selectively bound by the first member of a specific binding pair-binding protein in which they appear. Common or universal light chains include those derived from a human Vκ1-39Jκ gene or a human Vκ3-20Jκ gene, and include somatically mutated (e.g., affinity matured) versions of the same. Exemplary human V_{L} segments include a human Vκ1-39 gene segment, a human Vκ3-20 gene segment, a human Vλ1-40 gene segment, a human Vλ1-44 gene segment, a human Vλ2-8 gene segment, a human Vλ2-14 gene segment, and human Vλ3-21 gene segment, and include somatically mutated (e.g., affinity matured) versions of the same. Light chains can be made that comprise a variable domain from one organism (e.g., human or rodent, e.g., rat or mouse; or bird, e.g., chicken) and a constant region from the same or a different organism (e.g., human or rodent, e.g., rat or mouse; or bird, e.g., chicken).

The term “about” or “approximately” includes being within a statistically meaningful range of a value. Such a range can be within an order of magnitude, preferably within 50%, more preferably within 20%, still more preferably within 10%, and even more preferably within 5% of a given value or range. The allowable variation encompassed by the term “about” or “approximately” depends on the particular system under study, and can be readily appreciated by one of ordinary skill in the art.

The phrase “heavy chain,” or “immunoglobulin heavy chain” includes an immunoglobulin heavy chain sequence, including immunoglobulin heavy chain constant region sequence, from any organism. Heavy chain variable domains include three heavy chain CDRs and four FR regions, unless otherwise specified. Fragments of heavy chains include CDRs, CDRs and FRs, and combinations thereof. A typical heavy chain has, following the variable domain (from N-terminal to C-terminal), a C_{H}1 domain, a hinge, a C_{H}2 domain, and a C_{H}3 domain. A functional fragment of a heavy chain includes a fragment that is capable of specifically recognizing an first member of a specific binding pair (e.g., recognizing the first member of a specific binding pair with a K_{D} in the micromolar, nanomolar, or picomolar range), that is capable of expressing and secreting from a cell, and that comprises at least one CDR. Heavy chain variable domains are encoded by variable region nucleotide sequence, which generally comprises V_{H}, D_{H}, and J_{H} segments derived from a repertoire of V_{H}, D_{H}, and J_{H} segments present in the germline. Sequences, locations and nomenclature for V, D, and J heavy chain segments for various organisms can be found in IMGT database, which is accessible via the internet on the world wide web (www) at the URL “imgt.org.”

The term “heavy chain only antibody,” “heavy chain only antigen binding protein,” “single domain antigen binding protein,” “single domain binding protein” or the like refers to a monomeric or homodimeric immunoglobulin molecule comprising an immunoglobulin-like chain comprising a variable domain operably linked to a heavy chain constant region, that is unable to associate with a light chain because the heavy chain constant region typically lacks a functional C_{H}1 domain. Accordingly, the term “heavy chain only antibody,” “heavy chain only antigen binding protein,” “single domain antigen binding protein,” “single domain binding protein” or the like encompasses a both (i) a monomeric single domain antigen binding protein comprising one of the immunoglobulin-like chain comprising a variable domain operably linked to a heavy chain constant region lacking a functional C_{H}1 domain, or (ii) a homodimeric single domain antigen binding protein comprising two immunoglobulin-like chains, each of which comprising a variable domain operably linked to a heavy chain constant region lacking a functional C_{H}1 domain. In various aspects, a homodimeric single domain antigen binding protein comprises two identical immunoglobulin-like chains, each of which comprising an identical variable domain operably linked to an identical heavy chain constant region lacking a functional C_{H}1 domain. Additionally, each immunoglobulin-like chain of a single domain antigen binding protein comprises a variable domain, which may be derived from heavy chain variable region gene segments (e.g., V_{H}, D_{H}, J_{H}), light chain gene segments (e.g., V_{L}, J_{L}), or a combination thereof, linked to a heavy chain constant region (C_{H}) gene sequence comprising a deletion or inactivating mutation in a C_{H}1 encoding sequence (and, optionally, a hinge region) of a heavy chain constant region gene, e.g., IgG, IgA, IgE, IgD, or a combination thereof. A single domain antigen binding protein comprising a variable domain derived from heavy chain gene segments may be referred to as a “V_{H}-single domain antibody” or “V_{H}-single domain antigen binding protein”, see, e.g., U.S. Pat. No. 8,754,287; U.S. Patent Publication Nos. 20140289876; 20150197553; 20150197554; 20150197555; 20150196015; 20150197556 and 20150197557, each of which is incorporated in its entirety by reference. A single domain antigen binding protein comprising a variable domain derived from light chain gene segments may be referred to as a or “V_{L}-single domain antigen binding protein,” see, e.g., U.S. Publication No. 20150289489, incorporated in its entirety by reference.

The phrase “light chain” includes an immunoglobulin light chain sequence from any organism, and unless otherwise specified includes human kappa (κ) and lambda (λ) light chains and a VpreB, as well as surrogate light chains. Light chain variable domains typically include three light chain CDRs and four framework (FR) regions, unless otherwise specified. Generally, a full-length light chain includes, from amino terminus to carboxyl terminus, a variable domain that includes FR1-CDR1-FR2-CDR2-FR3-CDR3-FR4, and a light chain constant region amino acid sequence. Light chain variable domains are encoded by the light chain variable region nucleotide sequence, which generally comprises light chain V_{L} and light chain J_{L} gene segments, derived from a repertoire of light chain V and J gene segments present in the germline. Sequences, locations and nomenclature for light chain V and J gene segments for various organisms can be found in IMGT database, which is accessible via the internet on the world wide web (www) at the URL “imgt.org.” Light chains include those, e.g., that do not selectively bind either a first or a second first member of a specific binding pair selectively bound by the first member of a specific binding pair-binding protein in which they appear. Light chains also include those that bind and recognize, or assist the heavy chain with binding and recognizing, one or more first member of a specific binding pairs selectively bound by the first member of a specific binding pair-binding protein in which they appear. Light chains also include those that bind and recognize, or assist the heavy chain with binding and recognizing, one or more first member of a specific binding pairs selectively bound by the first member of a specific binding pair-binding protein in which they appear. Common or universal light chains include those derived from a human Vκ1-39Jκ5 gene or a human Vκ3-20Jκ1 gene, and include somatically mutated (e.g., affinity matured) versions of the same.

The phrase “operably linked”, as used herein, includes a physical juxtaposition (e.g., in three-dimensional space) of components or elements that interact, directly or indirectly with one another, or otherwise coordinate with each other to participate in a biological event, which juxtaposition achieves or permits such interaction and/or coordination. To give but one example, a regulatory element (e.g., an expression control sequence) in a nucleic acid is said to be “operably linked” to a coding sequence when it is located relative to the coding sequence such that its presence or absence impacts expression and/or activity of the coding sequence. In many embodiments, “operable linkage” involves covalent linkage of relevant components or elements with one another. Those skilled in the art will readily appreciate that, in some embodiments, covalent linkage is not required to achieve effective operable linkage. For example, proteins operably linked together may be associated with each other, e.g., via a covalent bond or a non-covalent bond. As a non-limiting example, a capsid protein as described herein may be operably linked to a targeting ligand, where the capsid protein is non-covalently bound to the targeting ligand, or covalently bound to the targeting ligand, optionally with or without a scaffold and/or adaptor between the capsid protein and the targeting ligand. As another example, in some embodiments, nucleic acid regulatory elements that are operably linked with coding sequences that they control are contiguous with the nucleotide of interest. Alternatively or additionally, in some embodiments, one or more such regulatory elements acts in trans or at a distance to control a coding sequence of interest. In some embodiments, the term “regulatory element” as used herein refers to polynucleotide sequences which are necessary and/or sufficient to effect the expression and processing of coding sequences to which they are ligated. In some embodiments, a regulatory element may be or comprise appropriate transcription initiation, termination, promoter and/or enhancer sequences; efficient RNA processing signals such as splicing and polyadenylation signals; sequences that stabilize cytoplasmic mRNA; sequences that enhance translation efficiency (e.g., Kozak consensus sequence); sequences that enhance protein stability; and/or, in some embodiments, sequences that enhance protein secretion. In some embodiments, one or more regulatory elements are preferentially or exclusively active in a particular host cell or organism, or type thereof. To give but one example, in prokaryotes, regulatory elements may typically include promoter, ribosomal binding site, and transcription termination sequence; in eukaryotes, in many embodiments, regulatory elements may typically include promoters, enhancers, and/or transcription termination sequences. Those of ordinary skill in the art will appreciate from context that, in many embodiments, the term “regulatory elements” refers to components whose presence is essential for expression and processing, and in some embodiments includes components whose presence is advantageous for expression (including, for example, leader sequences, targeting sequences, and/or fusion partner sequences).

“Retargeting” or “redirecting” may include a scenario in which the wildtype particle targets several cells within a tissue and/or several organs within an organism, and general targeting of the tissue or organs is reduced or abolished by insertion of the heterologous amino acid, and retargeting to more a specific cell in the tissue or a specific organ in the organism is achieved with the targeting ligand (e.g., via a targeting ligand) that binds a marker expressed by the specific cell. Such retargeting or redirecting may also include a scenario in which the wildtype particle targets a tissue, and targeting of the tissue is reduced to or abolished by insertion of the heterologous amino acid, and retargeting to a completely different tissue is achieved with the targeting ligand.

“Specific binding pair,” “binding pair,” “protein:protein binding pair” and the like includes two members (e.g., a first member (e.g., a first polypeptide) and a second cognate member (e.g., a second polypeptide)) that interact to form a bond (e.g., a non-covalent bond between a first member epitope and a second member antigen-binding portion of an antibody that recognizes the epitope; a covalent bond between e.g., proteins capable of forming isopeptide bonds; split inteins that recognize each other and, through the process of protein trans-splicing, mediate ligation of the flanking proteins and their own removal). In some embodiments, the term “cognate” refers to components that function together. Epitopes and cognate antibodies thereto, particularly epitopes that may also act as a detectable label (e.g., c-myc) are well-known in the art. Specific protein:protein binding pairs capable of interacting to form a covalent isopeptide bond are reviewed in Veggiani et al. (2014) Trends Biotechnol. 32:506, and include peptide:peptide binding pairs such as SpyTag:SpyCatcher, SpyTag002:SpyCatcher002; SpyTag:KTag; isopeptag:pilin C, SnoopTag:SnoopCatcher, etc., and variants thereof, e.g., SpyTag003:SpyCatcher003. Generally, a first member of a protein:protein binding pair refers to member of a protein:protein binding pair, which is generally less than 30 amino acids in length, and which forms a spontaneous covalent isopeptide bond with the second cognate protein, wherein the second cognate protein is generally larger, but may also be less than 30 amino acids in length such as in the SpyTag:KTag system.

The term “isopeptide bond” refers to an amide bond between a carboxyl or carboxamide group and an amino group at least one of which is not derived from a protein main chain or alternatively viewed is not part of the protein backbone. An isopeptide bond may form within a single protein or may occur between two peptides or a peptide and a protein. Thus, an isopeptide bond may form intramolecularly within a single protein or intermolecularly i.e. between two peptide/protein molecules, e.g. between two peptide linkers. Typically, an isopeptide bond may occur between a lysine residue and an asparagine, aspartic acid, glutamine, or glutamic acid residue or the terminal carboxyl group of the protein or peptide chain or may occur between the alpha-amino terminus of the protein or peptide chain and an asparagine, aspartic acid, glutamine or glutamic acid. Each residue of the pair involved in the isopeptide bond is referred to herein as a reactive residue. In preferred embodiments of the invention, an isopeptide bond may form between a lysine residue and an asparagine residue or between a lysine residue and an aspartic acid residue. Particularly, isopeptide bonds can occur between the side chain amine of lysine and carboxamide group of asparagine or carboxyl group of an aspartate.

The Spy Tag:SpyCatcher system is described in U.S. Pat. No. 9,547,003 and Zaveri et al. (2012) PNAS 109:E690-E697, each of which is incorporated herein in its entirety by reference, and is derived from the CnaB2 domain of the Streptococcus pyogenes fibronecting-binding protein FbaB. By splitting the domain, Zakeri et al. obtained a peptide “SpyTag” having the sequence AHIVMVDAYKPTK (SEQ ID NO:243) which forms an amide bond to its cognate protein “SpyCatcher,” an 112 amino acid polypeptide having the amino acid sequence set forth in SEQ ID NO:244. (Zakeri (2012), supra). An additional specific binding pair derived from CnaB2 domain is SpyTag:KTag, which forms an isopeptide bond in the presence of SpyLigase. (Fierer (2014) PNAS 111:E1176-1181) SpyLigase was engineered by excising the β strand from SpyCatcher that contains a reactive lysine, resulting in KTag, 10-residue first member of a protein:protein binding pair having the amino acid sequence ATHIKFSKRD (SEQ ID NO:245). The SpyTag002:SpyCatcher002 system is described in Keeble et al (2017) Angew Chem Int Ed Engl 56:16521-25, incorporated herein in its entirety by reference. SpyTag002 has the amino acid sequence VPTIVMVDAYKRYK, set forth as SEQ ID NO:255, and binds SpyCatcher002. SpyTag003 has the amino acid sequence RGVPHIVMVDAYKRYK, set forth as SEQ ID NO:259, and binds SpyCatcher003.

The Snoop Tag:SnoopCatcher system is described in Veggiani (2016) PNAS 113:1202-07. The D4 Ig-like domain of RrgA, an adhesion from Streptococcus pneumoniae, was split to form SnoopTag (residues 734-745) and SnoopCatcher (residues 749-860). Incubation of SnoopTag and SnoopCatcher results in a spontaneous isopeptide bond that is specific between the complementary proteins. Veggiani (2016)), supra.

The isopeptag:pilin-C specific binding pair was derived from the major pilin protein Spy0128 from Streptococcus pyogenes. (Zakeir and Howarth (2010) J. Am. Chem. Soc. 132:4526-27). Isopeptag has the amino acid sequence TDKDMTITFTNKKDAE, set forth as SEQ ID NO:254, and binds pilin-C (residues 18-299 of Spy0128). Incubation of SnoopTag and SnoopCatcher results in a spontaneous isopeptide bond that is specific between the complementary proteins. Zakeir and Howarth (2010), supra.

The term “detectable label” includes a polypeptide sequence that is a member of a specific binding pair, e.g., that specifically binds via a non-covalent bond with another polypeptide sequence, e.g., an antibody paratope, with high affinity. Exemplary and non-limiting detectable labels include hexahistidine tag, FLAG tag, Strep II tag, streptavidin-binding peptide (SBP) tag, calmodulin-binding peptide (CBP), glutathione S-transferase (GST), maltose-binding protein (MBP), S-tag, HA tag, and the myc tag from c-myc (SEQ ID NO: 246). (Reviewed in Zhao et al. (2013) J. Analytical Meth. Chem. 1-8; incorporated herein by reference). A common detectable label for primate AAV is the B1 epitope (SEQ ID NO: 247). Some AAV capsid proteins described herein, which do not naturally comprise the B1 epitope, may be modified herein to comprise a B1 epitope. Generally, AAV capsid proteins described herein may comprise a sequence with substantial homology to the B1 epitope within the last 10 amino acids of the capsid protein. Accordingly, in some embodiments, a non-primate AAV capsid protein of the invention may be modified with one but less than five point mutations within the last 10 amino acids of the capsid protein such that the AAV capsid protein comprises a B1 epitope.

The term “target cells” includes any cells in which expression of a nucleotide of interest is desired. Preferably, target cells exhibit a receptor on their surface that allows the cell to be targeted with a targeting ligand, as described below.

The term “transduction” or “infection” or the like refers to the introduction of a nucleic acid into a target cell nucleus by a viral particle. The term efficiency in relation to transduction or the like, e.g., “transduction efficiency” refers to the fraction (e.g., percentage) of cells expressing a nucleotide of interest after incubation with a set number of viral particles comprising the nucleotide of interest. Well-known methods of determining transduction efficiency include flow cytometry of cells transduced with a fluorescent reporter gene, RT-PCR for expression of the nucleotide of interest, etc.

Generally “reference” viral capsid protein/capsid/particle are identical to test viral capsid protein/capsid/particle but for the change for which the effect is to be tested. For example, to determine the effect, e.g., on transduction efficiency, of inserting a first member of a specific binding pair into a test viral particle, the transduction efficiencies of the test viral particle (in the absence or presence of an appropriate targeting ligand) can be compared to the transduction efficiencies of a reference viral particle (in the absence or presence of an appropriate targeting ligand if necessary) which is identical to the test viral particle in every instance (e.g., additional point mutations, nucleotide of interest, numbers of viral particles and target cells, etc.) except for the presence of a first member of a specific binding pair. In some embodiments, a reference viral capsid protein is one that is able to form a capsid with a second viral capsid protein modified to comprise at least a first member of a protein:protein binding pair, where the reference viral capsid protein does not comprise the first member of a protein:protein binding pair, preferably wherein the capsid formed by the reference viral capsid protein and the modified viral capsid protein is a mosaic capsid.

### Adeno-Associated Viruses (AAV)

“AAV” is an abbreviation for adeno-associated virus and may be used to refer to the virus itself or derivatives thereof. AAVs are small, non-enveloped, single-stranded DNA viruses. Generally, a wildtype AAV genome is 4.7 kb and is characterized by two inverted terminal repeats (ITR) and two open reading frames (ORFs), rep and cap. The wildtype rep reading frame encodes four proteins of molecular weight 78 kD (“Rep78”), 68 kD (“Rep68”), 52 kD (“Rep52”) and 40 kD (“Rep 40”). Rep78 and Rep68 are transcribed from the p5 promoter, and Rep52 and Rep40 are transcribed from the p19 promoter. These proteins function mainly in regulating the transcription and replication of the AAV genome. The wildtype cap reading frame encodes three structural (capsid) viral proteins (VPs) having molecular weights of 83-85 kD (VP1), 72-73 kD (VP2) and 61-62 kD (VP3). More than 80% of total proteins in an AAV virion (capsid) comprise VP3; in mature virions VP1, VP2 and VP3 are found at relative abundance of approximately 1:1:10, although ratios of 1:1:8 have been reported. Padron et al. (2005) J. Virology 79:5047-58.

The genomic sequences of various serotypes of AAV, as well as the sequences of the native inverted terminal repeats (ITRs), Rep proteins, and capsid subunits are known in the art. Such sequences may be found in the literature or in public databases such as GenBank. See, e.g., GenBank Accession Numbers NC_002077 (AAV1), AF063497 (AAV1), NC001401 (AAV-2), AF043303 (AAV2), NC_001729 (AAV3), NC_001829 (AAV4), U89790 (AAV4), NC_006152 (AAV5), AF513851 (AAV7), AF513852 (AAV8), and NC_006261 (AAV8); the disclosures of which are incorporated by reference herein for teaching AAV nucleic acid and amino acid sequences. See also, e.g., Srivistava et al. (1983) J. Virology 45:555; Chiorini et al. (1998) J. Virology 71:6823; Chiorini et al. (1999) J. Virology 73:1309; Bantel-Schaal et al. (1999) J. Virology 73:939; Xiao et al. (1999) J. Virology 73:3994; Muramatsu et al. (1996) Virology 221:208; Shade et al., (1986) J. Virol. 58:921; Gao et al. (2002) Proc. Nat. Acad. Sci. USA 99:11854; Moris et al. (2004) Virology 33:375-383; US Patent Publication 20170130245; international patent publications WO 00/28061, WO 99/61601, WO 98/11244; and U.S. Pat. No. 6,156,303, each of which is incorporated by reference in its entirety by reference. Table 2 herein provides sequences of various non-primate AAV.

“AAV” encompasses all subtypes and both naturally occurring and modified forms that are well-known in the art. AAV includes primate AAV (e.g., AAV type 1 (AAV1), primate AAV type 2 (AAV2), primate AAV type 3 (AAV3B), primate AAV type 4 (AAV4), primate AAV type 5 (AAV5), primate AAV type 6 (AAV6), primate AAV type 7 (AAV7), primate AAV type 8 (AAV8), primate AAV type 9 (AAV9), AAV10, AAV11, AAV12, AAV13, AAVDJ, Anc80L65, AAV2G9, AAV-LK03, primate AAV type rh10 (AAV rh10), AAV type h10 (AAV h10), AAV type hu11 (AAV hu11), AAV type rh32.33 (AAV rh32.33), AAV retro (AAV retro), AAV PHP.B, AAV PHP.eB, AAV PHP.S, AAV2/8, etc., non-primate animal AAV (e.g., avian AAV (AAAV)) and other non-primate animal AAV such as mammalian AAV (e.g., bat AAV, sea lion AAV, bovine AAV, canine AAV, equine AAV, caprine AAV, and ovine AAV etc.), squamate AAV (e.g., snake AAV, bearded dragon AAV), etc. “Primate AAV” refers to AAV generally isolated from primates. Similarly, “non-primate animal AAV” refers to AAV isolated from non-primate animals.

As used herein, “of a [specified] AAV” in relation to a gene (e.g., rep, cap, etc.), capsid protein (e.g., a VP1 capsid protein, a VP2 capsid protein, a VP3 capsid protein, etc.), region of a capsid protein of a specified AAV (e.g., PLA₂ region, VP1-u region, VP1/VP2 common region, VP3 region), nucleotide sequence (e.g., ITR sequence), e.g., a cap gene or capsid protein of AAV etc., encompasses, in addition to the gene or the polypeptide respectively comprising a nucleic acid sequence or amino acid sequence set forth herein for the specified AAV, also variants of the gene or polypeptide, including variants comprising the least number of nucleotides or amino acids required to retain one or more biological functions. As used herein, a variant gene or a variant polypeptide comprises a nucleic acid sequence or amino acid sequence that differs from the nucleic acid sequence or amino acid sequence set forth herein for the gene or polypeptide of a specified AAV, wherein the difference(s) does not generally alter at least one biological function of the gene or polypeptide, and/or the phylogenetic characterization of the gene or polypeptide, e.g., where the difference(s) may be due to degeneracy of the genetic code, isolate variations, length of the sequence, etc. For example, rep gene and the cap gene as used here may encompass rep and cap genes that differ from the wildtype gene in that the genes may encode one or more Rep proteins and Cap proteins, respectively. In some embodiments, a Rep gene encodes at least Rep78 and/or Rep68. In some embodiments, cap gene includes those may differ from the wildtype in that one or more alternative start codons or sequences between one or more alternative start codons are removed such that the cap gene encodes only a single Cap protein, e.g., wherein the VP2 and/or VP3 start codons are removed or substituted such that the cap gene encodes a functional VP1 capsid protein but not a VP2 capsid protein or a VP3 capsid protein. Accordingly, as used herein, a rep gene encompasses any sequence that encodes a functional Rep protein. A cap gene encompasses any sequence that encodes at least one functional cap gene.

It is well-known that the wildtype cap gene expresses all three VP1, VP2, and VP3 capsid proteins from a single open reading frame of the cap gene under control of the p40 promoter found in the rep ORF. The term “capsid protein,” “Cap protein” and the like includes a protein that is part of the capsid of the virus. For adeno-associated viruses, the capsid proteins are generally referred to as VP1, VP2 and/or VP3, and may be encoded by the single cap gene. For AAV, the three AAV capsid proteins are produced in nature an overlapping fashion from the cap ORF alternative translational start codon usage, although all three proteins use a common stop codon. The ORF of a wildtype cap gene encodes from 5′ to 3′ three alternative start codons: “the VP1 start codon,” “the VP2 start codon,” and “the VP3 start codon”; and one “common stop codon”. The largest viral protein, VP1, is generally encoded from the VP1 start codon to the “common stop codon.” VP2 is generally encoded from the VP2 start codon to the common stop codon. VP3 is generally encoded from the VP3 start codon to the common stop codon. Accordingly, VP1 comprises at its N-terminus sequence that it does not share with the VP2 or VP3, referred to as the VP1-unique region (VP1-u). The VP1-u region is generally encoded by the sequence of a wildtype cap gene starting from the VP1 start codon to the “VP2 start codon.” VP1-u comprises a phospholipase A2 domain (PLA₂), which may be important for infection, as well as nuclear localization signals which may aid the virus in targeting to the nucleus for uncoating and genome release. The VP1, VP2, and VP3 capsid proteins share the same C-terminal sequence that makes up the entirety of VP3, which may also be referred to herein as the VP3 region. The VP3 region is encoded from the VP3 start codon to the common stop codon. VP2 has an additional ˜60 amino acids that it shares with the VP1. This region is called the VP1/VP2 common region.

In some embodiments, one or more of the Cap proteins of the invention may be encoded by one or more cap genes having one or more ORFs. In some embodiments, the VP proteins of the invention may be expressed from more than one ORF comprising nucleotide sequence encoding any combination of VP1, VP2, and/or VP3 by use of separate nucleotide sequences operably linked to at least one expression control sequence for expression in packaging cell, each producing one or more of VP1, VP2, and/or VP3 capsid proteins of the invention. In some embodiments, a VP capsid protein of the invention may be expressed individually from an ORF comprising nucleotide sequence encoding any one of VP1, VP2, or VP3 by use of separate nucleotide sequences operably linked to one expression control sequence for expression in a viral replication cell, each producing only one of VP1, VP2, or VP3 capsid protein. In another embodiment, VP proteins may be expressed from one ORF comprising nucleotide sequences encoding VP1, VP2, and VP3 capsid proteins operably linked to at least one expression control sequence for expression in a viral replication cell, each producing VP1, VP2, and VP3 capsid protein. Accordingly, although amino acid positions provided herein may be provided in relation to the VP1 capsid protein of the referenced AAV, a skilled artisan would be able to respectively and readily determine the position of that same amino acid within the VP2 and/or VP3 capsid protein of the AAV, and the corresponding position of amino acids among different AAV.

The phrase “Inverted terminal repeat” or “ITR” includes symmetrical nucleic acid sequences in the genome of adeno-associated viruses required for efficient replication. ITR sequences are located at each end of the AAV DNA genome. The ITRs serve as the origins of replication for viral DNA synthesis and are essential cis components for generating AAV particles, e.g., packaging into AAV particles.

AAV ITR comprise recognition sites for replication proteins Rep78 or Rep68. A “D” region of the ITR comprises the DNA nick site where DNA replication initiates and provides directionality to the nucleic acid replication step. An AAV replicating in a mammalian cell typically comprises two ITR sequences.

A single ITR may be engineered with Rep binding sites on both strands of the “A” regions and two symmetrical D regions on each side of the ITR palindrome. Such an engineered construct on a double-stranded circular DNA template allows Rep78 or Rep68 initiated nucleic acid replication that proceeds in both directions. A single ITR is sufficient for AAV replication of a circular particle. In methods of producing an AAV viral particle of the invention, the rep encoding sequence encodes a Rep protein or Rep protein equivalent that is capable of binding an ITR comprised on the transfer plasmid.

The Cap proteins of the invention, when expressed with appropriate Rep proteins by a packaging cell, may encapsidate a transfer plasmid comprising a nucleotide of interest and an even number of two or more ITR sequences. In some embodiments, a transfer plasmid comprises one ITR sequence. In some embodiments, a transfer plasmid comprises two ITR sequences.

Either Rep78 and/or Rep68 bind to unique and known sites on the sequence of the ITR hairpin, and act to break and unwind the hairpin structures on the end of an AAV genome, thereby providing access to replication machinery of the viral replication cell. As is well-known, Rep proteins may be expressed from more than one ORF comprising nucleotide sequence encoding any combination of Rep78, Rep68, Rep 52 and/or Rep40 by use of separate nucleotide sequences operably linked to at least one expression control sequence for expression in a viral replication cell, each producing one or more of Rep78, Rep68, Rep 52 and/or Rep40 Rep proteins. Alternatively, Rep proteins may be expressed individually from an ORF comprising a nucleotide sequence encoding any one of Rep78, Rep68, Rep 52, or Rep40 by use of separate nucleotide sequences operably linked to one expression control sequence for expression in a packaging cell, each producing only one Rep78, Rep68, Rep 52, or Rep40 Rep protein. In another embodiment, Rep proteins may be expressed from one ORF comprising nucleotide sequences encoding Rep78 and Rep52 Rep proteins operably linked to at least one expression control sequence for expression in a viral replication cell each producing Rep78 and Rep52 Rep protein.

In a method of producing an AAV virion, e.g., viral particle, of the invention, a rep encoding sequence and a cap gene of the invention may be provided a single packaging plasmid. However, a skilled artisan will recognize that such proviso is not necessary. Such viral particles may or may not include a genome.

A “chimeric AAV capsid protein” includes an AAV capsid protein that comprises amino acid sequences, e.g., portions, from two or more different AAV and that is capable of forming and/or forms an AAV viral capsid/viral particle. A chimeric AAV capsid protein is encoded by a chimeric AAV capsid gene, e.g., a chimeric nucleotide comprising a plurality, e.g., at least two, nucleic acid sequences, each of which plurality is identical to a portion of a capsid gene encoding a capsid protein of distinct AAV, and which plurality together encodes a functional chimeric AAV capsid protein. Association of a chimeric capsid protein to a specific AAV indicates that the capsid protein comprises one or more portions from a capsid protein of that AAV and one or more portions from a capsid protein of a different AAV. For example, a chimeric AAV2 capsid protein includes a capsid protein comprising one or more portions of a VP1, VP2, and/or VP3 capsid protein of AAV2 and one or more portions of a VP1, VP2, and/or VP3 capsid protein of a different AAV.

The term “portion” refers to at least 5 amino acids or at least 15 nucleotides, but less than the full-length polypeptide or nucleic acid molecule, with 100% identity to a sequence from which the portion is derived, see Penzes (2015) J. General Virol. 2769. A “portion” encompasses any contiguous segment of amino acids or nucleotides sufficient to determine that the polypeptide or nucleic acid molecule form which the portion is derived is “of a [specified] AAV” or has “significant identity” to a particular AAV, e.g., a non-primate animal AAV or remote AAV. In some embodiments, a portion comprises at least 5 amino acids or 15 nucleotides with 100% identity to a sequence associated with the specified AAV. In some embodiments, a portion comprises at least 10 amino acids or 30 nucleotides with 100% identity to a sequence associated with the specified AAV. In some embodiments, a portion comprises at least 15 amino acids or 45 nucleotides with 100% identity to a sequence associated with the specified AAV. In some embodiments, a portion comprises at least 20 amino acids or 60 nucleotides with 100% identity to a sequence associated with the specified AAV. In some embodiments, a portion comprises at least 25 amino acids or 75 nucleotides with 100% identity to a sequence associated with the specified AAV. In some embodiments, a portion comprises at least 30 amino acids or 90 nucleotides with 100% identity to a sequence associated with the specified AAV. In some embodiments, a portion comprises at least 35 amino acids or 105 nucleotides with 100% identity to a sequence associated with the specified AAV. In some embodiments, a portion comprises at least 40 amino acids or 120 nucleotides with 100% identity to a sequence associated with the specified AAV. In some embodiments, a portion comprises at least 45 amino acids or 135 nucleotides with 100% identity to a sequence associated with the specified AAV. In some embodiments, a portion comprises at least 50 amino acids or 150 nucleotides with 100% identity to a sequence associated with the specified AAV. In some embodiments, a portion comprises at least 60 amino acids or 180 nucleotides with 100% identity to a sequence associated with the specified AAV. In some embodiments, a portion comprises at least 70 amino acids or 210 nucleotides with 100% identity to a sequence associated with the specified AAV. In some embodiments, a portion comprises at least 80 amino acids or 240 nucleotides with 100% identity to a sequence associated with the specified AAV. In some embodiments, a portion comprises at least 90 amino acids or 270 nucleotides with 100% identity to a sequence associated with the specified AAV. In some embodiments, a portion comprises at least 100 amino acids or 300 nucleotides with 100% identity to a sequence associated with the specified AAV.

### Modified Virus Capsid Proteins, Viral Particles, Viral Nucleic Acids

In some embodiments, a Cap protein, e.g., a VP1 capsid protein as described herein, a VP2 capsid protein as described herein, and/or a VP3 capsid protein as described herein, is modified to comprise any one or combination of e.g., insertion of a targeting ligand, a chemical modification, a first member of a binding pair, a detectable label, point mutation, etc.

Generally, modification of gene or a polypeptide of a specified AAV, or variants thereof, results in nucleic acid sequence or an amino acid sequence that differs from the nucleic acid sequence or amino acid sequence set forth herein for the specified AAV, wherein the modification alters, confers, or removes one or more biological functions, but does not change the phylogenetic characterization of, the gene or polypeptide as an AAV gene or AAV polypeptide. Modifications may include any one or a combination of: substitution of sequences of a first AAV serotype with sequences of a second AAV serotype to create chimerism; chemical modification; an insertion of: a first member of a binding pair, and/or a point mutation; etc., such that the natural tropism of the capsid protein is reduced to abolished, the tropism of the capsid protein may be more easily redirected, and/or such that the capsid protein comprises a detectable label. Modifications as described herein generally do not alter and preferably decrease the low to no recognition of the modified capsid by pre-existing antibodies found in the general population that were produced during the course of infection with another AAV, e.g., infection with serotypes such as AAV1, AAV2, AAV3, AAV4, AAV5, AAV6, AAV7, AAV8, AAV9, AAV10, AAV11, AAV12, AAV13, AAVDJ, Anc80L65, AAV2G9, AAV-LK03, virions based on such serotypes, virions from currently used AAV gene therapy modalities, or a combination thereof.

### Targeting Ligand

Modifications described herein may pertain to the association (e.g, display, operable linkage, binding) of a targeting ligand to a modified capsid protein and/or capsid comprising a modified capsid protein. Generally, a targeting ligand as described herein binds a surface protein expressed by a mammalian muscle cell, e.g., a protein that is expressed on the surface of a mammalian muscle cell, e.g., a mammalian muscle cell-specific surface protein. In some embodiments, a modified capsid protein and/or modified capsid comprises a targeting ligand that binds mammalian CACNG1, e.g., a human CACNG1.

[1] Table 1 provides a summary of the SEQ ID NO for each binding portion (e.g., heavy chain variable domain (HCVR), light chain variable domain (LCVR), and CDR1, CDR2, and CDR3) of non-limiting and exemplary anti-human-CACNG1 monoclonal antibodies (mAb ID) that may be used to redirect an AAV capsid as described herein. In some embodiments, an AAV capsid as described herein comprises a targeting ligand that binds human CACNG1, wherein the targeting ligand comprises heavy chain variable domain, light chain variable domain, heavy chain variable domain/light chain variable domain pair, HCDR1, HCDR2, HCDR3, LCDR1, LCDR2, LCDR3, and/or set of HCDR1-HCDR2-HCDR3-LCDR1-LCDR2-LCDR3 amino acid sequence(s) at least 90% identical to, respectively, an amino acid sequence of a heavy chain variable domain, light chain variable domain, heavy chain variable domain/light chain variable domain pair, HCDR1, HCDR2, HCDR3, LCDR1, LCDR2, LCDR3, and/or set of HCDR1-HCDR2-HCDR3-LCDR1-LCDR2-LCDR3 as set forth in any one of SEQ ID NOs: 1-240. In some embodiments, an AAV capsid as described herein comprises a targeting ligand that binds human CACNG1, wherein the targeting ligand comprises a heavy chain variable domain, light chain variable domain, heavy chain variable domain/light chain variable domain pair, HCDR1, HCDR2, HCDR3, LCDR1, LCDR2, LCDR3, and/or set of HCDR1-HCDR2-HCDR3-LCDR1-LCDR2-LCDR3 amino acid sequence at least 95% identical to, respectively, amino acid sequence(s) of a heavy chain variable domain, light chain variable domain, heavy chain variable domain/light chain variable domain pair, HCDR1, HCDR2, HCDR3, LCDR1, LCDR2, LCDR3, and/or set of HCDR1-HCDR2-HCDR3-LCDR1-LCDR2-LCDR3 set forth in any one of SEQ ID NOs: 1-240. In some embodiments, an AAV capsid as described herein comprises a targeting ligand that binds human CACNG1, wherein the targeting ligand comprises a heavy chain variable domain, light chain variable domain, heavy chain variable domain/light chain variable domain pair, HCDR1, HCDR2, HCDR3, LCDR1, LCDR2, LCDR3, and/or set of HCDR1-HCDR2-HCDR3-LCDR1-LCDR2-LCDR3 amino acid sequence at least 97% identical to amino acid sequence(s) of a heavy chain variable domain, light chain variable domain, heavy chain variable domain/light chain variable domain pair, HCDR1, HCDR2, HCDR3, LCDR1, LCDR2, LCDR3, and/or set of HCDR1-HCDR2-HCDR3-LCDR1-LCDR2-LCDR3 set forth in any one of SEQ ID NOs: 1-240. In some embodiments, an AAV capsid as described herein comprises a targeting ligand that binds human CACNG1, wherein the targeting ligand comprises a heavy chain variable domain, light chain variable domain, heavy chain variable domain/light chain variable domain pair, HCDR1, HCDR2, HCDR3, LCDR1, LCDR2, LCDR3, and/or set of HCDR1-HCDR2-HCDR3-LCDR1-LCDR2-LCDR3 amino acid sequence(s) at least 98% identical to amino acid sequence(s) of a heavy chain variable domain, light chain variable domain, heavy chain variable domain/light chain variable domain pair, HCDR1, HCDR2, HCDR3, LCDR1, LCDR2, LCDR3, and/or set of HCDR1-HCDR2-HCDR3-LCDR1-LCDR2-LCDR3 set forth in any one of SEQ ID NOs: 1-240. In some embodiments, an AAV capsid as described herein comprises a targeting ligand that binds human CACNG1, wherein the targeting ligand comprises a heavy chain variable domain, light chain variable domain, heavy chain variable domain/light chain variable domain pair, HCDR1, HCDR2, CDR3, LCDR1, LCDR2, LCDR3, and/or set of HCDR1-HCDR2-HCDR3-LCDR1-LCDR2-LCDR3 amino acid sequences 99% identical to amino acid sequences of a heavy chain variable domain, light chain variable domain, heavy chain variable domain/light chain variable domain pair, HCDR1, HCDR2, HCDR3, LCDR1, LCDR2, LCDR3, and/or set of HCDR1-HCDR2-HCDR3-LCDR1-LCDR2-LCDR3 set forth in any one of SEQ ID NOs: 1-240. Also described herein are antibodies, or antigen-binding fragments thereof, comprising a set of six CDRs (i.e., HCDR1-HCDR2-HCDR3-LCDR1-LCDR2-LCDR3) contained within an HCVR/LCVR amino acid sequence pair as defined by any of the exemplary anti-hCACNG1 antibodies listed in Table 1. In some embodiments, a targeting ligand as described herein comprises the HCDR1-HCDR2-HCDR3-LCDR1-LCDR2-LCDR3 amino acid sequences set contained within an HCVR/LCVR amino acid sequence pair selected from the group consisting of SEQ ID NOs: 2/10, SEQ ID NOs: 18/26, SEQ ID NOs: 34/42, SEQ ID NOs: 50/58, SEQ ID NOs: 66/74, SEQ ID NOs: 82/90, SEQ ID NOs: 98/106, SEQ ID NOs: 114/122, SEQ ID NOs: 130/138, SEQ ID NOs: 146/154, SEQ ID NOs: 162/170, and SEQ ID NOs: 178/186. In certain embodiments, a targeting ligand as described herein comprises an HCVR/LCVR amino acid sequence pair is selected from the group consisting of SEQ ID NOs: 2/10, SEQ ID NOs: 18/26, SEQ ID NOs: 34/42, SEQ ID NOs: 50/58, SEQ ID NOs: 66/74, SEQ ID NOs: 82/90, SEQ ID NOs: 98/106, SEQ ID NOs: 114/122, SEQ ID NOs: 130/138, SEQ ID NOs: 146/154, SEQ ID NOs: 162/170, and SEQ ID NOs: 178/186.

TABLE 1
SEQ ID NOs of Domains in Antibodies, Antigen-binding Fragments (e.g., Fabs)
or scFv Molecules that may be used to retarget AAV to human CACNG1.

| | HCVR | HCDR1 | HCDR2 | HCDR3 | LCVR | LCDR1 | LCDR2 | LCDR3 |
|---|---|---|---|---|---|---|---|---|
| Antibody | NA | NA | NA | NA | NA | NA | NA | NA |
| Designation | (AA) | (AA) | (AA) | (AA) | (AA) | (AA) | (AA) | (AA) |
| 1 | 1 | 3 | 5 | 7 | 9 | 11 | 13 | 15 |
| 31929/14647/10728 | (2) | (4) | (6) | (8) | (10) | (12) | (14) | (16) |
| 2 | 17 | 19 | 21 | 23 | 25 | 27 | 29 | 31 |
| 14570/10715 | (18) | (20) | (22) | (24) | (26) | (28) | (30) | (32) |
| 3 | 33 | 35 | 37 | 39 | 41 | 43 | 45 | 47 |
| 14572/10717 | (34) | (36) | (38) | (40) | (42) | (44) | (46) | (48) |
| 4 | 49 | 51 | 53 | 55 | 57 | 59 | 61 | 63 |
| 14571/10716 | (50) | (52) | (54) | (56) | (58) | (60) | (62) | (64) |
| 5 | 65 | 67 | 69 | 71 | 73 | 75 | 77 | 79 |
| 14574/10783 | (66) | (68) | (70) | (72) | (74) | (76) | (78) | (80) |
| 6 | 81 | 83 | 85 | 87 | 89 | 91 | 93 | 95 |
| 31944 | (82) | (84) | (86) | (88) | (90) | (92) | (94) | (96) |
| 7 | 97 | 99 | 101 | 103 | 105 | 107 | 109 | 111 |
| 31265/5972 | (98) | (100) | (102) | (104) | (106) | (108) | (110) | (112) |
| 8 | 113 | 115 | 117 | 119 | 121 | 123 | 125 | 127 |
| 31941 | (114) | (116) | (118) | (120) | (122) | (124) | (126) | (128) |
| 9 | 129 | 131 | 133 | 135 | 137 | 139 | 141 | 143 |
| 7660 | (130) | (132) | (134) | (136) | (138) | (140) | (142) | (144) |
| 10 | 145 | 147 | 149 | 151 | 153 | 155 | 157 | 159 |
| 9909 | (146) | (148) | (150) | (152) | (154) | (156) | (158) | (160) |
| 11 | 161 | 163 | 165 | 167 | 169 | 171 | 173 | 175 |
| 14573/10713 | (162) | (164) | (166) | (168) | (170) | (172) | (174) | (176) |

HCVR Nucleic Acid Sequence
(SEQ ID NO: 1)
CAGGTGCAGCTGGTGGAGTCTGGGGGAGGCGTGGTCCAGCCTGGGAGGTCCCTGAGACTC
TCCTGTACAGCGTCTGGAATCACCTTCAGAAATTATGGCATGCACTGGGTCCGCCAGGCT
CCAGGCAAGGGGCTGGAGTGGGTGGCAGTTATGTGGTATGATGGAAGTAATAAGTACTAT
GCAGACTCCGTGAAGGGCCGTTTCACCATCTCCGGAGACAATTCCAAGGTGTATCTGCAA
ATGAACAGCCTGAGAGCCGAGGACACGGCTGTATATTACTGTGCGAGAAGGGGCACTATA
AGAACAGCTGCCCCTTTTGACTACTGGGGTCAGGGAACCCTGGTCACCGTCTCCTCA
HCVR Amino Acid Sequence
(SEQ ID NO: 2)
QVQLVESGGGVVQPGRSLRLSCTASGITFRNYGMHWVRQAPGKGLEWVAVMWYDGSNKYY
ADSVKGRFTISGDNSKVYLQMNSLRAEDTAVYYCARRGTIRTAAPFDYWGQGTLVTVSS
HCDR1 Nucleic Acid Sequence
(SEQ ID NO: 3)
GGA ATC ACC TTC AGA AAT TAT GGC
HCDR1 Amino Acid Sequence
(SEQ ID NO: 4)
G I T F R N Y G
HCDR2 Nucleic Acid Sequence
(SEQ ID NO: 5)
ATG TGG TAT GAT GGA AGT AAT AAG
HCDR2 Amino Acid Sequence
(SEQ ID NO: 6)
M W Y D G S N K
HCDR3 Nucleic Acid Sequence
(SEQ ID NO: 7)
GCG AGA AGG GGC ACT ATA AGA ACA GCT GCC CCT TTT GAC TAC
HCDR3 Amino Acid Sequence
(SEQ ID NO: 8)
A R R G T I R T A A P F D Y
LCVR Nucleic Acid Sequence
(SEQ ID NO: 9)
GACATCCAGATGACCCAGTCTCCATCCTCCCTGTCTGCATCTGTAGGAGACAGAGTCACC
ATCACTTGCCGGGCAAGTCAGAGCATTAGCAGCTATTTAAATTGGTATCAGCAGAAACCA
GGGAAAGCCCCTAAGCTCCTGATCTATGCTGCATCCAGTTTGCAAAGTGGGGTCCCGTCA
AGGTTCAGTGGCAGTGGATCTGGGACAGATTTCACTCTCACCATCAGCAGTCTGCAACCT
GAAGATTTTGCAACTTACTACTGTCAACAGAGTTACAGTACCCCTCCGATCACCTTCGGC
CAAGGGACACGACTGGAGATTAAA
LCVR Amino Acid Sequence
(SEQ ID NO: 10)
DIQMTQSPSSLSASVGDRVTITCRASQSISSYLNWYQQKPGKAPKLLIYAASSLQSGVPS
RFSGSGSGTDFTLTISSLQPEDFATYYCQQSYSTPPITFGQGTRLEIK
LCDR1 Nucleic Acid Sequence
(SEQ ID NO: 11)
CAG AGC ATT AGC AGC TAT
LCDR1 Amino Acid Sequence
(SEQ ID NO: 12)
Q S I S S Y
LCDR2 Nucleic Acid Sequence
(SEQ ID NO: 13)
GCT GCA TCC
LCDR2 Amino Acid Sequence
(SEQ ID NO: 14)
A A S
LCDR3 Nucleic Acid Sequence
(SEQ ID NO: 15)
CAA CAG AGT TAC AGT ACC CCT CCG ATC ACC
LCDR3 Amino Acid Sequence
(SEQ ID NO: 16)
Q Q S Y S T P P I T
HC Nucleic Acid Sequence
(SEQ ID NO: 193)
CAGGTGCAGCTGGTGGAGTCTGGGGGAGGCGTGGTCCAGCCTGGGAGGTCCCTGAGACTC
TCCTGTACAGCGTCTGGAATCACCTTCAGAAATTATGGCATGCACTGGGTCCGCCAGGCT
CCAGGCAAGGGGCTGGAGTGGGTGGCAGTTATGTGGTATGATGGAAGTAATAAGTACTAT
GCAGACTCCGTGAAGGGCCGTTTCACCATCTCCGGAGACAATTCCAAGGTGTATCTGCAA
ATGAACAGCCTGAGAGCCGAGGACACGGCTGTATATTACTGTGCGAGAAGGGGCACTATA
AGAACAGCTGCCCCTTTTGACTACTGGGGTCAGGGAACCCTGGTCACCGTCTCCTCAGCC
TCCACCAAGGGCCCATCGGTCTTCCCCCTGGCGCCCTGCTCCAGGAGCACCTCCGAGAGC
ACAGCCGCCCTGGGCTGCCTGGTCAAGGACTACTTCCCCGAACCGGTGACGGTGTCGTGG
AACTCAGGCGCCCTGACCAGCGGCGTGCACACCTTCCCGGCTGTCCTACAGTCCTCAGGA
CTCTACTCCCTCAGCAGCGTGGTGACCGTGCCCTCCAGCAGCTTGGGCACGAAGACCTAC
ACCTGCAACGTAGATCACAAGCCCAGCAACACCAAGGTGGACAAGAGAGTTGAGTCCAAA
TATGGTCCCCCATGCCCACCGTGCCCAGCACCAGGCGGTGGCGGACCATCAGTCTTCCTG
TTCCCCCCAAAACCCAAGGACACTCTCATGATCTCCCGGACCCCTGAGGTCACGTGCGTG
GTGGTGGACGTGAGCCAGGAAGACCCCGAGGTCCAGTTCAACTGGTACGTGGATGGCGTG
GAGGTGCATAATGCCAAGACAAAGCCGCGGGAGGAGCAGTTCAACAGCACGTACCGTGTG
GTCAGCGTCCTCACCGTCCTGCACCAGGACTGGCTGAACGGCAAGGAGTACAAGTGCAAG
GTCTCCAACAAAGGCCTCCCGTCCTCCATCGAGAAAACCATCTCCAAAGCCAAAGGGCAG
CCCCGAGAGCCACAGGTGTACACCCTGCCCCCATCCCAGGAGGAGATGACCAAGAACCAG
GTCAGCCTGACCTGCCTGGTCAAAGGCTTCTACCCCAGCGACATCGCCGTGGAGTGGGAG
AGCAATGGGCAGCCGGAGAACAACTACAAGACCACGCCTCCCGTGCTGGACTCCGACGGC
TCCTTCTTCCTCTACAGCAGGCTCACCGTGGACAAGAGCAGGTGGCAGGAGGGGAATGTC
TTCTCATGCTCCGTGATGCATGAGGCTCTGCACAACCACTACACACAGAAGTCCCTCTCC
CTGTCTCTGGGTAAATGA
HC Amino Acid Sequence
(SEQ ID NO: 194)
QVQLVESGGGVVQPGRSLRLSCTASGITFRNYGMHWVRQAPGKGLEWVAVMWYDGSNKYY
ADSVKGRFTISGDNSKVYLQMNSLRAEDTAVYYCARRGTIRTAAPFDYWGQGTLVTVSSA
STKGPSVFPLAPCSRSTSESTAALGCLVKDYFPEPVTVSWNSGALTSGVHTFPAVLQSSG
LYSLSSVVTVPSSSLGTKTYTCNVDHKPSNTKVDKRVESKYGPPCPPCPAPGGGGPSVFL
FPPKPKDTLMISRTPEVTCVVVDVSQEDPEVQFNWYVDGVEVHNAKTKPREEQFNSTYRV
VSVLTVLHQDWLNGKEYKCKVSNKGLPSSIEKTISKAKGQPREPQVYTLPPSQEEMTKNQ
VSLTCLVKGFYPSDIAVEWESNGQPENNYKTTPPVLDSDGSFFLYSRLTVDKSRWQEGNV
FSCSVMHEALHNHYTQKSLSLSLGK
*underlined and bolded asparagine (N) may be mutated to a
glutamine (Q) for conjugation by transglutaminase,
see, e.g., SEQ ID NO: 269
LC Nucleic Acid Sequence
(SEQ ID NO: 195)
GACATCCAGATGACCCAGTCTCCATCCTCCCTGTCTGCATCTGTAGGAGACAGAGTCACC
ATCACTTGCCGGGCAAGTCAGAGCATTAGCAGCTATTTAAATTGGTATCAGCAGAAACCA
GGGAAAGCCCCTAAGCTCCTGATCTATGCTGCATCCAGTTTGCAAAGTGGGGTCCCGTCA
AGGTTCAGTGGCAGTGGATCTGGGACAGATTTCACTCTCACCATCAGCAGTCTGCAACCT
GAAGATTTTGCAACTTACTACTGTCAACAGAGTTACAGTACCCCTCCGATCACCTTCGGC
CAAGGGACACGACTGGAGATTAAACGAACTGTGGCTGCACCATCTGTCTTCATCTTCCCG
CCATCTGATGAGCAGTTGAAATCTGGAACTGCCTCTGTTGTGTGCCTGCTGAATAACTTC
TATCCCAGAGAGGCCAAAGTACAGTGGAAGGTGGATAACGCCCTCCAATCGGGTAACTCC
CAGGAGAGTGTCACAGAGCAGGACAGCAAGGACAGCACCTACAGCCTCAGCAGCACCCTG
ACGCTGAGCAAAGCAGACTACGAGAAACACAAAGTCTACGCCTGCGAAGTCACCCATCAG
GGCCTGAGCTCGCCCGTCACAAAGAGCTTCAACAGGGGAGAGTGTTAG
LC Amino Acid Sequence
(SEQ ID NO: 196)
DIQMTQSPSSLSASVGDRVTITCRASQSISSYLNWYQQKPGKAPKLLIYAASSLQSGVPS
RFSGSGSGTDFTLTISSLQPEDFATYYCQQSYSTPPITFGQGTRLEIKRTVAAPSVFIFP
PSDEQLKSGTASVVCLLNNFYPREAKVQWKVDNALQSGNSQESVTEQDSKDSTYSLSSTL
TLSKADYEKHKVYACEVTHQGLSSPVTKSFNRGEC
10715 (wildtype hIgG1) /14570 (IgG1 N180Q):
HCVR Nucleic Acid Sequence
(SEQ ID NO: 17)
CAGGTGCAGCTACAGCAGTGGGGCGCAGGACTGTTGAAGCCTTCGGCGACCCTGTCCCGC
ACCTGCGCTGTCTATGGTGGGTCCTTCAGTGGTTACTACTGGAACTGGATCCGCCAGTCC
CCAGGGAAGGGGCTGGAATGGATTGGGGAAATCCTTCATAGTGGAAGAACCAACTACAAC
CCGTCCCTCAAGAGTCGAGTCACCATATCAGTAGACACGTCCAAGAACCAGTTCTCCCTG
AAGCTGACCTCTGTGACCGCCGCGGACACGGCTGTATATTACTGTGCGGGAAGGATAGCA
GCTCGTCACGGCTGGTTCGACCCCTGGGGCCAGGGAACCCTGGTCACCGTCTCCTCA
HCVR Amino Acid Sequence
(SEQ ID NO: 18)
QVQLQQWGAGLLKPSATLSRTCAVYGGSFSGYYWNWIRQSPGKGLEWIGEILHSGRTNYN
PSLKSRVTISVDTSKNQFSLKLTSVTAADTAVYYCAGRIAARHGWFDPWGQGTLVTVSS
HCDR1 Nucleic Acid Sequence
(SEQ ID NO: 19)
GGT GGG TCC TTC AGT GGT TAC TAC
HCDR1 Amino Acid Sequence
(SEQ ID NO: 20)
G G S F S G Y Y
HCDR2 Nucleic Acid Sequence
(SEQ ID NO: 21)
ATC CTT CAT AGT GGA AGA ACC
HCDR2 Amino Acid Sequence
(SEQ ID NO: 22)
I L H S G R T
HCDR3 Nucleic Acid Sequence
(SEQ ID NO: 23)
GCG GGA AGG ATA GCA GCT CGT CAC GGC TGG TTC GAC CCC
HCDR3 Amino Acid Sequence
(SEQ ID NO: 24)
A G R I A A R H G W F D P
LCVR Nucleic Acid Sequence
(SEQ ID NO: 25)
GACATCCAGATGACCCAGTCTCCATCTTCCGTGTCTACATCTGTAGGAGACAGAGTCACC
ATCTCTTGTCGGGCGAGTCAGGATATTCGCAAGTGGTTAGCCTGGTATCAACAGAAACCA
GGAAAAGCCCCTAAACTCCTGATCTATGCTACATCCAGTTTGCAAAGTGGGGTCCCTTCA
AGGTTCAGCGGCAGTGGATCTGGGACAGATTTCACTCTCACCATCAGCAGCCTGCAGCCT
GAGGATTTTGCAACTTACTTTTGTCAACAGGCTAACAGTTTCCCGTTCACTTTTGGCCAG
GGGACCAAGCTGGAGATCAAA
LCVR Amino Acid Sequence
(SEQ ID NO: 26)
DIQMTQSPSSVSTSVGDRVTISCRASQDIRKWLAWYQQKPGKAPKLLIYATSSLQSGVPS
RFSGSGSGTDFTLTISSLQPEDFATYFCQQANSFPFTFGQGTKLEIK
LCDR1 Nucleic Acid Sequence
(SEQ ID NO: 27)
CAG GAT ATT CGC AAG TGG
LCDR1 Amino Acid Sequence
(SEQ ID NO: 28)
Q D I R K W
LCDR2 Nucleic Acid Sequence
(SEQ ID NO: 29)
GCT ACA TCC
LCDR2 Amino Acid Sequence
(SEQ ID NO: 30)
A T S
LCDR3 Nucleic Acid Sequence
(SEQ ID NO: 31)
CAA CAG GCT AAC AGT TTC CCG TTC ACT
LCDR3 Amino Acid Sequence
(SEQ ID NO: 32)
Q Q A N S F P F T
HC Nucleic Acid Sequence
(SEQ ID NO: 197)
CAGGTGCAGCTACAGCAGTGGGGCGCAGGACTGTTGAAGCCTTCGGCGACCCTGTCCCGC
ACCTGCGCTGTCTATGGTGGGTCCTTCAGTGGTTACTACTGGAACTGGATCCGCCAGTCC
CCAGGGAAGGGGCTGGAATGGATTGGGGAAATCCTTCATAGTGGAAGAACCAACTACAAC
CCGTCCCTCAAGAGTCGAGTCACCATATCAGTAGACACGTCCAAGAACCAGTTCTCCCTG
AAGCTGACCTCTGTGACCGCCGCGGACACGGCTGTATATTACTGTGCGGGAAGGATAGCA
GCTCGTCACGGCTGGTTCGACCCCTGGGGCCAGGGAACCCTGGTCACCGTCTCCTCAGCC
TCCACCAAGGGCCCATCGGTCTTCCCCCTGGCGCCCTGCTCCAGGAGCACCTCCGAGAGC
ACAGCCGCCCTGGGCTGCCTGGTCAAGGACTACTTCCCCGAACCGGTGACGGTGTCGTGG
AACTCAGGCGCCCTGACCAGCGGCGTGCACACCTTCCCGGCTGTCCTACAGTCCTCAGGA
CTCTACTCCCTCAGCAGCGTGGTGACCGTGCCCTCCAGCAGCTTGGGCACGAAGACCTAC
ACCTGCAACGTAGATCACAAGCCCAGCAACACCAAGGTGGACAAGAGAGTTGAGTCCAAA
TATGGTCCCCCATGCCCACCCTGCCCAGCACCTGAGTTCCTGGGGGGACCATCAGTCTTC
CTGTTCCCCCCAAAACCCAAGGACACTCTCATGATCTCCCGGACCCCTGAGGTCACGTGC
GTGGTGGTGGACGTGAGCCAGGAAGACCCCGAGGTCCAGTTCAACTGGTACGTGGATGGC
GTGGAGGTGCATAATGCCAAGACAAAGCCGCGGGAGGAGCAGTTCAACAGCACGTACCGT
GTGGTCAGCGTCCTCACCGTCCTGCACCAGGACTGGCTGAACGGCAAGGAGTACAAGTGC
AAGGTCTCCAACAAAGGCCTCCCGTCCTCCATCGAGAAAACCATCTCCAAAGCCAAAGGG
CAGCCCCGAGAGCCACAGGTGTACACCCTGCCCCCATCCCAGGAGGAGATGACCAAGAAC
CAGGTCAGCCTGACCTGCCTGGTCAAAGGCTTCTACCCCAGCGACATCGCCGTGGAGTGG
GAGAGCAATGGGCAGCCGGAGAACAACTACAAGACCACGCCTCCCGTGCTGGACTCCGAC
GGCTCCTTCTTCCTCTACAGCAGGCTCACCGTGGACAAGAGCAGGTGGCAGGAGGGGAAT
GTCTTCTCATGCTCCGTGATGCATGAGGCTCTGCACAACCACTACACACAGAAGTCCCTC
TCCCTGTCTCTGGGTAAATGA
HC Amino Acid Sequence
(SEQ ID NO: 198)
QVQLQQWGAGLLKPSATLSRTCAVYGGSFSGYYWNWIRQSPGKGLEWIGEILHSGRTNYN
PSLKSRVTISVDTSKNQFSLKLTSVTAADTAVYYCAGRIAARHGWFDPWGQGTLVTVSSA
STKGPSVFPLAPCSRSTSESTAALGCLVKDYFPEPVTVSWNSGALTSGVHTFPAVLQSSG
LYSLSSVVTVPSSSLGTKTYTCNVDHKPSNTKVDKRVESKYGPPCPPCPAPEFLGGPSVF
LFPPKPKDTLMISRTPEVTCVVVDVSQEDPEVQFNWYVDGVEVHNAKTKPREEQFNSTYR
VVSVLTVLHQDWLNGKEYKCKVSNKGLPSSIEKTISKAKGQPREPQVYTLPPSQEEMTKN
QVSLTCLVKGFYPSDIA VEWESNGQPENNYKTTPPVLDSDGSFFLYSRLTVDKSRWQEG
NVFSCSVMHEALHNHYTQKSLSLSLGK
*underlined and bolded asparagine (N) may be mutated to a glutamine
(Q) for conjugation by transglutaminase, see, e.g., SEQ ID NO: 269
LC Nucleic Acid Sequence
(SEQ ID NO: 199)
GACATCCAGATGACCCAGTCTCCATCTTCCGTGTCTACATCTGTAGGAGACAGAGTCACC
ATCTCTTGTCGGGCGAGTCAGGATATTCGCAAGTGGTTAGCCTGGTATCAACAGAAACCA
GGAAAAGCCCCTAAACTCCTGATCTATGCTACATCCAGTTTGCAAAGTGGGGTCCCTTCA
AGGTTCAGCGGCAGTGGATCTGGGACAGATTTCACTCTCACCATCAGCAGCCTGCAGCCT
GAGGATTTTGCAACTTACTTTTGTCAACAGGCTAACAGTTTCCCGTTCACTTTTGGCCAG
GGGACCAAGCTGGAGATCAAACGAACTGTGGCTGCACCATCTGTCTTCATCTTCCCGCCA
TCTGATGAGCAGTTGAAATCTGGAACTGCCTCTGTTGTGTGCCTGCTGAATAACTTCTAT
CCCAGAGAGGCCAAAGTACAGTGGAAGGTGGATAACGCCCTCCAATCGGGTAACTCCCAG
GAGAGTGTCACAGAGCAGGACAGCAAGGACAGCACCTACAGCCTCAGCAGCACCCTGACG
CTGAGCAAAGCAGACTACGAGAAACACAAAGTCTACGCCTGCGAAGTCACCCATCAGGGC
CTGAGCTCGCCCGTCACAAAGAGCTTCAACAGGGGAGAGTGTTAG
LC Amino Acid Sequence
(SEQ ID NO: 200)
DIQMTQSPSSVSTSVGDRVTISCRASQDIRKWLAWYQQKPGKAPKLLIYATSSLQSGVPS
RFSGSGSGTDFTLTISSLQPEDFATYFCQQANSFPFTFGQGTKLEIKRTVAAPSVFIFPP
SDEQLKSGTASVVCLLNNFYPREAKVQWKVDNALQSGNSQESVTEQDSKDSTYSLSSTLT
LSKADYEKHKVYACEVTHQGLSSPVTKSFNRGEC
10717 (wildtype hIgG1)/ 14572 (hIgG1 N180Q)
HCVR Nucleic Acid Sequence
(SEQ ID NO: 33)
CAGGTGCAGCTGGTGGAGTCTGGGGGAGGCGTGGTCCAGCCTGGGAGGTCCCTGAGACTC
TCCTGTGCAGCGTCTGGATTCACCTTCAGTACATATGGCATGCACTGGGTCCGCCAGGCT
CCAGGCAAGGGGCTGGAGTGGGTGGCAGTTATTTGGCATGATGGAAGTGATAAATATTAT
GTAGACTCCGTGAAGGGCCGATTCTCCATCGCCAGAGACAATTCCAAGAACACGCTTTAT
CTGCAAATGAATAGTCTGAGAGTCGAGGACACGGGTATATATTACTGTGCGAGAAGGGGT
ATACGTGGAACCGTTTTTGACCACTGGGGCCTGGGAACCCTGGTCACCGTCTCCTCA
HCVR Amino Acid Sequence
(SEQ ID NO: 34)
QVQLVESGGGVVQPGRSLRLSCAASGFTFSTYGMHWVRQAPGKGLEWVAVIWHDGSDKYY
VDSVKGRFSIARDNSKNTLYLQMNSLR VEDTGIYYCARRGIRGTVFDHWGLGTLVTVSS
HCDR1 Nucleic Acid Sequence
(SEQ ID NO: 35)
GGA TTC ACC TTC AGT ACA TAT GGC
HCDR1 Amino Acid Sequence
(SEQ ID NO: 36)
G F T F S T Y G
HCDR2 Nucleic Acid Sequence
(SEQ ID NO: 37)
ATT TGG CAT GAT GGA AGT GAT AAA
HCDR2 Amino Acid Sequence
(SEQ ID NO: 38)
I W H D G S D K
HCDR3 Nucleic Acid Sequence
(SEQ ID NO: 39)
GCG AGA AGG GGT ATA CGT GGA ACC GTT TTT GAC CAC
HCDR3 Amino Acid Sequence
(SEQ ID NO: 40)
A R R G I R G T V F D H
LCVR Nucleic Acid Sequence
(SEQ ID NO: 41)
GACATCCAGATGACCCAGTCTCCTTCCACCCTGTCTGCATCTGTAGGAGACAGAGTCACC
CTCACTTGTCGGGCCAGTCAGAGTATTAGTAACAAGTTGGCCTGGTATCAGCAGAAACCA
GGGAAAGCCCCTAACCTCCTGATCTATAAGGCGTCTAATTTAGAAAGTGGGGTCCCATCA
AGGTTCAGCGGCAGTGGATCTGGGACAGAATTCACTCTCACCATCAGCAGCCTGCAGCCT
GATGATTTTGCAACTTATTACTGCCAACAGTATAATAGTTATTCGTGGACGTTCGGCCAA
GGGACCAAGGTGGAAATCAAA
LCVR Amino Acid Sequence
(SEQ ID NO: 42)
DIQMTQSPSTLSASVGDRVTLTCRASQSISNKLAWYQQKPGKAPNLLIYKASNLESGVPS
RFSGSGSGTEFTLTISSLQPDDFATYYCQQYNSYSWTFGQGTKVEIK
LCDR1 Nucleic Acid Sequence
(SEQ ID NO: 43)
CAG AGT ATT AGT AAC AAG
LCDR1 Amino Acid Sequence
(SEQ ID NO: 44)
Q S I S N K
LCDR2 Nucleic Acid Sequence
(SEQ ID NO: 45)
AAG GCG TCT
LCDR2 Amino Acid Sequence
(SEQ ID NO: 46)
K A S
LCDR3 Nucleic Acid Sequence
(SEQ ID NO: 47)
CAA CAG TAT AAT AGT TAT TCG TGG ACG
LCDR3 Amino Acid Sequence
(SEQ ID NO: 48)
Q Q Y N S Y S W T
HC Nucleic Acid Sequence
(SEQ ID NO: 201)
CAGGTGCAGCTGGTGGAGTCTGGGGGAGGCGTGGTCCAGCCTGGGAGGTCCCTGAGACTC
TCCTGTGCAGCGTCTGGATTCACCTTCAGTACATATGGCATGCACTGGGTCCGCCAGGCT
CCAGGCAAGGGGCTGGAGTGGGTGGCAGTTATTTGGCATGATGGAAGTGATAAATATTAT
GTAGACTCCGTGAAGGGCCGATTCTCCATCGCCAGAGACAATTCCAAGAACACGCTTTAT
CTGCAAATGAATAGTCTGAGAGTCGAGGACACGGGTATATATTACTGTGCGAGAAGGGGT
ATACGTGGAACCGTTTTTGACCACTGGGGCCTGGGAACCCTGGTCACCGTCTCCTCAGCC
TCCACCAAGGGCCCATCGGTCTTCCCCCTGGCGCCCTGCTCCAGGAGCACCTCCGAGAGC
ACAGCCGCCCTGGGCTGCCTGGTCAAGGACTACTTCCCCGAACCGGTGACGGTGTCGTGG
AACTCAGGCGCCCTGACCAGCGGCGTGCACACCTTCCCGGCTGTCCTACAGTCCTCAGGA
CTCTACTCCCTCAGCAGCGTGGTGACCGTGCCCTCCAGCAGCTTGGGCACGAAGACCTAC
ACCTGCAACGTAGATCACAAGCCCAGCAACACCAAGGTGGACAAGAGAGTTGAGTCCAAA
TATGGTCCCCCATGCCCACCCTGCCCAGCACCTGAGTTCCTGGGGGGACCATCAGTCTTC
CTGTTCCCCCCAAAACCCAAGGACACTCTCATGATCTCCCGGACCCCTGAGGTCACGTGC
GTGGTGGTGGACGTGAGCCAGGAAGACCCCGAGGTCCAGTTCAACTGGTACGTGGATGGC
GTGGAGGTGCATAATGCCAAGACAAAGCCGCGGGAGGAGCAGTTCAACAGCACGTACCGT
GTGGTCAGCGTCCTCACCGTCCTGCACCAGGACTGGCTGAACGGCAAGGAGTACAAGTGC
AAGGTCTCCAACAAAGGCCTCCCGTCCTCCATCGAGAAAACCATCTCCAAAGCCAAAGGG
CAGCCCCGAGAGCCACAGGTGTACACCCTGCCCCCATCCCAGGAGGAGATGACCAAGAAC
CAGGTCAGCCTGACCTGCCTGGTCAAAGGCTTCTACCCCAGCGACATCGCCGTGGAGTGG
GAGAGCAATGGGCAGCCGGAGAACAACTACAAGACCACGCCTCCCGTGCTGGACTCCGAC
GGCTCCTTCTTCCTCTACAGCAGGCTCACCGTGGACAAGAGCAGGTGGCAGGAGGGGAAT
GTCTTCTCATGCTCCGTGATGCATGAGGCTCTGCACAACCACTACACACAGAAGTCCCTC
TCCCTGTCTCTGGGTAAATGA
HC Amino Acid Sequence
(SEQ ID NO: 202)
QVQLVESGGGVVQPGRSLRLSCAASGFTFSTYGMHWVRQAPGKGLEWVAVIWHDGSDKYY
VDSVKGRFSIARDNSKNTLYLQMNSLRVEDTGIYYCARRGIRGTVFDHWGLGTLVTVSSA
STKGPSVFPLAPCSRSTSESTAALGCLVKDYFPEPVTVSWNSGALTSGVHTFPAVLQSSG
LYSLSSVVTVPSSSLGTKTYTCNVDHKPSNTKVDKRVESKYGPPCPPCPAPEFLGGPSVF
LFPPKPKDTLMISRTPEVTCVVVDVSQEDPEVQFNWYVDGVEVHNAKTKPREEQFNSTYR
VVSVLTVLHQDWLNGKEYKCKVSNKGLPSSIEKTISKAKGQPREPQVYTLPPSQEEMTKN
QVSLTCLVKGFYPSDIA VEWESNGQPENNYKTTPPVLDSDGSFFLYSRLTVDKSRWQEG
NVFSCSVMHEALHNHYTQKSLSLSLGK
*underlined and bolded asparagine (N)
may be mutated to a glutamine (Q) for
conjugation by transglutaminase, see, e.g., SEQ ID NO: 269
LC Nucleic Acid Sequence
(SEQ ID NO: 203)
GACATCCAGATGACCCAGTCTCCTTCCACCCTGTCTGCATCTGTAGGAGACAGAGTCACC
CTCACTTGTCGGGCCAGTCAGAGTATTAGTAACAAGTTGGCCTGGTATCAGCAGAAACCA
GGGAAAGCCCCTAACCTCCTGATCTATAAGGCGTCTAATTTAGAAAGTGGGGTCCCATCA
AGGTTCAGCGGCAGTGGATCTGGGACAGAATTCACTCTCACCATCAGCAGCCTGCAGCCT
GATGATTTTGCAACTTATTACTGCCAACAGTATAATAGTTATTCGTGGACGTTCGGCCAA
GGGACCAAGGTGGAAATCAAACGAACTGTGGCTGCACCATCTGTCTTCATCTTCCCGCCA
TCTGATGAGCAGTTGAAATCTGGAACTGCCTCTGTTGTGTGCCTGCTGAATAACTTCTAT
CCCAGAGAGGCCAAAGTACAGTGGAAGGTGGATAACGCCCTCCAATCGGGTAACTCCCAG
GAGAGTGTCACAGAGCAGGACAGCAAGGACAGCACCTACAGCCTCAGCAGCACCCTGACG
CTGAGCAAAGCAGACTACGAGAAACACAAAGTCTACGCCTGCGAAGTCACCCATCAGGGC
CTGAGCTCGCCCGTCACAAAGAGCTTCAACAGGGGAGAGTGTTAG
LC Amino Acid Sequence
(SEQ ID NO: 204)
DIQMTQSPSTLSASVGDRVTLTCRASQSISNKLAWYQQKPGKAPNLLIYKASNLESGVPS
RFSGSGSGTEFTLTISSLQPDDFATYYCQQYNSYSWTFGQGTK VEIKRTVAAPSVFIFP
PSDEQLKSGTASVVCLLNNFYPREAKVQWKVDNALQSGNSQESVTEQDSKDSTYSLSSTL
TLSKADYEKHKVYACEVTHQGLSSPVTKSFNRGEC
10716 (wildtype hIgG1)/ 14571 (hIgG1 N180Q)
HCVR Nucleic Acid Sequence
(SEQ ID NO: 49)
CAGGTGCAGCTGCAGGAGTCGGGCCCAGGACTGGTGAAGCCTTCGGAGACCCTGTCCCTC
ACCTGCACTGTCTCTGGTGACTCCATCAATAATTACTACTGGACCTGGCTCCGGCAGCCC
CCAGGGAAGGGACTGGAGTGGATTGGTTATATCTATTACAGTGGGAGCGCCAACTACAAC
CCCTCCCTCAAGAGTCGAGTCACCATATCAGTAGACACGTCCAAGAACCAGTTCTCCCTG
AAGCTAAATTCTGTGACCGCTGCGGACACGGCCGTGTATTACTGTGCGAGAGGGGCGGTC
AAGTACTTCCGGCATTGGGGCCAGGGCACCCTGGTCACCGTCTCCTCA
HCVR Amino Acid Sequence
(SEQ ID NO: 50)
QVQLQESGPGLVKPSETLSLTCTVSGDSINNYYWTWLRQPPGKGLEWIGYIYYSGSANYN
PSLKSRVTISVDTSKNQFSLKLNSVTAADTAVYYCARGAVKYFRHWGQGTLVTVSS
HCDR1 Nucleic Acid Sequence
(SEQ ID NO: 51)
GGT GAC TCC ATC AAT AAT TAC TAC
HCDR1 Amino Acid Sequence
(SEQ ID NO: 52)
G D S I N N Y Y
HCDR2 Nucleic Acid Sequence
(SEQ ID NO: 53)
ATC TAT TAC AGT GGG AGC GCC
HCDR2 Amino Acid Sequence
(SEQ ID NO: 54)
I Y Y S G S A
HCDR3 Nucleic Acid Sequence
(SEQ ID NO: 55)
GCG AGA GGG GCG GTC AAG TAC TTC CGG CAT
HCDR3 Amino Acid Sequence
(SEQ ID NO: 56)
A R G A V K Y F R H
LCVR Nucleic Acid Sequence
(SEQ ID NO: 57)
GAAATTGTGTTGACGCAGTCTCCGGGCACCCTCTCTTTGTCTCCAGGGGAAAGAGCCACC
CTCTCCTGCAGGGCCAGTCAGACTATTAACCACAACAACTTAGCCTGGTACCAGCAGAGA
CCTGGCCAGGCTCCCAGGCTCCTCATCTATGGTGCATCCAACAGGGCCACTGCCATCCCA
GACAGGTTCAGTGGCAGTGGGTCTGGGACAGACTTCACTCTCACCATCAGCAGACTGGAG
CCTGAAGATTTTGAAGTGTATTCTTGTCAGCAGTATGGTAGCTTGCCGCTCACTTTCGGC
GGAGGGACCAAGGTGGAGATCAAA
LCVR Amino Acid Sequence
(SEQ ID NO: 58)
EIVLTQSPGTLSLSPGERATLSCRASQTINHNNLAWYQQRPGQAPRLLIYGASNRATAIP
DRFSGSGSGTDFTLTISRLEPEDFEVYSCQQYGSLPLTFGGGTKVEIK
LCDR1 Nucleic Acid Sequence
(SEQ ID NO: 59)
CAG ACT ATT AAC CAC AAC AAC
LCDR1 Amino Acid Sequence
(SEQ ID NO: 60)
Q T I N H N N
LCDR2 Nucleic Acid Sequence
(SEQ ID NO: 61)
GGT GCA TCC
LCDR2 Amino Acid Sequence
(SEQ ID NO: 62)
G A S
LCDR3 Nucleic Acid Sequence
(SEQ ID NO: 63)
CAG CAG TAT GGT AGC TTG CCG CTC ACT
LCDR3 Amino Acid Sequence
(SEQ ID NO: 64)
Q Q Y G S L P L T
HC Nucleic Acid Sequence
(SEQ ID NO: 205)
CAGGTGCAGCTGCAGGAGTCGGGCCCAGGACTGGTGAAGCCTTCGGAGACCCTGTCCCTC
ACCTGCACTGTCTCTGGTGACTCCATCAATAATTACTACTGGACCTGGCTCCGGCAGCCC
CCAGGGAAGGGACTGGAGTGGATTGGTTATATCTATTACAGTGGGAGCGCCAACTACAAC
CCCTCCCTCAAGAGTCGAGTCACCATATCAGTAGACACGTCCAAGAACCAGTTCTCCCTG
AAGCTAAATTCTGTGACCGCTGCGGACACGGCCGTGTATTACTGTGCGAGAGGGGCGGTC
AAGTACTTCCGGCATTGGGGCCAGGGCACCCTGGTCACCGTCTCCTCAGCCTCCACCAAG
GGCCCATCGGTCTTCCCCCTGGCGCCCTGCTCCAGGAGCACCTCCGAGAGCACAGCCGCC
CTGGGCTGCCTGGTCAAGGACTACTTCCCCGAACCGGTGACGGTGTCGTGGAACTCAGGC
GCCCTGACCAGCGGCGTGCACACCTTCCCGGCTGTCCTACAGTCCTCAGGACTCTACTCC
CTCAGCAGCGTGGTGACCGTGCCCTCCAGCAGCTTGGGCACGAAGACCTACACCTGCAAC
GTAGATCACAAGCCCAGCAACACCAAGGTGGACAAGAGAGTTGAGTCCAAATATGGTCCC
CCATGCCCACCCTGCCCAGCACCTGAGTTCCTGGGGGGACCATCAGTCTTCCTGTTCCCC
CCAAAACCCAAGGACACTCTCATGATCTCCCGGACCCCTGAGGTCACGTGCGTGGTGGTG
GACGTGAGCCAGGAAGACCCCGAGGTCCAGTTCAACTGGTACGTGGATGGCGTGGAGGTG
CATAATGCCAAGACAAAGCCGCGGGAGGAGCAGTTCAACAGCACGTACCGTGTGGTCAGC
GTCCTCACCGTCCTGCACCAGGACTGGCTGAACGGCAAGGAGTACAAGTGCAAGGTCTCC
AACAAAGGCCTCCCGTCCTCCATCGAGAAAACCATCTCCAAAGCCAAAGGGCAGCCCCGA
GAGCCACAGGTGTACACCCTGCCCCCATCCCAGGAGGAGATGACCAAGAACCAGGTCAGC
CTGACCTGCCTGGTCAAAGGCTTCTACCCCAGCGACATCGCCGTGGAGTGGGAGAGCAAT
GGGCAGCCGGAGAACAACTACAAGACCACGCCTCCCGTGCTGGACTCCGACGGCTCCTTC
TTCCTCTACAGCAGGCTCACCGTGGACAAGAGCAGGTGGCAGGAGGGGAATGTCTTCTCA
TGCTCCGTGATGCATGAGGCTCTGCACAACCACTACACACAGAAGTCCCTCTCCCTGTCT
CTGGGTAAATGA
HC Amino Acid Sequence
(SEQ ID NO: 206)
QVQLQESGPGLVKPSETLSLTCTVSGDSINNYYWTWLRQPPGKGLEWIGYIYYSGSANYN
PSLKSRVTISVDTSKNQFSLKLNSVTAADTAVYYCARGAVKYFRHWGQGTLVTVSSASTK
GPSVFPLAPCSRSTSESTAALGCLVKDYFPEPVTVSWNSGALTSGVHTFPAVLQSSGLYS
LSSVVTVPSSSLGTKTYTCNVDHKPSNTKVDKRVESKYGPPCPPCPAPEFLGGPSVFLFP
PKPKDTLMISRTPEVTCVVVDVSQEDPEVQFNWYVDGVEVHNAKTKPREEQFNSTYRVVS
VLTVLHQDWLNGKEYKCKVSNKGLPSSIEKTISKAKGQPREPQVYTLPPSQEEMTKNQVS
LTCLVKGFYPSDIAVEWESNGQPENNYKTTPPVLDSDGSFFLYSRLTVDKSRWQEGNVFS
CSVMHEALHNHYTQKSLSLSLGK
*underlined and bolded asparagine (N) may be mutated to a glutamine
(Q) for conjugation by transglutaminase, see, e.g., SEQ ID NO: 269
LC Nucleic Acid Sequence
(SEQ ID NO: 207)
GAAATTGTGTTGACGCAGTCTCCGGGCACCCTCTCTTTGTCTCCAGGGGAAAGAGCCACC
CTCTCCTGCAGGGCCAGTCAGACTATTAACCACAACAACTTAGCCTGGTACCAGCAGAGA
CCTGGCCAGGCTCCCAGGCTCCTCATCTATGGTGCATCCAACAGGGCCACTGCCATCCCA
GACAGGTTCAGTGGCAGTGGGTCTGGGACAGACTTCACTCTCACCATCAGCAGACTGGAG
CCTGAAGATTTTGAAGTGTATTCTTGTCAGCAGTATGGTAGCTTGCCGCTCACTTTCGGC
GGAGGGACCAAGGTGGAGATCAAACGAACTGTGGCTGCACCATCTGTCTTCATCTTCCCG
CCATCTGATGAGCAGTTGAAATCTGGAACTGCCTCTGTTGTGTGCCTGCTGAATAACTTC
TATCCCAGAGAGGCCAAAGTACAGTGGAAGGTGGATAACGCCCTCCAATCGGGTAACTCC
CAGGAGAGTGTCACAGAGCAGGACAGCAAGGACAGCACCTACAGCCTCAGCAGCACCCTG
ACGCTGAGCAAAGCAGACTACGAGAAACACAAAGTCTACGCCTGCGAAGTCACCCATCAG
GGCCTGAGCTCGCCCGTCACAAAGAGCTTCAACAGGGGAGAGTGTTAG
LC Amino Acid Sequence
(SEQ ID NO: 208)
EIVLTQSPGTLSLSPGERATLSCRASQTINHNNLAWYQQRPGQAPRLLIYGASNRATAIP
DRFSGSGSGTDFTLTISRLEPEDFEVYSCQQYGSLPLTFGGGTKVEIKRTVAAPSVFIFP
PSDEQLKSGTASVVCLLNNFYPREAKVQWKVDNALQSGNSQESVTEQDSKDSTYSLSSTL
TLSKADYEKHKVYACEVTHQGLSSPVTKSFNRGEC
10783 (wildtype hIgG1)/14574 (hIgG1 N180Q)
HCVR Nucleic Acid Sequence
(SEQ ID NO: 65)
CAGGTGCAGCTGGTGGAGTCTGGGGGAGGCGTGGTCCAGCCTGGGACGTCCCTGAGACTC
TCCTGTGCAGCGTCAGGATTCACCTTCAGTAGCTATGGCATGCACTGGGTCCGCCAGGCT
CCAGGCAAGGGGCTGGAGTGGGTGGCAGTTATATGGATTGATGGAAGTAATAAATATTAT
GCAGACTCCGTGAAGGGCCGATTCACCATCTCCAGAGACAATTCCAAGAACACGCTGTAT
CTGCAAATGAACAGCCTGAGAGCCGAGGACACGGCTGTGTATTACTGTGCGAGAAGGGGG
GGTATAGTAGTAGCTGCCCCCTTTGACTACTGGGGCCAGGGAACCCTGGTCACCGTCTCC
TCA
HCVR Amino Acid Sequence
(SEQ ID NO: 66)
QVQLVESGGGVVQPGTSLRLSCAASGFTFSSYGMHWVRQAPGKGLEWVAVIWIDGSNKYY
ADSVKGRFTISRDNSKNTLYLQMNSLRAEDTAVYYCARRGGIVVAAPFDYWGQGTLVTVS
S
HCDR1 Nucleic Acid Sequence
(SEQ ID NO: 67)
GGA TTC ACC TTC AGT AGC TAT GGC
HCDR1 Amino Acid Sequence
(SEQ ID NO: 68)
G F T F S S Y G
HCDR2 Nucleic Acid Sequence
(SEQ ID NO: 69)
ATA TGG ATT GAT GGA AGT AAT AAA
HCDR2 Amino Acid Sequence
(SEQ ID NO: 70)
I W I D G S N K
HCDR3 Nucleic Acid Sequence
(SEQ ID NO: 71)
GCG AGA AGG GGG GGT ATA GTA GTA GCT GCC CCC TTT GAC TAC
HCDR3 Amino Acid Sequence
(SEQ ID NO: 72)
A R R G G I V V A A P F D Y
LCVR Nucleic Acid Sequence
(SEQ ID NO: 73)
GACATCCAGATGACCCAGTCTCCATCCTCCCTGTCTGCATCTGTAGGAGACAGAGTCACC
ATCACTTGCCGGGCAAGTCAGAGCATTAGCAGCTATTTAAATTGGTATCAGCAGAAACCA
GGGAAAGCCCCTAAGCTCCTGATCTATGCTGCATCCAGTTTGCAAAGTGGGGTCCCGTCA
AGGTTCAGTGGCAGTGGATCTGGGACAGATTTCACTCTCACCATCAGCAGTCTGCAACCT
GAAGATTTTGCAACTTACTACTGTCAACAGAGTTACAGTACCCCTCCGATCACCTTCGGC
CAAGGGACACGACTGGAGATTAAA
LCVR Amino Acid Sequence
(SEQ ID NO: 74)
DIQMTQSPSSLSASVGDRVTITCRASQSISSYLNWYQQKPGKAPKLLIYAASSLQSGVPS
RFSGSGSGTDFTLTISSLQPEDFATYYCQQSYSTPPITFGQGTRLEIK
LCDR1 Nucleic Acid Sequence
(SEQ ID NO: 75)
CAG AGC ATT AGC AGC TAT
LCDR1 Amino Acid Sequence
(SEQ ID NO: 76)
Q S I S S Y
LCDR2 Nucleic Acid Sequence
(SEQ ID NO: 77)
GCT GCA TCC
LCDR2 Amino Acid Sequence
(SEQ ID NO: 78)
A A S
LCDR3 Nucleic Acid Sequence
(SEQ ID NO: 79)
CAA CAG AGT TAC AGT ACC CCT CCG ATC ACC
LCDR3 Amino Acid Sequence
(SEQ ID NO: 80)
Q Q S Y S T P P I T
HC Nucleic Acid Sequence
(SEQ ID NO: 209)
CAGGTGCAGCTGGTGGAGTCTGGGGGAGGCGTGGTCCAGCCTGGGACGTCCCTGAGACTC
TCCTGTGCAGCGTCAGGATTCACCTTCAGTAGCTATGGCATGCACTGGGTCCGCCAGGCT
CCAGGCAAGGGGCTGGAGTGGGTGGCAGTTATATGGATTGATGGAAGTAATAAATATTAT
GCAGACTCCGTGAAGGGCCGATTCACCATCTCCAGAGACAATTCCAAGAACACGCTGTAT
CTGCAAATGAACAGCCTGAGAGCCGAGGACACGGCTGTGTATTACTGTGCGAGAAGGGGG
GGTATAGTAGTAGCTGCCCCCTTTGACTACTGGGGCCAGGGAACCCTGGTCACCGTCTCC
TCAGCCTCCACCAAGGGCCCATCGGTCTTCCCCCTGGCGCCCTGCTCCAGGAGCACCTCC
GAGAGCACAGCCGCCCTGGGCTGCCTGGTCAAGGACTACTTCCCCGAACCGGTGACGGTG
TCGTGGAACTCAGGCGCCCTGACCAGCGGCGTGCACACCTTCCCGGCTGTCCTACAGTCC
TCAGGACTCTACTCCCTCAGCAGCGTGGTGACCGTGCCCTCCAGCAGCTTGGGCACGAAG
ACCTACACCTGCAACGTAGATCACAAGCCCAGCAACACCAAGGTGGACAAGAGAGTTGAG
TCCAAATATGGTCCCCCATGCCCACCCTGCCCAGCACCTGAGTTCCTGGGGGGACCATCA
GTCTTCCTGTTCCCCCCAAAACCCAAGGACACTCTCATGATCTCCCGGACCCCTGAGGTC
ACGTGCGTGGTGGTGGACGTGAGCCAGGAAGACCCCGAGGTCCAGTTCAACTGGTACGTG
GATGGCGTGGAGGTGCATAATGCCAAGACAAAGCCGCGGGAGGAGCAGTTCAACAGCACG
TACCGTGTGGTCAGCGTCCTCACCGTCCTGCACCAGGACTGGCTGAACGGCAAGGAGTAC
AAGTGCAAGGTCTCCAACAAAGGCCTCCCGTCCTCCATCGAGAAAACCATCTCCAAAGCC
AAAGGGCAGCCCCGAGAGCCACAGGTGTACACCCTGCCCCCATCCCAGGAGGAGATGACC
AAGAACCAGGTCAGCCTGACCTGCCTGGTCAAAGGCTTCTACCCCAGCGACATCGCCGTG
GAGTGGGAGAGCAATGGGCAGCCGGAGAACAACTACAAGACCACGCCTCCCGTGCTGGAC
TCCGACGGCTCCTTCTTCCTCTACAGCAGGCTCACCGTGGACAAGAGCAGGTGGCAGGAG
GGGAATGTCTTCTCATGCTCCGTGATGCATGAGGCTCTGCACAACCACTACACACAGAAG
TCCCTCTCCCTGTCTCTGGGTAAATGA
HC Amino Acid Sequence
(SEQ ID NO: 210)
QVQLVESGGGVVQPGTSLRLSCAASGFTFSSYGMHWVRQAPGKGLEWVAVIWIDGSNKYY
ADSVKGRFTISRDNSKNTLYLQMNSLRAEDTAVYYCARRGGIVVAAPFDYWGQGTLVTVS
SASTKGPSVFPLAPCSRSTSESTAALGCLVKDYFPEPVTVSWNSGALTSGVHTFPAVLQS
SGLYSLSSVVTVPSSSLGTKTYTCNVDHKPSNTKVDKRVESKYGPPCPPCPAPEFLGGPS
VFLFPPKPKDTLMISRTPEVTCVVVDVSQEDPEVQFNWYVDGVEVHNAKTKPREEQFNST
YRVVSVLTVLHQDWLNGKEYKCKVSNKGLPSSIEKTISKAKGQPREPQVYTLPPSQEEMT
KNQVSLTCLVKGFYPSDIA VEWESNGQPENNYKTTPPVLDSDGSFFLYSRLTVDKSRWQ
EGNVFSCSVMHEALHNHYTQKSLSLSLGK
*underlined and bolded asparagine (N) may be mutated to a
glutamine (Q) for conjugation by transglutaminase,
see, e.g., SEQ ID NO: 269
LC Nucleic Acid Sequence
(SEQ ID NO: 211)
GACATCCAGATGACCCAGTCTCCATCCTCCCTGTCTGCATCTGTAGGAGACAGAGTCACC
ATCACTTGCCGGGCAAGTCAGAGCATTAGCAGCTATTTAAATTGGTATCAGCAGAAACCA
GGGAAAGCCCCTAAGCTCCTGATCTATGCTGCATCCAGTTTGCAAAGTGGGGTCCCGTCA
AGGTTCAGTGGCAGTGGATCTGGGACAGATTTCACTCTCACCATCAGCAGTCTGCAACCT
GAAGATTTTGCAACTTACTACTGTCAACAGAGTTACAGTACCCCTCCGATCACCTTCGGC
CAAGGGACACGACTGGAGATTAAACGAACTGTGGCTGCACCATCTGTCTTCATCTTCCCG
CCATCTGATGAGCAGTTGAAATCTGGAACTGCCTCTGTTGTGTGCCTGCTGAATAACTTC
TATCCCAGAGAGGCCAAAGTACAGTGGAAGGTGGATAACGCCCTCCAATCGGGTAACTCC
CAGGAGAGTGTCACAGAGCAGGACAGCAAGGACAGCACCTACAGCCTCAGCAGCACCCTG
ACGCTGAGCAAAGCAGACTACGAGAAACACAAAGTCTACGCCTGCGAAGTCACCCATCAG
GGCCTGAGCTCGCCCGTCACAAAGAGCTTCAACAGGGGAGAGTGTTAG
LC Amino Acid Sequence
(SEQ ID NO: 212)
DIQMTQSPSSLSASVGDRVTITCRASQSISSYLNWYQQKPGKAPKLLIYAASSLQSGVPS
RFSGSGSGTDFTLTISSLQPEDFATYYCQQSYSTPPITFGQGTRLEIKRTVAAPSVFIFP
PSDEQLKSGTASVVCLLNNFYPREAKVQWKVDNALQSGNSQESVTEQDSKDSTYSLSSTL
TLSKADYEKHKVYACEVTHQGLSSPVTKSFNRGEC
31944
HCVR Nucleic Acid Sequence
(SEQ ID NO: 81)
CAG GTG CAG TTG GTG GAG TCT GGG GGA GGC GTG GTC CAG CCT GGG AGG
TCC CTG AGA CTC TCC TGT GAA GCG TCT GGA ATC ACC TTC AGA AAC TAT
GGC ATG CAC TGG GTC CGC CAG GCT CCA GGC AAG GGG CTG GAG TGG GTG
GCA GTT ATG TGG TAT GAT GGA AGT AAT AAA TAC TAC GCA GAC TCC GTG
AAG GGC CGA TTC ACC ATC TCC AGA GAC AAT TCC AAG AAC ACG GTG TAT
CTG CAA ATG AAC AGC CTG AGA GCC GAA GAC ACG GCT GTG TAT TAC TGT
GCG AGA CGG GGT CAT ATA GCA ACA GCT GCT CCC TTT GAC TAC TGG GGC
CAG GGA ACC CTG GTC ACC GTC TCC TCA
HCVR Amino Acid Sequence
(SEQ ID NO: 82)
QVQLVESGGGVVQPGRSLRLSCEASGITFRNYGMHWVRQAPGKGLEWVAVMWYDGSNKYY
ADSVKGRFTISRDNSKNTVYLQMNSLRAEDTAVYYCARRGHIATAAPFDYWGQGTLVTVS
S
HCDR1 Nucleic Acid Sequence
(SEQ ID NO: 83)
GGA ATC ACC TTC AGA AAC TAT GGC
HCDR1 Amino Acid Sequence
(SEQ ID NO: 84)
G I T F R N Y G
HCDR2 Nucleic Acid Sequence
(SEQ ID NO: 85)
atg tgg tat gat gga agt aat aaa
HCDR2 Amino Acid Sequence
(SEQ ID NO: 86)
M W Y D G S N
HCDR3 Nucleic Acid Sequence
(SEQ ID NO: 87)
GCG AGA CGG GGT CAT ATA GCA ACA GCT GCT CCC TTT GAC TAC
HCDR3 Amino Acid Sequence
(SEQ ID NO: 88)
A R R G H I A T A A P F D
LCVR Nucleic Acid Sequence
(SEQ ID NO: 89)
GACATCCAGATGACCCAGTCTCCATCCTCCCTGTCTGCATCCGTAGGAGACAGAGTCACC
ATCAGTTGCCGGGCAAGTCAGAGCATTAGTAGTTATTTAAATTGGTATCAGCAGAAACCA
GGGAAAGCCCCTAAGGTCCTGATGTATGCTGCATCCAGTTTGCAAAGTGGGGTCCCATCA
AGGTTCAGTGGCAGTGGATCTGGGACAGATTTCACTCTCACCATCAGCAGTCTGCAACCT
GAGGATTTTGCAACTTACTACTGTCAACAGAGTTACAGTACCCCTCCGATCACCTTCGGC
CAAGGGACACGACTGGAGATTAAA
LCVR Amino Acid Sequence
(SEQ ID NO: 90)
DIQMTQSPSSLSASVGDRVTISCRASQSISSYLNWYQQKPGKAPKVLMYAASSLQSGVPS
RFSGSGSGTDFTLTISSLQPEDFATYYCQQSYSTPPITFGQGTRLEIK
LCDR1 Nucleic Acid Sequence
(SEQ ID NO: 91)
CAG AGC ATT AGT AGT TAT
LCDR1 Amino Acid Sequence
(SEQ ID NO: 92)
Q S I S S Y
LCDR2 Nucleic Acid Sequence
(SEQ ID NO: 93)
GCT GCA TCC
LCDR2 Amino Acid Sequence
(SEQ ID NO: 94)
A A S
LCDR3 Nucleic Acid Sequence
(SEQ ID NO: 95)
CAA CAG AGT TAC AGT ACC CCT CCG ATC ACC
LCDR3 Amino Acid Sequence
(SEQ ID NO: 96)
Q Q S Y S T P P I T
HC Nucleic Acid Sequence
(SEQ ID NO: 213)
CAG GTG CAG TTG GTG GAG TCT GGG GGA GGC GTG GTC CAG CCT GGG AGG
TCC CTG AGA CTC TCC TGT GAA GCG TCT GGA ATC ACC TTC AGA AAC TAT
GGC ATG CAC TGG GTC CGC CAG GCT CCA GGC AAG GGG CTG GAG TGG GTG
GCA GTT ATG TGG TAT GAT GGA AGT AAT AAA TAC TAC GCA GAC TCC GTG
AAG GGC CGA TTC ACC ATC TCC AGA GAC AAT TCC AAG AAC ACG GTG TAT
CTG CAA ATG AAC AGC CTG AGA GCC GAA GAC ACG GCT GTG TAT TAC TGT
GCG AGA CGG GGT CAT ATA GCA ACA GCT GCT CCC TTT GAC TAC TGG GGC
CAG GGA ACC CTG GTC ACC GTC TCC TCA
GCCAAAACAACAGCCCCATCGGTCTATCCACTGGCCCCTGTGTGTGGAGATACAACTGGC
TCCTCGGTGACTCTAGGATGCCTGGTCAAGGGTTATTTCCCTGAGCCAGTGACCTTGACC
TGGAACTCTGGATCCCTGTCCAGTGGTGTGCACACCTTCCCAGCTGTCCTGCAGTCTGAC
CTCTACACCCTCAGCAGCTCAGTGACTGTAACCTCGAGCACCTGGCCCAGCCAGTCCATC
ACCTGCAATGTGGCCCACCCGGCAAGCAGCACCAAGGTGGACAAGAAAATTGAGCCCAGA
GGGCCCACAATCAAGCCCTGTCCTCCATGCAAATGCCCAGCACCTAACCTCTTGGGTGGA
CCATCCGTCTTCATCTTCCCTCCAAAGATCAAGGATGTACTCATGATCTCCCTGAGCCCC
ATAGTCACATGTGTGGTGGTGGATGTGAGCGAGGATGACCCAGATGTCCAGATCAGCTGG
TTTGTGAACAACGTGGAAGTACACACAGCTCAGACACAAACCCATAGAGAGGATTACAAC
AGTACTCTCCGGGTGGTCAGTGCCCTCCCCATCCAGCACCAGGACTGGATGAGTGGCAAG
GAGTTCAAATGCAAGGTCAACAACAAAGACCTCCCAGCGCCCATCGAGAGAACCATCTCA
AAACCCAAAGGGTCAGTAAGAGCTCCACAGGTATATGTCTTGCCTCCACCAGAAGAAGAG
ATGACTAAGAAACAGGTCACTCTGACCTGCATGGTCACAGACTTCATGCCTGAAGACATT
TACGTGGAGTGGACCAACAACGGGAAAACAGAGCTAAACTACAAGAACACTGAACCAGTC
CTGGACTCTGATGGTTCTTACTTCATGTACAGCAAGCTGAGAGTGGAAAAGAAGAACTGG
GTGGAAAGAAATAGCTACTCCTGTTCAGTGGTCCACGAGGGTCTGCACAATCACCACACG
ACTAAGAGCTTCTCCCGGACTCCGGGTAAATGA
HC Amino Acid Sequence
(SEQ ID NO: 214)
QVQLVESGGG VVQPGRSLRL SCEASGITFR NYGMHWVRQA PGKGLEWVAV
MWYDGSNKYY ADSVKGRFTI SRDNSKNTVY LQMNSLRAED TAVYYCARRG
HIATAAPFDY WGQGTLVTVS S
AKTTAPSVYPLAPVCGDTTGSSVTLGCLVKGYFPEPVTLTWNSGSLSSGVHTFPAVLQSD
LYTLSSSVTVTSSTWPSQSITCNVAHPASSTKVDKKIEPRGPTIKPCPPCKCPAPNLLGG
PSVFIFPPKIKDVLMISLSPIVTCVVVDVSEDDPDVQISWFVNNVEVHTAQTQTHREDYN
STLRVVSALPIQHQDWMSGKEFKCKVNNKDLPAPIERTISKPKGSVRAPQVYVLPPPEEE
MTKKQVTLTCMVTDFMPEDIYVEWTNNGKTELNYKNTEPVLDSDGSYFMYSKLRVEKKNW
VERNSYSCSVVHEGLHNHHTTKSFSRTPGK
LC Nucleic Acid Sequence
(SEQ ID NO: 215)
GACATCCAGATGACCCAGTCTCCATCCTCCCTGTCTGCATCCGTAGGAGACAGAGTCACC
ATCAGTTGCCGGGCAAGTCAGAGCATTAGTAGTTATTTAAATTGGTATCAGCAGAAACCA
GGGAAAGCCCCTAAGGTCCTGATGTATGCTGCATCCAGTTTGCAAAGTGGGGTCCCATCA
AGGTTCAGTGGCAGTGGATCTGGGACAGATTTCACTCTCACCATCAGCAGTCTGCAACCT
GAGGATTTTGCAACTTACTACTGTCAACAGAGTTACAGTACCCCTCCGATCACCTTCGGC
CAAGGGACACGACTGGAGATTAAACGAGCTGATGCTGCACCAACTGTATCCATCTTCCCA
CCATCCAGTGAGCAGTTAACATCTGGAGGTGCCTCAGTCGTGTGCTTCTTGAACAACTTC
TACCCCAAAGACATCAATGTCAAGTGGAAGATTGATGGCAGTGAACGACAAAATGGCGTC
CTGAACAGTTGGACTGATCAGGACAGCAAAGACAGCACCTACAGCATGAGCAGCACCCTC
ACGTTGACCAAGGACGAGTATGAACGACATAACAGCTATACCTGTGAGGCCACTCACAAG
ACATCAACTTCACCCATTGTCAAGAGCTTCAACAGGGGAGAGTGTTGA
LC Amino Acid Sequence
(SEQ ID NO: 216)
DIQMTQSPSS LSASVGDRVT ISCRASQSIS SYLNWYQQKP GKAPKVLMYA
ASSLQSGVPS RFSGSGSGTD FTLTISSLQP EDFATYYCQQ SYSTPPITFG QGTRLEIK
RADAAPTVSIFPPSSEQLTSGGASVVCFLNNFYPKDINVKWKIDGSERQNGVLNSWTDQD
SKDSTYSMSSTLTLTKDEYERHNSYTCEATHKTSTSPIVKSFNRGEC
31265 (wildtype hIgG1)/5972 (hIgG1 N180Q)
HCVR Nucleic Acid Sequence
(SEQ ID NO: 97)
CAGGTGCAGCTGGTGGAGTCTGGGGGAGGCGTGGTCCAGCCTGGGAGGTCCCTGAGACTC
TCCTGTACAGCGTCTGGATTCACCTTCCGTTCCTATGGCATGCACTGGGTCCGCCAGGCT
CCAGGCAAGGGGCTGGAGTGGGTGTCAGTTATTTGGATTGATGGAAATAATATATACTAT
GCAGACTCCGTGAAGGGCCGATTCACCATCTCCAGAGACAATTCCAAGAACACGCTGTAT
CTGCAAATGGACAGCCTGAGAGCCGAGGACACGGCTGTTTATTACTGTGCGAGAAGACTG
GCTATAACATCAGCTGCCCCCTTTGACTACTGGGGCCAGGGAACCCTGGTCACCGTCTCC
TCA
HCVR Amino Acid Sequence
(SEQ ID NO: 98)
QVQLVESGGGVVQPGRSLRLSCTASGFTFRSYGMHWVRQAPGKGLEWVSVIWIDGNNIYY
ADSVKGRFTISRDNSKNTLYLQMDSLRAEDTAVYYCARRLAITSAAPFDYWGQGTLVTVS
S
HCDR1 Nucleic Acid Sequence
(SEQ ID NO: 99)
GGA TTC ACC TTC CGT TCC TAT GGC
HCDR1 Amino Acid Sequence
(SEQ ID NO: 100)
G F T F R S Y G
HCDR2 Nucleic Acid Sequence
(SEQ ID NO: 101)
ATT TGG ATT GAT GGA AAT AAT ATA
HCDR2 Amino Acid Sequence
(SEQ ID NO: 102)
I W I D G N N I
HCDR3 Nucleic Acid Sequence
(SEQ ID NO: 103)
GCG AGA AGA CTG GCT ATA ACA TCA GCT GCC CCC TTT GAC TAC
HCDR3 Amino Acid Sequence
(SEQ ID NO: 104)
A R R L A I T S A A P F D Y
LCVR Nucleic Acid Sequence
(SEQ ID NO: 105)
GACATCCAGATGACCCAGTCTCCATCCTCCCTGTCTGCATCTGTAGGAGACAGAGTCACC
ATCACTTGCCGGGCAAGTCAGAGCATTAGCAGCTATTTAAATTGGTATCAGCAGAAACCA
GGGAAAGCCCCTAAGCTCCTGATCTATGCTGCATCCAGTTTGCAAAGTGGGGTCCCGTCA
AGGTTCAGTGGCAGTGGATCTGGGACAGATTTCACTCTCACCATCAGCAGTCTGCAACCT
GAAGATTTTGCAACTTACTACTGTCAACAGAGTTACAGTACCCCTCCGATCACCTTCGGC
CAAGGGACACGACTGGAGATTAAA
LCVR Amino Acid Sequence
(SEQ ID NO: 106)
DIQMTQSPSSLSASVGDRVTITCRASQSISSYLNWYQQKPGKAPKLLIYAASSLQSGVPS
RFSGSGSGTDFTLTISSLQPEDFATYYCQQSYSTPPITFGQGTRLEIK
LCDR1 Nucleic Acid Sequence
(SEQ ID NO: 107)
CAG AGC ATT AGC AGC TAT
LCDR1 Amino Acid Sequence
(SEQ ID NO: 108)
Q S I S S Y
LCDR2 Nucleic Acid Sequence
(SEQ ID NO: 109)
GCT GCA TCC
LCDR2 Amino Acid Sequence
(SEQ ID NO: 110)
A A S
LCDR3 Nucleic Acid Sequence
(SEQ ID NO: 111)
CAA CAG AGT TAC AGT ACC CCT CCG ATC ACC
LCDR3 Amino Acid Sequence
(SEQ ID NO: 112)
Q Q S Y S T P P I T
HC Nucleic Acid Sequence
(SEQ ID NO: 217)
CAGGTGCAGCTGGTGGAGTCTGGGGGAGGCGTGGTCCAGCCTGGGAGGTCCCTGAGACTC
TCCTGTACAGCGTCTGGATTCACCTTCCGTTCCTATGGCATGCACTGGGTCCGCCAGGCT
CCAGGCAAGGGGCTGGAGTGGGTGTCAGTTATTTGGATTGATGGAAATAATATATACTAT
GCAGACTCCGTGAAGGGCCGATTCACCATCTCCAGAGACAATTCCAAGAACACGCTGTAT
CTGCAAATGGACAGCCTGAGAGCCGAGGACACGGCTGTTTATTACTGTGCGAGAAGACTG
GCTATAACATCAGCTGCCCCCTTTGACTACTGGGGCCAGGGAACCCTGGTCACCGTCTCC
TCAGCCTCCACCAAGGGCCCATCGGTCTTCCCCCTGGCGCCCTGCTCCAGGAGCACCTCC
GAGAGCACAGCCGCCCTGGGCTGCCTGGTCAAGGACTACTTCCCCGAACCGGTGACGGTG
TCGTGGAACTCAGGCGCCCTGACCAGCGGCGTGCACACCTTCCCGGCTGTCCTACAGTCC
TCAGGACTCTACTCCCTCAGCAGCGTGGTGACCGTGCCCTCCAGCAGCTTGGGCACGAAG
ACCTACACCTGCAACGTAGATCACAAGCCCAGCAACACCAAGGTGGACAAGAGAGTTGAG
TCCAAATATGGTCCCCCATGCCCACCCTGCCCAGCACCTGAGTTCCTGGGGGGACCATCA
GTCTTCCTGTTCCCCCCAAAACCCAAGGACACTCTCATGATCTCCCGGACCCCTGAGGTC
ACGTGCGTGGTGGTGGACGTGAGCCAGGAAGACCCCGAGGTCCAGTTCAACTGGTACGTG
GATGGCGTGGAGGTGCATAATGCCAAGACAAAGCCGCGGGAGGAGCAGTTCAACAGCACG
TACCGTGTGGTCAGCGTCCTCACCGTCCTGCACCAGGACTGGCTGAACGGCAAGGAGTAC
AAGTGCAAGGTCTCCAACAAAGGCCTCCCGTCCTCCATCGAGAAAACCATCTCCAAAGCC
AAAGGGCAGCCCCGAGAGCCACAGGTGTACACCCTGCCCCCATCCCAGGAGGAGATGACC
AAGAACCAGGTCAGCCTGACCTGCCTGGTCAAAGGCTTCTACCCCAGCGACATCGCCGTG
GAGTGGGAGAGCAATGGGCAGCCGGAGAACAACTACAAGACCACGCCTCCCGTGCTGGAC
TCCGACGGCTCCTTCTTCCTCTACAGCAGGCTCACCGTGGACAAGAGCAGGTGGCAGGAG
GGGAATGTCTTCTCATGCTCCGTGATGCATGAGGCTCTGCACAACCACTACACACAGAAG
TCCCTCTCCCTGTCTCTGGGTAAATGA
HC Amino Acid Sequence
(SEQ ID NO: 218)
QVQLVESGGGVVQPGRSLRLSCTASGFTFRSYGMHWVRQAPGKGLEWVSVIWIDGNNIYY
ADSVKGRFTISRDNSKNTLYLQMDSLRAEDTAVYYCARRLAITSAAPFDYWGQGTLVTVS
SASTKGPSVFPLAPCSRSTSESTAALGCLVKDYFPEPVTVSWNSGALTSGVHTFPAVLQS
SGLYSLSSVVTVPSSSLGTKTYTCNVDHKPSNTKVDKRVESKYGPPCPPCPAPEFLGGPS
VFLFPPKPKDTLMISRTPEVTCVVVDVSQEDPEVQFNWYVDGVEVHNAKTKPREEQFNST
YRVVSVLTVLHQDWLNGKEYKCKVSNKGLPSSIEKTISKAKGQPREPQVYTLPPSQEEMT
KNQVSLTCLVKGFYPSDIAVEWESNGQPENNYKTTPPVLDSDGSFFLYSRLTVDKSRWQE
GNVFSCSVMHEALHNHYTQKSLSLSLGK
*underlined and bolded asparagine (N) may be
mutated to a glutamine (Q) for conjugation by
transglutaminase, see, e.g., SEQ ID NO: 269
LC Nucleic Acid Sequence
(SEQ ID NO: 219)
GACATCCAGATGACCCAGTCTCCATCCTCCCTGTCTGCATCTGTAGGAGACAGAGTCACC
ATCACTTGCCGGGCAAGTCAGAGCATTAGCAGCTATTTAAATTGGTATCAGCAGAAACCA
GGGAAAGCCCCTAAGCTCCTGATCTATGCTGCATCCAGTTTGCAAAGTGGGGTCCCGTCA
AGGTTCAGTGGCAGTGGATCTGGGACAGATTTCACTCTCACCATCAGCAGTCTGCAACCT
GAAGATTTTGCAACTTACTACTGTCAACAGAGTTACAGTACCCCTCCGATCACCTTCGGC
CAAGGGACACGACTGGAGATTAAACGAACTGTGGCTGCACCATCTGTCTTCATCTTCCCG
CCATCTGATGAGCAGTTGAAATCTGGAACTGCCTCTGTTGTGTGCCTGCTGAATAACTTC
TATCCCAGAGAGGCCAAAGTACAGTGGAAGGTGGATAACGCCCTCCAATCGGGTAACTCC
CAGGAGAGTGTCACAGAGCAGGACAGCAAGGACAGCACCTACAGCCTCAGCAGCACCCTG
ACGCTGAGCAAAGCAGACTACGAGAAACACAAAGTCTACGCCTGCGAAGTCACCCATCAG
GGCCTGAGCTCGCCCGTCACAAAGAGCTTCAACAGGGGAGAGTGTTAG
LC Amino Acid Sequence
(SEQ ID NO: 220)
DIQMTQSPSSLSASVGDRVTITCRASQSISSYLNWYQQKPGKAPKLLIYAASSLQSGVPS
RFSGSGSGTDFTLTISSLQPEDFATYYCQQSYSTPPITFGQGTRLEIKRTVAAPSVFIFP
PSDEQLKSGTASVVCLLNNFYPREAKVQWKVDNALQSGNSQESVTEQDSKDSTYSLSSTL
TLSKADYEKHKVYACEVTHQGLSSPVTKSFNRGEC
31941
HCVR Nucleic Acid Sequence
(SEQ ID NO: 113)
CAGGTTCAGCTGGTGCAGTCTGGAGCTGAGGTGAAGAAGCCTGGGGCCTCAGTG
AAGGTCTCCTGCAAGGCTTCTGGTTACGCCTTCACCACCTATGGTATCACCTGGGTGCGA
CAGGCCCCTGGACAAGGACTTGAGTGGATGGGATGGATCAGCGCTTACAATGGAAATACA
AACTATGCAGAGAAGGTCCAGGGCAGATTCACCATGACCACAGACACATCCACGAATACA
GCCTACATGGAGCTGAGGAGCCTGAGATCCGACGACACGGCCGTGTATTTCTGTGCGAGA
AAGGGTCACTATGGTTCGGGGACTTATTATAACCCCTTTGGTTTTGATTTTTGGGGCCAA
GGGACAATGGTCACCGTCTCTTCA
HCVR Amino Acid Sequence
(SEQ ID NO: 114)
QVQLVQSGAEVKKPGASVKVSCKASGYAFTTYGITWVRQAPGQGLEWMGWISAYNGNTNY
AEKVQGRFTMTTDTSTNTAYMELRSLRSDDTAVYFCARKGHYGSGTYYNPFGFDFWGQGT
MVTVSS
HCDR1 Nucleic Acid Sequence
(SEQ ID NO: 115)
ggt tac gcc ttc acc acc tat ggt
HCDR1 Amino Acid Sequence
(SEQ ID NO: 116)
G Y A F T T Y G
HCDR2 Nucleic Acid Sequence
(SEQ ID NO: 117)
atc agc gct tac aat gga aat aca
HCDR2 Amino Acid Sequence
(SEQ ID NO: 118)
I S A Y N G N
HCDR3 Nucleic Acid Sequence
(SEQ ID NO: 119)
GCG AGA AAG GGT CAC TAT GGT TCG GGG ACT TAT TAT AAC CCC TTT
GGT TTT GAT TTT
HCDR3 Amino Acid Sequence
(SEQ ID NO: 120)
C A R K G H Y G S G T Y Y N P F G F D
LCVR Nucleic Acid Sequence
(SEQ ID NO: 121)
GAAATTATGTTGATGCAGTCTCCAGGCACCCTGTCTTTGTCTCCAGGGGAAAGAGCCACC
CTCTCCTGCAGGGCCAGTCAGAGTGTTAGCAGCAGCTACTTAGCCTGGTACCAACAGAAA
CCTGGCCAGGCTCCCAGGCTCCTCATCTATGGTGCATCCAGCAGGGCCACTGACATCCCA
GACAGGTTCAGTGGCAGTGGGTCTGGGACAGACTTCACTCTCACCATCAGCAGACTGGAG
CCTGAAGATTTTGCAGTTTATTTCTGTCAGCAGTATTATGGCTCACCTTGGACGTTCGGC
CAAGGGACCAAGGTGGAAATCAAG
LCVR Amino Acid Sequence
(SEQ ID NO: 122)
EIMLMQSPGTLSLSPGERATLSCRASQSVSSSYLAWYQQKPGQAPRLLIYGASSRATDIP
DRFSGSGSGTDFTLTISRLEPEDFAVYFCQQYYGSPWTFGQGTKVEIK
LCDR1 Nucleic Acid Sequence
(SEQ ID NO: 123)
cag agt gtt agc agc agc tac
LCDR1 Amino Acid Sequence
(SEQ ID NO: 124)
Q S V S S S Y
LCDR2 Nucleic Acid Sequence
(SEQ ID NO: 125)
ggt gca tcc
LCDR2 Amino Acid Sequence
(SEQ ID NO: 126)
G A
LCDR3 Nucleic Acid Sequence
(SEQ ID NO: 127)
cag cag tat tat ggc tca cct tgg acg
LCDR3 Amino Acid Sequence
(SEQ ID NO: 128)
C Q Q Y Y G S P W
HC Nucleic Acid Sequence
(SEQ ID NO: 221)
CAGGTTCAGCTGGTGCAGTCTGGAGCTGAGGTGAAGAAGCCTGGGGCCTCAGTGAAGGTC
TCCTGCAAGGCTTCTGGTTACGCCTTCACCACCTATGGTATCACCTGGGTGCGACAGGCC
CCTGGACAAGGACTTGAGTGGATGGGATGGATCAGCGCTTACAATGGAAATACAAACTAT
GCAGAGAAGGTCCAGGGCAGATTCACCATGACCACAGACACATCCACGAATACAGCCTAC
ATGGAGCTGAGGAGCCTGAGATCCGACGACACGGCCGTGTATTTCTGTGCGAGAAAGGGT
CACTATGGTTCGGGGACTTATTATAACCCCTTTGGTTTTGATTTTTGGGGCCAAGGGACA
ATGGTCACCGTCTCTTCAGCCAAAACGACACCCCCATCTGTCTATCCACTGGCCCCTGGA
TCTGCTGCCCAAACTAACTCCATGGTGACCCTGGGATGCCTGGTCAAGGGCTATTTCCCT
GAGCCAGTGACAGTGACCTGGAACTCTGGATCCCTGTCCAGCGGTGTGCACACCTTCCCA
GCTGTCCTGCAGTCTGACCTCTACACTCTGAGCAGCTCAGTGACTGTCCCCTCCAGCACC
TGGCCCAGCGAGACCGTCACCTGCAACGTTGCCCACCCGGCCAGCAGCACCAAGGTGGAC
AAGAAAATTGTGCCCAGGGATTGTGGTTGTAAGCCTTGCATATGTACAGTCCCAGAAGTA
TCATCTGTCTTCATCTTCCCCCCAAAGCCCAAGGATGTGCTCACCATTACTCTGACTCCT
AAGGTCACGTGTGTTGTGGTAGACATCAGCAAGGATGATCCCGAGGTCCAGTTCAGCTGG
TTTGTAGATGATGTGGAGGTGCACACAGCTCAGACGCAACCCCGGGAGGAGCAGTTCAAC
AGCACTTTCCGCTCAGTCAGTGAACTTCCCATCATGCACCAGGACTGGCTCAATGGCAAG
GAGTTCAAATGCAGGGTCAACAGTGCAGCTTTCCCTGCCCCCATCGAGAAAACCATCTCC
AAAACCAAAGGCAGACCGAAGGCTCCACAGGTGTACACCATTCCACCTCCCAAGGAGCAG
ATGGCCAAGGATAAAGTCAGTCTGACCTGCATGATAACAGACTTCTTCCCTGAAGACATT
ACTGTGGAGTGGCAGTGGAATGGGCAGCCAGCGGAGAACTACAAGAACACTCAGCCCATC
ATGGACACAGATGGCTCTTACTTCGTCTACAGCAAGCTCAATGTGCAGAAGTCCAACTGG
GAGGCAGGAAATACTTTCACCTGCTCTGTGTTACATGAGGGCCTGCACAACCACCATACT
GAGAAGTCCCTCTCCCACTCTCCTGGTAAATGA
HC Amino Acid Sequence
(SEQ ID NO: 222)
QVQLVQSGAEVKKPGASVKVSCKASGYAFTTYGITWVRQAPGQGLEWMGWISAYNGNTNY
AEKVQGRFTMTTDTSTNTAYMELRSLRSDDTAVYFCARKGHYGSGTYYNPFGFDFWGQGT
MVTVSSAKTTPPSVYPLAPGSAAQTNSMVTLGCLVKGYFPEPVTVTWNSGSLSSGVHTFP
AVLQSDLYTLSSSVTVPSSTWPSETVTCNVAHPASSTKVDKKIVPRDCGCKPCICTVPEV
SSVFIFPPKPKDVLTITLTPKVTCVVVDISKDDPEVQFSWFVDDVEVHTAQTQPREEQFN
STFRSVSELPIMHQDWLNGKEFKCRVNSAAFPAPIEKTISKTKGRPKAPQVYTIPPPKEQ
MAKDKVSLTCMITDFFPEDITVEWQWNGQPAENYKNTQPIMDTDGSYFVYSKLNVQKSNW
EAGNTFTCSVLHEGLHNHHTEKSLSHSPGK
LC Nucleic Acid Sequence
(SEQ ID NO: 223)
GAAATTATGTTGATGCAGTCTCCAGGCACCCTGTCTTTGTCTCCAGGGGAAAGAGCCACC
CTCTCCTGCAGGGCCAGTCAGAGTGTTAGCAGCAGCTACTTAGCCTGGTACCAACAGAAA
CCTGGCCAGGCTCCCAGGCTCCTCATCTATGGTGCATCCAGCAGGGCCACTGACATCCCA
GACAGGTTCAGTGGCAGTGGGTCTGGGACAGACTTCACTCTCACCATCAGCAGACTGGAG
CCTGAAGATTTTGCAGTTTATTTCTGTCAGCAGTATTATGGCTCACCTTGGACGTTCGGC
CAAGGGACCAAGGTGGAAATCAAGCGAGCTGATGCTGCACCAACTGTATCCATCTTCCCA
CCATCCAGTGAGCAGTTAACATCTGGAGGTGCCTCAGTCGTGTGCTTCTTGAACAACTTC
TACCCCAAAGACATCAATGTCAAGTGGAAGATTGATGGCAGTGAACGACAAAATGGCGTC
CTGAACAGTTGGACTGATCAGGACAGCAAAGACAGCACCTACAGCATGAGCAGCACCCTC
ACGTTGACCAAGGACGAGTATGAACGACATAACAGCTATACCTGTGAGGCCACTCACAAG
ACATCAACTTCACCCATTGTCAAGAGCTTCAACAGGGGAGAGTGTTGA
LC Amino Acid Sequence
(SEQ ID NO: 224)
EIMLMQSPGT LSLSPGERAT LSCRASQSVS SSYLAWYQQK PGQAPRLLIY
GASSRATDIP DRFSGSGSGT DFTLTISRLE PEDFAVYFCQ QYYGSPWTFG
QGTKVEIK
RADAAPTVSIFPPSSEQLTSGGASVVCFLNNFYPKDINVKWKIDGSERQNGVLNSWTDQD
SKDSTYSMSSTLTLTKDEYERHNSYTCEATHKTSTSPIVKSFNRGEC
7660
HCVR Nucleic Acid Sequence
(SEQ ID NO: 129)
GAGGTGCAGCTGGTGGAGTCTGGGGGAGGCTTGGTCCAGCCGGGGGGGTCCCTGAAACTC
TCCTGTACAGCCTCTGGGTTGACCCTCAGTGACTCTGCTATGCACTGGGTCCGCCAGGCT
TCCGGGAAAGGGCTGGAGTGGGTTGGCCGTATAAGAAATAAGGCTAATAGGTACGCGACA
GAATATGCTGCGTCGGTGAAAGGCAGGTTCACCATTTCAAGAGATGATTCAAAGAACACG
GCGTATCTACAAATGAACAGCCTGAAAACCGAGGACACGGCCGTGTATTATTGTACTAGA
AACTGGAAGATTTTCCTCTTTGACTACTGGGGCCAGGGAACCCTGGTCACCGTCTCCTCA
HCVR Amino Acid Sequence
(SEQ ID NO: 130)
EVQLVESGGGLVQPGGSLKLSCTASGLTLSDSAMHWVRQASGKGLEWVGRIRNKANRYAT
EYAASVKGRFTISRDDSKNTAYLQMNSLKTEDTAVYYCTRNWKIFLFDYWGQGTLVTVSS
HCDR1 Nucleic Acid Sequence
(SEQ ID NO: 131)
GGG TTG ACC CTC AGT GAC TCT GCT
HCDR1 Amino Acid Sequence
(SEQ ID NO: 132)
G L T L S D S A
HCDR2 Nucleic Acid Sequence
(SEQ ID NO: 133)
ATA AGA AAT AAG GCT AAT AGG TAC GCG ACA
HCDR2 Amino Acid Sequence
(SEQ ID NO: 134)
I R N K A N R Y A T
HCDR3 Nucleic Acid Sequence
(SEQ ID NO: 135)
ACT AGA AAC TGG AAG ATT TTC CTC TTT GAC TAC
HCDR3 Amino Acid Sequence
(SEQ ID NO: 136)
T R N W K I F L F D Y
LCVR Nucleic Acid Sequence
(SEQ ID NO: 137)
GAAATTGTGTTGACGCAGTCTCCAGGCACCCTGACTTTGTCTCCAGGGGAAAGAGCCACC
CTCTCCTGCAGGGCCAGTCAGAGTGTTGGCAGCAAATACTTAGCCTGGTTCCAGCAGAAA
CGTGGCCAGGCTCCCAGGCTCCTCATCTATGGTGCATCCAGCAGGACCAGTGGCATCCCC
GACAGGATCAGTGGCAGTGGGTCAGGGACAGACTTCACTCTCACCATCAGCAGACTGGAG
CCTGAAGATTTTGCAGTGTATTACTGTCAGCAGTATGGAAGTTCACCCTGGACGTTCGGC
CAAGGGACCAAGGTGGAAATCAAA
LCVR Amino Acid Sequence
(SEQ ID NO: 138)
EIVLTQSPGTLTLSPGERATLSCRASQSVGSKYLAWFQQKRGQAPRLLIYGASSRTSGIP
DRISGSGSGTDFTLTISRLEPEDFAVYYCQQYGSSPWTFGQGTKVEIK
LCDR1 Nucleic Acid Sequence
(SEQ ID NO: 139)
CAG AGT GTT GGC AGC AAA TAC
LCDR1 Amino Acid Sequence
(SEQ ID NO: 140)
Q S V G S K Y
LCDR2 Nucleic Acid Sequence
(SEQ ID NO: 141)
GGT GCA TCC
LCDR2 Amino Acid Sequence
(SEQ ID NO: 142)
G A S
LCDR3 Nucleic Acid Sequence
(SEQ ID NO: 143)
CAG CAG TAT GGA AGT TCA CCC TGG ACG
LCDR3 Amino Acid Sequence
(SEQ ID NO: 144)
Q Q Y G S S P W T
HC Nucleic Acid Sequence
(SEQ ID NO: 225)
GAGGTGCAGCTGGTGGAGTCTGGGGGAGGCTTGGTCCAGCCGGGGGGGTCCCTGAAACTC
TCCTGTACAGCCTCTGGGTTGACCCTCAGTGACTCTGCTATGCACTGGGTCCGCCAGGCT
TCCGGGAAAGGGCTGGAGTGGGTTGGCCGTATAAGAAATAAGGCTAATAGGTACGCGACA
GAATATGCTGCGTCGGTGAAAGGCAGGTTCACCATTTCAAGAGATGATTCAAAGAACACG
GCGTATCTACAAATGAACAGCCTGAAAACCGAGGACACGGCCGTGTATTATTGTACTAGA
AACTGGAAGATTTTCCTCTTTGACTACTGGGGCCAGGGAACCCTGGTCACCGTCTCCTCA
GCCTCCACCAAGGGCCCATCGGTCTTCCCCCTGGCGCCCTGCTCCAGGAGCACCTCCGAG
AGCACAGCCGCCCTGGGCTGCCTGGTCAAGGACTACTTCCCCGAACCGGTGACGGTGTCG
TGGAACTCAGGCGCCCTGACCAGCGGCGTGCACACCTTCCCGGCTGTCCTACAGTCCTCA
GGACTCTACTCCCTCAGCAGCGTGGTGACCGTGCCCTCCAGCAGCTTGGGCACGAAGACC
TACACCTGCAACGTAGATCACAAGCCCAGCAACACCAAGGTGGACAAGAGAGTTGAGTCC
AAATATGGTCCCCCATGCCCACCCTGCCCAGCACCTGAGTTCCTGGGGGGACCATCAGTC
TTCCTGTTCCCCCCAAAACCCAAGGACACTCTCATGATCTCCCGGACCCCTGAGGTCACG
TGCGTGGTGGTGGACGTGAGCCAGGAAGACCCCGAGGTCCAGTTCAACTGGTACGTGGAT
GGCGTGGAGGTGCATAATGCCAAGACAAAGCCGCGGGAGGAGCAGTTCAACAGCACGTAC
CGTGTGGTCAGCGTCCTCACCGTCCTGCACCAGGACTGGCTGAACGGCAAGGAGTACAAG
TGCAAGGTCTCCAACAAAGGCCTCCCGTCCTCCATCGAGAAAACCATCTCCAAAGCCAAA
GGGCAGCCCCGAGAGCCACAGGTGTACACCCTGCCCCCATCCCAGGAGGAGATGACCAAG
AACCAGGTCAGCCTGACCTGCCTGGTCAAAGGCTTCTACCCCAGCGACATCGCCGTGGAG
TGGGAGAGCAATGGGCAGCCGGAGAACAACTACAAGACCACGCCTCCCGTGCTGGACTCC
GACGGCTCCTTCTTCCTCTACAGCAGGCTCACCGTGGACAAGAGCAGGTGGCAGGAGGGG
AATGTCTTCTCATGCTCCGTGATGCATGAGGCTCTGCACAACCACTACACACAGAAGTCC
CTCTCCCTGTCTCTGGGTAAATGA
HC Amino Acid Sequence
(SEQ ID NO: 226)
EVQLVESGGGLVQPGGSLKLSCTASGLTLSDSAMHWVRQASGKGLEWVGRIRNKANRYAT
EYAASVKGRFTISRDDSKNTAYLQMNSLKTEDTAVYYCTRNWKIFLFDYWGQGTLVTVSS
ASTKGPSVFPLAPCSRSTSESTAALGCLVKDYFPEPVTVSWNSGALTSGVHTFPAVLQSS
GLYSLSSVVTVPSSSLGTKTYTCNVDHKPSNTKVDKRVESKYGPPCPPCPAPEFLGGPSV
FLFPPKPKDTLMISRTPEVTCVVVDVSQEDPEVQFNWYVDGVEVHNAKTKPREEQFNSTY
RVVSVLTVLHQDWLNGKEYKCKVSNKGLPSSIEKTISKAKGQPREPQVYTLPPSQEEMTK
NQVSLTCLVKGFYPSDIAVEWESNGQPENNYKTTPPVLDSDGSFFLYSRLTVDKSRWQEG
NVFSCSVMHEALHNHYTQKSLSLSLGK
LC Nucleic Acid Sequence
(SEQ ID NO: 227)
GAAATTGTGTTGACGCAGTCTCCAGGCACCCTGACTTTGTCTCCAGGGGAAAGAGCCACC
CTCTCCTGCAGGGCCAGTCAGAGTGTTGGCAGCAAATACTTAGCCTGGTTCCAGCAGAAA
CGTGGCCAGGCTCCCAGGCTCCTCATCTATGGTGCATCCAGCAGGACCAGTGGCATCCCC
GACAGGATCAGTGGCAGTGGGTCAGGGACAGACTTCACTCTCACCATCAGCAGACTGGAG
CCTGAAGATTTTGCAGTGTATTACTGTCAGCAGTATGGAAGTTCACCCTGGACGTTCGGC
CAAGGGACCAAGGTGGAAATCAAACGAACTGTGGCTGCACCATCTGTCTTCATCTTCCCG
CCATCTGATGAGCAGTTGAAATCTGGAACTGCCTCTGTTGTGTGCCTGCTGAATAACTTC
TATCCCAGAGAGGCCAAAGTACAGTGGAAGGTGGATAACGCCCTCCAATCGGGTAACTCC
CAGGAGAGTGTCACAGAGCAGGACAGCAAGGACAGCACCTACAGCCTCAGCAGCACCCTG
ACGCTGAGCAAAGCAGACTACGAGAAACACAAAGTCTACGCCTGCGAAGTCACCCATCAG
GGCCTGAGCTCGCCCGTCACAAAGAGCTTCAACAGGGGAGAGTGTTAG
LC Amino Acid Sequence
(SEQ ID NO: 228)
EIVLTQSPGTLTLSPGERATLSCRASQSVGSKYLAWFQQKRGQAPRLLIYGASSRTSGIP
DRISGSGSGTDFTLTISRLEPEDFAVYYCQQYGSSPWTFGQGTKVEIKRTVAAPSVFIFP
PSDEQLKSGTASVVCLLNNFYPREAKVQWKVDNALQSGNSQESVTEQDSKDSTYSLSSTL
TLSKADYEKHKVYACEVTHQGLSSPVTKSFNRGEC
9909
HCVR Nucleic Acid Sequence
(SEQ ID NO: 145)
GAGGTGCAGCTGTTGGAGTCTGGGGGAGGCTTGGTACAGCCGGGGGGGTCCCTGAGACTC
TCCTGTGCAGCCTCTGGATTCACCTTTAACAACTATGGCATGAGCTGGGTCCGCCAGGGT
CCAGGGAAGGGGCTGGAGTGGGTCTCATCTATTAGTGGTAGTGGTGGTACCACATTCTAC
GCAGACTCCGTGAAGGGCCGGTTCACCATCTCCAGAGACAATTCCAAGAACACGCTGTAT
CTGCAAATGAACAGCCTGAGAGCCGAGGACACGGCCGTATATTACTGTGGCAAAGGAGGA
TATTGTAGTAGTAGCGGCTGCCGTCACTACGGTATGGACGTCTGGGGCCAAGGGACCACG
GTCACCGTCTCCTCA
HCVR Amino Acid Sequence
(SEQ ID NO: 146)
EVQLLESGGGLVQPGGSLRLSCAASGFTFNNYGMSWVRQGPGKGLEWVSSISGSGGTTFY
ADSVKGRFTISRDNSKNTLYLQMNSLRAEDTAVYYCGKGGYCSSSGCRHYGMDVWGQGTT
VTVSS
HCDR1 Nucleic Acid Sequence
(SEQ ID NO: 147)
GGA TTC ACC TTT AAC AAC TAT GGC
HCDR1 Amino Acid Sequence
(SEQ ID NO: 148)
G F T F N N Y G
HCDR2 Nucleic Acid Sequence
(SEQ ID NO: 149)
ATT AGT GGT AGT GGT GGT ACC ACA
HCDR2 Amino Acid Sequence
(SEQ ID NO: 150)
S G S G G T
HCDR3 Nucleic Acid Sequence
(SEQ ID NO: 151)
GGC AAA GGA GGA TAT TGT AGT AGT AGC GGC TGC CGT CAC TAC GGT ATG
GAC GTC
HCDR3 Amino Acid Sequence
(SEQ ID NO: 152)
C G K G G Y C S S S G C R H
LCVR Nucleic Acid Sequence
(SEQ ID NO: 153)
CAGTCTGTGCTGACTCAGCCACCCTCAGCGTCTGGGACCCCCGGGCAGAGGGTCACCATC
TCTTGTTCTGGAAGCAGCTCCAACATCGGAAATAATTATATATACTGGTACCAGCGGCTC
CCAGGAACGACCCCCAAACTCCTCATCTATAGGAATAATCAGCGGCCCTCAGGGGTCCCT
GACCGATTCTCTGGCTCCAAGTCTGGCACCTCAGCCTCCCTGGCCATCAGTGGGCTCCGG
TCCGAGGATGAGGCTGATTATTACTGTGCAGCATGGGATGACACCCTGAGTGGGTATGTC
TTCGGAACTGGGACCAAGGTCACCGTCCTA
LCVR Amino Acid Sequence
(SEQ ID NO: 154)
QSVLTQPPSASGTPGQRVTISCSGSSSNIGNNYIYWYQRLPGTTPKLLIYRNNQRPSGVP
DRFSGSKSGTSASLAISGLRSEDEADYYCAAWDDTLSGYVFGTGTKVTVL
LCDR1 Nucleic Acid Sequence
(SEQ ID NO: 155)
AGC TCC AAC ATC GGA AAT AAT TAT
LCDR1 Amino Acid Sequence
(SEQ ID NO: 156)
S S N I G N N Y
LCDR2 Nucleic Acid Sequence
(SEQ ID NO: 157)
agg aat aat
LCDR2 Amino Acid Sequence
(SEQ ID NO: 158)
R N
LCDR3 Nucleic Acid Sequence
(SEQ ID NO: 159)
GCA GCA TGG GAT GAC ACC CTG AGT GGG TAT GTC
LCDR3 Amino Acid Sequence
(SEQ ID NO: 160)
C A A W D D T L S G Y
HC Nucleic Acid Sequence
(SEQ ID NO: 229)
GAGGTGCAGCTGTTGGAGTCTGGGGGAGGCTTGGTACAGCCGGGGGGGTCCCTGAGACTC
TCCTGTGCAGCCTCTGGATTCACCTTTAACAACTATGGCATGAGCTGGGTCCGCCAGGGT
CCAGGGAAGGGGCTGGAGTGGGTCTCATCTATTAGTGGTAGTGGTGGTACCACATTCTAC
GCAGACTCCGTGAAGGGCCGGTTCACCATCTCCAGAGACAATTCCAAGAACACGCTGTAT
CTGCAAATGAACAGCCTGAGAGCCGAGGACACGGCCGTATATTACTGTGGCAAAGGAGGA
TATTGTAGTAGTAGCGGCTGCCGTCACTACGGTATGGACGTCTGGGGCCAAGGGACCACG
GTCACCGTCTCCTCAGCCAAAACAACAGCCCCATCGGTCTATCCACTGGCCCCTGTGTGT
GGAGATACAACTGGCTCCTCGGTGACTCTAGGATGCCTGGTCAAGGGTTATTTCCCTGAG
CCAGTGACCTTGACCTGGAACTCTGGATCCCTGTCCAGTGGTGTGCACACCTTCCCAGCT
GTCCTGCAGTCTGACCTCTACACCCTCAGCAGCTCAGTGACTGTAACCTCGAGCACCTGG
CCCAGCCAGTCCATCACCTGCAATGTGGCCCACCCGGCAAGCAGCACCAAGGTGGACAAG
AAAATTGAGCCCAGAGGGCCCACAATCAAGCCCTGTCCTCCATGCAAATGCCCAGCACCT
AACCTCTTGGGTGGACCATCCGTCTTCATCTTCCCTCCAAAGATCAAGGATGTACTCATG
ATCTCCCTGAGCCCCATAGTCACATGTGTGGTGGTGGATGTGAGCGAGGATGACCCAGAT
GTCCAGATCAGCTGGTTTGTGAACAACGTGGAAGTACACACAGCTCAGACACAAACCCAT
AGAGAGGATTACAACAGTACTCTCCGGGTGGTCAGTGCCCTCCCCATCCAGCACCAGGAC
TGGATGAGTGGCAAGGAGTTCAAATGCAAGGTCAACAACAAAGACCTCCCAGCGCCCATC
GAGAGAACCATCTCAAAACCCAAAGGGTCAGTAAGAGCTCCACAGGTATATGTCTTGCCT
CCACCAGAAGAAGAGATGACTAAGAAACAGGTCACTCTGACCTGCATGGTCACAGACTTC
ATGCCTGAAGACATTTACGTGGAGTGGACCAACAACGGGAAAACAGAGCTAAACTACAAG
AACACTGAACCAGTCCTGGACTCTGATGGTTCTTACTTCATGTACAGCAAGCTGAGAGTG
GAAAAGAAGAACTGGGTGGAAAGAAATAGCTACTCCTGTTCAGTGGTCCACGAGGGTCTG
CACAATCACCACACGACTAAGAGCTTCTCCCGGACTCCGGGTAAATGA
HC Amino Acid Sequence
(SEQ ID NO: 230)
EVQLLESGGGLVQPGGSLRLSCAASGFTFNNYGMSWVRQGPGKGLEWVSSISGSGGTTFY
ADSVKGRFTISRDNSKNTLYLQMNSLRAEDTAVYYCGKGGYCSSSGCRHYGMDVWGQGTT
VTVSSAKTTAPSVYPLAPVCGDTTGSSVTLGCLVKGYFPEPVTLTWNSGSLSSGVHTFPA
VLQSDLYTLSSSVTVTSSTWPSQSITCNVAHPASSTKVDKKIEPRGPTIKPCPPCKCPAP
NLLGGPSVFIFPPKIKDVLMISLSPIVTCVVVDVSEDDPDVQISWFVNNVEVHTAQTQTH
REDYNSTLRVVSALPIQHQDWMSGKEFKCKVNNKDLPAPIERTISKPKGSVRAPQVYVLP
PPEEEMTKKQVTLTCMVTDFMPEDIYVEWTNNGKTELNYKNTEPVLDSDGSYFMYSKLRV
EKKNWVERNSYSCSVVHEGLHNHHTTKSFSRTPGK
LC Nucleic Acid Sequence
(SEQ ID NO: 231)
CAGTCTGTGCTGACTCAGCCACCCTCAGCGTCTGGGACCCCCGGGCAGAGGGTCACCATC
TCTTGTTCTGGAAGCAGCTCCAACATCGGAAATAATTATATATACTGGTACCAGCGGCTC
CCAGGAACGACCCCCAAACTCCTCATCTATAGGAATAATCAGCGGCCCTCAGGGGTCCCT
GACCGATTCTCTGGCTCCAAGTCTGGCACCTCAGCCTCCCTGGCCATCAGTGGGCTCCGG
TCCGAGGATGAGGCTGATTATTACTGTGCAGCATGGGATGACACCCTGAGTGGGTATGTC
TTCGGAACTGGGACCAAGGTCACCGTCCTACGAGCTGATGCTGCACCAACTGTATCCATC
TTCCCACCATCCAGTGAGCAGTTAACATCTGGAGGTGCCTCAGTCGTGTGCTTCTTGAAC
AACTTCTACCCCAAAGACATCAATGTCAAGTGGAAGATTGATGGCAGTGAACGACAAAAT
GGCGTCCTGAACAGTTGGACTGATCAGGACAGCAAAGACAGCACCTACAGCATGAGCAGC
ACCCTCACGTTGACCAAGGACGAGTATGAACGACATAACAGCTATACCTGTGAGGCCACT
CACAAGACATCAACTTCACCCATTGTCAAGAGCTTCAACAGGGGAGAGTGTTGA
LC Amino Acid Sequence
(SEQ ID NO: 232)
QSVLTQPPSASGTPGQRVTISCSGSSSNIGNNYIYWYQRLPGTTPKLLIYRNNQRPSGVP
DRFSGSKSGTSASLAISGLRSEDEADYYCAAWDDTLSGYVFGTGTKVTVLRADAAPTVSI
FPPSSEQLTSGGASVVCFLNNFYPKDINVKWKIDGSERQNGVLNSWTDQDSKDSTYSMSS
TLTLTKDEYERHNSYTCEATHKTSTSPIVKSFNRGEC
10713 (wildtype hIgG1)/14573 (hIgG1 N180Q)
HCVR Nucleic Acid Sequence
(SEQ ID NO: 161)
GAGGTGCAGCTGGTGGAGTCTGGGGGAAACTTGGTACAGCCTGGGGGGTCCCTGAGACTC
TCCTGTGCAGCCTCTGGATTCACCTTTACCAGCCATGCCATGAACTGGGTCCGCCAGGCT
CCAGGGAAGGGGCTGGAGTGGGTCTCAGTTATTACTGGTAGAGGTTTTGACACACACTAC
GCTGACTCCGTGAAGGGCCGGTTCACCATCTCCAGAGACATTTCCAAAAACACGCTGTAT
CTGCAAATGAACAGCCTGAGAGCCGAGGACACGGCCGTTTATTACTGTGCGAAAGGTCTC
TATGATTCGGGGAATTATTATATCGATTACTGGGGCCAGGGAACCCTGGTCACCGTCTCC
TCA
HCVR Amino Acid Sequence
(SEQ ID NO: 162)
EVQLVESGGNLVQPGGSLRLSCAASGFTFTSHAMNWVRQAPGKGLEWVSVITGRGFDTHY
ADSVKGRFTISRDISKNTLYLQMNSLRAEDTAVYYCAKGLYDSGNYYIDYWGQGTLVTVS
S
HCDR1 Nucleic Acid Sequence
(SEQ ID NO: 163)
GGA TTC ACC TTT ACC AGC CAT GCC
HCDR1 Amino Acid Sequence
(SEQ ID NO: 164)
G F T F T S H A
HCDR2 Nucleic Acid Sequence
(SEQ ID NO: 165)
ATT ACT GGT AGA GGT TTT GAC ACA
HCDR2 Amino Acid Sequence
(SEQ ID NO: 166)
I T G R G F D T
HCDR3 Nucleic Acid Sequence
(SEQ ID NO: 167)
GCG AAA GGT CTC TAT GAT TCG GGG AAT TAT TAT ATC GAT TAC
HCDR3 Amino Acid Sequence
(SEQ ID NO: 168)
A K G L Y D S G N Y Y I D Y
LCVR Nucleic Acid Sequence
(SEQ ID NO: 169)
CAGTCTGTGTTGACGCAGCCGCCCTCAGTGTCTGCGGCCCCAGGACAGAAGGTCACCATC
TCCTGCTCTGGAAGCAGCTCCAACATTGGGAATAATTATGTTTCCTGGTACCAGCAGCTC
CCAGGAACAGCCCCCAAACTCCTCATTTATGACAATAATAAGCGACCCTCAGGGATTCCT
GACCGATTCTCTGGCTCCAAGTCTGGCACGTCAGCCACCCTGGGCATCACCGGACTCCAG
ACTGGGGACGAGGCCGATTATTACTGCGGAACATGGGATCTCAGCCTGAGTTTCAATTGG
GTGTTCGGCGGAGGGACCAAGCTGACCGTCCTA
LCVR Amino Acid Sequence
(SEQ ID NO: 170)
QSVLTQPPSVSAAPGQKVTISCSGSSSNIGNNYVSWYQQLPGTAPKLLIYDNNKRPSGIP
DRFSGSKSGTSATLGITGLQTGDEADYYCGTWDLSLSFNWVFGGGTKLTVL
LCDR1 Nucleic Acid Sequence
(SEQ ID NO: 171)
AGC TCC AAC ATT GGG AAT AAT TAT
LCDR1 Amino Acid Sequence
(SEQ ID NO: 172)
S S N I G N N Y
LCDR2 Nucleic Acid Sequence
(SEQ ID NO: 173)
GAC AAT AAT
LCDR2 Amino Acid Sequence
(SEQ ID NO: 174)
D N N
LCDR3 Nucleic Acid Sequence
(SEQ ID NO: 175)
GGA ACA TGG GAT CTC AGC CTG AGT TTC AAT TGG GTG
LCDR3 Amino Acid Sequence
(SEQ ID NO: 176)
G T W D L S L S F N W V
HC Nucleic Acid Sequence
(SEQ ID NO: 233)
GAGGTGCAGCTGGTGGAGTCTGGGGGAAACTTGGTACAGCCTGGGGGGTCCCTGAGACTC
TCCTGTGCAGCCTCTGGATTCACCTTTACCAGCCATGCCATGAACTGGGTCCGCCAGGCT
CCAGGGAAGGGGCTGGAGTGGGTCTCAGTTATTACTGGTAGAGGTTTTGACACACACTAC
GCTGACTCCGTGAAGGGCCGGTTCACCATCTCCAGAGACATTTCCAAAAACACGCTGTAT
CTGCAAATGAACAGCCTGAGAGCCGAGGACACGGCCGTTTATTACTGTGCGAAAGGTCTC
TATGATTCGGGGAATTATTATATCGATTACTGGGGCCAGGGAACCCTGGTCACCGTCTCC
TCAGCCTCCACCAAGGGCCCATCGGTCTTCCCCCTGGCGCCCTGCTCCAGGAGCACCTCC
GAGAGCACAGCCGCCCTGGGCTGCCTGGTCAAGGACTACTTCCCCGAACCGGTGACGGTG
TCGTGGAACTCAGGCGCCCTGACCAGCGGCGTGCACACCTTCCCGGCTGTCCTACAGTCC
TCAGGACTCTACTCCCTCAGCAGCGTGGTGACCGTGCCCTCCAGCAGCTTGGGCACGAAG
ACCTACACCTGCAACGTAGATCACAAGCCCAGCAACACCAAGGTGGACAAGAGAGTTGAG
TCCAAATATGGTCCCCCATGCCCACCCTGCCCAGCACCTGAGTTCCTGGGGGGACCATCA
GTCTTCCTGTTCCCCCCAAAACCCAAGGACACTCTCATGATCTCCCGGACCCCTGAGGTC
ACGTGCGTGGTGGTGGACGTGAGCCAGGAAGACCCCGAGGTCCAGTTCAACTGGTACGTG
GATGGCGTGGAGGTGCATAATGCCAAGACAAAGCCGCGGGAGGAGCAGTTCAACAGCACG
TACCGTGTGGTCAGCGTCCTCACCGTCCTGCACCAGGACTGGCTGAACGGCAAGGAGTAC
AAGTGCAAGGTCTCCAACAAAGGCCTCCCGTCCTCCATCGAGAAAACCATCTCCAAAGCC
AAAGGGCAGCCCCGAGAGCCACAGGTGTACACCCTGCCCCCATCCCAGGAGGAGATGACC
AAGAACCAGGTCAGCCTGACCTGCCTGGTCAAAGGCTTCTACCCCAGCGACATCGCCGTG
GAGTGGGAGAGCAATGGGCAGCCGGAGAACAACTACAAGACCACGCCTCCCGTGCTGGAC
TCCGACGGCTCCTTCTTCCTCTACAGCAGGCTCACCGTGGACAAGAGCAGGTGGCAGGAG
GGGAATGTCTTCTCATGCTCCGTGATGCATGAGGCTCTGCACAACCACTACACACAGAAG
TCCCTCTCCCTGTCTCTGGGTAAATGA
HC Amino Acid Sequence
(SEQ ID NO: 234)
EVQLVESGGNLVQPGGSLRLSCAASGFTFTSHAMNWVRQAPGKGLEWVSVITGRGFDTHY
ADSVKGRFTISRDISKNTLYLQMNSLRAEDTAVYYCAKGLYDSGNYYIDYWGQGTLVTVS
SASTKGPSVFPLAPCSRSTSESTAALGCLVKDYFPEPVTVSWNSGALTSGVHTFPAVLQS
SGLYSLSSVVTVPSSSLGTKTYTCNVDHKPSNTKVDKRVESKYGPPCPPCPAPEFLGGPS
VFLFPPKPKDTLMISRTPEVTCVVVDVSQEDPEVQFNWYVDGVEVHNAKTKPREEQFNST
YRVVSVLTVLHQDWLNGKEYKCKVSNKGLPSSIEKTISKAKGQPREPQVYTLPPSQEEMT
KNQVSLTCLVKGFYPSDIAVEWESNGQPENNYKTTPPVLDSDGSFFLYSRLTVDKSRWQE
GNVFSCSVMHEALHNHYTQKSLSLSLGK
*underlined and bolded asparagine (N) may
be mutated to a glutamine (Q) for
conjugation by transglutaminase, see,
e.g., SEQ ID NO: 269
LC Nucleic Acid Sequence
(SEQ ID NO: 235)
CAGTCTGTGTTGACGCAGCCGCCCTCAGTGTCTGCGGCCCCAGGACAGAAGGTCACCATC
TCCTGCTCTGGAAGCAGCTCCAACATTGGGAATAATTATGTTTCCTGGTACCAGCAGCTC
CCAGGAACAGCCCCCAAACTCCTCATTTATGACAATAATAAGCGACCCTCAGGGATTCCT
GACCGATTCTCTGGCTCCAAGTCTGGCACGTCAGCCACCCTGGGCATCACCGGACTCCAG
ACTGGGGACGAGGCCGATTATTACTGCGGAACATGGGATCTCAGCCTGAGTTTCAATTGG
GTGTTCGGCGGAGGGACCAAGCTGACCGTCCTAGGCCAGCCCAAGGCCGCCCCCTCCGTG
ACCCTGTTCCCCCCCTCCTCCGAGGAGCTGCAGGCCAACAAGGCCACCCTGGTGTGCCTG
ATCTCCGACTTCTACCCCGGCGCCGTGACCGTGGCCTGGAAGGCCGACTCCTCCCCCGTG
AAGGCCGGCGTGGAGACCACCACCCCCTCCAAGCAGTCCAACAACAAGTACGCCGCCTCC
TCCTACCTGTCCCTGACCCCCGAGCAGTGGAAGTCCCACCGGTCCTACTCCTGCCAGGTG
ACCCACGAGGGCTCCACCGTGGAGAAGACCGTGGCCCCCACCGAGTGCTCCTGA
LC Amino Acid Sequence
(SEQ ID NO: 236)
QSVLTQPPSVSAAPGQKVTISCSGSSSNIGNNYVSWYQQLPGTAPKLLIYDNNKRPSGIP
DRFSGSKSGTSATLGITGLQTGDEADYYCGTWDLSLSFNWVFGGGTKLTVLGQPKAAPSV
TLFPPSSEELQANKATLVCLISDFYPGAVTVAWKADSSPVKAGVETTTPSKQSNNKYAAS
SYLSLTPEQWKSHRSYSCQVTHEGSTVEKTVAPTECS
7854
HCVR Nucleic Acid Sequence
(SEQ ID NO: 177)
CAGGTTCAGCTGGTGCAGTCTGGAGCTGAGGTGAAGAAGCCTGGGGC
CTCAGTGAAGGTCTCCTGCAAGGCTTCTGGTTACGCCTTCACCACCTATGGTATCACCTG
GGTGCGACAGGCCCCTGGACAAGGACTTGAGTGGATGGGATGGATCAGCGCTTACAATGG
AAATACAAACTATGCAGAGAAGGTCCAGGGCAGATTCACCATGACCACAGACACATCCAC
GAATACAGCCTACATGGAGCTGAGGAGCCTGAGATCCGACGACACGGCCGTGTATTTCTG
TGCGAGAAAGGGTCACTATGGTTCGGGGACTTATTATAACCCCTTTGGTTTTGATTTTTG
GGGCCAAGGGACAATGGTCACCGTCTCTTCA
HCVR Amino Acid Sequence
(SEQ ID NO: 178)
QVQLVQSGAEVKKPGASVKVSCKASGYAFTTYGITWVRQAPGQGLEWMGWISAYNGNTNY
AEKVQGRFTMTTDTSTNTAYMELRSLRSDDTAVYFCARKGHYGSGTYYNPFGFDFWGQGT
MVTVSS
HCDR1 Nucleic Acid Sequence
(SEQ ID NO: 179)
GGT TAC GCC TTC ACC ACC TAT GGT
HCDR1 Amino Acid Sequence
(SEQ ID NO: 180)
G Y A F T T Y G
HCDR2 Nucleic Acid Sequence
(SEQ ID NO: 181)
ATC AGC GCT TAC AAT GGA AAT ACA
HCDR2 Amino Acid Sequence
(SEQ ID NO: 182)
I S A Y N G N T
HCDR3 Nucleic Acid Sequence
(SEQ ID NO: 183)
GCG AGA AAG GGT CAC TAT GGT TCG GGG ACT TAT TAT AAC CCC TTT
GGT TTT GAT TTT
HCDR3 Amino Acid Sequence
(SEQ ID NO: 184)
A R K G H Y G S G T Y Y N P F G F D F
LCVR Nucleic Acid Sequence
(SEQ ID NO: 185)
GAAATTGTGTTGACGCAGTCTCCAGGCACCCTGTCTTTGTCTCCAGGGGAAAGAGCCACC
CTCTCCTGCAGGGCCAGTCAGAGTGTTAGCAGCAGCTACTTAGCCTGGTACCAACAGAAA
CCTGGCCAGGCTCCCAGGCTCCTCATCTATGGTGCATCCAGCAGGGCCACTGGCATCCCA
GACAGGTTCAGTGGCAGTGGGTCTGGGACAGACTTCACTCTCACCATCAGCAGACTGGAG
CCTGAAGATTTTGCTTTGTATTTCTGTCAGCAGTATTATGGCTCACCTTGGACGTTCGGC
CAAGGGACCAAGGTGGAAATCAAA
LCVR Amino Acid Sequence
(SEQ ID NO: 186)
EIVLTQSPGTLSLSPGERATLSCRASQSVSSSYLAWYQQKPGQAPRLLIYGASSRATGIP
DRFSGSGSGTDFTLTISRLEPEDFALYFCQQYYGSPWTFGQGTKVEIK
LCDR1 Nucleic Acid Sequence
(SEQ ID NO: 187)
CAG AGT GTT AGC AGC AGC TAC
LCDR1 Amino Acid Sequence
(SEQ ID NO: 188)
Q S V S S S Y
LCDR2 Nucleic Acid Sequence
(SEQ ID NO: 189)
GGT GCA TCC
LCDR2 Amino Acid Sequence
(SEQ ID NO: 190)
G A S
LCDR3 Nucleic Acid Sequence
(SEQ ID NO: 191)
CAG CAG TAT TAT GGC TCA CCT TGG ACG
LCDR3 Amino Acid Sequence
(SEQ ID NO: 192)
Q Q Y Y G S P W T
HC Nucleic Acid Sequence
(SEQ ID NO: 237)
CAGGTTCAGCTGGTGCAGTCTGGAGCTGAGGTGAAGAAGCCTGGGGCCTCAGTGAAGG
TCTCCTGCAAGGCTTCTGGTTACGCCTTCACCACCTATGGTATCACCTGGGTGCGA
CAGGCCCCTGGACAAGGACTTGAGTGGATGGGATGGATCAGCGCTTACAATGGAAATACA
AACTATGCAGAGAAGGTCCAGGGCAGATTCACCATGACCACAGACACATCCACGAATACA
GCCTACATGGAGCTGAGGAGCCTGAGATCCGACGACACGGCCGTGTATTTCTGTGCGAGA
AAGGGTCACTATGGTTCGGGGACTTATTATAACCCCTTTGGTTTTGATTTTTGGGGCCAA
GGGACAATGGTCACCGTCTCTTCAGCCTCCACCAAGGGCCCATCGGTCTTCCCCCTGGCG
CCCTGCTCCAGGAGCACCTCCGAGAGCACAGCCGCCCTGGGCTGCCTGGTCAAGGACTAC
TTCCCCGAACCGGTGACGGTGTCGTGGAACTCAGGCGCCCTGACCAGCGGCGTGCACACC
TTCCCGGCTGTCCTACAGTCCTCAGGACTCTACTCCCTCAGCAGCGTGGTGACCGTGCCC
TCCAGCAGCTTGGGCACGAAGACCTACACCTGCAACGTAGATCACAAGCCCAGCAACACC
AAGGTGGACAAGAGAGTTGAGTCCAAATATGGTCCCCCATGCCCACCCTGCCCAGCACCT
GAGTTCCTGGGGGGACCATCAGTCTTCCTGTTCCCCCCAAAACCCAAGGACACTCTCATG
ATCTCCCGGACCCCTGAGGTCACGTGCGTGGTGGTGGACGTGAGCCAGGAAGACCCCGAG
GTCCAGTTCAACTGGTACGTGGATGGCGTGGAGGTGCATAATGCCAAGACAAAGCCGCGG
GAGGAGCAGTTCAACAGCACGTACCGTGTGGTCAGCGTCCTCACCGTCCTGCACCAGGAC
TGGCTGAACGGCAAGGAGTACAAGTGCAAGGTCTCCAACAAAGGCCTCCCGTCCTCCATC
GAGAAAACCATCTCCAAAGCCAAAGGGCAGCCCCGAGAGCCACAGGTGTACACCCTGCCC
CCATCCCAGGAGGAGATGACCAAGAACCAGGTCAGCCTGACCTGCCTGGTCAAAGGCTTC
TACCCCAGCGACATCGCCGTGGAGTGGGAGAGCAATGGGCAGCCGGAGAACAACTACAAG
ACCACGCCTCCCGTGCTGGACTCCGACGGCTCCTTCTTCCTCTACAGCAGGCTCACCGTG
GACAAGAGCAGGTGGCAGGAGGGGAATGTCTTCTCATGCTCCGTGATGCATGAGGCTCTG
CACAACCACTACACACAGAAGTCCCTCTCCCTGTCTCTGGGTAAATGA
HC Amino Acid Sequence
(SEQ ID NO: 238)
QVQLVQSGAEVKKPGASVKVSCKASGYAFTTYGITWVRQAPGQGLEWMGWISAYNGNTNY
AEKVQGRFTMTTDTSTNTAYMELRSLRSDDTAVYFCARKGHYGSGTYYNPFGFDFWGQGT
MVTVSSASTKGPSVFPLAPCSRSTSESTAALGCLVKDYFPEPVTVSWNSGALTSGVHTFP
AVLQSSGLYSLSSVVTVPSSSLGTKTYTCNVDHKPSNTKVDKRVESKYGPPCPPCPAPEF
LGGPSVFLFPPKPKDTLMISRTPEVTCVVVDVSQEDPEVQFNWYVDGVEVHNAKTKPREE
QFNSTYRVVSVLTVLHQDWLNGKEYKCKVSNKGLPSSIEKTISKAKGQPREPQVYTLPPS
QEEMTKNQVSLTCLVKGFYPSDIAVEWESNGQPENNYKTTPPVLDSDGSFFLYSRLTVDK
SRWQEGNVFSCSVMHEALHNHYTQKSLSLSLGK
LC Nucleic Acid Sequence
(SEQ ID NO: 239)
GAAATTGTGTTGACGCAGTCTCCAGGCACCCTGTCTTTGTCTCCAGGGGAAAGAGCCACC
CTCTCCTGCAGGGCCAGTCAGAGTGTTAGCAGCAGCTACTTAGCCTGGTACCAACAGAAA
CCTGGCCAGGCTCCCAGGCTCCTCATCTATGGTGCATCCAGCAGGGCCACTGGCATCCCA
GACAGGTTCAGTGGCAGTGGGTCTGGGACAGACTTCACTCTCACCATCAGCAGACTGGAG
CCTGAAGATTTTGCTTTGTATTTCTGTCAGCAGTATTATGGCTCACCTTGGACGTTCGGC
CAAGGGACCAAGGTGGAAATCAAACGAACTGTGGCTGCACCATCTGTCTTCATCTTCCCG
CCATCTGATGAGCAGTTGAAATCTGGAACTGCCTCTGTTGTGTGCCTGCTGAATAACTTC
TATCCCAGAGAGGCCAAAGTACAGTGGAAGGTGGATAACGCCCTCCAATCGGGTAACTCC
CAGGAGAGTGTCACAGAGCAGGACAGCAAGGACAGCACCTACAGCCTCAGCAGCACCCTG
ACGCTGAGCAAAGCAGACTACGAGAAACACAAAGTCTACGCCTGCGAAGTCACCCATCAG
GGCCTGAGCTCGCCCGTCACAAAGAGCTTCAACAGGGGAGAGTGTTAG
LC Amino Acid Sequence
(SEQ ID NO: 240)
EIVLTQSPGTLSLSPGERATLSCRASQSVSSSYLAWYQQKPGQAPRLLIYGASSRATGIP
DRFSGSGSGTDFTLTISRLEPEDFALYFCQQYYGSPWTFGQGTKVEIKRTVAAPSVFIFP
PSDEQLKSGTASVVCLLNNFYPREAKVQWKVDNALQSGNSQESVTEQDSKDSTYSLSSTL
TLSKADYEKHKVYACEVTHQGLSSPVTKSFNRGEC

Non-limiting examples of targeting ligands that bind CACNG1 include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g. monovalent nanobodies, bivalent nanobodies, etc.), small modular immunopharmaceuticals (SMIPs), and shark variable IgNAR domains, are also encompassed within the expression “targeting ligand,” as used herein. In non-limiting embodiments, an anti-CACNG1 targeting ligand that binds CACNG1 useful for retargeting viral capsids as described herein comprise comprises an scFv. As a non-limiting example, an scFv sequences in V_{L}-(Gly₄Ser)₃-V_{H} format useful for retargeting viral capsids as described herein may comprise a heavy chain variable domain, light chain variable domain, heavy chain variable domain/light chain variable domain pair, HCDR1, HCDR2, HCDR3, LCDR1, LCDR2, LCDR3, and/or set of HCDR1-HCDR2-HCDR3-LCDR1-LCDR2-LCDR3 that is 90%, 95%, 97%, 98%, 99% or 100% identical, respectively, to any one of the amino acid sequences of a heavy chain variable domain, light chain variable domain, heavy chain variable domain/light chain variable domain pair, HCDR1, HCDR2, HCDR3, LCDR1, LCDR2, LCDR3, and/or set of HCDR1-HCDR2-HCDR3-LCDR1-LCDR2-LCDR3 as set forth in any one of SEQ ID NOs: 1-240.

A targeting ligand that binds a mammalian muscle cell-specific surface protein may be associated with (e.g., displayed by, operably linked to, bound to) a modified AAV capsid protein and resulting AAV capsids according to well-known methods, e.g., a direct approach in which the targeting ligand is directly inserted into (e.g., using recombinatorial methods) according to well-known methods. See, e.g., Stachler et al. (2006), supra; White et al. (2004), supra; Girod et al. (1999), supra; Grifman et al. (2001), supra; Shi et al. (2001), supra; Shi and Bartlett (2003), supra. A targeting ligand that binds a mammalian muscle cell-specific surface protein may be coupled to a modified AAV capsid protein and resulting AAV capsids using well-known chemical linkers, e.g., wherein the AAV capsid protein may be chemically modified to comprise a dibenzocycootyne group or an azide group, and optionally wherein a targeting ligand as described herein is attached to the dibenzocycootyne group or the azide group, see, e.g., U.S. 2022/028234, incorporated herein by reference in its entirety; wherein targeting ligand is covalently linked to a primary amino acid group of an AAV capsid protein, e.g, via a —CSNH— bond, etc. In some embodiments, a modified capsid as described herein comprises a targeting ligand, e.g., an anti-CACNG1 antibody or binding portion thereof, directly inserted into or coupled to it according to well-known direct recombinatorial methods.

### Binding Pairs

In some embodiments, a targeting ligand that binds a mammalian muscle cell-specific surface protein may be associated with (e.g., displayed by, operably linked to, bound to) a modified AAV capsid protein and resulting AAV capsids according to indirect recombinatorial approaches, wherein the AAV capsid protein is modified to comprise a first member of a binding pair (e.g., a heterologous scaffold), and optionally wherein the first member of the binding pair is linked to (e.g., covalently or non-covalently bound to) a second cognate member of the binding pair (e.g., an adaptor), further optionally wherein the second cognate member of the binding pair is fused to the targeting ligand. Non-limiting and exemplary binding pairs are listed in Buning and Srivastava (2019) Mol. Ther. Methods Clin Dev 12:248-265.

Accordingly, in some embodiments, modifications of a capsid protein as described herein include those that generally result from modifications at the genetic level, e.g., via modification of a cap gene, such as modifications that insert first member of a binding pair (e.g., a protein:protein binding pair, a protein:nucleic acid binding pair), a detectable label, etc., for display by the Cap protein.

In some embodiments, the first member forms a binding pair with an immunoglobulin constant domain. In some embodiments, the first member forms a binding pair with a metal ion, e.g., Ni²⁺, Co²⁺, Cu²⁺, Zn²⁺, Fe³⁺, etc. In some embodiments, the first member is selected from the group consisting of Streptavidin, Strep II, HA, L14, 4C-RGD, LH, and Protein A.

In some embodiments, the binding pair comprises an enzyme:nucleic acid binding pair. In some embodiments, the first member comprises a HUH-endonuclease or HUH-tag and the second member comprises a nucleic acid binding domain. In some embodiments, the first member comprises a HUH tag. See, e.g., U.S. 2021/0180082, incorporated herein in its entirety by reference.

In some embodiments, a capsid protein of the invention comprises at least a first member of a peptide:peptide binding pair.

In some embodiments, each of a first member and a second member of a peptide:peptide binding pair comprises an intein. See, e.g., Wagner et al., (2021) Adv. Sci. 8:2004018 (1 of 22); Muik et al. (2017) Biomaterials 144:84, each of which is incorporated herein in its entirety by reference.

In some embodiments, a first member is a B cell epitope, e.g., is between about 1 amino acid and about 35 amino acids in length, and forms a binding pair with an antibody paratope, e.g., an immunoglobulin variable domain. In some embodiments, a capsid protein of the invention may be modified to comprise a detectable label as a first member of a binding pair. Many detectable labels are known in the art. (See, e.g. Nilsson et al. (1997) “Affinity fusion strategies for detection, purification, and immobilization of modified proteins” Protein Expression and Purification 11:1-16, Terpe et al. (2003) “Overview of tag protein fusions: From molecular and biochemical fundamentals to commercial systems” Applied Microbiology and Biotechnology 60:523-533, and references therein). Detectable labels include, but are not limited to, a polyhistidine detectable labels (e.g., a His-6, His-8, or His-10) that binds immobilized divalent cations (e.g., Ni²⁺), a biotin moiety (e.g., on an in vivo biotinylated polypeptide sequence) that binds immobilized avidin, a GST (glutathione S-transferase) sequence that binds immobilized glutathione, an S tag that binds immobilized S protein, an antigen that binds an immobilized antibody or domain or fragment thereof (including, e.g., T7, myc, FLAG, and B tags that bind corresponding antibodies), a FLASH Tag (a high detectable label that couples to specific arsenic based moieties), a receptor or receptor domain that binds an immobilized ligand (or vice versa), protein A or a derivative thereof (e.g., Z) that binds immobilized IgG, maltose-binding protein (MBP) that binds immobilized amylose, an albumin-binding protein that binds immobilized albumin, a chitin binding domain that binds immobilized chitin, a calmodulin binding peptide that binds immobilized calmodulin, and a cellulose binding domain that binds immobilized cellulose. Another exemplary detectable label is a SNAP-tag, commercially available from Covalys (www.covalys.com). In some embodiments, a detectable label disclosed herein comprises a detectable label recognized by an antibody paratope, wherein the detectable label and the antibody paratope form a protein:protein binding pair.

In some embodiments, a capsid protein of the invention comprises a first member of a protein:protein binding pair comprising a detectable label, which may also be used for the detection and/or isolation of the Cap protein and/or as a first member of a protein:protein binding pair. In some embodiments, a detectable label acts as a first member of a protein:protein binding pair for the binding of a targeting ligand comprising a multispecific binding protein that may bind both the detectable label and a target expressed by a cell of interest. In some embodiments, a Cap protein of the invention comprises a first member of a protein:protein binding pair comprising c-myc (SEQ ID NO:246). Use of a detectable label as a first member of a protein:protein binding pair is described in, e.g., WO2019006043, incorporated herein in its entirety by reference.

In some embodiments, the first member comprises a B1 epitope (SEQ ID NO: 247). In some embodiments, a capsid protein is modified to comprise a B1 epitope in the VP3 region. In some embodiments, the first member is selected from the group consisting of FLAG, HA and c-myc (EQKLISEEDL; SEQ ID NO:246).

In some embodiments, a capsid protein comprises a first member of a protein:protein binding pair, wherein the protein:protein binding pair forms a covalent isopeptide bond. In some embodiments, the first member of a peptide:peptide binding pair is covalently bound via an isopeptide bond to a cognate second member of the peptide:peptide binding pair, and optionally wherein the cognate second member of the peptide:peptide binding pair is fused with a targeting ligand, which targeting ligand binds a target expressed by a cell of interest. In some embodiments, the protein:protein binding pair may be selected from the group consisting of SpyTag:SpyCatcher, SpyTag002:SpyCatcher002, SpyTag003:SpyCatcher003, SpyTag:KTag, Isopeptag:pilin-C, and SnoopTag:SnoopCatcher. In some embodiments, wherein the first member is SpyTag (or a biologically active portion or variant thereof) and the protein (second cognate member) is SpyCatcher (or a biologically active portion or variant thereof). In some embodiments, wherein the first member is SpyTag (or a biologically active portion or variant thereof) and the protein (second cognate member) is KTag (or a biologically active portion or variant thereof). In some embodiments, wherein the first member is KTag (or a biologically active portion or variant thereof) and the protein (second cognate member) is SpyTag (or a biologically active portion or variant thereof). In some embodiments, wherein the first member is SnoopTag (or a biologically active portion or variant thereof) and the protein (second cognate member) is SnoopCatcher (or a biologically active portion or variant thereof). In some embodiments, wherein the first member is Isopeptag (or a biologically active portion or variant thereof) and the protein (second cognate member) is Pilin-C (or a biologically active portion or variant thereof). In some embodiments, wherein the first member is SpyTag002 (or a biologically active portion or variant thereof) and the protein (second cognate member) is SpyCatcher002 (or a biologically active portion or variant thereof). In some embodiments, wherein the first member is Spy Tag003 (or a biologically active portion or variant thereof) and the protein (second cognate member) is SpyCatcher003 (or a biologically active portion or variant thereof). In some embodiments, a Cap protein of the invention comprises a SpyTag, or a biologically active portion or variant thereof. Use of a first member of a protein:protein binding pair is described in WO2019006046, incorporated herein in its entirety.

In some embodiments, a first member of a protein:protein binding pair and/or detectable label is operably linked to (translated in frame with, chemically attached to, and/or displayed by) a Cap protein of the invention via a first or second linker, e.g., an amino acid spacer that is at least one amino acid in length. In some embodiments, the first member of a protein:protein binding pair is flanked by a first and/or second linker, e.g., a first and/or second amino acid spacer, each of which spacer is at least one amino acid in length.

In some embodiments, the first and/or second linkers are not identical. In some embodiments, the first and/or second linker is each independently one or two amino acids in length. In some embodiments, the first and/or second linker is each independently one, two or three amino acids in length. In some embodiments, the first and/or second linker is each independently one, two, three, or four amino acids in length. In some embodiments, the first and/or second linker is each independently one, two, three, four, or five amino acids in length. In some embodiments, the first and/or second linker are each independently one, two, three, four, or five amino acids in length. In some embodiments, the first and/or second linker is each independently one, two, three, four, five, or six amino acids in length. In some embodiments, the first and/or second linker is each independently one, two, three, four, five, six, or seven amino acids in length. In some embodiments, the first and/or second linker is each independently one, two, three, four, five, six, seven, or eight amino acids in length. In some embodiments, the first and/or second linker is each independently one, two, three, four, five, six, seven, eight or nine amino acids in length. In some embodiments, the first and or second linker is each independently one, two, three, four, five, six, seven, eight, nine, or ten amino acids in length. In some embodiments, the first and or second linker is each independently one, two, three, four, five, six, seven, eight, nine, ten, or more amino acids in length.

In some embodiments, the first and second linkers are identical in sequence and/or in length and are each one amino acid in length. In some embodiments, the first and second linkers are identical in length, and are each one amino acid in length. In some embodiments, the first and second linkers are identical in length, and are each two amino acids in length. In some embodiments, the first and second linkers are identical in length, and are each three amino acids in length. In some embodiments, the first and second linkers are identical in length, and are each four amino acids in length, e.g., the linker is GLSG (SEQ ID NO: 248). In some embodiments, the first and second linkers are identical in length, and are each five amino acids in length. In some embodiments, the first and second linkers are identical in length, and are each six amino acids in length, e.g., the first and second linkers each comprise a sequence of GLSGSG (SEQ ID NO:249). In some embodiments, the first and second linkers are identical in length, and are each seven amino acids in length. In some embodiments, the first and second linkers are identical in length, and are each eight amino acids in length, e.g., the first and second linkers each comprise a sequence of GLSGLSGS (SEQ ID NO:250). In some embodiments, the first and second linkers are identical in length, and are each nine amino acids in length. In some embodiments, the first and second linkers are identical in length, and are each ten amino acids in length, e.g., the first and second linkers each comprise a sequence of GLSGLSGLSG (SEQ ID NO:251) or GLSGGSGLSG (SEQ ID NO:252). In some embodiments, the first and second linkers are identical in length, and are each more than ten amino acids in length.

Generally, a first member of a protein:protein binding pair amino acid sequence as described herein, e.g., comprising a first member of a specific binding pair by itself or in combination with one or more linkers, is between about 5 amino acids to about 50 amino acids in length. In some embodiments, the first member of a protein:protein binding pair amino acid sequence is at least 5 amino acids in length. In some embodiments, the first member of a protein:protein binding pair amino acid sequence is 6 amino acids in length. In some embodiments, the first member of a protein:protein binding pair amino acid sequence is 7 amino acids in length. In some embodiments, the first member of a protein:protein binding pair amino acid sequence is 8 amino acids in length. In some embodiments, the first member of a protein:protein binding pair amino acid sequence is 9 amino acids in length. In some embodiments, the first member of a protein:protein binding pair amino acid sequence is 10 amino acids in length. In some embodiments, the first member of a protein:protein binding pair amino acid sequence is 11 amino acids in length. In some embodiments, the first member of a protein:protein binding pair amino acid sequence is 12 amino acids in length. In some embodiments, the first member of a protein:protein binding pair amino acid sequence is 13 amino acids in length. In some embodiments, the first member of a protein:protein binding pair amino acid sequence is 14 amino acids in length. In some embodiments, the first member of a protein:protein binding pair amino acid sequence is 15 amino acids in length. In some embodiments, the first member of a protein:protein binding pair amino acid sequence is 16 amino acids in length. In some embodiments, the first member of a protein:protein binding pair amino acid sequence is 17 amino acids in length. In some embodiments, the first member of a protein:protein binding pair amino acid sequence is 18 amino acids in length. In some embodiments, the first member of a protein:protein binding pair amino acid sequence is 19 amino acids in length. In some embodiments, the first member of a protein:protein binding pair amino acid sequence is 20 amino acids in length. In some embodiments, the first member of a protein:protein binding pair amino acid sequence is 21 amino acids in length. In some embodiments, the first member of a protein:protein binding pair amino acid sequence is 22 amino acids in length. In some embodiments, the first member of a protein:protein binding pair amino acid sequence is 23 amino acids in length. In some embodiments, the first member of a protein:protein binding pair amino acid sequence is 24 amino acids in length. In some embodiments, the first member of a protein:protein binding pair amino acid sequence is 25 amino acids in length. In some embodiments, the first member of a protein:protein binding pair amino acid sequence is 26 amino acids in length. In some embodiments, the first member of a protein:protein binding pair amino acid sequence is 27 amino acids in length. In some embodiments, the first member of a protein:protein binding pair amino acid sequence is 28 amino acids in length. In some embodiments, the first member of a protein:protein binding pair amino acid sequence is 29 amino acids in length. In some embodiments, the first member of a protein:protein binding pair amino acid sequence is 30 amino acids in length. In some embodiments, the first member of a protein:protein binding pair amino acid sequence is 31 amino acids in length. In some embodiments, the first member of a protein:protein binding pair amino acid sequence is 32 amino acids in length. In some embodiments, the first member of a protein:protein binding pair amino acid sequence is 33 amino acids in length. In some embodiments, the first member of a protein:protein binding pair amino acid sequence is 34 amino acids in length. In some embodiments, the first member of a protein:protein binding pair amino acid sequence is 35 amino acids in length. In some embodiments, the first member of a protein:protein binding pair amino acid sequence is 36 amino acids in length. In some embodiments, the first member of a protein:protein binding pair amino acid sequence is 37 amino acids in length. In some embodiments, the first member of a protein:protein binding pair amino acid sequence is 38 amino acids in length. In some embodiments, the first member of a protein:protein binding pair amino acid sequence is 39 amino acids in length. In some embodiments, the first member of a protein:protein binding pair amino acid sequence is 40 amino acids in length. In some embodiments, the first member of a protein:protein binding pair amino acid sequence is 41 amino acids in length. In some embodiments, the first member of a protein:protein binding pair amino acid sequence is 42 amino acids in length. In some embodiments, the first member of a protein:protein binding pair amino acid sequence is 43 amino acids in length. In some embodiments, the first member of a protein:protein binding pair amino acid sequence is 44 amino acids in length. In some embodiments, the first member of a protein:protein binding pair amino acid sequence is 45 amino acids in length. In some embodiments, the first member of a protein:protein binding pair amino acid sequence is 46 amino acids in length. In some embodiments, the first member of a protein:protein binding pair amino acid sequence is 47 amino acids in length. In some embodiments, the first member of a protein:protein binding pair amino acid sequence is 48 amino acids in length. In some embodiments, the first member of a protein:protein binding pair amino acid sequence is 49 amino acids in length. In some embodiments, the first member of a protein:protein binding pair amino acid sequence is 50 amino acids in length.

### Modified Capsids Comprising Modified Capsid Proteins

In some embodiments a viral capsid comprising a modified viral capsid protein as described herein is a mosaic capsid, e.g., comprises at least two sets of VP1, VP2, and/or VP3 proteins, each set of which is encoded by a different cap gene. A mosaic capsid herein generally refers to a mosaic of a first viral capsid protein modified to comprise a first member of a binding pair and a second corresponding viral capsid protein lacking the first member of a binding pair. In relation to a mosaic capsid, the second viral capsid protein lacking the first member of a binding pair may be referred to as a reference capsid protein encoded by a reference cap gene. In some mosaic capsid embodiments, preferably when the VP1, VP2, and/or VP3 capsid proteins modified with a first member of protein:protein pair is not a chimeric capsid protein, a VP1, VP2, and/or VP3 reference capsid protein may comprise an amino acid sequence identical to that of the viral VP1, VP2, and/or VP3 capsid protein modified with a first member of a binding pair, except that the reference capsid protein lacks the first member of a binding pair. In some mosaic capsid embodiments, a VP1, VP2, and/or VP3 reference capsid protein corresponds to the viral VP1, VP2, and/or VP3 capsid protein modified with a first member of a binding pair, except that the reference capsid protein lacks the first member of a binding pair. In some embodiments, a VP1 reference capsid protein corresponds to the viral VP1 capsid protein modified with a first member of a binding pair, except that the reference capsid protein lacks the first member of a binding pair. In some embodiments, a VP2 reference capsid protein corresponds to the viral VP2 capsid protein modified with a first member of a binding pair, except that the reference capsid protein lacks the first member of a binding pair. In some embodiments, a VP3 reference capsid protein corresponds to the viral VP3 capsid protein modified with a first member of a binding pair, except that the reference capsid protein lacks the first member of a binding pair. In some mosaic capsid embodiments comprising a chimeric VP1, VP2, and/or VP3 capsid protein further modified to comprise a first member of a binding pair, a reference protein may be a corresponding capsid protein from which portions thereof form part of the chimeric capsid protein. As a non-limiting example in some embodiments, mosaic capsid comprising a chimeric AAV2/AAAV VP1 capsid protein modified to comprise a first member of a binding pair may further comprise as a reference capsid protein: an AAV2 VP1 capsid protein lacking the first member, an AAAV VP1 capsid protein lacking the first member, a chimeric AAV2/AAAV VP1 capsid protein lacking the first member. Similarly, in some embodiments, a mosaic capsid comprising a chimeric AAV2/AAAV VP2 capsid protein modified to comprise a first member of a binding pair may further comprise as a reference capsid protein: an AAV2 VP2 capsid protein lacking the first member, an AAAV VP1 capsid protein lacking the first member, a chimeric AAV2/AAAV VP2 capsid protein lacking the first member. In some embodiments, a mosaic capsid comprising a chimeric AAV2/AAAV VP3 capsid protein modified to comprise a first member of a binding pair may further comprise as a reference capsid protein: an AAV2 VP2 capsid protein lacking the first member, an AAAV VP1 capsid protein lacking the first member, a chimeric AAV2/AAAV VP3 capsid protein lacking the first member. In some mosaic capsid embodiments, a reference capsid protein may be any capsid protein so long as it that lacks the first member of the binding pair and is able to form a capsid with the first capsid protein modified with the first member of a binding pair.

Generally mosaic particles may be generated by transfecting mixtures of the modified and reference Cap genes into production cells at the indicated ratios. The protein subunit ratios, e.g., modified VP protein:unmodified VP protein ratios, in the particle may, but do not necessarily, stoichiometrically reflect the ratios of the at least two species of the cap gene encoding the first capsid protein modified with a first member of a binding pair and the one or more reference cap genes, e.g., modified cap gene:reference cap gene(s) transfected into packaging cells. In some embodiments, the protein subunit ratios in the particle do not stoichiometrically reflect the modified cap gene:reference cap gene(s) ratio transfected into packaging cells.

In some mosaic viral particle embodiments, the protein subunit ratio ranges from about 1:59 to about 59:1. In some mosaic viral particle embodiments, the protein subunit is at least about 1:1 (e.g., the mosaic viral particle comprises about 30 modified capsid proteins and about 30 reference capsid protein). In some mosaic viral particle embodiments, the protein subunit ratio is at least about 1:2 (e.g., the mosaic viral particle comprises about 20 modified capsid proteins and about 40 reference capsid proteins). In some mosaic viral particle embodiments, the protein subunit ratio is at least about 3:5. In some mosaic viral particle embodiments, the protein subunit ratio is at least about 1:3 (e.g., the mosaic viral particle comprises about 15 modified capsid proteins and about 45 reference capsid proteins). In some mosaic viral particle embodiments, the protein subunit ratio is at least about 1:4 (e.g., the mosaic viral particle comprises about 12 modified capsid proteins and 48 reference capsid proteins). In some mosaic viral particle embodiments, the protein subunit ratio is at least about 1:5 (e.g., the mosaic viral particle comprises 10 modified capsid proteins and 50 reference capsid proteins). In some mosaic viral particle embodiments, the protein subunit ratio is at least about 1:6. In some mosaic viral particle embodiments, the protein subunit ratio is at least about 1:7. In some mosaic viral particle embodiments, the protein subunit ratio is at least about 1:8. In some mosaic viral particle embodiments, the protein subunit ratio is at least about 1:9 (e.g., the mosaic viral particle comprises about 6 modified capsid proteins and about 54 reference capsid proteins). In some mosaic viral particle embodiments, the protein subunit ratio is at least about 1:10. In some mosaic viral particle embodiments, the protein subunit ratio is at least about 1:11 (e.g., the mosaic viral particle comprises about 5 modified capsid proteins and about 55 reference capsid proteins). In some mosaic viral particle embodiments, the protein subunit ratio is at least about 1:12. In some mosaic viral particle embodiments, the protein subunit ratio is at least about 1:13. In some mosaic viral particle embodiments, the protein subunit ratio is at least about 1:14 (e.g., the mosaic viral particle comprises about 4 modified capsid proteins and about 56 reference capsid proteins). In some mosaic viral particle embodiments, the protein subunit ratio is at least about 1:15. In some mosaic viral particle embodiments, the protein subunit ratio is at least about 1:19 (e.g., the mosaic viral particle comprises about 3 modified capsid proteins and about 57 reference capsid proteins). In some mosaic viral particle embodiments, the protein subunit ratio is at least about 1:29 (e.g., the mosaic viral particle comprises about 2 modified capsid proteins and about 58 reference capsid proteins). In some mosaic viral particle embodiments, the protein subunit ratio is at least about 1:59. In some mosaic viral particle embodiments, the protein subunit ratio is at least about 2:1 (e.g., the mosaic viral particle comprises about 40 modified capsid proteins and about 20 reference capsid proteins). In some mosaic viral particle embodiments, the protein subunit ratio is at least about 5:3. In some mosaic viral particle embodiments, the protein subunit ratio is at least about 3:1 (e.g., the mosaic viral particle comprises about 45 modified capsid proteins and about 15 reference capsid proteins). In some mosaic viral particle embodiments, the protein subunit ratio is at least about 4:1 (e.g., the mosaic viral particle comprises about 48 modified capsid proteins and 12 reference capsid proteins). In some mosaic viral particle embodiments, the protein subunit ratio is at least about 5:1 (e.g., the mosaic viral particle comprises 50 modified capsid proteins and 10 reference capsid proteins). In some mosaic viral particle embodiments, the protein subunit ratio is at least about 6:1. In some mosaic viral particle embodiments, the protein subunit ratio is at least about 7:1. In some mosaic viral particle embodiments, the protein subunit ratio is at least about 8:1. In some mosaic viral particle embodiments, the protein subunit ratio is at least about 9:1 (e.g., the mosaic viral particle comprises about 54 modified capsid proteins and about 6 reference capsid proteins). In some mosaic viral particle embodiments, the protein subunit ratio is at least about 10:1. In some mosaic viral particle embodiments, the protein subunit ratio is at least about 11:1 (e.g., the mosaic viral particle comprises about 55 modified capsid proteins and about 5 reference capsid proteins). In some mosaic viral particle embodiments, the protein subunit ratio is at least about 12:1. In some mosaic viral particle embodiments, the protein subunit ratio is at least about 13:1. In some mosaic viral particle embodiments, the protein subunit ratio is at least about 14:1 (e.g., the mosaic viral particle comprises about 56 modified capsid proteins and about 4 reference capsid proteins). In some mosaic viral particle embodiments, the protein subunit ratio is at least about 15:1. In some mosaic viral particle embodiments, the protein subunit ratio is at least about 19:1 (e.g., the mosaic viral particle comprises about 57 modified capsid proteins and about 3 reference capsid proteins). In some mosaic viral particle embodiments, the protein subunit ratio is at least about 29:1 (e.g., the mosaic viral particle comprises about 58 modified capsid proteins and about 2 reference capsid proteins). In some mosaic viral particle embodiments, the protein subunit ratio is at least about 59:1.

In some non-mosaic viral particle embodiments, the protein subunit ratio may be 1:0 wherein each capsid protein of the non-mosaic viral particle is modified with a first member of a binding pair. In some non-mosaic viral particle embodiments, the protein subunit ratio may be 0:1 wherein each capsid protein of the non-mosaic viral particle is not modified with a first member of a binding pair.

### Insertion Sites

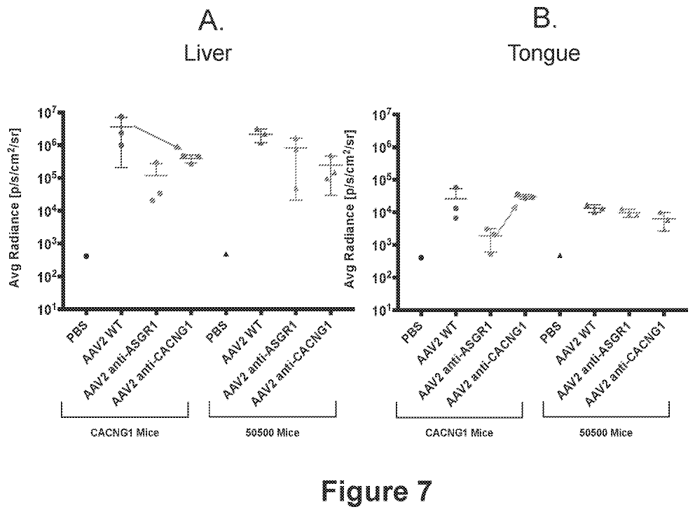

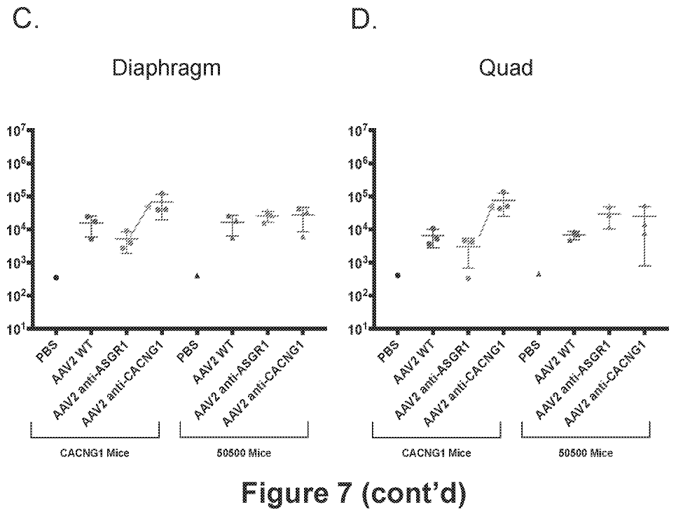

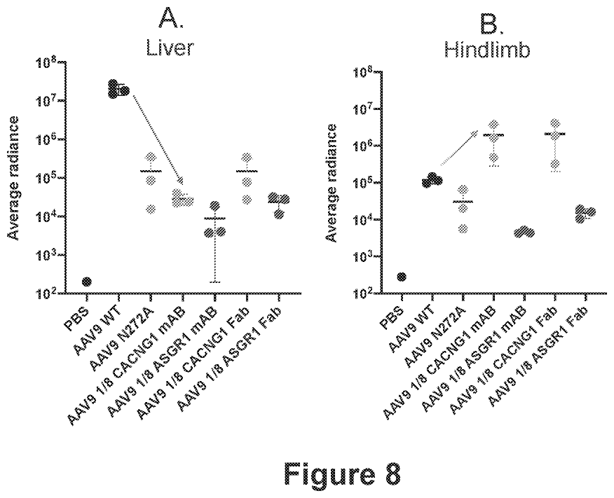

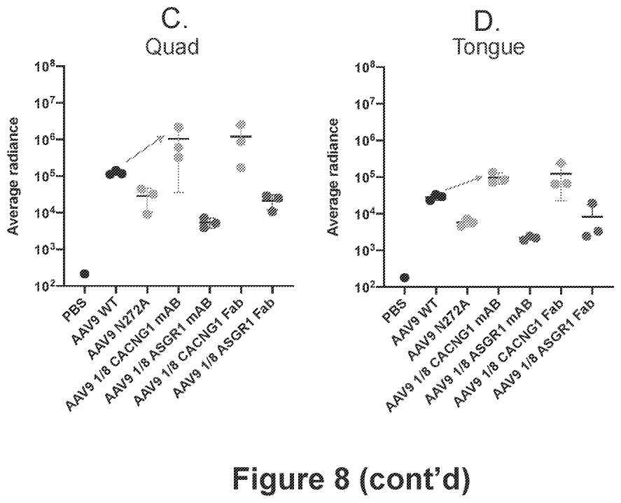

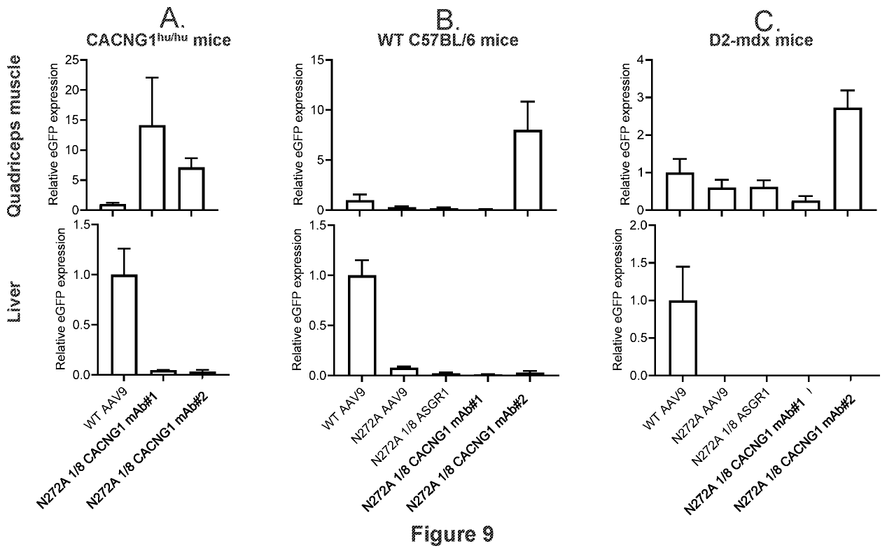

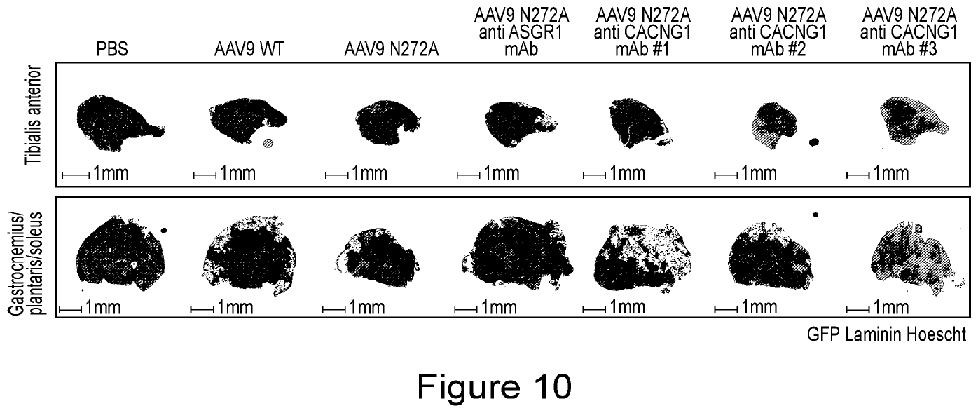

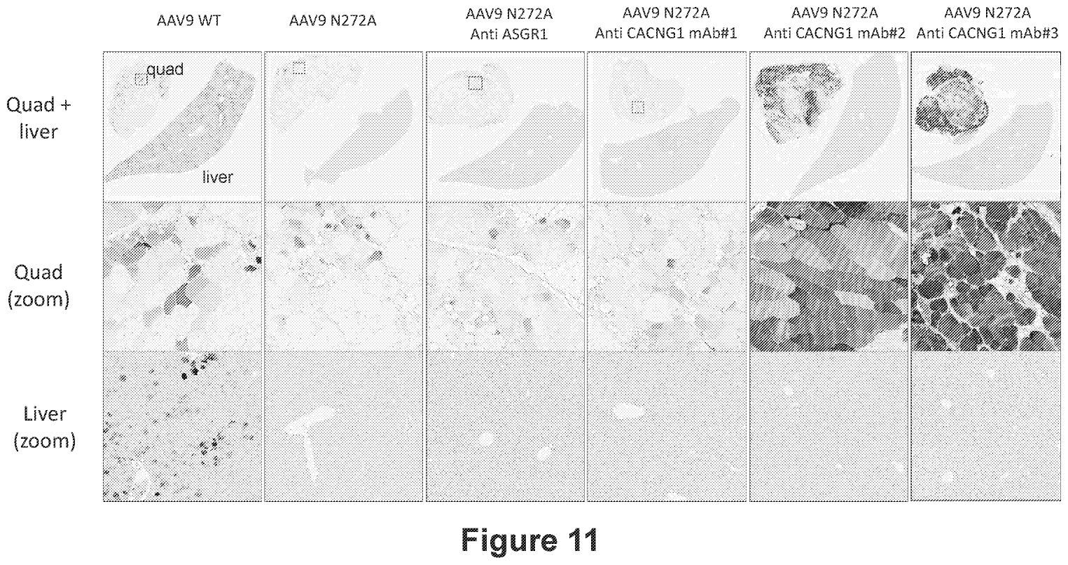

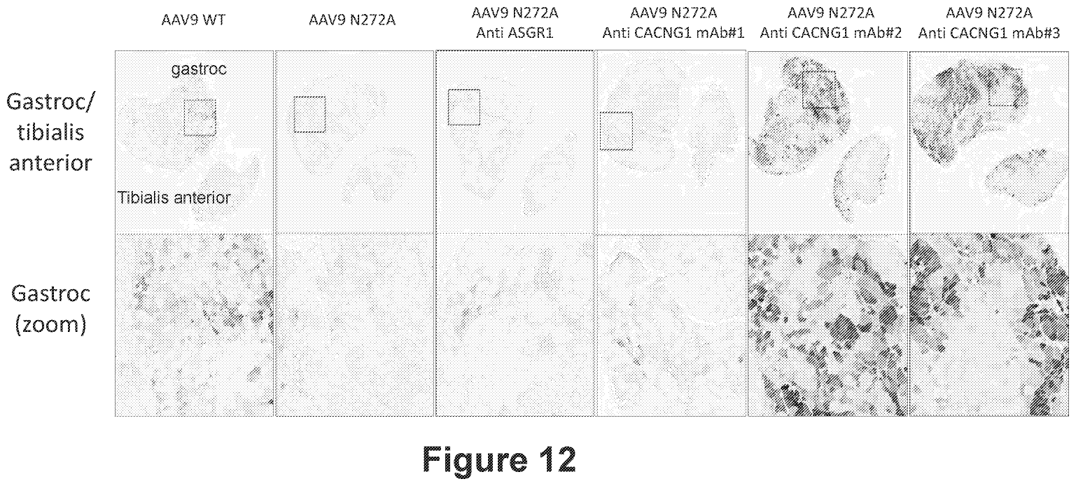

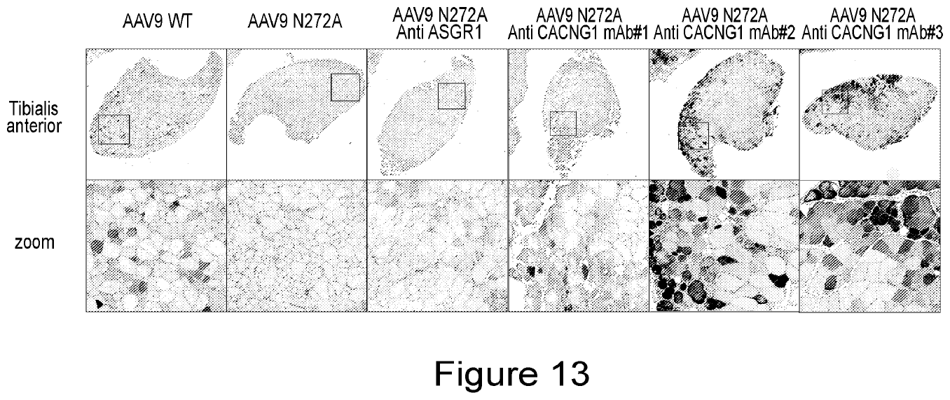

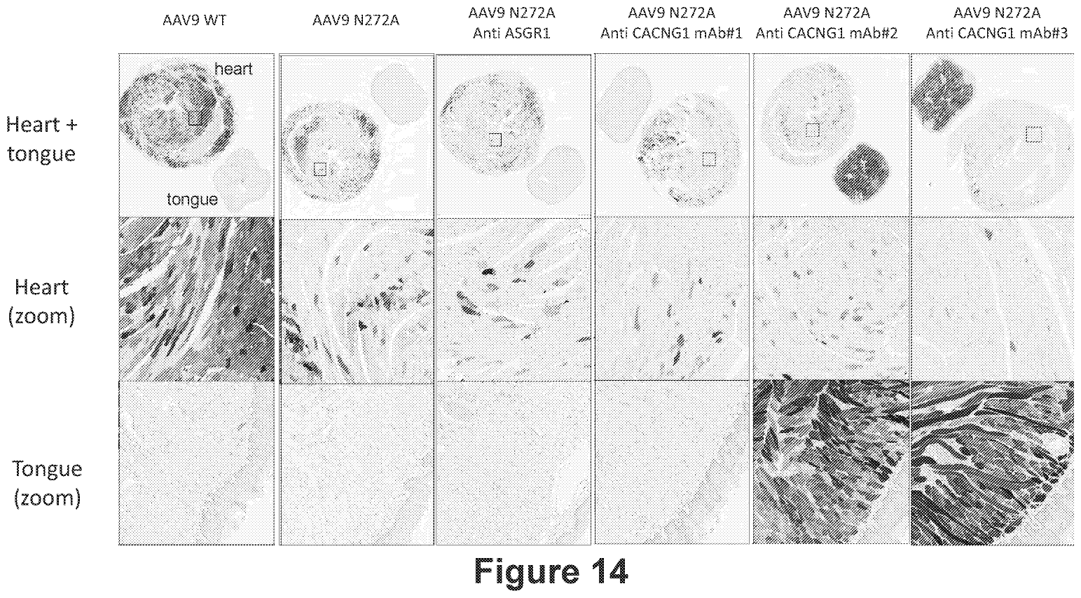

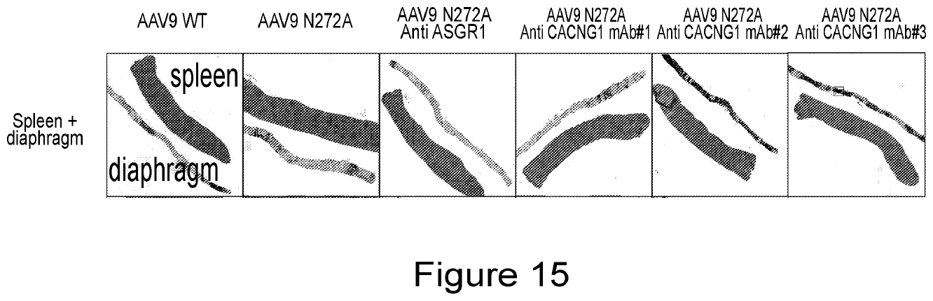

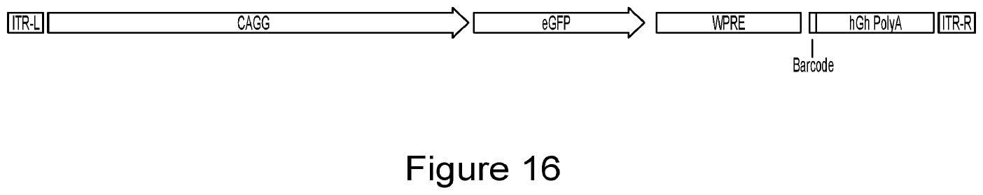

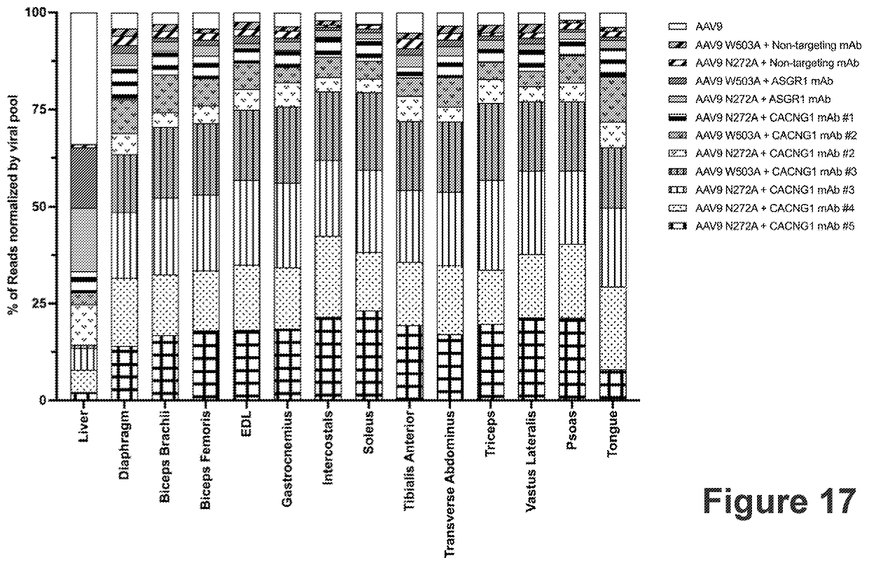

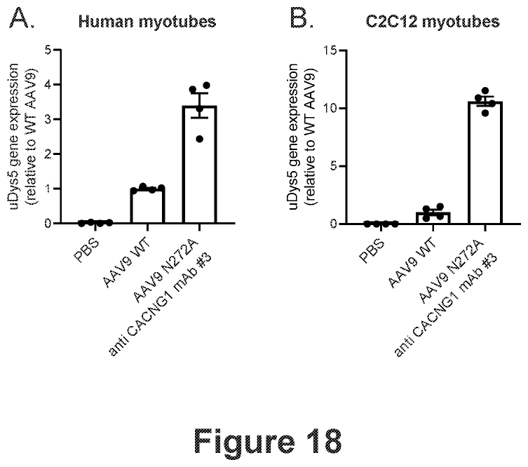

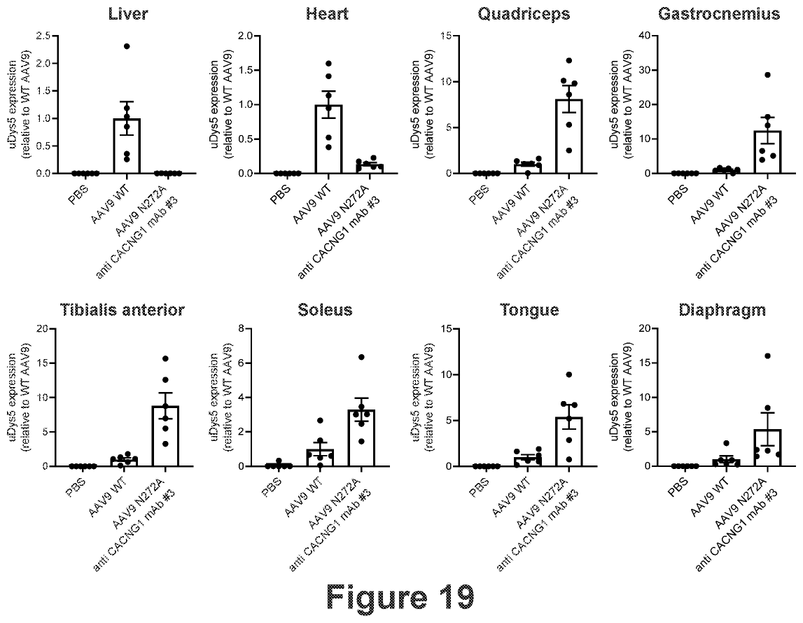

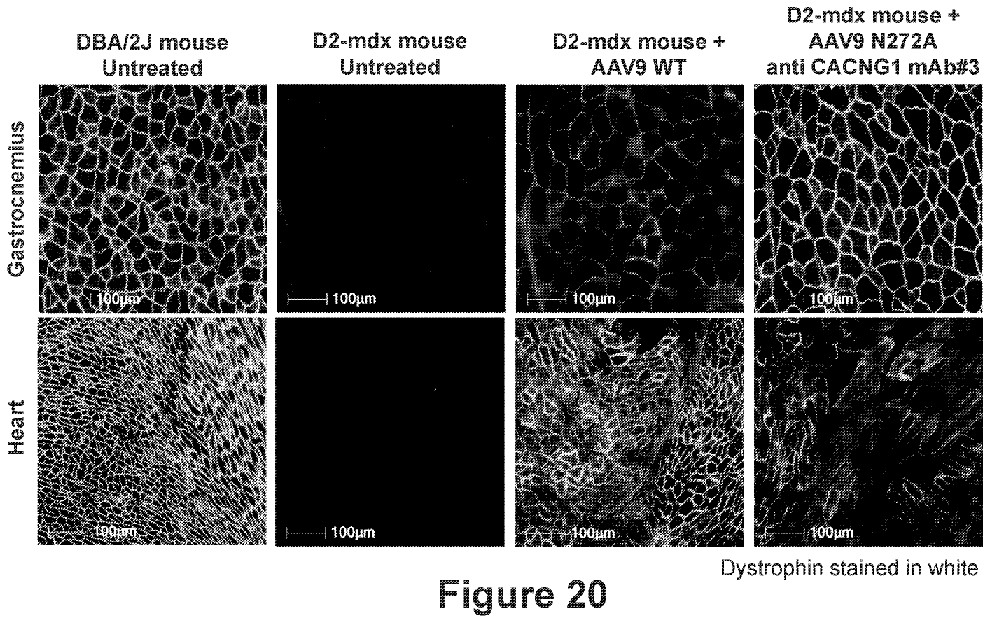

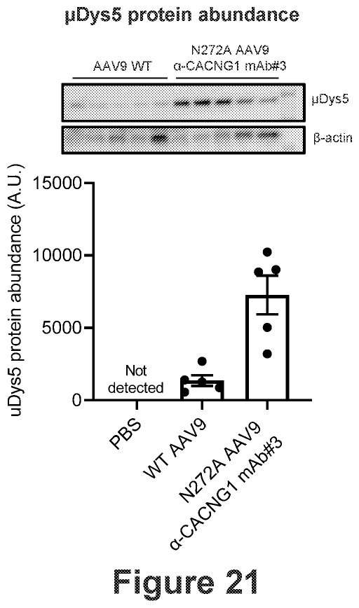

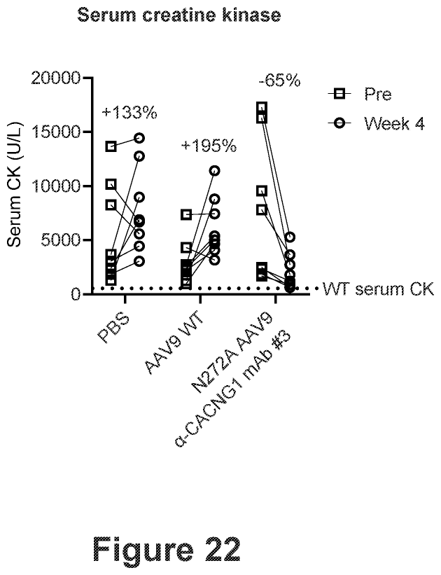

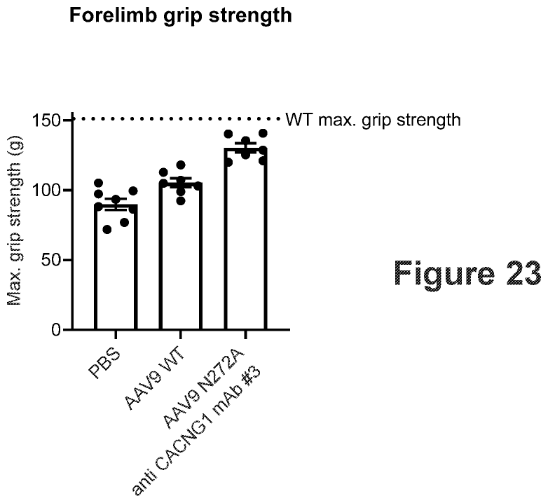

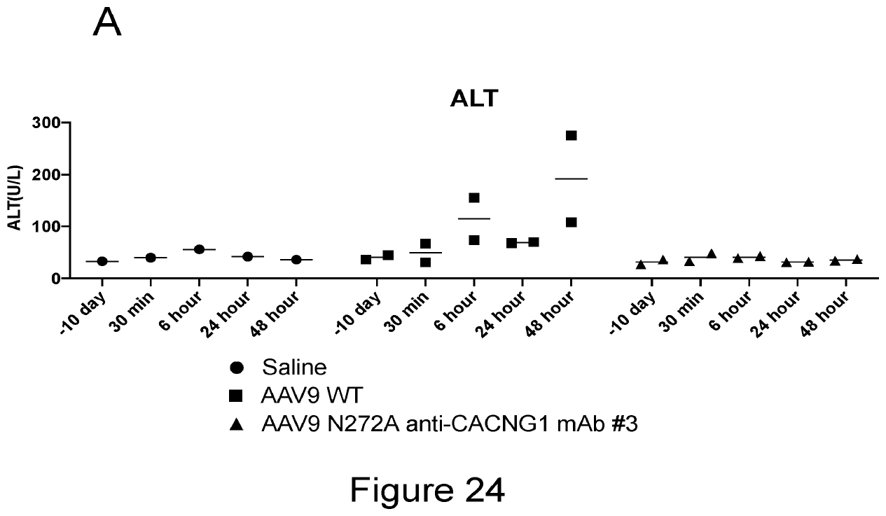

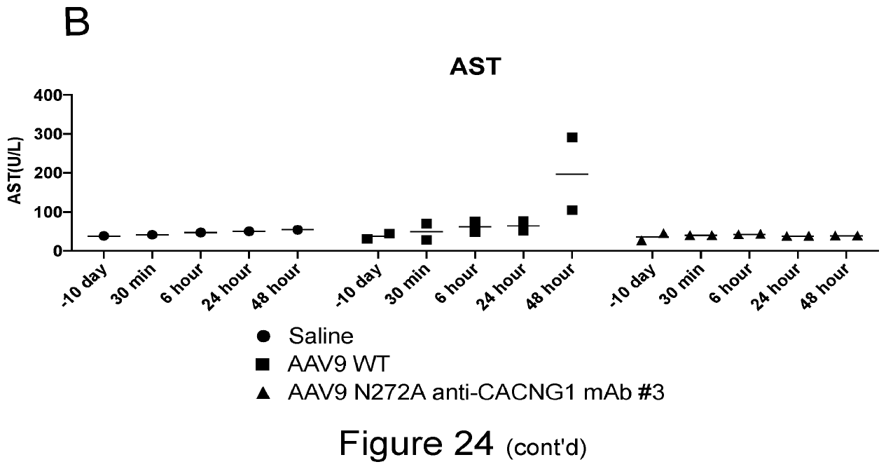

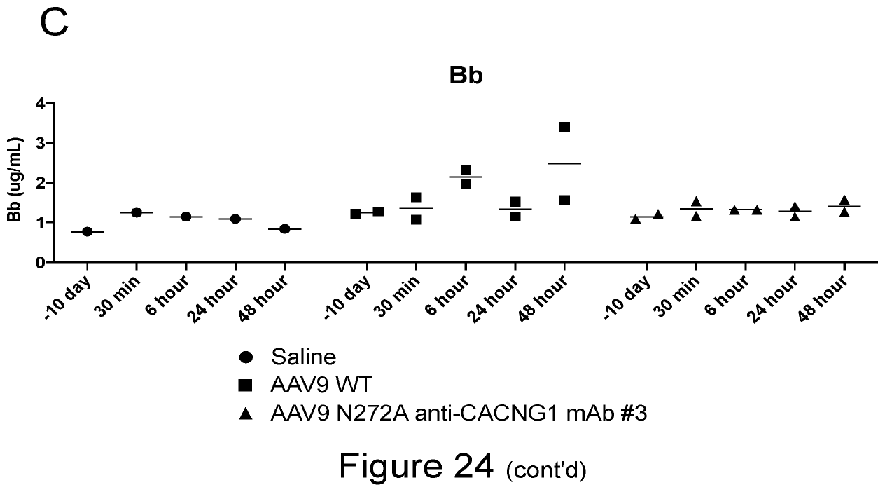

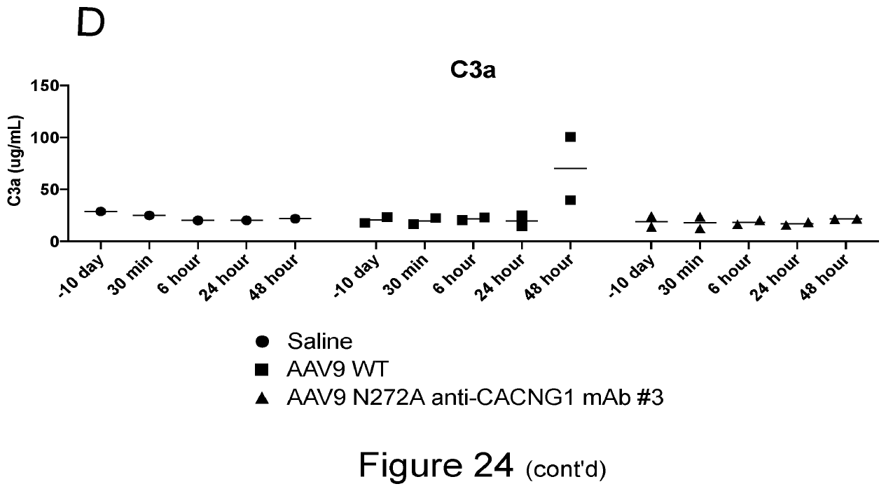

Due to the high conservation of at least large stretches and the large member of closely related family members, the corresponding insertion sites for AAV other than the enumerated AAV can be identified by performing an amino acid alignment or by comparison of the capsid structures. See, e.g., Rutledge et al. (1998) J. Virol. 72:309-19; Mietzsch et al. (2019) Viruses 11, 362, 1-34, and U.S. Pat. No. 9,624,274 for exemplary alignments of different AAV capsid proteins, each of which is incorporated herein by reference in its entirety. For example, Mietzcsh et al. (2019) provide an overlay of ribbons from different dependoparvovirus at FIG. 7, depicting the variable regions VR I to VR IX. Using such structural analysis as described therein, and sequence analysis, a skilled artisan may determine which amino acids within the variable region correspond to amino acid sequence of AAV that can accommodate the insertion of, e.g., a targeting ligand as described herein, a first member of a binding pair and/or detectable label.

Generally, the targeting ligand, first member of a binding pair, and/or detectable label may be inserted into a variable region or variable loop of an AAV capsid protein, a GH loop of an AAV capsid protein, etc.

In some embodiments, the first member of a binding pair and/or detectable label is inserted in a VP1 capsid protein of a non-primate animal AAV after an amino acid position corresponding with an amino acid position selected from the group consisting of G453 of AAV2 capsid protein VP1, N587 of AAV2 capsid protein VP1, G453 of AAV9 capsid protein VP1, and A589 of AAV9 capsid protein VP1. In some embodiments, the first member of a binding pair and/or detectable label is inserted in a VP1 capsid protein of a non-primate animal AAV between amino acids that correspond with N587 and R588 of an AAV2 VP1 capsid. Additional suitable insertion sites of a non-primate animal VP1 capsid protein include those corresponding to I-1, I-34, I-138, I-139, I-161, I-261, I-266, I-381, I-447, I-448, I-459, I-471, I-520, I-534, I-570, I-573, I-584, I-587, I-588, I-591, I-657, I-664, I-713 and I-716 of the VP1 capsid protein of AAV2 (Wu et al. (2000) J. Virol. 74:8635-8647). A modified virus capsid protein as described herein may be a non-primate animal capsid protein comprising a first member of a binding pair and/or detectable label inserted into a position corresponding with a position of an AAV2 capsid protein selected from the group consisting of I-1, I-34, I-138, I-139, I-161, I-261, I-266, I-381, I-447, I-448, I-459, I-471, I-520, I-534, I-570, I-573, I-584, I-587, I-588, I-591, I-657, I-664, I-713, I-716, and a combination thereof. Additional suitable insertion sites of a non-primate animal AAV that include those corresponding to I-587 or I-590 of AAV1, I-589 of AAV1, I-585 of AAV3, I-584 or I-585 of AAV4, and I-575 or I-585 of AAV5. In some embodiments, a modified virus capsid protein as described herein may be a non-primate animal capsid protein comprising a targeting ligand, first member of a binding pair and/or detectable label inserted into a position corresponding with a position selected from the group consisting of I-587 (AAV1), I-589 (AAV1), I-585 (AAV3), I-585 (AAV4), I-585 (AAV5), and a combination thereof.

In some embodiments, the first member of a binding pair and/or detectable label is inserted in a VP1 capsid protein of a non-primate animal AAV after an amino acid position corresponding with an amino acid position selected from the group consisting of 1444 of an avian AAV capsid protein VP1, 1580 of an avian AAV capsid protein VP1, 1573 of a bearded dragon AAV capsid protein VP1, 1436 of a bearded dragon AAV capsid protein VP1, 1429 of a sea lion AAV capsid protein VP1, 1430 of a sea lion AAV capsid protein VP1, I431 of a sea lion AAV capsid protein VP1, 1432 of a sea lion AAV capsid protein VP1, 1433 of a sea lion AAV capsid protein VP1, 1434 of a sea lion AAV capsid protein VP1, 1436 of a sea lion AAV capsid protein VP1, 1437 of a sea lion AAV capsid protein VP1, and 1565 of a sea lion AAV capsid protein VP1.

The nomenclature I-###, I # or the like herein refers to the insertion site (I) with ### naming the amino acid number relative to the VP1 protein of an AAV capsid protein, however such the insertion may be located directly N- or C-terminal, preferably C-terminal of one amino acid in the sequence of 5 amino acids N- or C-terminal of the given amino acid, preferably 3, more preferably 2, especially 1 amino acid(s) N- or C-terminal of the given amino acid. Additionally, the positions referred to herein are relative to the VP1 protein encoded by an AAV capsid gene, and corresponding positions (and point mutations thereof) may be easily identified for the VP2 and VP3 capsid proteins encoding by the capsid gene by performing a sequence alignment of the VP1, VP2 and VP3 proteins encoded by the appropriate AAV capsid gene.

Accordingly, an insertion into the corresponding position of the coding nucleic acid of one of these sites of the cap gene leads to an insertion into VP1, VP2 and/or VP3, as the capsid proteins are encoded by overlapping reading frames of the same gene with staggered start codons. Therefore, for AAV2, for example, according to this nomenclature insertions between amino acids 1 and 138 are only inserted into VP1, insertions between 138 and 203 are inserted into VP1 and VP2, and insertions between 203 and the C-terminus are inserted into VP1, VP2 and VP3, which is of course also the case for the insertion site I-587. Therefore, the present invention encompasses structural genes of AAV with corresponding insertions in the VP1, VP2 and/or VP3 proteins.

Also provided herein are nucleic acids that encode a VP3 capsid protein of the invention. AAV capsid proteins may be, but are not necessarily, encoded by overlapping reading frames of the same gene with staggered start codons. In some embodiments, a nucleic acid that encodes a VP3 capsid protein of the invention does not also encode a VP2 capsid protein or VP1 capsid protein of the invention. In some embodiments, a nucleic acid that encodes a VP3 capsid protein of the invention may also encode a VP2 capsid protein of the invention but does not also encode a VP1 capsid of the invention. In some embodiments, a nucleic acid that encodes a VP3 capsid protein of the invention may also encode a VP2 capsid protein of the invention and a VP1 capsid of the invention.

In some embodiments, a viral capsid comprising the modified viral capsid protein comprising the first and second members of a binding pair (e.g., wherein the second member is operably linked to a targeting ligand, comprises a multispecific binding protein, etc.) is able to infect a specific cell, e.g., has an enhanced capacity to target and bind a specific cell compared to that of a control viral capsid that is identical to the modified viral capsid protein except that it lacks either or both the first and second members of a binding pair, e.g., comprises a control capsid protein. In some embodiments, a viral capsid comprising a modified viral capsid protein as described herein bound to the first and second members of a binding pair linked to a targeting ligand exhibits a detectable transduction efficiency compared to the undetectable transduction efficiency of a control viral capsid. In some embodiments, a viral capsid comprising a modified viral capsid protein as described herein bound to the first and second members of a binding pair linked to a targeting ligand exhibits a transduction efficiency that is 10% greater than the transduction efficiency of a control viral capsid. In some embodiments, a viral capsid comprising a modified viral capsid protein as described herein bound to the first and second members of a binding pair linked to a targeting ligand exhibits a transduction efficiency that is 20% greater than the transduction efficiency of a control viral capsid. In some embodiments, a viral capsid comprising a modified viral capsid protein as described herein bound to an appropriate the first and second members of a binding pair linked to a targeting ligand exhibits a transduction efficiency that is 30% greater than the transduction efficiency of a control viral capsid. In some embodiments, a viral capsid comprising a modified viral capsid protein as described herein bound to an appropriate the first and second members of a binding pair linked to a targeting ligand exhibits a transduction efficiency that is 40% greater than the transduction efficiency of a control viral capsid. In some embodiments, a viral capsid comprising a modified viral capsid protein as described herein bound to an appropriate the first and second members of a binding pair linked to a targeting ligand exhibits a transduction efficiency that is 50% greater than the transduction efficiency of a control viral capsid. In some embodiments, a viral capsid comprising a modified viral capsid protein as described herein bound to an appropriate the first and second members of a binding pair linked to a targeting ligand exhibits a transduction efficiency that is 60% greater than the transduction efficiency of a control viral capsid. In some embodiments, a viral capsid comprising a modified viral capsid protein as described herein bound to an appropriate the first and second members of a binding pair linked to a targeting ligand exhibits a transduction efficiency that is 70% greater than the transduction efficiency of a control viral capsid. In some embodiments, a viral capsid comprising a modified viral capsid protein as described herein bound to an appropriate the first and second members of a binding pair linked to a targeting ligand exhibits a transduction efficiency that is 75% greater than the transduction efficiency of a control viral capsid. In some embodiments, a viral capsid comprising a modified viral capsid protein as described herein bound to an appropriate the first and second members of a binding pair linked to a targeting ligand exhibits a transduction efficiency that is 80% greater than the transduction efficiency of a control viral capsid. In some embodiments, a viral capsid comprising a modified viral capsid protein as described herein bound to an appropriate the first and second members of a binding pair linked to a targeting ligand exhibits a transduction efficiency that is 85% greater than the transduction efficiency of a control viral capsid. In some embodiments, a viral capsid comprising a modified viral capsid protein as described herein bound to an appropriate the first and second members of a binding pair linked to a targeting ligand exhibits a transduction efficiency that is 90% greater than the transduction efficiency of a control capsid. In some embodiments, a viral capsid comprising a modified viral capsid protein as described herein bound to an appropriate the first and second members of a binding pair linked to a targeting ligand exhibits a transduction efficiency that is 95% greater than the transduction efficiency of a control viral capsid. In some embodiments, a viral capsid comprising a modified viral capsid protein as described herein bound to the first and second members of a binding pair linked to a targeting ligand exhibits a transduction efficiency that is 99% greater than the transduction efficiency of a control viral capsid.

In some embodiments, a viral capsid comprising the modified viral capsid protein comprising the first and second members of a binding pair (e.g., wherein the second member is operably linked to a targeting ligand, comprises a multispecific binding protein, etc.) is able to infect a specific cell, e.g., has an enhanced capacity to target and bind a specific cell compared to that of a control viral capsid that is identical to the modified viral capsid protein except that it lacks either or both the first and second members of a binding pair, e.g., comprises a control capsid protein. In some embodiments, a viral capsid comprising a modified viral capsid protein as described herein bound to the first and second members of a binding pair linked to a targeting ligand exhibits a detectable transduction efficiency compared to the undetectable transduction efficiency of a control viral capsid. In some embodiments, a viral capsid comprising a modified viral capsid protein as described herein bound to the first and second members of a binding pair linked to a targeting ligand exhibits a transduction efficiency that is 10% greater than the transduction efficiency of a control viral capsid. In some embodiments, a viral capsid comprising a modified viral capsid protein as described herein bound to the first and second members of a binding pair linked to a targeting ligand exhibits a transduction efficiency that is 20% greater than the transduction efficiency of a control viral capsid. In some embodiments, a viral capsid comprising a modified viral capsid protein as described herein bound to an appropriate the first and second members of a binding pair linked to a targeting ligand exhibits a transduction efficiency that is 30% greater than the transduction efficiency of a control viral capsid. In some embodiments, a viral capsid comprising a modified viral capsid protein as described herein bound to an appropriate the first and second members of a binding pair linked to a targeting ligand exhibits a transduction efficiency that is 40% greater than the transduction efficiency of a control viral capsid. In some embodiments, a viral capsid comprising a modified viral capsid protein as described herein bound to an appropriate the first and second members of a binding pair linked to a targeting ligand exhibits a transduction efficiency that is 50% greater than the transduction efficiency of a control viral capsid. In some embodiments, a viral capsid comprising a modified viral capsid protein as described herein bound to an appropriate the first and second members of a binding pair linked to a targeting ligand exhibits a transduction efficiency that is 60% greater than the transduction efficiency of a control viral capsid. In some embodiments, a viral capsid comprising a modified viral capsid protein as described herein bound to an appropriate the first and second members of a binding pair linked to a targeting ligand exhibits a transduction efficiency that is 70% greater than the transduction efficiency of a control viral capsid. In some embodiments, a viral capsid comprising a modified viral capsid protein as described herein bound to an appropriate the first and second members of a binding pair linked to a targeting ligand exhibits a transduction efficiency that is 75% greater than the transduction efficiency of a control viral capsid. In some embodiments, a viral capsid comprising a modified viral capsid protein as described herein bound to an appropriate the first and second members of a binding pair linked to a targeting ligand exhibits a transduction efficiency that is 80% greater than the transduction efficiency of a control viral capsid. In some embodiments, a viral capsid comprising a modified viral capsid protein as described herein bound to an appropriate the first and second members of a binding pair linked to a targeting ligand exhibits a transduction efficiency that is 85% greater than the transduction efficiency of a control viral capsid. In some embodiments, a viral capsid comprising a modified viral capsid protein as described herein bound to an appropriate the first and second members of a binding pair linked to a targeting ligand exhibits a transduction efficiency that is 90% greater than the transduction efficiency of a control capsid. In some embodiments, a viral capsid comprising a modified viral capsid protein as described herein bound to an appropriate the first and second members of a binding pair linked to a targeting ligand exhibits a transduction efficiency that is 95% greater than the transduction efficiency of a control viral capsid. In some embodiments, a viral capsid comprising a modified viral capsid protein as described herein bound to the first and second members of a binding pair linked to a targeting ligand exhibits a transduction efficiency that is 99% greater than the transduction efficiency of a control viral capsid. In some embodiments, a viral capsid comprising a modified viral capsid protein as described herein bound to the first and second members of a binding pair linked to a targeting ligand exhibits a transduction efficiency that is at least1.5-fold greater than the transduction efficiency of a control viral capsid. In some embodiments, a viral capsid comprising a modified viral capsid protein as described herein bound to the first and second members of a binding pair linked to a targeting ligand exhibits a transduction efficiency that is at least 2-fold greater than the transduction efficiency of a control viral capsid. In some embodiments, a viral capsid comprising a modified viral capsid protein as described herein bound to an appropriate the first and second members of a binding pair linked to a targeting ligand exhibits a transduction efficiency that is at least 3-fold greater than the transduction efficiency of a control viral capsid. In some embodiments, a viral capsid comprising a modified viral capsid protein as described herein bound to an appropriate the first and second members of a binding pair linked to a targeting ligand exhibits a transduction efficiency that is at least 4-fold greater than the transduction efficiency of a control viral capsid. In some embodiments, a viral capsid comprising a modified viral capsid protein as described herein bound to an appropriate the first and second members of a binding pair linked to a targeting ligand exhibits a transduction efficiency that is at least 5-fold greater than the transduction efficiency of a control viral capsid. In some embodiments, a viral capsid comprising a modified viral capsid protein as described herein bound to an appropriate the first and second members of a binding pair linked to a targeting ligand exhibits a transduction efficiency that is at least 6-fold greater than the transduction efficiency of a control viral capsid. In some embodiments, a viral capsid comprising a modified viral capsid protein as described herein bound to an appropriate the first and second members of a binding pair linked to a targeting ligand exhibits a transduction efficiency that is at least 7-fold greater than the transduction efficiency of a control viral capsid. In some embodiments, a viral capsid comprising a modified viral capsid protein as described herein bound to an appropriate the first and second members of a binding pair linked to a targeting ligand exhibits a transduction efficiency that is at least 8-fold greater than the transduction efficiency of a control viral capsid. In some embodiments, a viral capsid comprising a modified viral capsid protein as described herein bound to an appropriate the first and second members of a binding pair linked to a targeting ligand exhibits a transduction efficiency that is at least 9-fold greater than the transduction efficiency of a control viral capsid. In some embodiments, a viral capsid comprising a modified viral capsid protein as described herein bound to an appropriate the first and second members of a binding pair linked to a targeting ligand exhibits a transduction efficiency that is at least 10-fold greater than the transduction efficiency of a control viral capsid. In some embodiments, a viral capsid comprising a modified viral capsid protein as described herein bound to an appropriate the first and second members of a binding pair linked to a targeting ligand exhibits a transduction efficiency that is at least 20-fold greater than the transduction efficiency of a control capsid. In some embodiments, a viral capsid comprising a modified viral capsid protein as described herein bound to an appropriate the first and second members of a binding pair linked to a targeting ligand exhibits a transduction efficiency that is at least 30-fold greater than the transduction efficiency of a control viral capsid. In some embodiments, a viral capsid comprising a modified viral capsid protein as described herein bound to the first and second members of a binding pair linked to a targeting ligand exhibits a transduction efficiency that is at least 40-fold greater than the transduction efficiency of a control viral capsid. In some embodiments, a viral capsid comprising a modified viral capsid protein as described herein bound to the first and second members of a binding pair linked to a targeting ligand exhibits a transduction efficiency that is at least 50-fold greater than the transduction efficiency of a control viral capsid. In some embodiments, a viral capsid comprising a modified viral capsid protein as described herein bound to the first and second members of a binding pair linked to a targeting ligand exhibits a transduction efficiency that is at least 60-fold greater than the transduction efficiency of a control viral capsid. In some embodiments, a viral capsid comprising a modified viral capsid protein as described herein bound to the first and second members of a binding pair linked to a targeting ligand exhibits a transduction efficiency that is at least 70-fold greater than the transduction efficiency of a control viral capsid. In some embodiments, a viral capsid comprising a modified viral capsid protein as described herein bound to the first and second members of a binding pair linked to a targeting ligand exhibits a transduction efficiency that is at least 80-fold greater than the transduction efficiency of a control viral capsid. In some embodiments, a viral capsid comprising a modified viral capsid protein as described herein bound to the first and second members of a binding pair linked to a targeting ligand exhibits a transduction efficiency that is at least 90-fold greater than the transduction efficiency of a control viral capsid. In some embodiments, a viral capsid comprising a modified viral capsid protein as described herein bound to the first and second members of a binding pair linked to a targeting ligand exhibits a transduction efficiency that is at least 100-fold greater than the transduction efficiency of a control viral capsid In some embodiments, a viral particle of the invention comprising a viral capsid protein comprising an amino acid sequence of a capsid protein of a non-primate animal AAV, a remote AAV, or a combination thereof, and optionally comprising a first and second members of a binding pair (e.g., wherein the second member is operably linked to a targeting ligand, comprises a multispecific binding protein, etc.) is better able to evade neutralization by pre-existing antibodies in serum isolated from a human patient compared to an appropriate control viral particle (e.g., comprising a viral capsid of an AAV serotype from which a portion is included in the viral capsid of the invention, e.g., as part of the viral capsid protein comprising an amino acid sequence of a capsid protein of a non-primate animal AAV, a remote AAV, or a combination thereof), which also optionally comprises a first and second members of a binding pair (e.g., wherein the second member is operably linked to a targeting ligand, comprises a multispecific binding protein, etc.). In some embodiments, a viral particle of the invention comprising a viral capsid protein comprising an amino acid sequence of a capsid protein of a non-primate animal AAV, a remote AAV, or a combination thereof requires at least 2-fold more total IVIG or IgG for neutralization (e.g., 50% or more infection inhibition) compared to an appropriate control viral particle, e.g., (e.g., a viral particle of the invention has an IC50 value that is at least 2-fold that of a control virus particle).

In some embodiments of the invention comprising a detectable label, a targeting ligand comprises a multispecific binding molecule comprising (i) an antibody paratope that specifically binds the detectable label and (ii) a second binding domain that specifically binds a receptor, which may be conjugated to the surface of a bead (e.g., for purification) or expressed by a target cell. Accordingly, a multispecific binding molecule comprising (i) an antibody paratope that specifically binds the detectable label and (ii) a second binding domain that specifically binds a receptor targets the viral particle. Such “targeting” or “directing” may include a scenario in which the wildtype viral particle targets several cells within a tissue and/or several organs within an organism, which broad targeting of the tissue or organs is reduced to abolished by insertion of the detectable label, and which retargeting to more specific cells in the tissue or more specific organ in the organism is achieved with the multispecific binding molecule. Such retargeting or redirecting may also include a scenario in which the wildtype viral particle targets a tissue, which targeting of the tissue is reduced to abolished by insertion of the detectable label, and which retargeting to a completely different tissue is achieved with the multispecific binding molecule. An antibody paratope as described herein generally comprises at a minimum a complementarity determining region (CDR) that specifically recognizes the detectable label, e.g., a CDR3 region of a heavy and/or light chain variable domain. In some embodiments, a multispecific binding molecule comprises an antibody (or portion thereof) that comprises the antibody paratope that specifically binds the detectable label. For example, a multispecific binding molecule may comprise a single domain heavy chain variable region or a single domain light chain variable region, wherein the single domain heavy chain variable region or single domain light chain variable region comprises an antibody paratope that specifically binds the detectable label. In some embodiments, a multispecific binding molecule may comprise an Fv region, e.g., a multispecific binding molecule may comprise an scFv, that comprises an antibody paratope that specifically binds the detectable label. In some embodiments, a multispecific binding molecule as described herein comprises an antibody paratope that specifically binds c-myc (SEQ ID NO:246).

One embodiment of the present invention is a multimeric structure comprising a modified viral capsid protein of the present invention. A multimeric structure comprises at least 5, preferably at least 10, more preferably at least 30, most preferably at least 60 modified viral capsid proteins comprising a first member of a specific binding pair as described herein. They can form regular viral capsids (empty viral particles) or viral particles (capsids encapsidating a nucleotide of interest). The formation of viral particles comprising a viral genome is a highly preferred feature for use of the modified viral capsids described herein.

A further embodiment of the present invention is the use of at least one modified viral capsid protein and/or a nucleic acid encoding same, preferably at least one multimeric structure (e.g., viral particle) for the manufacture of and use in transfer of a nucleotide of interest to a target cell.

### Methods of Use and Making

A further embodiment of the modified viral capsids described herein is their use for delivering a nucleotide of interest, e.g., a reporter gene or a therapeutic gene, to a target cell. Generally, packaging of a nucleotide of interest comprises replacing an AAV genome between AAV ITR sequences with a gene of interest to create a transfer plasmid, which is then encapsulated in an AAV capsid according to well-known methods Thus, a modified viral capsid as described herein may encapsulate a transfer plasmid and/or a nucleotide of interest, which may generally comprise 5′ and 3′ inverted terminal repeat (ITR) sequences flanking a gene of interest, e.g., reporter gene(s) or therapeutic gene(s), or a portion of the gene of interest (which may be under the control of a viral or non-viral promoter). According to well-known methods of packaging AAV viral particles, the modified viral capsids, the 5′ ITR, and the 3′ ITR need not be of the same AAV serotype. In one embodiment, a transfer plasmid and/or nucleotide of interest comprises from 5′ to 3′: a 5′ ITR, a promoter, a gene (e.g., a reporter and/or therapeutic gene) and a 3′ITR.

A consideration for AAV transfer plasmid design is that a wildtype AAV genome is ˜4.7 kb. Thus, included herein are the well-known strategies that provide for packaging nucleotides of interest that exceed the packaging capacity of an individual AAV. Such strategies include, but are not limited to dual-vector strategies that exploit ITR-mediated recombination to express genes of interest that are larger than a wildtype AAV genome by way of transcript splicing across intermolecularly recombined ITRs from two complementary vector genomes, vector recombination by homology, RNA trans-splicing, and/or protein “trans-splicing” via split intein designs. See, e.g., Nakai, H. et al. (2000) Nat. Biotechnol. 18:527-532; Sun, L. (2000) Nat. Med. 6:599-602 (2000); Ghosh, A., et al. (2008) Mol. Ther. 16:124-130 (2008); Lai, Y (2005) Nat. Biotechnol. 23:1435-1439; Chew, W. L. et al. (2016) Nat. Methods 13:868-874; Li, J. (2008) Hum. Gene Ther. 19:958-964, each of which reference is incorporated herein in its entirety by reference.

Dual AAV vector strategies to transfer of a large gene into target cells have been described, which rely on different mechanisms including, but not limited to, trans-splicing, including overlapping regions in the dual vectors, and a hybrid of the two. Tornabene and Trapani (2020) Human Gene Ther. 31:47-56; see also U.S. Pat. No. 8,236,557, each of which is incorporated herein by reference in its entirety.

A trans-splicing approach takes advantage of the ability of AAV ITR sequences to concatemerized to reconstitute full-length genomes, wherein each of two or more viral capsids respectively encapsulate one of two or more transfer plasmids, each of which transfer plasmid comprises a portion of the gene of interest. For example, in a dual vector approach, the two transfer plasmids may be designed as follows: the 5′-transfer plasmid comprises the promoter, the 5′ portion of the coding sequence of the gene of interest, and a splicing donor (SD) signal; the 3′-transfer plasmid comprises a splicing acceptor (SA) signal, the 3′ portion of the gene of interest, and the polyA signal. Upon tail-to-head ITR-mediated concatemerization of the two AAV genomes, the SD and SA signals will allow splicing of the recombined genome.

A large gene of interest is also split when taking an overlapping region approach. In the overlapping region approach, the 5′ and 3′ portions (and thus the 5′ transfer plasmid and 3′ transfer plasmid) share a recombinogenic sequence, e.g., region of homology, e.g., each portion comprises an overlapping sequence. The gene of interest is made whole in a targeted cell via homologous recombination mediated by the recombinogenic sequence, e.g., homology/overlapping region.

In a hybrid approach, the 5′-transfer plasmid and 3′-transfer plasmid each comprise a highly recombinogenic sequence, wherein the recombinogenic sequence is placed downstream of an SD signal of a 5′ portion of the coding sequence of the gene of interest and upstream of an SA signal of a 3′ portion of the coding sequence of the gene of interest. In this hybrid system, the gene of interest may be made whole either via ITR-mediated concatemerization and splicing and/or by homologous recombination.

Trans-splicing at the RNA or protein levels may also be utilized. In an RNA trans-splicing approach, two transfer plasmids may respectively encode for 5′ and 3′ fragments of the pre-mRNA of a large gene and share an intronic hybridization domain that can favor trans-splicing, leading to joining of the two half-transcripts into an intact full-length mRNA.

Protein trans-splicing occurs post-translationally and is catalized by intervening proteins called split-inteins. Split-inteins are expressed as two independent polypeptides (N-intein and C-intein) at the extremities of two host proteins. The N-intein and C-intein polypeptides remain catalytically inactive until they encounter each other. Upon encountering each other, each intein precisely excises itself from the host protein while mediating ligation of the N- and C-host polypeptides via a peptide bond. Split-intein use has been used in AAV-based delivery of therapeutic genes of interest in muscle, liver, and retinal diseases. For example, on co-delivery of two halves of the mini-dystrophin cDNA fused to N- and C-intein coding sequences, efficient production of the two polypeptides was shown. Li et al. (2008) Hum Gene Ther 19:958-64. Similarly, AAV-split-inteins have been widely used for the expression and ligation of the clustered regularly interspaced short palindromic repeats (CRISPR)-Cas9 nuclease.

The above dual vector approaches are well-known in the art. See, e.g., Tornabene and Trapani (2020), supra; U.S. Pat. No. 8,236,557. Thus, in some embodiments, a modified viral capsid described herein encapsulates a nucleotide of interest, wherein the nucleotide of interest comprises a portion of a gene of interest. In some embodiments, a nucleotide of interest comprising a portion of a gene of interest further comprises a splicing donor signal or a splicing acceptor signal and/or a recombinogenic sequence. In some embodiments, a nucleotide of interest comprising a portion of a gene of interest comprises an intronic hybridization domain encoding sequence. In some embodiments, a nucleotide of interest comprising a portion of a gene of interest comprises a N-intein or C-intein encoding sequence.

Design of the transfer plasmid/nucleotide of interest includes including one or more regulatory elements, e.g., promoter and/or enhancer elements, that will control expression of the gene of interest. Non-limiting examples of useful promoters include, e.g., cytomegalovirus (CMV)-promoter, the spleen focus forming virus (SFFV)-promoter, the elongation factor 1 alpha (EF1a)-promoter (the 1.2 kb EF1a-promoter or the 0.2 kb EF1a-promoter), the chimeric EF 1 a/IF4-promoter, and the phospho-glycerate kinase (PGK)-promoter. An internal enhancer may also be present in the viral construct to increase expression of the gene of interest. For example, the CMV enhancer (Karasuyama et al. 1989. J. Exp. Med. 169:13, which is incorporated herein by reference in its entirety) may be used. In some embodiments, the CMV enhancer can be used in combination with the chicken β-actin promoter. In some embodiments, tissue specific regulatory elements, e.g., a muscle specific promoter and/or regulatory element may be used to drive the expression of the gene of interest. For example, the use of muscle-specific regulatory elements based on the muscle creatine kinase gene has been employed for muscle gene therapy treatments, such as Duchenne muscular dystrophy (DMD) and limb-girdle muscular dystrophy (LGMD). See, e.g., Salva, M. Z. et al. (2007) Mol. Ther. 15:320-329, incorporated herein in its entirety by reference. In some embodiments, a transfer plasmid and/or nucleotide of interest herein comprises an enhancer and/or promoter of muscle creatine kinase (MCK), wherein the enhancer and/or promoter of MCK drives expression of the gene of interest. In some embodiments, a transfer plasmid and/or nucleotide of interest herein comprises an enhancer and/or promoter element that recruits RNA Polymerase II, wherein the enhancer and/or promoter of MCK drives expression of the gene of interest. In some embodiments, a transfer plasmid and/or nucleotide of interest herein comprises an enhancer and/or promoter element that recruits RNA Polymerase III, wherein the enhancer and/or promoter of MCK drives expression of the gene of interest.

In some embodiments, bidirectional promoters vectors have also been employed for delivery of dual therapeutic gene cassettes. An example of this is the bidirectional chicken β-actin ubiquitous promoter that drives the simultaneous expression of the hexosaminidase α- and β-subunits of the HexA enzyme, the two respective genes involved in Tay-Sachs and Sandhoff diseases. Lahey, et al. (2020) Mol. Ther. 28:2150-2160, incorporated herein in its entirety by reference. In some embodiments, a transfer plasmid and/or nucleotide of interest herein comprises a bidirectional promoter, wherein the bidirectional promoter drives the expression of two different genes of interest

A variety of reporter genes (or detectable moieties) can be encapsidated in a multimeric structure comprising the modified viral capsid proteins described herein. Exemplary reporter genes include, for example, β-galactosidase (encoded lacZ gene), Green Fluorescent Protein (GFP), enhanced Green Fluorescent Protein (eGFP), MmGFP, blue fluorescent protein (BFP), enhanced blue fluorescent protein (eBFP), mPlum, mCherry, tdTomato, mStrawberry, J-Red, DsRed, mOrange, mKO, mCitrine, Venus, YPet, yellow fluorescent protein (YFP), enhanced yellow fluorescent protein (eYFP), Emerald, CyPet, cyan fluorescent protein (CFP), Cerulean, T-Sapphire, luciferase, alkaline phosphatase, or a combination thereof. The methods described herein demonstrate the construction of targeting particles that employ the use of a reporter gene that encodes green fluorescent protein, however, persons of skill upon reading this disclosure will understand that the viral capsids described herein can be generated in the absence of a reporter gene or with any reporter gene known in the art.

A variety of therapeutic genes can also be encapsidated in a multimeric structure comprising the modified viral capsid proteins described herein, e.g., as part of a transfer particle. Non-limiting examples of a therapeutic gene include those that encode a toxin (e.g., a suicide gene), a therapeutic antibody or fragment thereof, a CRISPR/Cas system or portion(s) thereof, antisense RNA, siRNA, shRNA, etc.

A further embodiment of the present invention is a process for the preparation of a modified capsid protein, the method comprising the steps of:
a) expressing a nucleic acid encoding the modified capsid protein under suitable conditions, and
b) isolating the expressed capsid protein of step a).

In some embodiments, a viral particle as described herein comprises a mosaic capsid, e.g., a capsid comprising capsid proteins genetically modified as described herein (in the absence or presence of a covalent bond with a targeting ligand) in a certain ratio with reference capsid proteins. A method for making such a mosaic viral particle comprises
a) expressing a nucleic acid encoding the modified capsid protein and a nucleotide encoding a reference capsid protein at a ratio (wt/wt) of at least about 60:1 to about 1:60, e.g., 2:1, 1:1, 3:5, 1:2, 1:3, etc. under suitable conditions, and
b) isolating the expressed capsid protein of step a).

In some embodiments, a composition described herein comprises, or a method described herein combines, a modified cap gene:reference cap gene (or combination of reference cap genes) at a ratio that ranges from at least about 1:60 to about 60:1, e.g., 2:1, 1:1, 3:5, 1:2, 1:3, etc. In some embodiments, the ratio is at least about 1:2. In some embodiments, the ratio is at least about 1:3. In some embodiments, the ratio is at least about 1:4. In some embodiments, the ratio is at least about 1:5. In some embodiments, the ratio is at least about 1:6. In some embodiments, the ratio is at least about 1:7. In some embodiments, the ratio is at least about 1:8. In some embodiments, the ratio is at least about 1:9. In some embodiments, the ratio is at least about 1:10. In some embodiments, the ratio is at least about 1:11. In some embodiments, the ratio is at least about 1:12. In some embodiments, the ratio is at least about 1:13. In some embodiments, the ratio is at least about 1:14. In some embodiments, the ratio is at least about 1:15. In some embodiments, the ratio is at least about 1:16. In some embodiments, the ratio is at least about 1:17. In some embodiments, the ratio is at least about 1:18. In some embodiments, the ratio is at least about 1:19. In some embodiments, the ratio is at least about 1:20. In some embodiments, the ratio is at least about 1:25. In some embodiments, the ratio is at least about 1:30. In some embodiments, the ratio is at least about 1:35. In some embodiments, the ratio is at least about 1:40. In some embodiments, the ratio is at least about 1:45. In some embodiments, the ratio is at least about 1:50. In some embodiments, the ratio is at least about 1:55. In some embodiments, the ratio is at least about 1:60. In some embodiments, the ratio is at least about 2:1. In some embodiments, the ratio is at least about 3:1. In some embodiments, the ratio is at least about 4:1. In some embodiments, the ratio is at least about 5:1. In some embodiments, the ratio is at least about 6:1. In some embodiments, the ratio is at least about 7:1. In some embodiments, the ratio is at least about 8:1. In some embodiments, the ratio is at least about 9:1. In some embodiments, the ratio is at least about 10:1. In some embodiments, the ratio is at least about 11:1. In some embodiments, the ratio is at least about 12:1. In some embodiments, the ratio is at least about 13:1. In some embodiments, the ratio is at least about 14:1. In some embodiments, the ratio is at least about 15:1. In some embodiments, the ratio is at least about 16:1. In some embodiments, the ratio is at least about 17:1. In some embodiments, the ratio is at least about 18:1. In some embodiments, the ratio is at least about 19:1. In some embodiments, the ratio is at least about 20:1. In some embodiments, the ratio is at least about 25:1. In some embodiments, the ratio is at least about 30:1. In some embodiments, the ratio is at least about 35:1. In some embodiments, the ratio is at least about 40:1. In some embodiments, the ratio is at least about 45:1. In some embodiments, the ratio is at least about 50:1. In some embodiments, the ratio is at least about 55:1. In some embodiments, the ratio is at least about 60:1.

In some embodiments, VP protein subunit ratios in the mosaic viral particle may, but do not necessarily, stoichiometrically reflect the ratios of modified cap gene:reference cap gene. As a non-limiting exemplary embodiment, a mosaic capsid formed according to the method may be considered to, but does not necessarily, have a modified capsid protein:reference capsid protein ratio similar to the ratio (wt:wt) of nucleic acids encoding same used to produce the mosaic capsid. In some embodiments, a mosaic capsid comprises a protein subunit ratio of about 1:59 to about 59:1.

Further embodiments of the present invention is a method for altering the tropism of a virus, the method comprising the steps of: (a) inserting a nucleic acid encoding an amino acid sequence into a nucleic acid sequence encoding an viral capsid protein to form a nucleotide sequence encoding a genetically modified capsid protein comprising the amino acid sequence and/or (b) culturing a packaging cell in conditions sufficient for the production of viral particles, wherein the packaging cell comprises the nucleic acid. A further embodiment of the present invention is a method for displaying a targeting ligand on the surface of a capsid protein, the method comprising the steps of: (a) expressing a nucleic acid encoding a modified viral capsid protein as described herein (and optionally with a nucleotide encoding a reference capsid protein) under suitable conditions, wherein the nucleic acid encodes a capsid protein comprising a first member of a specific binding pair, (b) isolating the expressed capsid protein comprising a first member of a specific binding pair of step (a) or capsid comprising same, and (c) incubating the capsid protein or capsid with a second cognate member of the specific binding pair under conditions suitable for allowing the formation of an isopeptide bond between the first and second member, wherein the second cognate member of the specific binding pair is fused with a targeting ligand.

In some embodiments, the packaging cell further comprises a helper plasmid and/or a transfer plasmid comprising a nucleotide of interest. In some embodiments, the methods further comprise isolating self-complementary adeno-associated viral particles from culture supernatant. In some embodiments, the methods further comprise lysing the packaging cell and isolating single-stranded adeno-associated viral particles from the cell lysate. In some embodiments, the methods further comprise (a) clearing cell debris, (b) treating the supernatant containing viral particles with nucleases, e.g., DNase I and MgCl₂, (c) concentrating viral particles, (d) purifying the viral particles, and (e) any combination of (a)-(d).

Packaging cells useful for production of the viral particles described herein include, e.g., animal cells permissive for the virus, or cells modified to be permissive for the virus; or the packaging cell construct, for example, with the use of a transformation agent such as calcium phosphate. Non-limiting examples of packaging cell lines useful for producing viral particles described herein include, e.g., human embryonic kidney 293 (HEK-293) cells (e.g., American Type Culture Collection [ATCC] No. CRL-1573), HEK-293 cells that contain the SV40 Large T-antigen (HEK-293T or 293T), HEK293T/17 cells, human sarcoma cell line HT-1080 (CCL-121), lymphoblast-like cell line Raji (CCL-86), glioblastoma-astrocytoma epithelial-like cell line U87-MG (HTB-14), T-lymphoma cell line HuT78 (TIB-161), NIH/3T3 cells, Chinese Hamster Ovary cells (CHO) (e.g., ATCC Nos. CRL9618, CCL61, CRL9096), Hela cells (e.g., ATCC No. CCL-2), Vero cells, NIH 3T3 cells (e.g., ATCC No. CRL-1658), Huh-7 cells, BHK cells (e.g., ATCC No. CCL10), PC12 cells (ATCC No. CRL1721), COS cells, COS-7 cells (ATCC No. CRL1651), RATI cells, mouse L cells (ATCC No. CCLI.3), HLHepG2 cells, CAP cells, CAP-T cells, and the like.

L929 cells, the FLY viral packaging cell system outlined in Cosset et al (1995) J Virol 69,7430-7436, NS0 (murine myeloma) cells, human amniocytic cells (e.g., CAP, CAP-T), yeast cells (including, but not limited to, S. cerevisiae, Pichia pastoris), plant cells (including, but not limited to, Tobacco NTl, BY-2), insect cells (including but not limited to SF9, S2, SF21, Tni (e.g. High 5)) or bacterial cells (including, but not limited to, E. coli).

For additional packaging cells and systems, packaging techniques and particles for packaging the nucleic acid genome into the pseudotyped viral particle see, for example, Polo, et al, Proc Natl Acad Sci USA, (1999) 96:4598-4603. Methods of packaging include using packaging cells that permanently express the viral components, or by transiently transfecting cells with plasmids.

Further embodiments include methods comprising contacting a modified Cap protein as described herein with the targeting vector in conditions sufficient to operably link the modified Cap protein with the targeting vector, e.g., in conditions sufficient to promote association of the targeting vector to the modified Cap protein, e.g., via chemical linkage and/or association of first and second members of a specific binding pair, wherein the first member is inserted into the modified Cap protein the first member and the targeting vector is fused to the second member of the specific binding pair.

Further embodiments include methods of redirecting a virus and/or delivering a reporter or therapeutic gene to a target cell, the method comprising a method for transducing cells in vitro (e.g., ex vivo) or in vivo, the method comprising the steps of: contacting the target cell with a viral particle comprising a capsid described herein, wherein the capsid comprises a targeting ligand that specifically binds a receptor expressed by the target cell. In some embodiments, the target cell is in vitro (e.g., ex vivo). In other embodiments, the target cell is in vivo in a subject, e.g., a human.

### Target Cells

A wide variety of cells may be targeted in order to deliver a nucleotide of interest using a modified viral particle as disclosed herein. The target cells will generally be chosen based upon the nucleotide of interest and the desired effect.

In some embodiments, a nucleotide of interest may be delivered to enable a target cell to produce a protein that makes up for a deficiency in an organism, such as an enzymatic deficiency, or immune deficiency, such as X-linked severe combined immunodeficiency. Thus, in some embodiments, cells that would normally produce the protein in the animal are targeted. In other embodiments, cells in the area in which a protein would be most beneficial are targeted.

In other embodiments, a nucleotide of interest, such as a gene encoding an siRNA, may inhibit expression of a particular gene in a target cell. The nucleotide of interest may, for example, inhibit expression of a gene involved in a pathogen life cycle. Thus, cells susceptible to infection from the pathogen or infected with the pathogen may be targeted. In other embodiments, a nucleotide of interest may inhibit expression of a gene that is responsible for production of a toxin in a target cell.

In other embodiments a nucleotide of interest may encode a toxic protein that kills cells in which it is expressed. In this case, tumor cells or other unwanted cells may be targeted.

In still other embodiments a nucleotide of interest that encodes a therapeutic protein.

Once a particular population of target cells is identified in which expression of a nucleotide of interest is desired, a target receptor is selected that is specifically expressed on that population of target cells. The target receptor may be expressed exclusively on that population of cells or to a greater extent on that population of cells than on other populations of cells. The more specific the expression, the more specifically delivery can be directed to the target cells. Depending on the context, the desired amount of specificity of the marker (and thus of the gene delivery) may vary. For example, for introduction of a toxic gene, a high specificity is most preferred to avoid killing non-targeted cells. For expression of a protein for harvest, or expression of a secreted product where a global impact is desired, less marker specificity may be needed.

As discussed above, the target receptor may be any receptor for which a targeting ligand can be identified or created. Preferably the target receptor is a peptide or polypeptide, such as a receptor. However, in other embodiments the target receptor may be a carbohydrate or other molecule that can be recognized by a binding partner. If a binding partner, e.g., ligand, for the target receptor is already known, it may be used as the affinity molecule. However, if a binding molecule is not known, antibodies to the target receptor may be generated using standard procedures. The antibodies can then be used as a targeting ligand.

Thus, target cells may be chosen based on a variety of factors, including, for example, (1) the application (e.g., therapy, expression of a protein to be collected, and conferring disease resistance) and (2) expression of a marker with the desired amount of specificity.

Target cells are not limited in any way and include both germline cells and cell lines and somatic cells and cell lines. When the target cells are germline cells, the target cells are preferably selected from the group consisting of single-cell embryos and embryonic stem cells (ES).

### Pharmaceutical Compositions, Dosage Forms and Administration

A further embodiment provides a medicament comprising at least one modified viral capsid protein and appropriate targeting ligand according to this invention and/or a nucleic acid according to this invention. Preferably such medicament is useful as a gene transfer particle.

Also disclosed herein are pharmaceutical compositions comprising the viral particles described herein and a pharmaceutically acceptable carrier and/or excipient. In addition, disclosed herein are pharmaceutical dosage forms comprising the viral particle described herein.

As discussed herein, the viral particles described herein can be used for various therapeutic applications (in vivo and ex vivo) and as research tools.

Pharmaceutical compositions based on the viral particles disclosed herein can be formulated in any conventional manner using one or more physiologically acceptable carriers and/or excipients. The viral particles may be formulated for administration by, for example, injection, inhalation or insulation (either through the mouth or the nose) or by oral, buccal, parenteral or rectal administration, or by administration directly to a tumor.

The pharmaceutical compositions can be formulated for a variety of modes of administration, including systemic, topical or localized administration. Techniques and formulations can be found in, for example, Remmington's Pharmaceutical Sciences, Meade Publishing Co., Easton, Pa. For systemic administration, injection is preferred, including intramuscular, intravenous, intraperitoneal, and subcutaneous. For the purposes of injection, the pharmaceutical compositions can be formulated in liquid solutions, preferably in physiologically compatible buffers, such as Hank's solution or Ringer's solution. In addition, the pharmaceutical compositions may be formulated in solid form and redissolved or suspended immediately prior to use. Lyophilized forms of the pharmaceutical composition are also suitable.

For oral administration, the pharmaceutical compositions may take the form of, for example, tablets or capsules prepared by conventional means with pharmaceutically acceptable excipients such as binding agents (e.g. pregelatinized maize starch, polyvinylpyrrolidone or hydroxypropyl methylcellulose); fillers (e.g. lactose, microcrystalline cellulose or calcium hydrogen phosphate); lubricants (e.g. magnesium stearate, talc or silica); disintegrants (e.g. potato starch or sodium starch glycolate); or wetting agents (e.g. sodium lauryl sulfate). The tablets can also be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g. sorbitol syrup, cellulose derivatives or hydrogenated edible fats); emulsifying agents (e.g. lecithin or acacia); non-aqueous vehicles (e.g. oil, oily esters, ethyl alcohol or fractionated vegetable oils); and preservatives (e.g. methyl or propyl-p-hydroxybenzoates or sorbic acid). The preparations can also contain buffer salts, flavoring, coloring and sweetening agents as appropriate.

The pharmaceutical compositions can be formulated for parenteral administration by injection, e.g. by bolus injection or continuous infusion. Formulations for injection can be presented in a unit dosage form, e.g. in ampoules or in multi-dose containers, with an optionally added preservative. The pharmaceutical compositions can further be formulated as suspensions, solutions or emulsions in oily or aqueous vehicles, and may contain other agents including suspending, stabilizing and/or dispersing agents.

Additionally, the pharmaceutical compositions can also be formulated as a depot preparation. These long acting formulations can be administered by implantation (e.g. subcutaneously or intramuscularly) or by intramuscular injection. Thus, for example, the compounds may be formulated with suitable polymeric or hydrophobic materials (e.g. as an emulsion in an acceptable oil) or ion exchange resins, or as sparingly soluble derivatives, for example, as a sparingly soluble salt. Other suitable delivery systems include microspheres, which offer the possibility of local noninvasive delivery of drugs over an extended period of time. This technology can include microspheres having a precapillary size, which can be injected via a coronary catheter into any selected part of an organ without causing inflammation or ischemia. The administered therapeutic is men slowly released from the microspheres and absorbed by the surrounding cells present in the selected tissue.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, bile salts, and fusidic acid derivatives. In addition, detergents may be used to facilitate permeation. Transmucosal administration can occur using nasal sprays or suppositories. For topical administration, the viral particles described herein can be formulated into ointments, salves, gels, or creams as generally known in the art. A wash solution can also be used locally to treat an injury or inflammation in order to accelerate healing.

Pharmaceutical forms suitable for injectable use can include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid. It must be stable under the conditions of manufacture and certain storage parameters (e.g. refrigeration and freezing) and must be preserved against the contaminating action of microorganisms, such as bacteria and fungi.

If formulations disclosed herein are used as a therapeutic to boost an immune response in a subject, a therapeutic agent can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

A carrier can also be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents known in the art. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions can be prepared by incorporating the active compounds or constructs in the required amount in the appropriate solvent with various of the other ingredients enumerated above, as required, followed by filtered sterilization.

Upon formulation, solutions can be administered in a manner compatible with the dosage formulation and in such amount as is therapeutically effective. The formulations are easily administered in a variety of dosage forms, such as the type of injectable solutions described above, but slow release capsules or microparticles and microspheres and the like can also be employed.

For parenteral administration in an aqueous solution, for example, the solution should be suitably buffered if necessary and the liquid diluent first rendered isotonic with sufficient saline or glucose. These particular aqueous solutions are especially suitable for intravenous, intratumorally, intramuscular, subcutaneous and intraperitoneal administration. In this context, sterile aqueous media that can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion.

The person responsible for administration will, in any event, determine the appropriate dose for the individual subject. For example, a subject may be administered viral particles described herein on a daily or weekly basis for a time period or on a monthly, bi-yearly or yearly basis depending on need or exposure to a pathogenic organism or to a condition in the subject (e.g. cancer).

In addition to the compounds formulated for parenteral administration, such as intravenous, intratumorally, intradermal or intramuscular injection, other pharmaceutically acceptable forms include, e.g., tablets or other solids for oral administration; liposomal formulations; time release capsules; biodegradable and any other form currently used.

One may also use intranasal or inhalable solutions or sprays, aerosols or inhalants. Nasal solutions can be aqueous solutions designed to be administered to the nasal passages in drops or sprays. Nasal solutions can be prepared so that they are similar in many respects to nasal secretions. Thus, the aqueous nasal solutions usually are isotonic and slightly buffered to maintain a pH of 5.5 to 7.5. In addition, antimicrobial preservatives, similar to those used in ophthalmic preparations, and appropriate drug stabilizers, if required, may be included in the formulation. Various commercial nasal preparations are known and can include, for example, antibiotics and antihistamines and are used for asthma prophylaxis.

Oral formulations can include excipients as, for example, pharmaceutical grades of mannitol, lactose, starch, magnesium stearate, sodium saccharine, cellulose, magnesium carbonate and the like. These compositions take the form of solutions, suspensions, tablets, pills, capsules, sustained release formulations or powders. In certain defined embodiments, oral pharmaceutical compositions will include an inert diluent or assimilable edible carrier, or they may be enclosed in hard or soft shell gelatin capsule, or they may be compressed into tablets, or they may be incorporated directly with the food of the diet. For oral therapeutic administration, the active compounds may be incorporated with excipients and used in the form of ingestible tablets, buccal tablets, troches, capsules, elixirs, suspensions, syrups, wafers, and the like.

The tablets, troches, pills, capsules and the like may also contain the following: a binder, as gum tragacanth, acacia, cornstarch, or gelatin; excipients, such as dicalcium phosphate; a disintegrating agent, such as corn starch, potato starch, alginic acid and the like; a lubricant, such as magnesium stearate; and a sweetening agent, such as sucrose, lactose or saccharin may be added or a flavoring agent, such as peppermint, oil of wintergreen, or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup of elixir may contain the active compounds sucrose as a sweetening agent methyl and propylparabens as preservatives, a dye and flavoring, such as cherry or orange flavor.

Further embodiments disclosed herein can concern kits for use with methods and compositions. Kits can also include a suitable container, for example, vials, tubes, mini- or microfuge tubes, test tube, flask, bottle, syringe or other container. Where an additional component or agent is provided, the kit can contain one or more additional containers into which this agent or component may be placed. Kits herein will also typically include a means for containing the viral particles and any other reagent containers in close confinement for commercial sale. Such containers may include injection or blow-molded plastic containers into which the desired vials are retained. Optionally, one or more additional active agents such as, e.g., anti-inflammatory agents, anti-viral agents, anti-fungal or anti-bacterial agents or anti-tumor agents may be needed for compositions described.

Compositions disclosed herein may be administered by any means known in the art. For example, compositions may include administration to a subject intravenously, intratumorally, intradermally, intraarterially, intraperitoneally, intralesionally, intracranially, intraarticularly, intraprostaticaly, intrapleurally, intratracheally, intranasally, intravitreally, intravaginally, intrarectally, topically, intratumorally, intramuscularly, intrathecally, subcutaneously, subconjunctival, intravesicularlly, mucosally, intrapericardially, intraumbilically, intraocularly, orally, locally, by inhalation, by injection, by infusion, by continuous infusion, by localized perfusion, via a catheter, via a lavage, in a cream, or in a lipid composition.

Any method known to one skilled in the art may be used for large scale production of viral particles, packaging cells and particle constructs described herein. For example, master and working seed stocks may be prepared under GMP conditions in qualified primary CEFs or by other methods. Packaging cells may be plated on large surface area flasks, grown to near confluence and viral particles purified. Cells may be harvested and viral particles released into the culture media isolated and purified, or intracellular viral particles released by mechanical disruption (cell debris can be removed by large-pore depth filtration and host cell DNA digested with endonuclease). Virus particles may be subsequently purified and concentrated by tangential-flow filtration, followed by diafiltration. The resulting concentrated bulk may be formulated by dilution with a buffer containing stabilizers, filled into vials, and lyophilized. Compositions and formulations may be stored for later use. For use, lyophilized viral particles may be reconstituted by addition of diluent.

Certain additional agents used in the combination therapies can be formulated and administered by any means known in the art.

Compositions as disclosed herein can also include adjuvants such as aluminum salts and other mineral adjuvants, tensoactive agents, bacterial derivatives, vehicles and cytokines. Adjuvants can also have antagonizing immunomodulating properties. For example, adjuvants can stimulate Th1 or Th2 immunity. Compositions and methods as disclosed herein can also include adjuvant therapy.

### Skeletal Muscle Related Disorders

Also provided herein are methods of treating a skeletal muscle related disorder, e.g., a muscle wasting disease and/or a genetic muscle disease, e.g., X-linked myotubular myopathy (XLMTM), Duchenne muscular dystrophy (DMD), myotonic dystrophy (DM1), Facioscapulohumeral muscular dystrophy Type 1 (FSHD), congenital muscular dystrophy type 1A (MDC1A), Limb girdle muscular dystrophy, dystroglycanopathy, muscle atrophy conditions, metabolic diseases, etc. Generally, such methods comprise administering to a patient suffering from or at risk for such skeletal related disorder a viral particle or pharmaceutical composition as described herein, wherein the viral particle comprises
(i) a viral capsid modified to comprise a first member of a protein:protein binding pair,
(ii) a second member of the protein:protein binding pair, wherein the second member of the protein:protein binding pair comprises a targeting ligand that binds a muscle-specific surface protein that is expressed on the surface of a muscle cell (e.g., CACNG1), wherein the first member of the protein:protein binding pair and the second member of the protein:protein binding pair are associated to direct the tropism of the viral capsid to the muscle cell in the patient thereof, and
(iii) a nucleotide of interest encapsidated within the viral capsid.

In some embodiments, the nucleotide of interest encodes a therapeutic protein, a suicide gene, an antibody or a fragment thereof, a CRISPR/Cas system or a portion(s) thereof, an antisense oligonucleotide, a ribozyme, an RNAi molecule, or a shRNA molecule. For example, in some embodiments, the nucleotide of interest may encode a growth factor, neurotrophic factor, a disease modifying muscle protein, a metabolic protein, e.g., for muscle atrophy conditions or metabolic diseases.

TABLE 2

| | | | | |
|---|---|---|---|---|
| | | | Region encoding the | |
| | | | non-structural/ | Region encoding the |
| | Accession | | rep protein | capsid protein |
| | Number/ | | (Accession Number/ | (Accession Number/ |
| Description | GI Number | ITR | GI Number of protein) | GI Number of protein) |

Avian adeno-associated virus

| | | | | |
|---|---|---|---|---|
| Avian adeno-associated | GQ368252.1/ | | 243-2237 | 2255-4471 |
| virus isolate YZ-1, | GI: 255710032 | | (ACU30841.1/ | (ACU30842.1/ |
| complete genome | | | GI: 255710033) | GI: 255710034) |
| Avian adeno-associated | KF937794.1/ | 1-142 | 244-2235 | 2253-4469 |
| virus isolate ZN1, | GI: 588284633 | 4540-4682 | (AHK22792.1/ | (AHK22793.1/ |
| complete genome | | | GI: 588284634) | GI: 588284635) |

Avian adeno-associated virus ATCC VR-865

| | | | | |
|---|---|---|---|---|
| Avian adeno-associated | AY186198.1/ | 1-142 | 244-2232 | 2250-4481 |
| virus ATCC VR-865, | GI: 31414777 | 4552-4694 | (AAO32086.1/ | (AAO32087.1/ |
| complete genome | | | GI: 31414778) | G1: 31414779) |
| Avian adeno-associated | NC_004828.1/ | 1-142 | 244-2232 | 2250-4481 |
| virus ATCC VR-865, | GI: 31543992 | 4552-4694 | (NP_852780.1/ | (NP_852781.1/ |
| complete genome | | | GI: 31543993) | GI: 31543994) |
| Avian adeno-associated | AY629582.1/ | 1-142 | 244-2232 | 2250-4481 |
| virus ATCC VR-865, | GI: 48996102 | 4552-4694 | (AAT48612.1/ | (AAT48613.1/ |
| complete genome | | | GI: 48996103) | GI: 48996104) |

Avian adeno-associated virus strain DA-1

| | | | | |
|---|---|---|---|---|
| Avian adeno-associated | AY629583.1/ | 1-142 | 244-2235 | 2253-4469 |
| virus strain DA-1, | GI: 48996105 | 4540-4682 | (AAT48614.1/ | (AAT48615.1/ |
| complete genome | | | GI: 48996106) | GI: 48996107) |
| Avian adeno-associated | NC_006263.1/ | 1-142 | 244-2235 | 2253-4469 |
| virus strain DA-1, | GI: 51949968 | 4540-4682 | (YP_077182.1/ | (YP_077183.1/ |
| complete genome | | | GI: 51949969) | GI: 51949970) |

Bat adeno-associated virus YNM

| | | | |
|---|---|---|---|
| Bat adeno-associated | GU226971.1/ | 200-2041 | 2059-4233 |
| virus YNM, complete | GI: 289719008 | (ADD17085.1/ | (ADD17086.1/ |
| sequence | | GI: 289719009) | GI: 289719010) |
| Bat adeno-associated | NC_014468.1/ | 200-2041 | 2059-4233 |
| virus YNM, complete | GI: 304422916 | (YP_003858571.1/ | (YP_003858572.1/ |
| genome | | GI: 304422917) | GI: 304422918) |

Bovine adeno-associated virus

| | | | |
|---|---|---|---|
| Bovine adeno- | AY388617.1/ | 368-2200 | 2216-4426 |
| associated virus, | GI: 38679253 | (AAR26464.1/ | (AAR26465.1/ |
| complete genome | | GI: 38679254) | GI: 38679255) |
| Bovine adeno- | NC_005889.1/ | 368-2200 | 2216-4426 |
| associated vitus, | GI: 48696557 | (YP_024970.1/ | (YP_024971.1/ |
| complete genome | | GI: 48696558) | GI: 48696559) |

Bovine parvovirus - 2

| | | | |
|---|---|---|---|
| Bovine parvovirus 2 | AF406966.1/ | 306-1919 | 2268-5384 |
| pulative non-structural | GI: 15825370 | (AAL09671.1/ | (AAL09672.1/ |
| protein and putative | | GI: 15825371) | GI: 15825372) |
| capsid protein genes, | | | |
| complete cds. | | | |
| Bovine parvovirus 2, | NC_006259.1/ | 306-1919 | 2268-5384 |
| complete genome | GI: 51949957 | (YP_077175.1/ | (YP_077176.1/ |
| | | GI: 51949958) | GI: 51949959) |

California sea lion adeno-associated virus 1

| | | | |
|---|---|---|---|
| California sea lion | JN420371.1/ | 284-2086 | 2117-4273 |
| adeno-associated virus | GI: 343458966 | (AEM37641.1/ | (AEM37642.1/ |
| 1 isolate 1136 Rep78 | | GI: 343458967) | GI: 343458968) |
| and VP1 genes, | | | |
| complete cds. | | | |
| California sea lion | JN420372.1/ | 291-2093 | 2124-4280 |
| adeno-associated virus | GI: 343458969 | (AEM37643.1/ | (AEM37644.1/ |
| 1 isolate 1187 Rep78 | | GI: 343458970) | GI: 343458971) |
| and VP1 genes, | | | |
| complete cds. | | | |

Goose parvovirus

| | | | | |
|---|---|---|---|---|
| Goose parvovirus | (U25749.1/ | 1-444 | 537-2420 | 2439-4637 |
| virulent B strain, | GI: 1113795) | 4663-5106 | (AAA83229.1/ | (AAA83230.1/ |
| complete genome | | | GI: 1113796) | GI: 1113797) |
| | | | | 2874-4637 |
| | | | | (AAA83231.1/ |
| | | | | GI: 1113798) |
| | | | | 3033-4637 |
| | | | | (AAA83232.1/ |
| | | | | GI: 1113799) |
| Goose parvovirus, | (NC_001701.1/ | 1-444 | 537-2420 | 2439-4637 |
| complete genome | GI: 9628649) | 4663-5106 | (NP_043514.1/ | (NP_043515.1/ |
| | | | GI: 9628650) | GI: 9628651) |
| | | | | 2874-4637 |
| | | | | (NP_043516.1/ |
| | | | | GI: 9628652) |
| | | | | 3033-4637 |
| | | | | (NP_043517.1/ |
| | | | | GI: 9628653) |
| Goose parvovirus strain | (EU583389.1/ | 1-381 | 474-2357 | 2376-4574 |
| 82-0321V, complete | GI: 190888188) | 4600-4980 | (ACE95848.1/ | (ACE95849.1/ |
| genome | | | GI: 190888189) | GI: 190888190) |
| Goose parvovirus strain | (EU583390.1/ | 1-416 | 509-2392 | 2411-4609 |
| 82-0321, complete | GI: 190888191) | 4635-5050 | (ACE95850.1/ | (ACE95851.1/ |
| genome | | | GI: 190888192) | GI: 190888193) |
| Goose parvovirus strain | (EU583391.1/ | 1-418 | 511-2394 | 2413-4611 |
| 06-0329, complete | GI: 190888194) | 4637-5054 | (ACE95852.1/ | ACE95853.1/ |
| genome | | | GI: 190888195) | GI: 190888196) |
| Goose parvovirus strain | (EU583392.1/ | 1-443 | 536-2419 | 2438-4636 |
| VG32/1, complete | GI: 190888197) | 4662-5104 | (ACE95854.1/ | (ACE95855.1/ |
| genome | | | GI: 190888198) | GI: 190888199) |
| Goose parvovirus strain | (JF333590.1/ | 1-444 | 537-2420 | 2439-4637 |
| SH, complete genome | GT:343113618) | 4663-5106 | (AEL87777.1/ | (AEL87778.1/ |
| | | | GI: 343113619) | G1:343113620) |
| | | | | 2874-4637 |
| | | | | (AEL87779.1/ |
| | | | | GI: 343113621) |
| | | | | 3033-4637 |
| | | | | (AEL87780.1/ |
| | | | | GI: 343113622) |
| Goose parvovirus strain | (HQ891825.1/ | 1-444 | 537-2420) | 2439-4637 |
| GDaGPV, complete | GI: 359843307) | 4663-5106 | (AEV89789.1/ | (AEV89790.1/ |
| genome | | | GI: 359843308) | GI: 359843309) |
| | | | | 2874-4637 |
| | | | | (AEV89791.1/ |
| | | | | GI: 359843310) |
| | | | | 3033-4637 |
| | | | | (ARV89792.1/ |
| | | | | GI: 359843311) |
| Goose parvovirus strain | (KC478066.1/ | 1-416 | 509-2392 | 2411-4609 |
| SHFX1201, complete | GI: 459360514) | | (AGG56527.1/ | (AGG56528.1/ |
| genome | | | GI: 459360515) | GI: 459360516) |
| Goose parvovirus strain | (KC178571.1/ | 1-444 | 537-2420 | 2439-4637 |
| Y, complete genome | GI: 513129761) | | (AGO17637.1/ | (AGO17638.1/ |
| | | | GI: 513129762) | GI: 513129763) |
| Goose parvovirus strain | (KC184133.1/ | 1-443 | 536-2419 | 2438-4636 |
| E, complete genome | GI: 513129764) | | (AGO17639.1/ | (AGO17640.1/ |
| | | | GI: 513129765) | GI: 513129766) |
| Goose parvovirus strain | (KC996729.1/ | 1-442 | 535-2418 | 2437-4635 |
| SYG61v, complete | G1:531997217) | | (AGT62580.1/ | (AGT62581.1/ |
| genome | | | GT: 531997218) | GT: 531997219) |
| Goose parvovirus strain | (KC996730.1/ | 1-414 | 507-2390 | 2409-4607 |
| YZ99-6, complete | GI: 531997220) | | (AGT62582.1/ | (AGT62583.1/ |
| genome | | | GI: 531997221) | GI: 531997222) |
| Goose parvovirus | (AF416726.1/ | | 1-1884 | 1903-4101 |
| nonstructural protein | GI: 17226299) | | (AAL37721.1/ | (AAL37722.1/ |
| NS (ns) and capsid | | | GI: 17226300) | GI: 17226301) |
| protein VP (vp) genes, | | | 529-1884 | |
| complete cds. | | | (ABP93843.1/ | |
| | | | GI: 145694447) | |
| Goose parvovirus strain | (EF515837.1/ | | 1-1884 | 1903-4101 |
| DY NS1 (NS1), NS2 | GI: 14569444 | | (ABP93842.1/ | (ABP82770.1/ |
| (NS2), VP1 (VP1), | | | GI: 145694446) | GI: 145573169) |
| VP2 (VP2), and VP3 | | | | 2338-4101 |
| (VP3) genes, complete | | | | (ABP93844.1/ |
| cds. | | | | GI: 145694448) |
| | | | | 2497-4101 |
| | | | | (ABP93845.1/ |
| | | | | GI: 145694449) |
| Goose parvovirus strain | (JF926695.1/ | | 9-1892 | 1911-4109 |
| PT NS1 protein and | GI: 354463162) | | (AER25356.1/ | (AER25357.1/ |
| VP1 protein genes, | | | GI: 354463163) | GI: 354463164) |
| complete cds. | | | | |
| Goose parvovirus strain | (JF926696.1/ | | 6-1889 | 1908-4106 |
| D NS1 protein and VP1 | GI: 354463165) | | (AER25358.1/ | (AER25359.1/ |
| protein genes, complete | | | GI: 364463166) | GI: 354463167) |
| cds. | | | | |

Mouse adeno-associated virus 1

| | | | |
|---|---|---|---|
| Mouse adeno- | DQ100362.1/ | 1-327 | 344-2485 |
| associated virus 1 rep | GI: 73665994 | (AAZ79671.1/ | (AAZ79672.1/ |
| gene, partial cds; and | | GI: 73665995) | GI: 73665996) |
| VP1 capsid, VP2 | | | 731-2485 |
| capsid, and VP3 capsid | | | (AAZ79673.1/ |
| genes, complete cds. | | | GI: 73665997) |
| | | | 911-2485 |
| | | | (AAZ79674.1/ |
| | | | GI: 73665998) |

Muscovy duck parvovirus

| | | | | |
|---|---|---|---|---|
| Barbarie duck | U22967.1/ | 1-437 | 548-2431 | 2450-4648 |
| parvovirus REP protein | GI: 1113784 | 4678-5132 | (AAA83224.1/ | (AAA83225.1/ |
| (rep) and three capsid | | | GI: 1113785) | GI: 1113786) |
| protein VP (vp) genes, | | | | 2885-4648 |
| complete cds | | | | (AAA83226.1/ |
| | | | | GI: 1113787) |
| | | | | 3044-4648 |
| | | | | (AAA83227.1/ |
| | | | | GI: 1113788) |
| Muscovy duck | NC_006147.2/ | 1-457 | 548-2431 | 2450-4648 |
| parvovirus, complete | GI: 51593841 | 4678-5132 | (YP_068410.1/ | (YP_068411.17 |
| genome | | | GI: 51593842) | G1: 51593843) |
| | | | | 2885-4648 |
| | | | | (YP_068412.1; |
| | | | | GI: 51593844) |
| | | | | 3044-4648 |
| | | | | (YP_068413.1/ |
| | | | | GI: 51593845) |
| Muscovy duck | JF926697.1/ | | 61-1944 | 1963-4161 |
| parvovims strain P REP | GI: 354463168 | | (AER25360.1/ | (AER25361.1/ |
| protein and VP1 protein | | | GI: 354463169) | GI: 354463170) |
| genes, complete cds. | | | | |
| Muscovy duck | JP926698.1/ | | 61-1944 | 1963-4161 |
| parvovirus strain P1 | GI: 354463171 | | (AER25362.1/ | (AER25363.1/ |
| REP protein and VP1 | | | GI: 354463172) | GI: 354463173) |
| protein genes, complete | | | | |
| cds. | | | | |
| Muscovy duck | KC171936.1/ | 1-381 | 512-2395 | 2414-4612 |
| parvovirus isolate | GI: 459256867 | 4681-5061 | (AGG53766.1/ | (AGG53768.1/ |
| SAAS-SHNH, | | | GI: 459256868) | GI: 459256870) |
| complete genome | | | | 2849-4612 |
| | | | | (AGG53769.1/ |
| | | | | GI: 459256871) |
| | | | | 3008-4612 |
| | | | | (AGG53767.1/ |
| | | | | GI: 459256869) |

Serpentine adeno-associated virus 2

| | | | |
|---|---|---|---|
| Serpentine adeno- | EU872429.1/ | 1-642 | 661-1297 |
| associated virus 2 non- | GI: 215401981 | (ACJ66590.1/ | (ACJ66591.1/ |
| structural protein 1 and | | GI: 215401982) | GI: 215401983) |
| capsid protein genes, | | | |
| partial cds | | | |

Snake parvovirus 1

| | | | |
|---|---|---|---|
| Snake parvovirus 1 | AY349010.1/ | 324-2012 | 2030-4210 |
| non-structural protein 1 | GI: 38017148 | (AAR07954.1/ | (AAR07955.1/ |
| (NS1) and capsid | | GI: 38017149) | GI: 38017150) |
| protein (VP1) genes, | | | |
| complete cds | | | |
| Snake parvovirus 1, | NC_006148.1/ | 324-2012 | 2030-4210 |
| complete genome | GI: 51555744 | (YP_068093.1/ | (YP_068094.1/ |
| | | GI: 51555745) | GI: 51555746) |

TABLE 3
BRIEF DESCRIPTION OF SEQUENCES
IN THE SEQUENCE LISTING

| SEQ ID NO | BRIEF DESCRIPTION |
|---|---|
| 1-240 | Anti-human CACNG1 antibody or binding portions thereof |
| 241 | Human CACNG1 CDS |
| 242 | Human CACNG1 Protein |
| 243 | SpyTag Protein |
| 244 | SpyCatcher Protein |
| 245 | KTag Protein |
| 246 | c-myc protein |
| 247 | B1 epitope protein |
| 248 | GLSG protein linker |
| 249 | GLSGSG protein linker |
| 250 | GLSGLSGS protein linker |
| 251 | GLSGLSGLSG protein linker |
| 252 | GLSGGSGLSG protein linker |
| 253 | GSGESG protein linker |
| 254 | Isopeptide protein |
| 255 | SpyTag002 |
| 256 | Artificial hGH pA-specific primer |
| 257 | Artificial 5′ barcode primer |
| 258 | Artificial 3′ barcode primer |
| 259 | SpyTag003 |

### EXAMPLES

The following examples are provided for illustrative purposes only and are not intended to limit the scope of the invention.

### Methods

The following examples are provided for illustrative purposes only and are not intended to limit the scope of the invention.

### Preparation of AAV Viral Vectors

Virus was generated by transfecting 293T packaging cells using PEI Pro with the following plasmids: pAd Helper, an AAV2 ITR-containing genome plasmid encoding a reporter protein, and a pAAV-CAP plasmid encoding AAV Rep and Cap genes, either with or without additional plasmids encoding either the heavy and light chains of an antibody. The antibody heavy chain constructs are all fused to SpyCatcher at their C terminus as described in WO2019006046, incorporated herein in its entirety by reference. Transfection complexes were prepared in incomplete DMEM (no additional supplements) and incubated at room temperature for 10 minutes.

Each virus was generated by transfecting 15 cm plates of 293T packaging cells with the following plasmids and quantities:

WT AAV2/HBM GFP

| | | |
|---|---|---|
| pAd Helper | 16 | ug |
| pAAV-CAG-eGFP | 8 | ug |
| pAAV2-CAP WT or pAAV2 R585A R588A HBM | 8 | ug |

AAV2 Anti-Human ASGR1/anti-Human CACNG1 GFP

| | | |
|---|---|---|
| pAd Helper | 16 | ug |
| pAAV-CAG-eGFP | 8 | ug |
| pAAV2-CAP G453 Linker 10 Spy Tag HBM | 1 | ug |
| pAAV2-CAP R585A R588A HBM | 7 | ug |
| With | 1.5 | ug |
| pAnti-ASGR1 or Anti-CACNG1 hIgG4US SpyCatcher Vh | | |
| ULC 1-39 Vk | 3 | ug |

WT AAV2/HBM Luciferase

| | | |
|---|---|---|
| pAd Helper | 16 | ug |
| pAAV-UbC- Firefly Luciferase | 8 | ug |
| pAAV2-CAP WT or pAAV2 R585A R588A HBM | 8 | ug |

AAV2 Anti-Human ASGR1/anti-Human CACNG1 Luciferase

| | | |
|---|---|---|
| pAd Helper | 16 | ug |
| pAAV-UbC- Firefly Luciferase | 8 | ug |
| pAAV2 CAP G453 Linker 10 SpyTag HBM | 1 | ug |
| pAAV2-CAP R585A R588A HBM | 7 | ug |
| With | 1.5 | ug |
| pAnti-ASGR1 or Anti-CACNG1 hIgG4US SpyCatcher Vh | | |
| ULC 1-39 Vk | 3 | ug |

WT AAV9/N272A GFP

| | | |
|---|---|---|
| pAd Helper | 16 | ug |
| pAAV-CAG-eGFP | 8 | ug |
| pAAV9-CAP or pAAV9 N272A | 8 | ug |

AAV9 Anti-Human ASGR1/anti-Human CACNG1 GFP

| | | |
|---|---|---|
| pAd Helper | 16 | ug |
| pAAV-CAG-eGFP | 8 | ug |
| pAAV9 CAP G453 Linker 10 SpyTag W503A | 1 | ug |
| pAAV9-CAP N272A | 7 | ug |
| With | 1.5 | ug |
| pAnti-ASGR1 or Anti-CACNG1 hIgG4US SpyCatcher Vh | | |
| ULC 1-39 Vk | 3 | ug |

WT AAV9/N297A Luciferase

| | | |
|---|---|---|
| pAd Helper | 16 | ug |
| pAAV-UbC- Firefly Luciferase | 8 | ug |
| pAAV9-CAP or pAAV9 N272A | 8 | ug |

AAV9 Anti-Human ASGR1/anti-Human CACNG1 Luciferase

| | | |
|---|---|---|
| pAd Helper | 16 | ug |
| pAAV-UbC- Firefly Luciferase | 8 | ug |
| pAAV9-CAP G453 Linker 10 SpyTag W503A | 1 | ug |
| pAAV9-CAP N272A | 7 | ug |
| With | 1.5 | ug |
| pAnti-ASGR1 or Anti-CACNG1 hIgG4US SpyCatcher | | |
| Vh (or SpyCatcher Fab) | | |
| ULC 1-39 Vk | 3 | ug |

WT AAV9-uDys5

| | | |
|---|---|---|
| pAd Helper | 16 | ug |
| pAAV-CK8-uDys5 | 8 | ug |
| pAAV9-CAP | 8 | ug |

AAV9 anti-Human CACNG1 uDys5

| | | |
|---|---|---|
| pAd Helper | 16 | ug |
| pAAV-CK8-uDys5 | 8 | ug |
| pAAV9 CAP G453 Linker 10 SpyTag | 1 | ug |
| pAAV9-CAP N272A | 7 | ug |
| With | 1.5 | ug |
| pAnti-CACNG1 10717 SpyCatcher Vh | | |
| pAnti-CACNG1 10717 Vk | 3.0 | ug |

CK8-uDys5 is described in U.S. Pat. No. 10,479,821B2, incorporated herein in its entirety by reference.

Post incubation, complexes are added to DMEM supplemented with 10% FBS, 1XNEAA, 1% Pen/Strep, and 1% L-Glutamine.

Transfected packaging cells were incubated for 3 days at 37° C., then virus was collected from cell lysates using a standard freeze-thaw protocol. In brief, packaging cells were lifted by scraping and pelleted. Supernatant was removed, and cells were resuspended in a solution of 50 mM Tris-HCl; 150 mM NaCl; and 2 mM MgCl2 [pH 8.0]. Intracellular virus particles were released by inducing cell lysis via three consecutive freeze-thaw cycles, consisting of shuttling cell suspension between dry ice/ethanol bath and 37° C. water bath with vigorous vortexing. Viscosity was reduced by treating lysate with EMD Millipore Benzonase (50 U/ml of cell lysate) for 60 min at 37° C., with occasional mixing. Debris was then pelleted by centrifugation, and the resulting supernatant was filtered through a 0.22 μm PVDF Millex-GV Filter. For crude virus to be tested in vitro, the filtered lysate is added directly to an Amicon Ultra-15 Centrifugal Filter Unit with Ultracel-100 membrane (100 KDa MWCO) filter cartridge. The filter unit was centrifuged at 5-10 minute intervals until desired volume was reached in the upper chamber, then concentrated crude virus was pipetted into a low-protein-binding tube and stored at 4° C. For virus to be tested in vivo, the clarified lysate is further purified using a four step iodixanol density gradient. Gradients are loaded into a Beckman 70Ti rotor and spun at 66,100 rpm for 1.5 h at 10 C using and max acceleration and deceleration. After ultracentrifugation, iodixanol purified virions are extracted from the 40-60% interface. AAVs in iodixanol solution are diluted in DPBS+/+0.001% pluronic F68 so that the iodixanol is concentration is less than 1%. Purified virus is then concentrated to desired volume using a 100 kDa MWCO Amicon ultrafiltration unit.

Titer (viral genomes per milliliter or vg/mL) was determined by qPCR using a standard curve of a virus of known concentration.

### Cell Lines

All 293 cell lines were maintained in DMEM supplemented with 10% FBS, 1XNEAA, 1% Pen/Strep, and 1% L-glutamine. 293 hASGR1/2 and 293hCACNG1 cell lines were generated by lentiviral transduction of the parental 293 cell line with a vector expressing the corresponding cDNA. All cell lines were obtained from the Regeneron TC core facility.

Human skeletal myoblasts were purchased from Cook Myosite (SkMDC; Lot #P01059-14M) and maintained in MyoTonic Basal Media (MB-2222) supplemented with MyoTonic Growth Supplement (MS-3333) and grown in a 37° C. incubator with 5% CO₂.

C2C12 mouse myoblasts were purchased from ATCC and maintained in DMEM with 10% FBS and penicillin-streptomycin supplement and grown in a 37° C. incubator with 5% CO₂.

### AAV Capsid Protein Constructs

GeneBlocks encoding the desired SpyTag insertions, flanking linker amino acids, and additional mutations were purchased from IDT and cloned into BsiWI and XcmI-digested pAAV9-CAP wt using Gibson Assembly according to the manufacturer's protocol (NEB).

### Cloning SpyCatcher to Antibodies

GeneBlocks encoding antibody heavy chain variable regions were purchased from IDT, and cloned into CMV hIgG4US Sap1 SpyCatcher backbone using Gibson assembly.

### Cell Infection/Transduction and Flow Cytometric Analysis.

To infect cells, viral particles were added directly to the media of cells in culture, and the mixture was incubated at 37° C. Three days post-infection, cells were trypsinized, resuspended in PBS with 2% FBS, and the percentage of GFP+ cells was collected on a BD FACSCanto flow cytometer and analyzed using FlowJo software.

### Cell Infection/Transduction and Luciferase Assay Readout

To infect cells, viral particles were added directly to the media of cells in culture, and the mixture was incubated at 37° C. Three days post-infection, media was removed and cells were lysed using Promega Glo Lysis buffer. Luciferin substrate (Promega Bright Glo kit) was added to lysed cells and luminescence was measured using a luminometer.

### Myotube Transduction

Human skeletal myoblasts were seeded in collagen-coated 96 well plates with a clear base and black walls at 8500 cells/well. After 24 hours, growth media was changed to MyoTonic Differentiation Media (MD-5555) and replaced every 2 days. After 4 days of differentiation, myotubes formed, and differentiation media supplemented with virus preps was added to respective wells. After 3 days, myotubes were fixed with 4% PFA for subsequent GFP detection, or were lysed with Trizol for detection of uDys5 mRNA.

C2C12 cells were seeded in collagen-coated 96 well plates with a clear base and black walls at 10,000 cells/well. After 24 hours, growth media was replaced with differentiation media (DMEM with 2% horse serum) and placed in a 37° C. incubator with 7.5% CO₂. After 24 hours of differentiation, cells were transduced using virus preps diluted in differentiation media. After 3 days, myotubes were fixed with 4% PFA for subsequent GFP detection, or were lysed with Trizol for detection of uDys5 mRNA.

### Myotube Transduction Analysis

Following 15 minutes of fixation at room temperature, cells were washed with PBS and then blocked with 20% goat serum in PBS with 0.3% Triton-X for one hour at room temperature. Primary antibody against myosin heavy chain (MF20-c, Developmental Studies Hybridoma Bank) was diluted at 1:200 in blocking buffer and added to all wells for overnight incubation at 4° C. Wells were then washed gently with PBS 3 times, and Alexa 647 conjugated anti-mouse secondary antibody was diluted at 1:500 in blocking buffer and added to all wells for an hour at room temperature. Cells were then washed with PBS and stained with DAPI before imaging using an Axio Observer microscope (Ziess). GFP intensity within the myosin heavy chain positive areas was analyzed using HALO software (Indica Labs). For analysis of transduction with uDys5, RNA was isolated using a RNeasy Mini Kit (Qiagen) according to the manufacturer's protocol. RNA purification was completed following the manufacturer's protocol, and quantity and quality of the RNA was validated with a NanoDrop 2000. Equal amounts of RNA were reverse transcribed using the SuperScript VILO cDNA synthesis MasterMix (ThermoFisher) and diluted 10× with nuclease-free water. Gene expression was evaluated using Taqman primers/probe: AGGGTAGCTAGCATGGAAAAACA (uDys5 fwd), GGGCTTGTGAGACATGAGTGAT (uDys5 rev), ATTTACATTCTTATGTGCCT (uDys5 probe), with endogenous controls for human and mouse Hprt: Hs02800695_ml and Mm03024075_m1, respectively (Thermo Fisher)

### Mouse Lines

Humanized CACNG1 mice (CACNG1^{hu/hu}) were generated by replacing part of coding exon 1, intron 1, coding exons 2-4 (and intervening introns), and 82 bp of 3′ untranslated region (UTR) mouse Cacng1 with the orthologous partial coding exon 1 sequence, intron 1, coding exons 2-4 (and intervening introns), complete 3′ UTR and an additional 158 bp after the 3′ UTR of human CACNG1. Humanized ASGR1 mice were generated by according to the methods described in WO/2019006034. Strain-matched (50500) mice were used as controls and bred in-house. Wildtype C57BL/6, DBA/2J, and dystrophic D2-mdx mice (MDX) were purchased from the Jackson Laboratory (Stock #s 000664, 000671 and 013141, respectively).

### In Vivo Analysis AAV2 Luciferase Vectors

Male 50500 and humanized CACNG1 mice ranging from 3-4 months of age were injected intravenously via tail vein with PBS or 5E11 vgs of either wildtype AAV2, AAV2 HBM anti-ASGR1, or AAV2 HBM anti-CACNG1 mAb #1 carrying a luciferase reporter. Five weeks post IV injection, mice were anesthetized using isoflurane, injected with a Luciferin substrate and euthanized 7-10 minutes later. Liver, tongue, diaphragm, and quad were harvested and imaged ex vivo using IVIS Spectrum in vivo Imaging System. The raw data was analyzed using living image software to determine average radiance (photons/sec/cm2/sr).

### In Vivo Analysis of AAV9 Luciferase Vectors

Female humanized CACNG1 and humanized ASGR1 mice 10 weeks of age were injected intravenously via tail vein with PBS or 5E10 vgs of either wildtype AAV9, AAV9 N272A, AAV9 N272A anti ASGR1 mAb, AAV9 anti ASGR1 Fab, AAV9 N272A anti CACNG1 mAb #1, or AAV9 N272A anti CACNG1 Fab carrying a luciferase reporter. Three weeks post injection, mice were anesthetized using isoflurane, injected with a Luciferin substrate and euthanized 7-10 minutes later. Liver, hindlimb, quad, and tongue were harvested and imaged ex vivo using IVIS Spectrum in vivo Imaging System. The raw data was analyzed using living image software to determine average radiance (photons/sec/cm2/sr).

### In Vivo Analysis of AAV9 GFP Vectors

Adult (3-7 months old) male CACNG1^{hu/hu}, WT C57BL/6, and D2-mdx mice were tail vein injected with 1E+11 vg/mouse of AAV9 (WT AAV9, AAV9 N272A, AAV9 N272A anti-ASGR1 mAb, and AAV9 N272A anti-CACNG1 mAbs “#1” and “#2” and “#3”). Mice were sacrificed 3 weeks post injection, and the following organs were harvested for immunohistochemistry: liver, spleen, heart, tongue, diaphragm, quadriceps, gastrocnemius/plantaris/soleus complex, and tibialis anterior.

For the pooled AAV characterization experiment in cynomolgus monkey, control AAVs and AAV9 variants conjugated to the indicated antibodies were produced individually using the methods described above, but with barcoded pITR-CAG-GFP-hGHpA plasmids as the viral genome plasmids; each of the 12 viruses present in the pool was packaged with a version of pITR-CAG-GFP-hGHpA that carried a unique 32 nucleotide long barcode that was used to quantify transgene expression by that capsid variant. Two male cynomolgus macaques were given an intravenous bolus injection of 3E+13 vg/kg of the pooled virus mix. Two weeks after injection, animals were euthanized, and a set of tissues and organs were harvested for barcoding analysis. See, e.g., WO2018144813; Stoeckius et al. (2018) Genome Biol. 19:224; Stoeckius et al. (2017) Nat. Method 9:2579-10, each incorporated herein in its entirety by reference. Table 4 provides barcode number (BC #) associated with different viral particles (e.g. AAV9 cap mutation and corresponding antibody numbers where applicable).

TABLE 4

| BC# | AAV |
|---|---|
| 1 | AAV9 |
| 2 | AAV9 W503A + Non-targeting mAb |
| 3 | AAV9 W503A + ASGR1 mAb |
| 4 | AAV9 W503A + CACNG1 mAb #3 |
| 5 | AAV9 W503A + CACNG1 mAb #2 |
| 6 | AAV9 N272A + Non-targeting mAb |
| 7 | AAV9 N272A + ASGR1 mAb |
| 8 | AAV9 N272A + CACNG1 mAb #3 |
| 9 | AAV9 N272A + CACNG1 mAb #2 |
| 10 | AAV9 N272A + CAONG1 mAb #4 |
| 11 | AAV9 N272A + CACNG1 mAb #5 |
| 12 | AAV9 N272A + CACNG1 mAb #1 |

To assess serum readouts of liver health and complement activation (ALT, AST, Bb and C3a) following administration of wildtype and retargeted AAVs, AAV9 and AAV9 N272A anti-CACNG1 mAb #3 were produced according to the methods described above and packaged with pAAV CAG eGFP. Male cynomolgus macaques were given an intravenous bolus injection of 3E+13 vg/kg of either AAV9 wt (2 animals) or AAV9 N272A anti-CACNG1 mAb #3 (2 animals) or saline as a control (1 animal). Serum readouts of ALT, AST, Bb and C3a were collected at baseline (10 days prior to dosing) as well as 30 minutes, 6 hours, 24 hours and 48 hours post-dosing. Two weeks after injection, animals were euthanized, and a set of tissues and organs were harvested for analysis.

For immunohistochemistry staining, tissues were fixed in neutral buffered formalin solution and transferred to 70% ethanol 24 hours later. Organs were stored in 70% ethanol at room temperature for prior to paraffin embedding. Tissues were sectioned using standardized plane sectioning at 5 μm thickness. Anti-GFP IHC staining for eGFP expression was done using Benchmark ULTRA Ventana IHC/ISH system. Image analysis was done using HALO analysis software.

For immunofluorescence staining, muscle tissue was submerged in OCT embedding medium and frozen in liquid nitrogen-cooled isopentane. Tissues were cryosectioned at 12 μm thickness and subsequently fixed with 4% PFA and stained for laminin (Sigma-Aldrich), followed by Alexa 647-conjugated anti-rabbit secondary antibody and DAPI (Thermo Fisher Scientific). Slides were mounted with Fluoromount (Thermo Fisher Scientific) and imaged with an Axioscan slide scanner (Zeiss).

For barcoding analysis, total RNA isolated from cynomolgus monkey tissues and organs was purified using MagMAX-96 for Microarrays Total RNA Isolation Kit according to manufacturer's specifications. RNA was then treated with Turbo DNase and cDNA synthesis was performed using SuperScript IV reverse transcriptase and a hGH pA-specific primer (5′-GTCATGCATGCCTGGAATC-3′; SEQ ID NO:256). Barcoded GFP transcripts were amplified from cDNA samples with primers binding upstream (5′-TCGTCGGCAGCGTCAGATGTGTATAAGAGACAGCGAGCGCTGCTCGAGAG-3′; SEQ ID NO:257) and downstream (5′-GTCTCGTGGGCTCGGAGATGTGTATAAGAGACAGGGGTCACAGGGATGCCAC-3′; SEQ ID NO:258) of the barcodes using the Q5 High Fidelity 2× master mix. The pooled virus mix was included amongst the samples. Each sample was prepared in three technical replicates for the duration of the library preparation. Amplicons containing the Illumina adapters and unique dual indices (UDI-Illumina) were quantified using qubit and Tapestation, pooled at equimolar ratio, and sequenced on a Nextseq550 using the 300 cycles high output kit.

In Vivo Analysis of AAV9 uDys5 Vectors

6-week old male D2-mdx mice were tail vein injected with 1E+12 vg/mouse of WT AAV9 or AAV9 N272A anti-CACNG1 mAb #3 expressing uDys5 under the CK8 promoter. Mice were sacrificed 5 weeks post injection, and the following organs were harvested for qPCR analysis and stored in RNAlater (Thermo Fisher): liver, heart, quadriceps, gastrocnemius, tibialis anterior, soleus, tongue, and diaphragm. Tissues were then homogenized in Trizol, and aqueous phase was purified using MagMAX-96 total RNA Isolation Kit (Life Technologies), and gDNA was removed using RNase-free Dnase Set (Qiagen). mRNA was reverse transcribed into cDNA using SuperScript VILO Master Mix (Life Technologies) and qPCR was performed using the following Taqman primer/probe: AGGGTAGCTAGCATGGAAAAACA (uDys5 fwd), GGGCTTGTGAGACATGAGTGAT (uDys5 rev), ATTTACATTCTTATGTGCCT (uDys5 probe), with endogenous control: AAGGCCGTGGTGCTGATG (Rplp0 fwd), TCTCCAGAGCTGGGTTGTTCT (Rplp0 rev), AAGAACACCATGATGCGCAAGGC (probe).

For the detection of uDys5 myofiber membrane localization, cryosections of gastrocnemius muscle and heart were fixed with 4% PFA and stained for dystrophin (Developmental Studies Hybridoma Bank), followed by Alexa 546-conjugated anti-mouse secondary antibody. Slides were mounted with Fluoromount (Thermo Fisher Scientific) and imaged with an Axioscan slide scanner (Zeiss).

For the detection of uDys5 protein, quadriceps muscle was snap frozen in liquid nitrogen and subsequently homogenized using the mouse leg muscle setting of the Fast Prep-24 5G homogenizer (MP Biomedicals). Muscle was then added to a lysing matrix tube (MP Biomedicals) with lysis buffer (50 mM Tris HCl, 100 mM NaCl, 1 mM EDTA dihydrate, 1% Triton 100x) containing protease and phosphatase inhibitors (Sigma). Following protein quantitation using the BCA Assay (Thermo Fisher Scientific), lysates were heated with sample buffer and reducing agent (Thermo Fisher Scientific) at 70° C. for 10 minutes. Equal amounts of protein were loaded onto a 4-20% Tris-glycine gel (Bio-Rad) and resolved, then transferred to a PVDF membrane (BioRad) using the TurboTransfer System (BioRad). Membranes were blocked with 5% non-fat milk (Cell Signaling) before overnight incubation with the following primary antibodies: dystrophin (Developmental Studies Hybridoma Bank, MANHINGE1A), beta actin (Abcam, ab8224). After washing, membranes were incubated with the appropriate HRP-conjugated secondary antibodies (Cell Signaling). Membranes were incubated in ECL reagent (Cell Signaling) and visualized (Amersham Imager 600). Blot images were quantified using ImageJ.

To assess the effects of muscle transduction with uDys5 on circulating biomarkers of muscle damage, blood was collected before dosing and then four weeks after dosing into a 1.1 mL Z-Gel microtube (Sarstedt), and the serum was allowed to clot for a minimum of 2 hours. The blood was spun down at 4 C at 12,000 RPM for 10 mins. The supernatant was then collected and frozen at −80 C. Once all samples were collected, they were all thawed and then diluted 1:4 with DI water. Serum was analyzed using the ADVIA® Chemistry Creatine Kinase (CK_L) Reagents (REF 10729780) on the ADVIA® Chemistry XPT system.

To assess the functional effects of muscle transduction with uDys5, 6-week old male D2-mdx mice were tail vein injected with 1E+12 vg/mouse of WT AAV9 or AAV9 N272A anti-CACNG1 mAb #3 expressing uDys5 under the CK8 promoter, and maximal grip strength was assessed 12 weeks following injection. Grip strength of the forelimbs was assessed with a computerized grip strength meter (Bioseb, BIO-GS3) by holding mice vertically by their tail and lowering until they grasped the pull bar. Mice were then slowly pulled upwards until they lost their grip. The peak force generated (grams) was recorded after five pulls with 3 minutes of rest between trials. After 3 trials, the maximum force was averaged over the trials and recorded as maximal grip strength.

While the invention has been particularly shown and described with reference to a number of embodiments, it would be understood by those skilled in the art that changes in the form and details may be made to the various embodiments disclosed herein without departing from the spirit and scope of the invention and that the various embodiments disclosed herein are not intended to act as limitations on the scope of the claims. Although any methods and materials similar or equivalent to those described herein can be used in the practice or testing of the present invention, some preferred methods and materials are now described. All publications cited herein are incorporated herein by reference to describe in their entirety. Unless defined otherwise, all technical and scientific terms used herein have the same meaning as commonly understood by one of ordinary skill in the art to which this invention belongs.

What is claimed is:

1. A recombinant adeno-associated virus (AAV) particle comprising:
(i) a modified AAV capsid protein, and
(ii) a targeting ligand that binds a mammalian muscle cell-specific surface protein, wherein the modified AAV capsid protein is operably linked with the targeting ligand.

2. The recombinant AAV particle of claim 1, wherein:
(a) the modified AAV capsid protein comprises a first member and a second member of a protein:protein binding pair, and
(b) the second member of the protein:protein binding pair comprises the targeting ligand that binds the mammalian muscle cell-specific surface protein,
wherein the first member of the protein:protein binding pair and the second member of the protein:protein binding pair are associated to direct the tropism of the viral particle to the mammalian muscle cell.

3. The recombinant AAV particle of claim 1 or claim 2, wherein the mammalian muscle-specific surface protein is a human muscle cell-specific surface protein.

4. The recombinant AAV particle of any one of claims 1-3, wherein the mammalian muscle cell is a mammalian skeletal muscle cell.

5. The recombinant AAV particle of any one of claims 1-4, comprising the modified AAV capsid protein bound to the mammalian muscle cell-specific surface protein expressed on the surface of the mammalian muscle cell.

6. The recombinant AAV particle of any one of claims 1-5, comprising the modified AAV capsid protein bound to the mammalian muscle cell-specific surface protein expressed on the surface of the mammalian muscle cell,
wherein the mammalian muscle cell-specific surface protein is a human muscle cell-specific surface protein, and
wherein the mammalian muscle cell is a non-human animal muscle cell genetically modified to express the human muscle cell-specific surface protein.

7. The recombinant AAV particle of any one of claims 1-6, comprising the AAV capsid protein bound to the mammalian muscle-specific surface protein,
wherein the mammalian muscle cell-specific surface protein is a human muscle cell-specific surface protein, and
wherein the mammalian muscle cell is a rodent muscle cell genetically modified to express the human muscle cell-specific surface protein.

8. The recombinant AAV particle of claim 7, wherein the rodent muscle cell is a rat muscle cell or a mouse muscle cell.

9. The recombinant AAV particle of any one of claims 1-5, comprising the AAV capsid protein bound to the mammalian muscle cell-specific surface protein,
wherein the mammalian muscle cell-specific surface protein is a human muscle cell-specific surface protein, and
wherein the mammalian muscle cell is a human muscle cell.

10. The recombinant AAV particle of any one of claims 1-9, wherein the recombinant AAV particle is in vitro.

11. The recombinant AAV particle of any one of claims 1-9, wherein the recombinant AAV particle is in vivo.

12. The recombinant AAV particle of any one of claims 1-11, wherein the mammalian muscle cell-specific surface protein is mammalian Calcium Voltage-Gaged Auxiliary Subunit Gamma 1 (CACNG1).

13. The recombinant AAV particle of claim 12, wherein the mammalian muscle cell-specific surface protein is human CACNG1.

14. The recombinant AAV particle of any one of claims 1-13, wherein the targeting ligand comprises a heavy chain variable domain, light chain variable domain, heavy chain variable domain/light chain variable domain pair, HCDR1, HCDR2, HCDR3, LCDR1, LCDR2, LCDR3, and/or set of HCDR1-HCDR2-HCDR3-LCDR1-LCDR2-LCDR3 amino acid sequence(s) at least 90% identical to, respectively, an amino acid sequence of a heavy chain variable domain, light chain variable domain, heavy chain variable domain/light chain variable domain pair, HCDR1, HCDR2, HCDR3, LCDR1, LCDR2, LCDR3, and/or set of HCDR1-HCDR2-HCDR3-LCDR1-LCDR2-LCDR3 as set forth in any one of SEQ ID NOs: 1-240.

15. The recombinant AAV particle of any one of claims 1-14, wherein:
(a) the first member of the protein:protein binding pair comprises SpyTag, Isopeptag, SnoopTag, SpyTag002, SpyTag003, or any biologically active portions or variants thereof,
(b) the second member of the protein:protein binding pair comprises
(i) a SpyCatcher, KTag, pilin-C, SnoopCatcher, SpyCatcher002, SpyCatcher003, or any biologically active portions or variants thereof, and
(ii) the targeting ligand that binds a mammalian muscle cell-specific surface protein, and
(c) the first member of the protein:protein binding pair and the second member of the protein:protein binding pair are linked by an isopeptide bond.

16. The recombinant AAV particle of claim 15, wherein:
(a) the first member of the protein:protein binding pair comprises SpyTag, or any biologically active portion or variant thereof, and
(b) the second member of the protein:protein binding pair comprises SpyCatcher, or any biologically active portions or variants thereof, fused to the targeting ligand that binds the mammalian muscle cell-specific surface protein.

17. The recombinant AAV particle of any one of claims 2-16, comprising a first and/or second linker operably linking the first member of the protein:protein binding pair to the viral capsid protein.

18. The recombinant AAV particle of claim 17, wherein the first and second linker are not identical.

19. The recombinant AAV particle of claim 17, wherein the first and second linker are identical.

20. The recombinant AAV particle of any one of claims 17-19, wherein the first linker is 10 amino acids in length and/or the second linker is 10 amino acids in length.

21. The recombinant AAV particle of any one of claims 1-20, wherein the modified AAV capsid protein comprises a modified VP1 capsid protein, modified VP2 capsid protein, and/or modified VP3 capsid protein, and
wherein the modified VP1 capsid protein, modified VP2 capsid protein, and/or modified VP3 capsid protein comprises an insertion of a first member of a protein:protein binding pair and/or the targeting ligand, and
and wherein a portion of the modified VP1 capsid protein, modified VP2 capsid protein, and/or modified VP3 capsid protein, that comprises the insertion of a first member of a protein:protein binding pair and/or the targeting ligand, further comprises an amino acid sequence at least 90% identical to a corresponding capsid protein of a wild-type AAV.

22. The recombinant AAV particle of claim 21, wherein the modified VP1 capsid protein, the modified VP2 capsid protein, and/or the modified VP3 capsid protein further comprises, in addition to the insertion of a first member of a protein:protein binding pair and/or the targeting ligand:
(i) a substitution, insertion, or deletion of an amino acid,
(ii) a chimeric amino acid sequence, or
(iii) any combination of (i) and (ii).

23. The recombinant AAV particle of claim 22, wherein the substitution, insertion, or deletion of an amino acid reduces the natural tropism of the viral particle and/or creates a detectable label.

24. The recombinant AAV particle of any one of claims 1-23, wherein the AAV is selected from the group consisting of AAV1, AAV2, AAV3, AAV4, AAV5, AAV6, AAV7, AAV8, AAV9, a non-primate animal AAV listed in Table 2, and any chimera thereof.

25. The recombinant AAV particle of any one of claims 1-24, wherein the AAV is AAV2.

26. The recombinant AAV particle of any one of claims 1-25, wherein the recombinant AAV particle comprises a modified AAV2 VP1 capsid protein that comprises a first member of a protein:protein binding pair inserted at an amino acid position I-453 and/or I-587, and optionally linked to the AAV sequence via a linker on one or both sides.

27. The recombinant AAV particle of claim 26, wherein the recombinant AAV particle comprises a modified AAV2 VP1 capsid protein that comprises the first member of the protein:protein binding pair inserted, optionally via a linker, at position G453, optionally wherein the modified AAV2 VP1 capsid protein further comprises a mutation selected from R585A, R588A, R484A, R487A, K532A, and any combination thereof.

28. The recombinant AAV particle or composition of claim 26 or claim 27, wherein the recombinant AAV particle comprises a mosaic AAV capsid comprising a second set of AAV2 VP1 capsid proteins lacking the first member of the protein:protein binding pair,
optionally wherein the second set of AAV2 VP1 capsid proteins comprises a mutation selected from R585A, R588A, R484A, R487A, K532A and any combination thereof.

29. The recombinant AAV particle of any one of claims 1-24, wherein the AAV is AAV9.

30. The recombinant AAV particle of claim 29, wherein the viral capsid comprises a modified AAV9 VP1 capsid protein that comprises a first member of a specific binding pair inserted, optionally via a linker, at position I-453 or I-589.

31. The recombinant AAV particle of claim 30, wherein the recombinant AAV particle comprises a modified AAV9 VP1 capsid protein that comprises a first member of a protein:protein binding pair inserted, optionally via a linker, at position G453,
optionally wherein the modified AAV9 VP1 capsid protein further comprises a mutation selected from N272A, W503A, and a combination thereof.

32. The recombinant AAV particle of claim 30 or claim 31, wherein the recombinant AAV particle is a mosaic viral capsid comprising a second set of AAV9 VP1 capsid proteins lacking the first member of the protein:protein binding pair,
optionally wherein the second set of AAV9 VP1 capsid proteins comprises a mutation selected from N272A, W503A, and a combination thereof.

33. The recombinant AAV particle any one of claims 1-24, wherein the AAV is an avian AAV (AAAV), a non-primate mammalian AAV or a squamate AAV.

34. The recombinant viral particle or composition of claim 33, wherein the non-primate animal AAV is an AAAV.

35. The recombinant viral particle or composition of claim 34, wherein the viral capsid comprises a modified AAAV VP1 capsid protein that comprises the first member of the protein:protein binding pair inserted, optionally via a linker, at position I-444 or I-580.

36. The recombinant viral particle or composition of claim 33, wherein the non-primate animal AAV is a squamate AAV.

37. The recombinant viral particle or composition of claim 33, wherein the non-primate animal AAV is a bearded dragon AAV.

38. The recombinant viral particle or composition of claim 37, wherein the viral capsid comprises a modified bearded dragon VP1 capsid protein that comprises the first member of the protein:protein binding pair inserted, optionally via a linker, at position 1573 or I436.

39. The recombinant viral particle or composition of claim 33, wherein the non-primate animal AAV is a non-primate mammalian AAV.

40. The recombinant viral particle or composition of claim 39, wherein the non-primate mammalian AAV is a sea lion AAV.

41. The recombinant viral particle or composition of claim 34, wherein the viral capsid comprises a modified AAAV VP1 capsid protein that comprises the first member of the protein:protein binding pair inserted, optionally via a linker, at a position selected from the group consisting of 1429, 1430, 1431, 1432, 1433, 1434, 1436, 1437, and I565.

42. The recombinant AAV particle of any one of claims 1-41, wherein the recombinant AAV particle is a mosaic viral capsid, optionally wherein the mosaic viral capsid comprises (i) a first plurality of reference capsid proteins, each of which is not associated with the targeting ligand, and (ii) a second plurality of capsid proteins, each of which is associated with the targeting ligand, optionally wherein the mosaic AAV particle comprises the first plurality of reference capsid proteins and the second plurality of capsid proteins at a ratio of 1:7.

43. The recombinant AAV particle of any one of claims 1-42, wherein the targeting ligand is an antibody or a portion thereof.

44. The recombinant AAV particle of any one of claims 1-43, further comprising a nucleotide of interest encapsidated within the viral capsid.

45. The recombinant AAV particle of claim 44, wherein the nucleotide of interest is a reporter gene.

46. The recombinant AAV particle of claim 44 or claim 45, wherein the nucleotide of interest encodes β-galactosidase, green fluorescent protein (GFP), enhanced Green Fluorescent Protein (eGFP), MmGFP, blue fluorescent protein (BFP), enhanced blue fluorescent protein (eBFP), mPlum, mCherry, tdTomato, mStrawberry, J-Red, DsRed, mOrange, mKO, mCitrine, Venus, YPet, yellow fluorescent protein (YFP), enhanced yellow fluorescent protein (eYFP), Emerald, CyPet, cyan fluorescent protein (CFP), Cerulean, T-Sapphire, luciferase, alkaline phosphatase, or a combination thereof.

47. The recombinant AAV particle of claim 44, wherein the nucleotide of interest encodes a therapeutic protein, a suicide gene, an antibody or a fragment thereof, a CRISPR/Cas system or a portion(s) thereof, an antisense oligonucleotide, a ribozyme, an RNAi molecule, or a shRNA molecule.

48. A pharmaceutical composition comprising (a) the recombinant AAV particle according to any one of claims 1-47 and (b) a pharmaceutically acceptable carrier or excipient.

49. A method of delivering a nucleotide of interest to a mammalian muscle cell comprising contacting the mammalian muscle cell with (a) the recombinant AAV particle according to any one of claims 1-47 or (b) the pharmaceutical composition of claim 48,
wherein the muscle cell expresses the mammalian muscle cell-specific surface protein.

50. The method of claim 49, wherein the contacting is performed ex vivo.

51. The method of claim 49, wherein the contacting is performed in a subject.

52. The method of claim 51, wherein the subject is a primate animal, preferably a human.

53. The method of any one of claims 49-51, wherein the mammalian muscle cell is a mammalian skeletal muscle cell.

54. The method of any one of claims 49-53, wherein the mammalian muscle cell-specific surface protein is CACNG1.

55. The method of any one of claims 49-54, wherein the nucleotide of interest encodes a therapeutic protein, a suicide gene, an antibody or a fragment thereof, a CRISPR/Cas system or a portion(s) thereof, an antisense oligonucleotide, a ribozyme, an RNAi molecule, or a shRNA molecule.

56. A method of treating a muscle wasting or genetic muscle disease in a subject in need thereof comprising
administering to the patient (a) recombinant AAV particle according to any one of claims 1-47 or (b) the pharmaceutical composition of claim 48,
wherein the viral particle comprises a nucleotide of interest encapsidated within the viral capsid, and
wherein the nucleotide of interest encodes a therapeutic protein, a suicide gene, an antibody or a fragment thereof, a CRISPR/Cas system or a portion(s) thereof, an antisense oligonucleotide, a ribozyme, an RNAi molecule, or a shRNA molecule.

57. Use of the viral particle or composition according to any one of claims 1-47 or the pharmaceutical composition of claim 48 for the manufacture of a medicament for the treatment of muscle wasting or a genetic muscle disease.

58. The method of claim 56 or use of claim 57, wherein the muscle wasting or genetic muscle disease is selected from the group consisting of X-linked myotubular myopathy (XLMTM), Duchenne muscular dystrophy (DMD), myotonic dystrophy (DM1), Facioscapulohumeral muscular dystrophy Type 1 (FSHD), congenital muscular dystrophy type 1A (MDC1A), Limb girdle muscular dystrophy, and dystroglycanopathy.

59. The method of any one of claims 56-58, wherein the administration to the patient of the recombinant AAV particle or the pharmaceutical composition does not result in an increased level of a liver enzyme (e.g. ALT, AST) or a complement component (e.g. Bb, C3a) that is more than 3 fold, preferably 1.5 fold, higher than the corresponding level of the liver enzyme or complement component prior to the administration.


SEQUENCE LISTING (258 sequences)

SEQ ID NO: 1 - DNA, 357 bp, synthetic construct
  source 1..357: mol_type = other DNA
caggtgcagctggtggagtctgggggaggcgtggtccagcctgggaggtccctgagactctcctgtacagcgtctggaatcaccttcagaaattatggcatgcactgggtccgccaggctccaggcaaggggctggagtgggtggcagttatgtggtatgatggaagtaataagtactatgcagactccgtgaagggccgtttcaccatctccggagacaattccaaggtgtatctgcaaatgaacagcctgagagccgaggacacggctgtatattactgtgcgagaaggggcactataagaacagctgccccttttgactactggggtcagggaaccctggtcaccgtctcctca

SEQ ID NO: 2 - protein, 119 aa, synthetic construct
QVQLVESGGGVVQPGRSLRLSCTASGITFRNYGMHWVRQAPGKGLEWVAVMWYDGSNKYYADSVKGRFTISGDNSKVYLQMNSLRAEDTAVYYCARRGTIRTAAPFDYWGQGTLVTVSS

SEQ ID NO: 3 - DNA, 24 bp, synthetic construct
  source 1..24: mol_type = other DNA
ggaatcaccttcagaaattatggc

SEQ ID NO: 4 - protein, 8 aa, synthetic construct
GITFRNYG

SEQ ID NO: 5 - DNA, 24 bp, synthetic construct
  source 1..24: mol_type = other DNA
atgtggtatgatggaagtaataag

SEQ ID NO: 6 - protein, 8 aa, synthetic construct
MWYDGSNK

SEQ ID NO: 7 - DNA, 42 bp, synthetic construct
  source 1..42: mol_type = other DNA
gcgagaaggggcactataagaacagctgccccttttgactac

SEQ ID NO: 8 - protein, 14 aa, synthetic construct
ARRGTIRTAAPFDY

SEQ ID NO: 9 - DNA, 324 bp, synthetic construct
  source 1..324: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccgtcaaggttcagtggcagtggatctgggacagatttcactctcaccatcagcagtctgcaacctgaagattttgcaacttactactgtcaacagagttacagtacccctccgatcaccttcggccaagggacacgactggagattaaa

SEQ ID NO: 10 - protein, 108 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIK

SEQ ID NO: 11 - DNA, 18 bp, synthetic construct
  source 1..18: mol_type = other DNA
cagagcattagcagctat

SEQ ID NO: 12 - protein, 6 aa, synthetic construct
QSISSY

SEQ ID NO: 13-14 - skipped (2 entries)

SEQ ID NO: 15 - DNA, 30 bp, synthetic construct
  source 1..30: mol_type = other DNA
caacagagttacagtacccctccgatcacc

SEQ ID NO: 16 - protein, 10 aa, synthetic construct
QQSYSTPPIT

SEQ ID NO: 17 - DNA, 356 bp, synthetic construct
  source 1..356: mol_type = other DNA
caggtgcagctacagcagtggggcgcaggactgttgaagccttcggcgaccctgtcccgcacctgcgctgtctatggtgggtccttcagtggttactactggaactggatccgccagtccccagggaaggggctggaatggattggggaaatccttcatagtggaagaaccaactacaacccgtccctcaagagtcgagtcaccatatcagtagacacgtccaagaaccagttctccctgaagctgacctctgtgaccgccgcggacacggctgtatattactgtgcggaaggatagcagctcgtcacggctggttcgacccctggggccagggaaccctggtcaccgtctcctca

SEQ ID NO: 18 - protein, 119 aa, synthetic construct
QVQLQQWGAGLLKPSATLSRTCAVYGGSFSGYYWNWIRQSPGKGLEWIGEILHSGRTNYNPSLKSRVTISVDTSKNQFSLKLTSVTAADTAVYYCAGRIAARHGWFDPWGQGTLVTVSS

SEQ ID NO: 19 - DNA, 24 bp, synthetic construct
  source 1..24: mol_type = other DNA
ggtgggtccttcagtggttactac

SEQ ID NO: 20 - protein, 8 aa, synthetic construct
GGSFSGYY

SEQ ID NO: 21 - DNA, 21 bp, synthetic construct
  source 1..21: mol_type = other DNA
atccttcatagtggaagaacc

SEQ ID NO: 22 - protein, 7 aa, synthetic construct
ILHSGRT

SEQ ID NO: 23 - DNA, 39 bp, synthetic construct
  source 1..39: mol_type = other DNA
gcgggaaggatagcagctcgtcacggctggttcgacccc

SEQ ID NO: 24 - protein, 13 aa, synthetic construct
AGRIAARHGWFDP

SEQ ID NO: 25 - DNA, 321 bp, synthetic construct
  source 1..321: mol_type = other DNA
gacatccagatgacccagtctccatcttccgtgtctacatctgtaggagacagagtcaccatctcttgtcgggcgagtcaggatattcgcaagtggttagcctggtatcaacagaaaccaggaaaagcccctaaactcctgatctatgctacatccagtttgcaaagtggggtcccttcaaggttcagcggcagtggatctgggacagatttcactctcaccatcagcagcctgcagcctgaggattttgcaacttacttttgtcaacaggctaacagtttcccgttcacttttggccaggggaccaagctggagatcaaa

SEQ ID NO: 26 - protein, 107 aa, synthetic construct
DIQMTQSPSSVSTSVGDRVTISCRASQDIRKWLAWYQQKPGKAPKLLIYATSSLQSGVPSRFSGSGSGTDFTLTISSLQPEDFATYFCQQANSFPFTFGQGTKLEIK

SEQ ID NO: 27 - DNA, 18 bp, synthetic construct
  source 1..18: mol_type = other DNA
caggatattcgcaagtgg

SEQ ID NO: 28 - protein, 6 aa, synthetic construct
QDIRKW

SEQ ID NO: 29-30 - skipped (2 entries)

SEQ ID NO: 31 - DNA, 27 bp, synthetic construct
  source 1..27: mol_type = other DNA
caacaggctaacagtttcccgttcact

SEQ ID NO: 32 - protein, 9 aa, synthetic construct
QQANSFPFT

SEQ ID NO: 33 - DNA, 357 bp, synthetic construct
  source 1..357: mol_type = other DNA
caggtgcagctggtggagtctgggggaggcgtggtccagcctgggaggtccctgagactctcctgtgcagcgtctggattcaccttcagtacatatggcatgcactgggtccgccaggctccaggcaaggggctggagtgggtggcagttatttggcatgatggaagtgataaatattatgtagactccgtgaagggccgattctccatcgccagagacaattccaagaacacgctttatctgcaaatgaatagtctgagagtcgaggacacgggtatatattactgtgcgagaaggggtatacgtggaaccgtttttgaccactggggcctgggaaccctggtcaccgtctcctca

SEQ ID NO: 34 - protein, 119 aa, synthetic construct
QVQLVESGGGVVQPGRSLRLSCAASGFTFSTYGMHWVRQAPGKGLEWVAVIWHDGSDKYYVDSVKGRFSIARDNSKNTLYLQMNSLRVEDTGIYYCARRGIRGTVFDHWGLGTLVTVSS

SEQ ID NO: 35 - DNA, 24 bp, synthetic construct
  source 1..24: mol_type = other DNA
ggattcaccttcagtacatatggc

SEQ ID NO: 36 - protein, 8 aa, synthetic construct
GFTFSTYG

SEQ ID NO: 37 - DNA, 24 bp, synthetic construct
  source 1..24: mol_type = other DNA
atttggcatgatggaagtgataaa

SEQ ID NO: 38 - protein, 8 aa, synthetic construct
IWHDGSDK

SEQ ID NO: 39 - DNA, 36 bp, synthetic construct
  source 1..36: mol_type = other DNA
gcgagaaggggtatacgtggaaccgtttttgaccac

SEQ ID NO: 40 - protein, 12 aa, synthetic construct
ARRGIRGTVFDH

SEQ ID NO: 41 - DNA, 321 bp, synthetic construct
  source 1..321: mol_type = other DNA
gacatccagatgacccagtctccttccaccctgtctgcatctgtaggagacagagtcaccctcacttgtcgggccagtcagagtattagtaacaagttggcctggtatcagcagaaaccagggaaagcccctaacctcctgatctataaggcgtctaatttagaaagtggggtcccatcaaggttcagcggcagtggatctgggacagaattcactctcaccatcagcagcctgcagcctgatgattttgcaacttattactgccaacagtataatagttattcgtggacgttcggccaagggaccaaggtggaaatcaaa

SEQ ID NO: 42 - protein, 107 aa, synthetic construct
DIQMTQSPSTLSASVGDRVTLTCRASQSISNKLAWYQQKPGKAPNLLIYKASNLESGVPSRFSGSGSGTEFTLTISSLQPDDFATYYCQQYNSYSWTFGQGTKVEIK

SEQ ID NO: 43 - DNA, 18 bp, synthetic construct
  source 1..18: mol_type = other DNA
cagagtattagtaacaag

SEQ ID NO: 44 - protein, 6 aa, synthetic construct
QSISNK

SEQ ID NO: 45-46 - skipped (2 entries)

SEQ ID NO: 47 - DNA, 27 bp, synthetic construct
  source 1..27: mol_type = other DNA
caacagtataatagttattcgtggacg

SEQ ID NO: 48 - protein, 9 aa, synthetic construct
QQYNSYSWT

SEQ ID NO: 49 - DNA, 348 bp, synthetic construct
  source 1..348: mol_type = other DNA
caggtgcagctgcaggagtcgggcccaggactggtgaagccttcggagaccctgtccctcacctgcactgtctctggtgactccatcaataattactactggacctggctccggcagcccccagggaagggactggagtggattggttatatctattacagtgggagcgccaactacaacccctccctcaagagtcgagtcaccatatcagtagacacgtccaagaaccagttctccctgaagctaaattctgtgaccgctgcggacacggccgtgtattactgtgcgagaggggcggtcaagtacttccggcattggggccagggcaccctggtcaccgtctcctca

SEQ ID NO: 50 - protein, 116 aa, synthetic construct
QVQLQESGPGLVKPSETLSLTCTVSGDSINNYYWTWLRQPPGKGLEWIGYIYYSGSANYNPSLKSRVTISVDTSKNQFSLKLNSVTAADTAVYYCARGAVKYFRHWGQGTLVTVSS

SEQ ID NO: 51 - DNA, 24 bp, synthetic construct
  source 1..24: mol_type = other DNA
ggtgactccatcaataattactac

SEQ ID NO: 52 - protein, 8 aa, synthetic construct
GDSINNYY

SEQ ID NO: 53 - DNA, 21 bp, synthetic construct
  source 1..21: mol_type = other DNA
atctattacagtgggagcgcc

SEQ ID NO: 54 - protein, 7 aa, synthetic construct
IYYSGSA

SEQ ID NO: 55 - DNA, 30 bp, synthetic construct
  source 1..30: mol_type = other DNA
gcgagaggggcggtcaagtacttccggcat

SEQ ID NO: 56 - protein, 10 aa, synthetic construct
ARGAVKYFRH

SEQ ID NO: 57 - DNA, 324 bp, synthetic construct
  source 1..324: mol_type = other DNA
gaaattgtgttgacgcagtctccgggcaccctctctttgtctccaggggaaagagccaccctctcctgcagggccagtcagactattaaccacaacaacttagcctggtaccagcagagacctggccaggctcccaggctcctcatctatggtgcatccaacagggccactgccatcccagacaggttcagtggcagtgggtctgggacagacttcactctcaccatcagcagactggagcctgaagattttgaagtgtattcttgtcagcagtatggtagcttgccgctcactttcggcggagggaccaaggtggagatcaaa

SEQ ID NO: 58 - protein, 108 aa, synthetic construct
EIVLTQSPGTLSLSPGERATLSCRASQTINHNNLAWYQQRPGQAPRLLIYGASNRATAIPDRFSGSGSGTDFTLTISRLEPEDFEVYSCQQYGSLPLTFGGGTKVEIK

SEQ ID NO: 59 - DNA, 21 bp, synthetic construct
  source 1..21: mol_type = other DNA
cagactattaaccacaacaac

SEQ ID NO: 60 - protein, 7 aa, synthetic construct
QTINHNN

SEQ ID NO: 61-62 - skipped (2 entries)

SEQ ID NO: 63 - DNA, 27 bp, synthetic construct
  source 1..27: mol_type = other DNA
cagcagtatggtagcttgccgctcact

SEQ ID NO: 64 - protein, 9 aa, synthetic construct
QQYGSLPLT

SEQ ID NO: 65 - DNA, 363 bp, synthetic construct
  source 1..363: mol_type = other DNA
caggtgcagctggtggagtctgggggaggcgtggtccagcctgggacgtccctgagactctcctgtgcagcgtcaggattcaccttcagtagctatggcatgcactgggtccgccaggctccaggcaaggggctggagtgggtggcagttatatggattgatggaagtaataaatattatgcagactccgtgaagggccgattcaccatctccagagacaattccaagaacacgctgtatctgcaaatgaacagcctgagagccgaggacacggctgtgtattactgtgcgagaagggggggtatagtagtagctgccccctttgactactggggccagggaaccctggtcaccgtctcctca

SEQ ID NO: 66 - protein, 121 aa, synthetic construct
QVQLVESGGGVVQPGTSLRLSCAASGFTFSSYGMHWVRQAPGKGLEWVAVIWIDGSNKYYADSVKGRFTISRDNSKNTLYLQMNSLRAEDTAVYYCARRGGIVVAAPFDYWGQGTLVTVSS

SEQ ID NO: 67 - DNA, 24 bp, synthetic construct
  source 1..24: mol_type = other DNA
ggattcaccttcagtagctatggc

SEQ ID NO: 68 - protein, 8 aa, synthetic construct
GFTFSSYG

SEQ ID NO: 69 - DNA, 24 bp, synthetic construct
  source 1..24: mol_type = other DNA
atatggattgatggaagtaataaa

SEQ ID NO: 70 - protein, 8 aa, synthetic construct
IWIDGSNK

SEQ ID NO: 71 - DNA, 42 bp, synthetic construct
  source 1..42: mol_type = other DNA
gcgagaagggggggtatagtagtagctgccccctttgactac

SEQ ID NO: 72 - protein, 14 aa, synthetic construct
ARRGGIVVAAPFDY

SEQ ID NO: 73 - DNA, 324 bp, synthetic construct; identical to SEQ ID NO: 9
  source 1..324: mol_type = other DNA

SEQ ID NO: 74 - protein, 108 aa, synthetic construct; identical to SEQ ID NO: 10

SEQ ID NO: 75 - DNA, 18 bp, synthetic construct
  source 1..18: mol_type = other DNA
cagagcattagcagctat

SEQ ID NO: 76 - protein, 6 aa, synthetic construct
QSISSY

SEQ ID NO: 77-78 - skipped (2 entries)

SEQ ID NO: 79 - DNA, 30 bp, synthetic construct; identical to SEQ ID NO: 15
  source 1..30: mol_type = other DNA

SEQ ID NO: 80 - protein, 10 aa, synthetic construct
QQSYSTPPIT

SEQ ID NO: 81 - DNA, 363 bp, synthetic construct
  source 1..363: mol_type = other DNA
caggtgcagttggtggagtctgggggaggcgtggtccagcctgggaggtccctgagactctcctgtgaagcgtctggaatcaccttcagaaactatggcatgcactgggtccgccaggctccaggcaaggggctggagtgggtggcagttatgtggtatgatggaagtaataaatactacgcagactccgtgaagggccgattcaccatctccagagacaattccaagaacacggtgtatctgcaaatgaacagcctgagagccgaagacacggctgtgtattactgtgcgagacggggtcatatagcaacagctgctccctttgactactggggccagggaaccctggtcaccgtctcctca

SEQ ID NO: 82 - protein, 121 aa, synthetic construct
QVQLVESGGGVVQPGRSLRLSCEASGITFRNYGMHWVRQAPGKGLEWVAVMWYDGSNKYYADSVKGRFTISRDNSKNTVYLQMNSLRAEDTAVYYCARRGHIATAAPFDYWGQGTLVTVSS

SEQ ID NO: 83 - DNA, 24 bp, synthetic construct
  source 1..24: mol_type = other DNA
ggaatcaccttcagaaactatggc

SEQ ID NO: 84 - protein, 8 aa, synthetic construct
GITFRNYG

SEQ ID NO: 85 - DNA, 24 bp, synthetic construct
  source 1..24: mol_type = other DNA
atgtggtatgatggaagtaataaa

SEQ ID NO: 86 - protein, 7 aa, synthetic construct
MWYDGSN

SEQ ID NO: 87 - DNA, 42 bp, synthetic construct
  source 1..42: mol_type = other DNA
gcgagacggggtcatatagcaacagctgctccctttgactac

SEQ ID NO: 88 - protein, 13 aa, synthetic construct
ARRGHIATAAPFD

SEQ ID NO: 89 - DNA, 324 bp, synthetic construct
  source 1..324: mol_type = other DNA
gacatccagatgacccagtctccatcctccctgtctgcatccgtaggagacagagtcaccatcagttgccgggcaagtcagagcattagtagttatttaaattggtatcagcagaaaccagggaaagcccctaaggtcctgatgtatgctgcatccagtttgcaaagtggggtcccatcaaggttcagtggcagtggatctgggacagatttcactctcaccatcagcagtctgcaacctgaggattttgcaacttactactgtcaacagagttacagtacccctccgatcaccttcggccaagggacacgactggagattaaa

SEQ ID NO: 90 - protein, 108 aa, synthetic construct
DIQMTQSPSSLSASVGDRVTISCRASQSISSYLNWYQQKPGKAPKVLMYAASSLQSGVPSRFSGSGSGTDFTLTISSLQPEDFATYYCQQSYSTPPITFGQGTRLEIK

SEQ ID NO: 91 - DNA, 18 bp, synthetic construct
  source 1..18: mol_type = other DNA
cagagcattagtagttat

SEQ ID NO: 92 - protein, 6 aa, synthetic construct
QSISSY

SEQ ID NO: 93-94 - skipped (2 entries)

SEQ ID NO: 95 - DNA, 30 bp, synthetic construct; identical to SEQ ID NO: 15
  source 1..30: mol_type = other DNA

SEQ ID NO: 96 - protein, 10 aa, synthetic construct
QQSYSTPPIT

SEQ ID NO: 97 - DNA, 363 bp, synthetic construct
  source 1..363: mol_type = other DNA
caggtgcagctggtggagtctgggggaggcgtggtccagcctgggaggtccctgagactctcctgtacagcgtctggattcaccttccgttcctatggcatgcactgggtccgccaggctccaggcaaggggctggagtgggtgtcagttatttggattgatggaaataatatatactatgcagactccgtgaagggccgattcaccatctccagagacaattccaagaacacgctgtatctgcaaatggacagcctgagagccgaggacacggctgtttattactgtgcgagaagactggctataacatcagctgccccctttgactactggggccagggaaccctggtcaccgtctcctca

SEQ ID NO: 98 - protein, 121 aa, synthetic construct
QVQLVESGGGVVQPGRSLRLSCTASGFTFRSYGMHWVRQAPGKGLEWVSVIWIDGNNIYYADSVKGRFTISRDNSKNTLYLQMDSLRAEDTAVYYCARRLAITSAAPFDYWGQGTLVTVSS

SEQ ID NO: 99 - DNA, 24 bp, synthetic construct
  source 1..24: mol_type = other DNA
ggattcaccttccgttcctatggc

SEQ ID NO: 100 - protein, 8 aa, synthetic construct
GFTFRSYG

SEQ ID NO: 101 - DNA, 24 bp, synthetic construct
  source 1..24: mol_type = other DNA
atttggattgatggaaataatata

SEQ ID NO: 102 - protein, 8 aa, synthetic construct
IWIDGNNI

SEQ ID NO: 103 - DNA, 42 bp, synthetic construct
  source 1..42: mol_type = other DNA
gcgagaagactggctataacatcagctgccccctttgactac

SEQ ID NO: 104 - protein, 14 aa, synthetic construct
ARRLAITSAAPFDY

SEQ ID NO: 105 - DNA, 324 bp, synthetic construct; identical to SEQ ID NO: 9
  source 1..324: mol_type = other DNA

SEQ ID NO: 106 - protein, 108 aa, synthetic construct; identical to SEQ ID NO: 10

SEQ ID NO: 107 - DNA, 18 bp, synthetic construct
  source 1..18: mol_type = other DNA
cagagcattagcagctat

SEQ ID NO: 108 - protein, 6 aa, synthetic construct
QSISSY

SEQ ID NO: 109-110 - skipped (2 entries)

SEQ ID NO: 111 - DNA, 30 bp, synthetic construct; identical to SEQ ID NO: 15
  source 1..30: mol_type = other DNA

SEQ ID NO: 112 - protein, 10 aa, synthetic construct
QQSYSTPPIT

SEQ ID NO: 113 - DNA, 378 bp, synthetic construct
  source 1..378: mol_type = other DNA
caggttcagctggtgcagtctggagctgaggtgaagaagcctggggcctcagtgaaggtctcctgcaaggcttctggttacgccttcaccacctatggtatcacctgggtgcgacaggcccctggacaaggacttgagtggatgggatggatcagcgcttacaatggaaatacaaactatgcagagaaggtccagggcagattcaccatgaccacagacacatccacgaatacagcctacatggagctgaggagcctgagatccgacgacacggccgtgtatttctgtgcgagaaagggtcactatggttcggggacttattataacccctttggttttgatttttggggccaagggacaatggtcaccgtctcttca

SEQ ID NO: 114 - protein, 126 aa, synthetic construct
QVQLVQSGAEVKKPGASVKVSCKASGYAFTTYGITWVRQAPGQGLEWMGWISAYNGNTNYAEKVQGRFTMTTDTSTNTAYMELRSLRSDDTAVYFCARKGHYGSGTYYNPFGFDFWGQGTMVTVSS

SEQ ID NO: 115 - DNA, 24 bp, synthetic construct
  source 1..24: mol_type = other DNA
ggttacgccttcaccacctatggt

SEQ ID NO: 116 - protein, 8 aa, synthetic construct
GYAFTTYG

SEQ ID NO: 117 - DNA, 24 bp, synthetic construct
  source 1..24: mol_type = other DNA
atcagcgcttacaatggaaataca

SEQ ID NO: 118 - protein, 7 aa, synthetic construct
ISAYNGN

SEQ ID NO: 119 - DNA, 57 bp, synthetic construct
  source 1..57: mol_type = other DNA
gcgagaaagggtcactatggttcggggacttattataacccctttggttttgatttt

SEQ ID NO: 120 - protein, 19 aa, synthetic construct
CARKGHYGSGTYYNPFGFD

SEQ ID NO: 121 - DNA, 324 bp, synthetic construct
  source 1..324: mol_type = other DNA
gaaattatgttgatgcagtctccaggcaccctgtctttgtctccaggggaaagagccaccctctcctgcagggccagtcagagtgttagcagcagctacttagcctggtaccaacagaaacctggccaggctcccaggctcctcatctatggtgcatccagcagggccactgacatcccagacaggttcagtggcagtgggtctgggacagacttcactctcaccatcagcagactggagcctgaagattttgcagtttatttctgtcagcagtattatggctcaccttggacgttcggccaagggaccaaggtggaaatcaag

SEQ ID NO: 122 - protein, 108 aa, synthetic construct
EIMLMQSPGTLSLSPGERATLSCRASQSVSSSYLAWYQQKPGQAPRLLIYGASSRATDIPDRFSGSGSGTDFTLTISRLEPEDFAVYFCQQYYGSPWTFGQGTKVEIK

SEQ ID NO: 123 - DNA, 21 bp, synthetic construct
  source 1..21: mol_type = other DNA
cagagtgttagcagcagctac

SEQ ID NO: 124 - protein, 7 aa, synthetic construct
QSVSSSY

SEQ ID NO: 125-126 - skipped (2 entries)

SEQ ID NO: 127 - DNA, 27 bp, synthetic construct
  source 1..27: mol_type = other DNA
cagcagtattatggctcaccttggacg

SEQ ID NO: 128 - protein, 9 aa, synthetic construct
CQQYYGSPW

SEQ ID NO: 129 - DNA, 360 bp, synthetic construct
  source 1..360: mol_type = other DNA
gaggtgcagctggtggagtctgggggaggcttggtccagccgggggggtccctgaaactctcctgtacagcctctgggttgaccctcagtgactctgctatgcactgggtccgccaggcttccgggaaagggctggagtgggttggccgtataagaaataaggctaataggtacgcgacagaatatgctgcgtcggtgaaaggcaggttcaccatttcaagagatgattcaaagaacacggcgtatctacaaatgaacagcctgaaaaccgaggacacggccgtgtattattgtactagaaactggaagattttcctctttgactactggggccagggaaccctggtcaccgtctcctca

SEQ ID NO: 130 - protein, 120 aa, synthetic construct
EVQLVESGGGLVQPGGSLKLSCTASGLTLSDSAMHWVRQASGKGLEWVGRIRNKANRYATEYAASVKGRFTISRDDSKNTAYLQMNSLKTEDTAVYYCTRNWKIFLFDYWGQGTLVTVSS

SEQ ID NO: 131 - DNA, 24 bp, synthetic construct
  source 1..24: mol_type = other DNA
gggttgaccctcagtgactctgct

SEQ ID NO: 132 - protein, 8 aa, synthetic construct
GLTLSDSA

SEQ ID NO: 133 - DNA, 30 bp, synthetic construct
  source 1..30: mol_type = other DNA
ataagaaataaggctaataggtacgcgaca

SEQ ID NO: 134 - protein, 10 aa, synthetic construct
IRNKANRYAT

SEQ ID NO: 135 - DNA, 33 bp, synthetic construct
  source 1..33: mol_type = other DNA
actagaaactggaagattttcctctttgactac

SEQ ID NO: 136 - protein, 11 aa, synthetic construct
TRNWKIFLFDY

SEQ ID NO: 137 - DNA, 324 bp, synthetic construct
  source 1..324: mol_type = other DNA
gaaattgtgttgacgcagtctccaggcaccctgactttgtctccaggggaaagagccaccctctcctgcagggccagtcagagtgttggcagcaaatacttagcctggttccagcagaaacgtggccaggctcccaggctcctcatctatggtgcatccagcaggaccagtggcatccccgacaggatcagtggcagtgggtcagggacagacttcactctcaccatcagcagactggagcctgaagattttgcagtgtattactgtcagcagtatggaagttcaccctggacgttcggccaagggaccaaggtggaaatcaaa

SEQ ID NO: 138 - protein, 108 aa, synthetic construct
EIVLTQSPGTLTLSPGERATLSCRASQSVGSKYLAWFQQKRGQAPRLLIYGASSRTSGIPDRISGSGSGTDFTLTISRLEPEDFAVYYCQQYGSSPWTFGQGTKVEIK

SEQ ID NO: 139 - DNA, 21 bp, synthetic construct
  source 1..21: mol_type = other DNA
cagagtgttggcagcaaatac

SEQ ID NO: 140 - protein, 7 aa, synthetic construct
QSVGSKY

SEQ ID NO: 141-142 - skipped (2 entries)

SEQ ID NO: 143 - DNA, 27 bp, synthetic construct
  source 1..27: mol_type = other DNA
cagcagtatggaagttcaccctggacg

SEQ ID NO: 144 - protein, 9 aa, synthetic construct
QQYGSSPWT

SEQ ID NO: 145 - DNA, 375 bp, synthetic construct
  source 1..375: mol_type = other DNA
gaggtgcagctgttggagtctgggggaggcttggtacagccgggggggtccctgagactctcctgtgcagcctctggattcacctttaacaactatggcatgagctgggtccgccagggtccagggaaggggctggagtgggtctcatctattagtggtagtggtggtaccacattctacgcagactccgtgaagggccggttcaccatctccagagacaattccaagaacacgctgtatctgcaaatgaacagcctgagagccgaggacacggccgtatattactgtggcaaaggaggatattgtagtagtagcggctgccgtcactacggtatggacgtctggggccaagggaccacggtcaccgtctcctca

SEQ ID NO: 146 - protein, 125 aa, synthetic construct
EVQLLESGGGLVQPGGSLRLSCAASGFTFNNYGMSWVRQGPGKGLEWVSSISGSGGTTFYADSVKGRFTISRDNSKNTLYLQMNSLRAEDTAVYYCGKGGYCSSSGCRHYGMDVWGQGTTVTVSS

SEQ ID NO: 147 - DNA, 24 bp, synthetic construct
  source 1..24: mol_type = other DNA
ggattcacctttaacaactatggc

SEQ ID NO: 148 - protein, 8 aa, synthetic construct
GFTFNNYG

SEQ ID NO: 149 - DNA, 24 bp, synthetic construct
  source 1..24: mol_type = other DNA
attagtggtagtggtggtaccaca

SEQ ID NO: 150 - protein, 6 aa, synthetic construct
SGSGGT

SEQ ID NO: 151 - DNA, 54 bp, synthetic construct
  source 1..54: mol_type = other DNA
ggcaaaggaggatattgtagtagtagcggctgccgtcactacggtatggacgtc

SEQ ID NO: 152 - protein, 14 aa, synthetic construct
CGKGGYCSSSGCRH

SEQ ID NO: 153 - DNA, 330 bp, synthetic construct
  source 1..330: mol_type = other DNA
cagtctgtgctgactcagccaccctcagcgtctgggacccccgggcagagggtcaccatctcttgttctggaagcagctccaacatcggaaataattatatatactggtaccagcggctcccaggaacgacccccaaactcctcatctataggaataatcagcggccctcaggggtccctgaccgattctctggctccaagtctggcacctcagcctccctggccatcagtgggctccggtccgaggatgaggctgattattactgtgcagcatgggatgacaccctgagtgggtatgtcttcggaactgggaccaaggtcaccgtccta

SEQ ID NO: 154 - protein, 110 aa, synthetic construct
QSVLTQPPSASGTPGQRVTISCSGSSSNIGNNYIYWYQRLPGTTPKLLIYRNNQRPSGVPDRFSGSKSGTSASLAISGLRSEDEADYYCAAWDDTLSGYVFGTGTKVTVL

SEQ ID NO: 155 - DNA, 24 bp, synthetic construct
  source 1..24: mol_type = other DNA
agctccaacatcggaaataattat

SEQ ID NO: 156 - protein, 8 aa, synthetic construct
SSNIGNNY

SEQ ID NO: 157-158 - skipped (2 entries)

SEQ ID NO: 159 - DNA, 33 bp, synthetic construct
  source 1..33: mol_type = other DNA
gcagcatgggatgacaccctgagtgggtatgtc

SEQ ID NO: 160 - protein, 11 aa, synthetic construct
CAAWDDTLSGY

SEQ ID NO: 161 - DNA, 363 bp, synthetic construct
  source 1..363: mol_type = other DNA
gaggtgcagctggtggagtctgggggaaacttggtacagcctggggggtccctgagactctcctgtgcagcctctggattcacctttaccagccatgccatgaactgggtccgccaggctccagggaaggggctggagtgggtctcagttattactggtagaggttttgacacacactacgctgactccgtgaagggccggttcaccatctccagagacatttccaaaaacacgctgtatctgcaaatgaacagcctgagagccgaggacacggccgtttattactgtgcgaaaggtctctatgattcggggaattattatatcgattactggggccagggaaccctggtcaccgtctcctca

SEQ ID NO: 162 - protein, 121 aa, synthetic construct
EVQLVESGGNLVQPGGSLRLSCAASGFTFTSHAMNWVRQAPGKGLEWVSVITGRGFDTHYADSVKGRFTISRDISKNTLYLQMNSLRAEDTAVYYCAKGLYDSGNYYIDYWGQGTLVTVSS

SEQ ID NO: 163 - DNA, 24 bp, synthetic construct
  source 1..24: mol_type = other DNA
ggattcacctttaccagccatgcc

SEQ ID NO: 164 - protein, 8 aa, synthetic construct
GFTFTSHA

SEQ ID NO: 165 - DNA, 24 bp, synthetic construct
  source 1..24: mol_type = other DNA
attactggtagaggttttgacaca

SEQ ID NO: 166 - protein, 8 aa, synthetic construct
ITGRGFDT

SEQ ID NO: 167 - DNA, 42 bp, synthetic construct
  source 1..42: mol_type = other DNA
gcgaaaggtctctatgattcggggaattattatatcgattac

SEQ ID NO: 168 - protein, 14 aa, synthetic construct
AKGLYDSGNYYIDY

SEQ ID NO: 169 - DNA, 333 bp, synthetic construct
  source 1..333: mol_type = other DNA
cagtctgtgttgacgcagccgccctcagtgtctgcggccccaggacagaaggtcaccatctcctgctctggaagcagctccaacattgggaataattatgtttcctggtaccagcagctcccaggaacagcccccaaactcctcatttatgacaataataagcgaccctcagggattcctgaccgattctctggctccaagtctggcacgtcagccaccctgggcatcaccggactccagactggggacgaggccgattattactgcggaacatgggatctcagcctgagtttcaattgggtgttcggcggagggaccaagctgaccgtccta

SEQ ID NO: 170 - protein, 111 aa, synthetic construct
QSVLTQPPSVSAAPGQKVTISCSGSSSNIGNNYVSWYQQLPGTAPKLLIYDNNKRPSGIPDRFSGSKSGTSATLGITGLQTGDEADYYCGTWDLSLSFNWVFGGGTKLTVL

SEQ ID NO: 171 - DNA, 24 bp, synthetic construct
  source 1..24: mol_type = other DNA
agctccaacattgggaataattat

SEQ ID NO: 172 - protein, 8 aa, synthetic construct
SSNIGNNY

SEQ ID NO: 173-174 - skipped (2 entries)

SEQ ID NO: 175 - DNA, 36 bp, synthetic construct
  source 1..36: mol_type = other DNA
ggaacatgggatctcagcctgagtttcaattgggtg

SEQ ID NO: 176 - protein, 12 aa, synthetic construct
GTWDLSLSFNWV

SEQ ID NO: 177 - DNA, 378 bp, synthetic construct; identical to SEQ ID NO: 113
  source 1..378: mol_type = other DNA

SEQ ID NO: 178 - protein, 126 aa, synthetic construct; identical to SEQ ID NO: 114

SEQ ID NO: 179 - DNA, 24 bp, synthetic construct
  source 1..24: mol_type = other DNA
ggttacgccttcaccacctatggt

SEQ ID NO: 180 - protein, 8 aa, synthetic construct
GYAFTTYG

SEQ ID NO: 181 - DNA, 24 bp, synthetic construct
  source 1..24: mol_type = other DNA
atcagcgcttacaatggaaataca

SEQ ID NO: 182 - protein, 8 aa, synthetic construct
ISAYNGNT

SEQ ID NO: 183 - DNA, 57 bp, synthetic construct; identical to SEQ ID NO: 119
  source 1..57: mol_type = other DNA

SEQ ID NO: 184 - protein, 19 aa, synthetic construct
ARKGHYGSGTYYNPFGFDF

SEQ ID NO: 185 - DNA, 324 bp, synthetic construct
  source 1..324: mol_type = other DNA
gaaattgtgttgacgcagtctccaggcaccctgtctttgtctccaggggaaagagccaccctctcctgcagggccagtcagagtgttagcagcagctacttagcctggtaccaacagaaacctggccaggctcccaggctcctcatctatggtgcatccagcagggccactggcatcccagacaggttcagtggcagtgggtctgggacagacttcactctcaccatcagcagactggagcctgaagattttgctttgtatttctgtcagcagtattatggctcaccttggacgttcggccaagggaccaaggtggaaatcaaa

SEQ ID NO: 186 - protein, 108 aa, synthetic construct
EIVLTQSPGTLSLSPGERATLSCRASQSVSSSYLAWYQQKPGQAPRLLIYGASSRATGIPDRFSGSGSGTDFTLTISRLEPEDFALYFCQQYYGSPWTFGQGTKVEIK

SEQ ID NO: 187 - DNA, 21 bp, synthetic construct
  source 1..21: mol_type = other DNA
cagagtgttagcagcagctac

SEQ ID NO: 188 - protein, 7 aa, synthetic construct
QSVSSSY

SEQ ID NO: 189-190 - skipped (2 entries)

SEQ ID NO: 191 - DNA, 27 bp, synthetic construct
  source 1..27: mol_type = other DNA
cagcagtattatggctcaccttggacg

SEQ ID NO: 192 - protein, 9 aa, synthetic construct
QQYYGSPWT

SEQ ID NO: 193 - DNA, 1338 bp, synthetic construct
  source 1..1338: mol_type = other DNA
caggtgcagctggtggagtctgggggaggcgtggtccagcctgggaggtccctgagactctcctgtacagcgtctggaatcaccttcagaaattatggcatgcactgggtccgccaggctccaggcaaggggctggagtgggtggcagttatgtggtatgatggaagtaataagtactatgcagactccgtgaagggccgtttcaccatctccggagacaattccaaggtgtatctgcaaatgaacagcctgagagccgaggacacggctgtatattactgtgcgagaaggggcactataagaacagctgccccttttgactactggggtcagggaaccctggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccgtgcccagcaccaggcggtggcggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 194 - protein, 445 aa, synthetic construct
QVQLVESGGGVVQPGRSLRLSCTASGITFRNYGMHWVRQAPGKGLEWVAVMWYDGSNKYYADSVKGRFTISGDNSKVYLQMNSLRAEDTAVYYCARRGTIRTAAPFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPGGGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 195 - DNA, 648 bp, synthetic construct
  source 1..648: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccgtcaaggttcagtggcagtggatctgggacagatttcactctcaccatcagcagtctgcaacctgaagattttgcaacttactactgtcaacagagttacagtacccctccgatcaccttcggccaagggacacgactggagattaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 196 - protein, 215 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 197 - DNA, 1341 bp, synthetic construct
  source 1..1341: mol_type = other DNA
caggtgcagctacagcagtggggcgcaggactgttgaagccttcggcgaccctgtcccgcacctgcgctgtctatggtgggtccttcagtggttactactggaactggatccgccagtccccagggaaggggctggaatggattggggaaatccttcatagtggaagaaccaactacaacccgtccctcaagagtcgagtcaccatatcagtagacacgtccaagaaccagttctccctgaagctgacctctgtgaccgccgcggacacggctgtatattactgtgcgggaaggatagcagctcgtcacggctggttcgacccctggggccagggaaccctggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 198 - protein, 446 aa, synthetic construct
QVQLQQWGAGLLKPSATLSRTCAVYGGSFSGYYWNWIRQSPGKGLEWIGEILHSGRTNYNPSLKSRVTISVDTSKNQFSLKLTSVTAADTAVYYCAGRIAARHGWFDPWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 199 - DNA, 645 bp, synthetic construct
  source 1..645: mol_type = other DNA
gacatccagatgacccagtctccatcttccgtgtctacatctgtaggagacagagtcaccatctcttgtcgggcgagtcaggatattcgcaagtggttagcctggtatcaacagaaaccaggaaaagcccctaaactcctgatctatgctacatccagtttgcaaagtggggtcccttcaaggttcagcggcagtggatctgggacagatttcactctcaccatcagcagcctgcagcctgaggattttgcaacttacttttgtcaacaggctaacagtttcccgttcacttttggccaggggaccaagctggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 200 - protein, 214 aa, synthetic construct
DIQMTQSPSSVSTSVGDRVTISCRASQDIRKWLAWYQQKPGKAPKLLIYATSSLQSGVPSRFSGSGSGTDFTLTISSLQPEDFATYFCQQANSFPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 201 - DNA, 1341 bp, synthetic construct
  source 1..1341: mol_type = other DNA
caggtgcagctggtggagtctgggggaggcgtggtccagcctgggaggtccctgagactctcctgtgcagcgtctggattcaccttcagtacatatggcatgcactgggtccgccaggctccaggcaaggggctggagtgggtggcagttatttggcatgatggaagtgataaatattatgtagactccgtgaagggccgattctccatcgccagagacaattccaagaacacgctttatctgcaaatgaatagtctgagagtcgaggacacgggtatatattactgtgcgagaaggggtatacgtggaaccgtttttgaccactggggcctgggaaccctggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 202 - protein, 446 aa, synthetic construct
QVQLVESGGGVVQPGRSLRLSCAASGFTFSTYGMHWVRQAPGKGLEWVAVIWHDGSDKYYVDSVKGRFSIARDNSKNTLYLQMNSLRVEDTGIYYCARRGIRGTVFDHWGLGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 203 - DNA, 645 bp, synthetic construct
  source 1..645: mol_type = other DNA
gacatccagatgacccagtctccttccaccctgtctgcatctgtaggagacagagtcaccctcacttgtcgggccagtcagagtattagtaacaagttggcctggtatcagcagaaaccagggaaagcccctaacctcctgatctataaggcgtctaatttagaaagtggggtcccatcaaggttcagcggcagtggatctgggacagaattcactctcaccatcagcagcctgcagcctgatgattttgcaacttattactgccaacagtataatagttattcgtggacgttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 204 - protein, 214 aa, synthetic construct
DIQMTQSPSTLSASVGDRVTLTCRASQSISNKLAWYQQKPGKAPNLLIYKASNLESGVPSRFSGSGSGTEFTLTISSLQPDDFATYYCQQYNSYSWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 205 - DNA, 1332 bp, synthetic construct
  source 1..1332: mol_type = other DNA
caggtgcagctgcaggagtcgggcccaggactggtgaagccttcggagaccctgtccctcacctgcactgtctctggtgactccatcaataattactactggacctggctccggcagcccccagggaagggactggagtggattggttatatctattacagtgggagcgccaactacaacccctccctcaagagtcgagtcaccatatcagtagacacgtccaagaaccagttctccctgaagctaaattctgtgaccgctgcggacacggccgtgtattactgtgcgagaggggcggtcaagtacttccggcattggggccagggcaccctggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 206 - protein, 443 aa, synthetic construct
QVQLQESGPGLVKPSETLSLTCTVSGDSINNYYWTWLRQPPGKGLEWIGYIYYSGSANYNPSLKSRVTISVDTSKNQFSLKLNSVTAADTAVYYCARGAVKYFRHWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 207 - DNA, 648 bp, synthetic construct
  source 1..648: mol_type = other DNA
gaaattgtgttgacgcagtctccgggcaccctctctttgtctccaggggaaagagccaccctctcctgcagggccagtcagactattaaccacaacaacttagcctggtaccagcagagacctggccaggctcccaggctcctcatctatggtgcatccaacagggccactgccatcccagacaggttcagtggcagtgggtctgggacagacttcactctcaccatcagcagactggagcctgaagattttgaagtgtattcttgtcagcagtatggtagcttgccgctcactttcggcggagggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 208 - protein, 215 aa, synthetic construct
EIVLTQSPGTLSLSPGERATLSCRASQTINHNNLAWYQQRPGQAPRLLIYGASNRATAIPDRFSGSGSGTDFTLTISRLEPEDFEVYSCQQYGSLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 209 - DNA, 1347 bp, synthetic construct
  source 1..1347: mol_type = other DNA
caggtgcagctggtggagtctgggggaggcgtggtccagcctgggacgtccctgagactctcctgtgcagcgtcaggattcaccttcagtagctatggcatgcactgggtccgccaggctccaggcaaggggctggagtgggtggcagttatatggattgatggaagtaataaatattatgcagactccgtgaagggccgattcaccatctccagagacaattccaagaacacgctgtatctgcaaatgaacagcctgagagccgaggacacggctgtgtattactgtgcgagaagggggggtatagtagtagctgccccctttgactactggggccagggaaccctggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 210 - protein, 448 aa, synthetic construct
QVQLVESGGGVVQPGTSLRLSCAASGFTFSSYGMHWVRQAPGKGLEWVAVIWIDGSNKYYADSVKGRFTISRDNSKNTLYLQMNSLRAEDTAVYYCARRGGIVVAAPFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 211 - DNA, 648 bp, synthetic construct; identical to SEQ ID NO: 195
  source 1..648: mol_type = other DNA

SEQ ID NO: 212 - protein, 215 aa, synthetic construct; identical to SEQ ID NO: 196

SEQ ID NO: 213 - DNA, 1356 bp, synthetic construct
  source 1..1356: mol_type = other DNA
caggtgcagttggtggagtctgggggaggcgtggtccagcctgggaggtccctgagactctcctgtgaagcgtctggaatcaccttcagaaactatggcatgcactgggtccgccaggctccaggcaaggggctggagtgggtggcagttatgtggtatgatggaagtaataaatactacgcagactccgtgaagggccgattcaccatctccagagacaattccaagaacacggtgtatctgcaaatgaacagcctgagagccgaagacacggctgtgtattactgtgcgagacggggtcatatagcaacagctgctccctttgactactggggccagggaaccctggtcaccgtctcctcagccaaaacaacagccccatcggtctatccactggcccctgtgtgtggagatacaactggctcctcggtgactctaggatgcctggtcaagggttatttccctgagccagtgaccttgacctggaactctggatccctgtccagtggtgtgcacaccttcccagctgtcctgcagtctgacctctacaccctcagcagctcagtgactgtaacctcgagcacctggcccagccagtccatcacctgcaatgtggcccacccggcaagcagcaccaaggtggacaagaaaattgagcccagagggcccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaatga

SEQ ID NO: 214 - protein, 451 aa, synthetic construct
QVQLVESGGGVVQPGRSLRLSCEASGITFRNYGMHWVRQAPGKGLEWVAVMWYDGSNKYYADSVKGRFTISRDNSKNTVYLQMNSLRAEDTAVYYCARRGHIATAAPFDYWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 215 - DNA, 648 bp, synthetic construct
  source 1..648: mol_type = other DNA
gacatccagatgacccagtctccatcctccctgtctgcatccgtaggagacagagtcaccatcagttgccgggcaagtcagagcattagtagttatttaaattggtatcagcagaaaccagggaaagcccctaaggtcctgatgtatgctgcatccagtttgcaaagtggggtcccatcaaggttcagtggcagtggatctgggacagatttcactctcaccatcagcagtctgcaacctgaggattttgcaacttactactgtcaacagagttacagtacccctccgatcaccttcggccaagggacacgactggagattaaacgagctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggggagagtgttga

SEQ ID NO: 216 - protein, 215 aa, synthetic construct
DIQMTQSPSSLSASVGDRVTISCRASQSISSYLNWYQQKPGKAPKVLMYAASSLQSGVPSRFSGSGSGTDFTLTISSLQPEDFATYYCQQSYSTPPITFGQGTRLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRGEC

SEQ ID NO: 217 - DNA, 1347 bp, synthetic construct
  source 1..1347: mol_type = other DNA
caggtgcagctggtggagtctgggggaggcgtggtccagcctgggaggtccctgagactctcctgtacagcgtctggattcaccttccgttcctatggcatgcactgggtccgccaggctccaggcaaggggctggagtgggtgtcagttatttggattgatggaaataatatatactatgcagactccgtgaagggccgattcaccatctccagagacaattccaagaacacgctgtatctgcaaatggacagcctgagagccgaggacacggctgtttattactgtgcgagaagactggctataacatcagctgccccctttgactactggggccagggaaccctggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 218 - protein, 448 aa, synthetic construct
QVQLVESGGGVVQPGRSLRLSCTASGFTFRSYGMHWVRQAPGKGLEWVSVIWIDGNNIYYADSVKGRFTISRDNSKNTLYLQMDSLRAEDTAVYYCARRLAITSAAPFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 219 - DNA, 648 bp, synthetic construct; identical to SEQ ID NO: 195
  source 1..648: mol_type = other DNA

SEQ ID NO: 220 - protein, 215 aa, synthetic construct; identical to SEQ ID NO: 196

SEQ ID NO: 221 - DNA, 1353 bp, synthetic construct
  source 1..1353: mol_type = other DNA
caggttcagctggtgcagtctggagctgaggtgaagaagcctggggcctcagtgaaggtctcctgcaaggcttctggttacgccttcaccacctatggtatcacctgggtgcgacaggcccctggacaaggacttgagtggatgggatggatcagcgcttacaatggaaatacaaactatgcagagaaggtccagggcagattcaccatgaccacagacacatccacgaatacagcctacatggagctgaggagcctgagatccgacgacacggccgtgtatttctgtgcgagaaagggtcactatggttcggggacttattataacccctttggttttgatttttggggccaagggacaatggtcaccgtctcttcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagtccaactgggaggcaggaaatactttcacctgctctgtgttacatgagggcctgcacaaccaccatactgagaagtccctctcccactctcctggtaaatga

SEQ ID NO: 222 - protein, 450 aa, synthetic construct
QVQLVQSGAEVKKPGASVKVSCKASGYAFTTYGITWVRQAPGQGLEWMGWISAYNGNTNYAEKVQGRFTMTTDTSTNTAYMELRSLRSDDTAVYFCARKGHYGSGTYYNPFGFDFWGQGTMVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 223 - DNA, 648 bp, synthetic construct
  source 1..648: mol_type = other DNA
gaaattatgttgatgcagtctccaggcaccctgtctttgtctccaggggaaagagccaccctctcctgcagggccagtcagagtgttagcagcagctacttagcctggtaccaacagaaacctggccaggctcccaggctcctcatctatggtgcatccagcagggccactgacatcccagacaggttcagtggcagtgggtctgggacagacttcactctcaccatcagcagactggagcctgaagattttgcagtttatttctgtcagcagtattatggctcaccttggacgttcggccaagggaccaaggtggaaatcaagcgagctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggggagagtgttga

SEQ ID NO: 224 - protein, 215 aa, synthetic construct
EIMLMQSPGTLSLSPGERATLSCRASQSVSSSYLAWYQQKPGQAPRLLIYGASSRATDIPDRFSGSGSGTDFTLTISRLEPEDFAVYFCQQYYGSPWTFGQGTKVEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRGEC

SEQ ID NO: 225 - DNA, 1344 bp, synthetic construct
  source 1..1344: mol_type = other DNA
gaggtgcagctggtggagtctgggggaggcttggtccagccgggggggtccctgaaactctcctgtacagcctctgggttgaccctcagtgactctgctatgcactgggtccgccaggcttccgggaaagggctggagtgggttggccgtataagaaataaggctaataggtacgcgacagaatatgctgcgtcggtgaaaggcaggttcaccatttcaagagatgattcaaagaacacggcgtatctacaaatgaacagcctgaaaaccgaggacacggccgtgtattattgtactagaaactggaagattttcctctttgactactggggccagggaaccctggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 226 - protein, 447 aa, synthetic construct
EVQLVESGGGLVQPGGSLKLSCTASGLTLSDSAMHWVRQASGKGLEWVGRIRNKANRYATEYAASVKGRFTISRDDSKNTAYLQMNSLKTEDTAVYYCTRNWKIFLFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 227 - DNA, 648 bp, synthetic construct
  source 1..648: mol_type = other DNA
gaaattgtgttgacgcagtctccaggcaccctgactttgtctccaggggaaagagccaccctctcctgcagggccagtcagagtgttggcagcaaatacttagcctggttccagcagaaacgtggccaggctcccaggctcctcatctatggtgcatccagcaggaccagtggcatccccgacaggatcagtggcagtgggtcagggacagacttcactctcaccatcagcagactggagcctgaagattttgcagtgtattactgtcagcagtatggaagttcaccctggacgttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 228 - protein, 215 aa, synthetic construct
EIVLTQSPGTLTLSPGERATLSCRASQSVGSKYLAWFQQKRGQAPRLLIYGASSRTSGIPDRISGSGSGTDFTLTISRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 229 - DNA, 1368 bp, synthetic construct
  source 1..1368: mol_type = other DNA
gaggtgcagctgttggagtctgggggaggcttggtacagccgggggggtccctgagactctcctgtgcagcctctggattcacctttaacaactatggcatgagctgggtccgccagggtccagggaaggggctggagtgggtctcatctattagtggtagtggtggtaccacattctacgcagactccgtgaagggccggttcaccatctccagagacaattccaagaacacgctgtatctgcaaatgaacagcctgagagccgaggacacggccgtatattactgtggcaaaggaggatattgtagtagtagcggctgccgtcactacggtatggacgtctggggccaagggaccacggtcaccgtctcctcagccaaaacaacagccccatcggtctatccactggcccctgtgtgtggagatacaactggctcctcggtgactctaggatgcctggtcaagggttatttccctgagccagtgaccttgacctggaactctggatccctgtccagtggtgtgcacaccttcccagctgtcctgcagtctgacctctacaccctcagcagctcagtgactgtaacctcgagcacctggcccagccagtccatcacctgcaatgtggcccacccggcaagcagcaccaaggtggacaagaaaattgagcccagagggcccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaatga

SEQ ID NO: 230 - protein, 455 aa, synthetic construct
EVQLLESGGGLVQPGGSLRLSCAASGFTFNNYGMSWVRQGPGKGLEWVSSISGSGGTTFYADSVKGRFTISRDNSKNTLYLQMNSLRAEDTAVYYCGKGGYCSSSGCRHYGMDVWGQGTTVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 231 - DNA, 654 bp, synthetic construct
  source 1..654: mol_type = other DNA
cagtctgtgctgactcagccaccctcagcgtctgggacccccgggcagagggtcaccatctcttgttctggaagcagctccaacatcggaaataattatatatactggtaccagcggctcccaggaacgacccccaaactcctcatctataggaataatcagcggccctcaggggtccctgaccgattctctggctccaagtctggcacctcagcctccctggccatcagtgggctccggtccgaggatgaggctgattattactgtgcagcatgggatgacaccctgagtgggtatgtcttcggaactgggaccaaggtcaccgtcctacgagctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggggagagtgttga

SEQ ID NO: 232 - protein, 217 aa, synthetic construct
QSVLTQPPSASGTPGQRVTISCSGSSSNIGNNYIYWYQRLPGTTPKLLIYRNNQRPSGVPDRFSGSKSGTSASLAISGLRSEDEADYYCAAWDDTLSGYVFGTGTKVTVLRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRGEC

SEQ ID NO: 233 - DNA, 1347 bp, synthetic construct
  source 1..1347: mol_type = other DNA
gaggtgcagctggtggagtctgggggaaacttggtacagcctggggggtccctgagactctcctgtgcagcctctggattcacctttaccagccatgccatgaactgggtccgccaggctccagggaaggggctggagtgggtctcagttattactggtagaggttttgacacacactacgctgactccgtgaagggccggttcaccatctccagagacatttccaaaaacacgctgtatctgcaaatgaacagcctgagagccgaggacacggccgtttattactgtgcgaaaggtctctatgattcggggaattattatatcgattactggggccagggaaccctggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 234 - protein, 448 aa, synthetic construct
EVQLVESGGNLVQPGGSLRLSCAASGFTFTSHAMNWVRQAPGKGLEWVSVITGRGFDTHYADSVKGRFTISRDISKNTLYLQMNSLRAEDTAVYYCAKGLYDSGNYYIDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 235 - DNA, 654 bp, synthetic construct
  source 1..654: mol_type = other DNA
cagtctgtgttgacgcagccgccctcagtgtctgcggccccaggacagaaggtcaccatctcctgctctggaagcagctccaacattgggaataattatgtttcctggtaccagcagctcccaggaacagcccccaaactcctcatttatgacaataataagcgaccctcagggattcctgaccgattctctggctccaagtctggcacgtcagccaccctgggcatcaccggactccagactggggacgaggccgattattactgcggaacatgggatctcagcctgagtttcaattgggtgttcggcggagggaccaagctgaccgtcctaggccagcccaaggccgccccctccgtgaccctgttccccccctcctccgaggagctgcaggccaacaaggccaccctggtgtgcctgatctccgacttctaccccggcgccgtgaccgtggcctggaaggccgactcctcccccgtgaaggccggcgtggagaccaccaccccctccaagcagtccaacaacaagtacgccgcctcctcctacctgtccctgacccccgagcagtggaagtcccaccggtcctactcctgccaggtgacccacgagggctccaccgtggagaagaccgtggcccccaccgagtgctcctga

SEQ ID NO: 236 - protein, 217 aa, synthetic construct
QSVLTQPPSVSAAPGQKVTISCSGSSSNIGNNYVSWYQQLPGTAPKLLIYDNNKRPSGIPDRFSGSKSGTSATLGITGLQTGDEADYYCGTWDLSLSFNWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 237 - DNA, 1362 bp, synthetic construct
  source 1..1362: mol_type = other DNA
caggttcagctggtgcagtctggagctgaggtgaagaagcctggggcctcagtgaaggtctcctgcaaggcttctggttacgccttcaccacctatggtatcacctgggtgcgacaggcccctggacaaggacttgagtggatgggatggatcagcgcttacaatggaaatacaaactatgcagagaaggtccagggcagattcaccatgaccacagacacatccacgaatacagcctacatggagctgaggagcctgagatccgacgacacggccgtgtatttctgtgcgagaaagggtcactatggttcggggacttattataacccctttggttttgatttttggggccaagggacaatggtcaccgtctcttcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 238 - protein, 453 aa, synthetic construct
QVQLVQSGAEVKKPGASVKVSCKASGYAFTTYGITWVRQAPGQGLEWMGWISAYNGNTNYAEKVQGRFTMTTDTSTNTAYMELRSLRSDDTAVYFCARKGHYGSGTYYNPFGFDFWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 239 - DNA, 648 bp, synthetic construct
  source 1..648: mol_type = other DNA
gaaattgtgttgacgcagtctccaggcaccctgtctttgtctccaggggaaagagccaccctctcctgcagggccagtcagagtgttagcagcagctacttagcctggtaccaacagaaacctggccaggctcccaggctcctcatctatggtgcatccagcagggccactggcatcccagacaggttcagtggcagtgggtctgggacagacttcactctcaccatcagcagactggagcctgaagattttgctttgtatttctgtcagcagtattatggctcaccttggacgttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 240 - protein, 215 aa, synthetic construct
EIVLTQSPGTLSLSPGERATLSCRASQSVSSSYLAWYQQKPGQAPRLLIYGASSRATGIPDRFSGSGSGTDFTLTISRLEPEDFALYFCQQYYGSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 241 - DNA, 666 bp, Homo sapiens
  source 1..666: mol_type = other DNA
atgtcacagaccaaaatgctgaaggtccgcgtgaccctcttctgcatcctggcaggcatcgtgctggccatgacagccgtggtaaccgaccactgggctgtgctgagcccccacatggagcaccacaacactacctgcgaggcggcccacttcggcctctggcggatttgtaccaagcgcatccccatggacgacagcaagacctgcgggcccatcaccctgcccggggagaagaactgttcctacttcaggcattttaaccccggcgagagctcggagatcttcgaattcaccactcagaaggagtacagcatctcggcagccgccatcgccatcttcagccttggcttcatcatcctgggcagcctctgtgtcctcctgtccctcgggaagaagagggactatctgctgcgacccgcgtccatgttctatgcctttgcaggtctctgcatcctcgtctcggtggaggtcatgcggcagtcggtgaagcgcatgattgacagtgaggacaccgtctggatcgagtactattactcctggtcctttgcctgcgcctgtgccgccttcatcctcctctttctcggcggtctcgccctcctgctgttctccctgcctcgaatgccccggaacccatgggagtcctgcatggatgctgagcccgagcac

SEQ ID NO: 242 - protein, 222 aa, Homo sapiens
MSQTKMLKVRVTLFCILAGIVLAMTAVVTDHWAVLSPHMEHHNTTCEAAHFGLWRICTKRIPMDDSKTCGPITLPGEKNCSYFRHFNPGESSEIFEFTTQKEYSISAAAIAIFSLGFIILGSLCVLLSLGKKRDYLLRPASMFYAFAGLCILVSVEVMRQSVKRMIDSEDTVWIEYYYSWSFACACAAFILLFLGGLALLLFSLPRMPRNPWESCMDAEPEH

SEQ ID NO: 243 - protein, 13 aa, synthetic construct
  REGION 1..13: note = SpyTag
AHIVMVDAYKPTK

SEQ ID NO: 244 - protein, 113 aa, synthetic construct
  REGION 1..113: note = SpyCatcher
VDTLSGLSSEQGQSGDMTIEEDSATHIKFSKRDEDGKELAGATMELRDSSGKTISTWISDGQVKDFYLYPGKYTFVETAAPDGYEVATAITFTVNEQGQVTVNGKATKGDAHI

SEQ ID NO: 245 - protein, 10 aa, synthetic construct
  REGION 1..10: note = KTag
ATHIKFSKRD

SEQ ID NO: 246 - protein, 10 aa, synthetic construct
  REGION 1..10: note = c-myc
EQKLISEEDL

SEQ ID NO: 247 - protein, 8 aa, synthetic construct
  REGION 1..8: note = B1 epitope
IGTRYLTR

SEQ ID NO: 248 - protein, 4 aa, synthetic construct
  REGION 1..4: note = GLSG linker
GLSG

SEQ ID NO: 249 - protein, 6 aa, synthetic construct
  REGION 1..6: note = GLSGSG linker
GLSGSG

SEQ ID NO: 250 - protein, 8 aa, synthetic construct
  REGION 1..8: note = GLSGLSGS Linker
GLSGLSGS

SEQ ID NO: 251 - protein, 10 aa, synthetic construct
  REGION 1..10: note = GLSGLSGLSG Linker
GLSGLSGLSG

SEQ ID NO: 252 - protein, 10 aa, synthetic construct
  REGION 1..10: note = GLSGGSGLSG
GLSGGSGLSG

SEQ ID NO: 253 - protein, 6 aa, synthetic construct
  REGION 1..6: note = GSGESG Linker
GSGESG

SEQ ID NO: 254 - protein, 16 aa, synthetic construct
  REGION 1..16: note = Isopeptide
TDKDMTITFTNKKDAE

SEQ ID NO: 255 - protein, 14 aa, synthetic construct
  REGION 1..14: note = SpyTag02
VPTIVMVDAYKRYK

SEQ ID NO: 256 - DNA, 19 bp, synthetic construct
  source 1..19: mol_type = other DNA
gtcatgcatgcctggaatc

SEQ ID NO: 257 - DNA, 50 bp, synthetic construct
  source 1..50: mol_type = other DNA
tcgtcggcagcgtcagatgtgtataagagacagcgagcgctgctcgagag

SEQ ID NO: 258 - DNA, 52 bp, synthetic construct
  source 1..52: mol_type = other DNA
gtctcgtgggctcggagatgtgtataagagacaggggtcacagggatgccac